(19) United States
(12) Patent Application Publication
(10) Pub. No.: US 2025/0281483 A1
(43) Pub. Date: Sep. 11, 2025

(54) TRANSCRIPTION FACTOR EB ACTIVATORS AND USES THEREOF

(71) Applicant: Astellas Engineered Small Molecules US, Incorporated, Cambridge, MA (US)

(72) Inventors: Bin Ma, Cambridge, MA (US); Takeru Furuya, Cambridge, MA (US)

(21) Appl. No.: 18/858,700

(22) PCT Filed: Apr. 21, 2023
(86) PCT No.: PCT/US2023/019398

§ 371 Date: Oct. 21, 2024

Related U.S. Application Data
(60) Provisional application No. 63/333,669, filed on Apr. 22, 2022.

(51) Int. Cl. A61K 31/501; A61K 31/4375; A61K 31/444; A61K 31/497; A61K 31/506; C07D 471/04

(52) U.S. Cl. CPC A61K 31/501; A61K 31/4375; A61K 31/444; A61K 31/497; A61K 31/506; C07D 471/04

(57) ABSTRACT

Provided are pharmaceutical compositions comprising a pharmaceutically acceptable carrier or diluent and a compound represented by Formula (I): or pharmaceutically acceptable salts thereof, which are useful for the activation of TFEB and in the treatment of a variety of diseases or conditions such as lysosomal storage diseases.

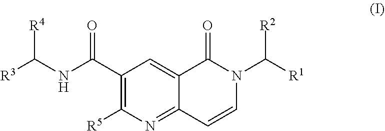

### RELATED APPLICATIONS

This application claims priority to U.S. Provisional Application No. 63/333,669, filed on Apr. 22, 2022. The entire contents of the foregoing application are expressly incorporated herein by reference.

### FIELD OF THE INVENTION

The present disclosure relates to activators of Transcription factor EB (TFEB), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering a TFEB activator to a warm-blooded animal, especially a human.

### BACKGROUND OF THE INVENTION

Autophagy is a process by which cells recycle intracellular material for energy, thus avoiding cell death due to stress, nutrient deprivation, or cell damage (Mizushima et al, 2008, Nature, 451 (7182), 1069-1075). In addition to nutrient provision, the autophagy pathway also acts as a “self-cleaning” pathway, breaking down misfolded or otherwise non-functioning proteins, cellular components, or any other materials either hazardous or no longer needed by the cell. During autophagy, the cellular components which have been tagged for degradation are encapsulated by a phagophore which forms an autophagosome, which next fuses with a lysosome, thus starting the degradation process. This catabolic process regenerates amino acid building blocks and other cellular materials which can be used in the synthesis of other proteins and also generates ATP which is used for energy in other cellular processes (Glick et al (2010) The Journal of Pathology, 221 (1), 3-12).

Transcription factor EB (TFEB) has been shown to be a master regulator of the autophagy process, activating genes involved in the expression of lysosomes as well as other related functions (Settembre et al, (2011), Science, 332 (6036), 1429-1433). When activated, TFEB translocates from the cycloplasm to the nucleus, where it activates the synthesis of the autophagy genes (Sardiello et al, (2009), Science, 325 (5939), 473-477). Because of its role in autophagy stimulation, activation of TFEB is a potential treatment option for a variety of diseases which have either a buildup of non-functioning or harmful proteins, such as Alzheimer's disease, or which have improper lysosomal activity, such as lysosomal storage diseases (Napolitano et al, 2016, J. Cell. Sci., 129 (13), 2475-2481).

Thus, there is a need for TFEB activators as potential therapeutic agents for treating diseases or disorders that are responsive to TFEB activation.

### SUMMARY OF THE INVENTION

The present disclosure provides compounds, or pharmaceutical compositions thereof, that are TFEB activators. Compared to known compounds, it is unexpected to find that the compounds disclosed herein have better potency and/or lower in vitro clearance. In one aspect, the present disclosure relates to a pharmaceutical composition comprising a pharmaceutically acceptable carrier or diluent and a compound represented by Formula (I):

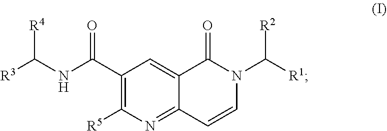

or a pharmaceutically acceptable salt thereof. The definition of each variable is described herein.

In another aspect, the present disclosure provides a method of treating a disease or disorder that is responsive to activation of TFEB in a subject comprising administering to said subject an effective amount of at least one compound described herein, or a pharmaceutically acceptable salt thereof, or a pharmaceutical composition comprising at least one compound described herein, or a pharmaceutically acceptable salt thereof, and a pharmaceutically acceptable carrier or diluent.

Another aspect of the present disclosure relates to the use of at least one compound described herein, or a pharmaceutically acceptable salt thereof, for the manufacture of a medicament for the treatment of a disease or disorder responsive to activation of TFEB. Also provided is a compound described herein or a pharmaceutically acceptable salt thereof, or a pharmaceutical composition comprising at least one compound described herein, or a pharmaceutically acceptable salt thereof, and a pharmaceutically acceptable carrier or diluent, for use in treating a disease or disorder responsive to activation of TFEB.

### DETAILED DESCRIPTION OF THE INVENTION

The present disclosure provides compounds and pharmaceutical compositions thereof that may be useful in the treatment of diseases or disorders through activation of TFEB function/activity. In some embodiments, the compounds of present disclosure are activators of TFEB.

### Compounds and Compositions

In a first embodiment, the present disclosure provides a pharmaceutical composition comprising a pharmaceutically acceptable carrier or diluent and a compound represented by Formula (I):

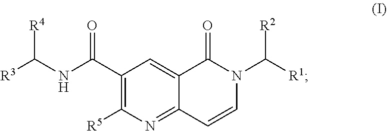

or a pharmaceutically acceptable salt thereof, wherein
R¹ is H, halo, C₁₋₆alkyl, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the C₁₋₆alkyl is optionally substituted with one to three R^{1a}, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{1c};
R^{1a}, for each occurrence, is independently halo, —OR^{1b}, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{1c};
R^{1b} is H, C₁₋₆alkyl, or —[CH₂—CH₂—O]_{z}—CH₃;
z is 1 to 3;
R^{1c}, for each occurrence, is independently halo, C₁₋₆alkyl, C₁₋₆haloalkyl, —OR^{1b}, —NR^{1d}R^{1d}, —CN, —NO₂, —C(O)R^{1b}, —C(O)OR^{1b}, —C(O)NR^{1d}R^{1d}, or —NR^{1d}C(O)R^{1b};
R^{1d} is H or C₁₋₆alkyl;
R² is H or C₁₋₆alkyl;
or R¹ and R², together with the carbon to which they are attached, form a non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, each of which is optionally substituted with one to three R^{1c};
R³ is H, halo, C₁₋₆alkyl, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the C₁₋₆alkyl is optionally substituted with one to three R^{3a}, wherein the non-armoatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{3c};
R^{3a}, for each occurrence, is independently halo, —OR^{3b}, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{3c};
R^{3b} is H, C₁₋₆alkyl, or —[CH₂—CH₂—O]_{y}—CH₃;
y is 1 to 3;
R^{3c}, for each occurrence, is independently halo, C₁₋₆alkyl, C₁₋₆haloalkyl, —OR^{3b}, —NR^{3d}R^{3d}, —CN, —NO₂, —C(O)R^{3b}, —C(O)OR^{3b}, —C(O)NR^{3d}R^{3d}, or —NR^{3d}C(O)R^{3b};
R^{3d} is H or C₁₋₆alkyl;
R⁴ is H or C₁₋₆alkyl;
R⁵ is H, halo, —NR^{5a}R^{5b}, C₁₋₆alkyl, or C₁₋₆haloalkyl;
R^{5a} and R^{5b} are each independently H or C₁₋₆alkyl;
provided that the pharmaceutical composition does not comprise a compound of the formulas below:

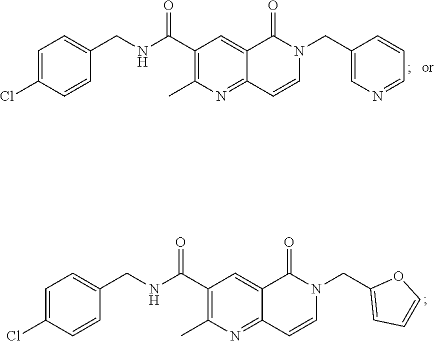 [SMILES: Cc1nc2ccn(Cc3cccnc3)c(=O)c2cc1C(=O)NCc1ccc(Cl)cc1 ; Cc1nc2ccn(Cc3ccco3)c(=O)c2cc1C(=O)NCc1ccc(Cl)cc1]

or a pharmaceutically acceptable salt thereof.

In a second embodiment, the present disclosure provides a pharmaceutical composition according to the first embodiment, wherein R⁵ is —CH₃; and the remaining variables are as described in the first embodiment.

In a third embodiment, the present disclosure provides a pharmaceutical composition according to the first or second embodiment, wherein R¹ is H, C₁₋₆alkyl, non-aromatic C₃₋₈carbocyclyl, 4 to 6-membered monocyclic heterocyclyl, phenyl, or 5 or 6-membered monocyclic heteroaryl, wherein the C₁₋₆alkyl is optionally substituted with one to three R^{1a}, wherein the non-aromatic C₃₋₈carbocyclyl, 4 to 6-membered monocyclic heterocyclyl, phenyl, or 5 or 6-membered monocyclic heteroaryl are each optionally substituted with one or two R^{1c}, or wherein R¹ and R², together with the carbon to which they are attached, form phenyl optionally substituted with one or two R^{1c}; and the remaining variables in the first or second embodiment are as described in the first or second embodiment, respectively.

In a fourth embodiment, the present disclosure provides a pharmaceutical composition according to the first or second embodiment, wherein R¹ is non-aromatic C₃₋₈carbocyclyl, 4 to 6-membered monocyclic heterocyclyl, phenyl, or 5 or 6-membered monocyclic heteroaryl, wherein the non-aromatic C₃₋₈carbocyclyl, 4 to 6-membered monocyclic heterocyclyl, phenyl, or 5 or 6-membered monocyclic heteroaryl are each optionally substituted with one or two R^{1c}; and the remaining variables are as described in the first or second embodiment.

In a fifth embodiment, the present disclosure provides a pharmaceutical composition according to the first or second embodiment, wherein R¹ is selected from the group consisting of bicyclo[1.1.1]pentanyl, phenyl, pyridyl, thiophenyl, tetrahydropyranyl, tetrahydrofuranyl, and oxetanyl, each of which is optionally substituted with one or two R^{1c}; and the remaining variables are as described in the first or second embodiment.

In a sixth embodiment, the present disclosure provides a pharmaceutical composition according to the first or second embodiment, wherein R¹ is selected from the group consisting of bicyclo[1.1.1]pentanyl, phenyl, thiophenyl, tetrahydropyranyl, tetrahydrofuranyl, and oxetanyl, each of which is optionally substituted with one or two R^{1c}; and the remaining variables are as described in the first or second embodiment.

In a seventh embodiment, the present disclosure provides a pharmaceutical composition according to the first, second, third, fourth, fifth, or sixth embodiment, wherein R^{1c}, for each occurrence, is C₁₋₆alkyl, —OR^{1b}, or halo, and wherein R^{1b} is —[CH₂—CH₂—O]_{z}—CH₃; and the remaining variables are as described in the first, second, third, fourth, fifth, or sixth embodiment.

In an eighth embodiment, the present disclosure provides a pharmaceutical composition according to the first, second, third, fourth, fifth, sixth, or seventh embodiment, wherein R³ is H, C₁₋₆alkyl, 6 to 10-membered bicyclic heterocyclyl, phenyl, or 5 or 6-membered monocyclic heteroaryl, wherein the C₁₋₆alkyl is optionally substituted with one to three R^{3a}, wherein the 6 to 10-membered bicyclic heterocyclyl, phenyl, or 5 or 6-membered monocyclic heteroaryl are each optionally substituted with one to three R^{3c}; and the remaining variables are as described in the first, second, third, fourth, fifth, sixth, or seventh embodiment.

In a ninth embodiment, the present disclosure provides a pharmaceutical composition according to the first, second, third, fourth, fifth, sixth, or seventh embodiment, wherein R³ is 6 to 10-membered bicyclic heterocyclyl, phenyl, or 5 or 6-membered monocyclic heteroaryl, wherein the 6 to 10-membered bicyclic heterocyclyl, phenyl, or 5 or 6-membered monocyclic heteroaryl are each optionally substituted with one or two R^{3c}; and the remaining variables are as described in the first, second, third, fourth, fifth, sixth, or seventh embodiment.

In a tenth embodiment, the present disclosure provides a pharmaceutical composition according to the first, second, third, fourth, fifth, sixth, or seventh embodiment, wherein R³ is 6 to 10-membered bicyclic heterocyclyl, or 5 or 6-membered monocyclic heteroaryl, wherein the 6 to 10-membered bicyclic heterocyclyl, or 5 or 6-membered monocyclic heteroaryl are each optionally substituted with one or two R^{3c}; and the remaining variables are as described in the first, second, third, fourth, fifth, sixth, or seventh embodiment.

In an eleventh embodiment, the present disclosure provides a pharmaceutical composition according to the first, second, third, fourth, fifth, sixth, seventh, eighth, or ninth embodiment, wherein R³ is phenyl, pyridyl, pyrimidinyl, pyridazinyl, pyrazinyl, thiophenyl, benzodioxoyl, furanyl, oxazoyl, oxadiazoyl, pyrazoyl, or triazoyl, wherein the phenyl, pyridyl, pyrimidinyl, pyridazinyl, pyrazinyl, thiophenyl, benzodioxoyl, furanyl, oxazoyl, oxadiazoyl, pyrazoyl, or triazoyl, are each optionally substituted with one to two R^{3c}; and the remaining variables are as described in the first, second, third, fourth, fifth, sixth, seventh, eighth, or ninth embodiment.

In a twelfth embodiment, the present disclosure provides a pharmaceutical composition according to the first embodiment, wherein the compound is represented by Formula (II):

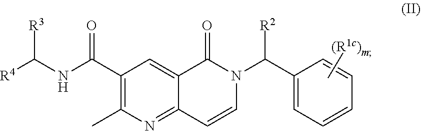

or a pharmaceutically acceptable salt thereof, wherein:
m is 0 to 2;
R^{1c} is independently halo, C₁₋₆alkyl, or —OR^{1b};
R^{1b} is —[CH₂—CH₂—O]_{z}—CH₃;
R³ is 5 or 6-membered monocyclic heteroaryl optionally substituted with one or two R^{3c};
R^{3c} is halo or C₁₋₆alkyl; and the remaining variables are as described in the first embodiment.

In a thirteenth embodiment, the present disclosure provides a pharmaceutical composition according to the first, second, third, fourth, fifth, sixth, seventh, eighth, ninth, tenth, eleventh, or twelfth embodiment, wherein R³ is pyridyl; and the remaining variables are as described in the first, second, third, fourth, fifth, sixth, seventh, eighth, ninth, tenth, eleventh, or twelfth embodiment.

In a fourteenth embodiment, the present disclosure provides a pharmaceutical composition according to the first, second, third, fourth, fifth, sixth, seventh, eighth, ninth, tenth, or eleventh embodiment, wherein R^{3c}, for each occurrence, is halo, C₁₋₆alkyl, C₁₋₆ haloalkyl, —OR^{3b}, or —C(O)NR^{3b}R^{3d}; and the remaining variables are as described in the first, second, third, fourth, fifth, sixth, seventh, eighth, ninth, tenth, or eleventh embodiment.

In a fifteenth embodiment, the present disclosure provides a pharmaceutical composition according to the first, second, third, fourth, fifth, sixth, seventh, eighth, ninth, tenth, eleventh, twelfth, thirteenth, or fourteenth embodiment, wherein R^{3c}, for each occurrence, is —CH₃, —CF₃, —Cl, —F, —OCH₃, —OCH(CH₃)₂, or —C(O)NH₂; and the remaining variables are as described in the first, second, third, fourth, fifth, sixth, seventh, eighth, ninth, tenth, eleventh, thirteenth, or fourteenth embodiment.

In a sixteenth embodiment, the present disclosure provides a pharmaceutical composition according to the first, second, third, fourth, fifth, sixth, seventh, eighth, ninth, tenth, eleventh, twelfth, thirteenth, fourteenth, or fifteenth embodiment, wherein R² and R⁴ are both H; and the remaining variables are as described in the first, second, third, fourth, fifth, sixth, seventh, eighth, ninth, tenth, eleventh, twelfth, thirteenth, fourteenth, or fifteenth embodiment.

In a seventeenth embodiment, the present disclosure provides a pharmaceutical composition according to the first, second, third, embodiment, wherein R^{1a} is —OR^{1b}, and R^{3a}, for each occurrence, is independently halo, —OR^{3b}, or 5 or 6-membered monocyclic heteroaryl; and the remaining variables are as described in the first, second, or third, embodiment.

In an eighteenth embodiment, the present disclosure provides a pharmaceutical composition according to the first, second, third, fourth, fifth, sixth, seventh, eighth, ninth, tenth, eleventh, twelfth, thirteenth, fourteenth, fifteenth, or sixteenth embodiment, wherein R^{1c} is —F, —Cl, or —CH₃ and R^{3c} is —F, Cl, or —CH₃; and the remaining variables are as described in the first, second, third, fourth, fifth, sixth, seventh, eighth, ninth, tenth, eleventh, twelfth, thirteenth, fourteenth, fifteenth, or sixteenth embodiment.

In a nineteenth embodiment, the present disclosure provides a pharmaceutical composition comprising a compound described herein (e.g., a compound of any one of Examples 1 to 81), or a pharmaceutically acceptable salt thereof, and a pharmaceutically acceptable carrier or diluent.

In another embodiment, the present disclosure provides a pharmaceutical composition comprising a compound selected from the group consisting of.
6-benzyl-2-methyl-N-((5-methylfuran-2-yl)methyl)-5-oxo-5,6-dihydro-1,6-naphthyridine-3-carboxamide;
6-((5-fluoropyridin-2-yl)methyl)-2-methyl-N-((5-methylfuran-2-yl)methyl)-5-oxo-5,6-dihydro-1,6-naphthyridine-3-carboxamide;
6-benzyl-2-methyl-N-((5-methyloxazol-2-yl)methyl)-5-oxo-5,6-dihydro-1,6-naphthyridine-3-carboxamide;
6-benzyl-2-methyl-N-((2-methyloxazol-5-yl)methyl)-5-oxo-5,6-dihydro-1,6-naphthyridine-3-carboxamide;
6-benzyl-2-methyl-N-(3-methylbenzyl)-5-oxo-5,6-dihydro-1,6-naphthyridine-3-carboxamide;
6-benzyl-2-methyl-N-((6-methylpyridin-2-yl)methyl)-5-oxo-5,6-dihydro-1,6-naphthyridine-3-carboxamide;
6-benzyl-2-methyl-5-oxo-N-(pyridin-2-ylmethyl)-5,6-dihydro-1,6-naphthyridine-3-carboxamide;
6-benzyl-2-methyl-N-((1-methyl-1H-1,2,3-triazol-4-yl)methyl)-5-oxo-5,6-dihydro-1,6-naphthyridine-3-carboxamide;
6-benzyl-2-methyl-N-((2-methyl-2H-1,2,3-triazol-4-yl)methyl)-5-oxo-5,6-dihydro-1,6-naphthyridine-3-carboxamide;
6-benzyl-2-methyl-N-((3-methyl-1,2,4-oxadiazol-5-yl)methyl)-5-oxo-5,6-dihydro-1,6-naphthyridine-3-carboxamide;
N-(4-chlorobenzyl)-2-methyl-5-oxo-6-((tetrahydrofuran-2-yl)methyl)-5,6-dihydro-1,6-naphthyridine-3-carboxamide;
6-benzyl-2-methyl-5-oxo-N-(pyridin-4-ylmethyl)-5,6-dihydro-1,6-naphthyridine-3-carboxamide;
6-benzyl-2-methyl-N-((5-methyl-1,3,4-oxadiazol-2-yl)methyl)-5-oxo-5,6-dihydro-1,6-naphthyridine-3-carboxamide;
6-benzyl-2-methyl-N-((1-methyl-1H-pyrazol-3-yl)methyl)-5-oxo-5,6-dihydro-1,6-naphthyridine-3-carboxamide;
6-benzyl-2-methyl-N-((1-methyl-1H-pyrazol-4-yl)methyl)-5-oxo-5,6-dihydro-1,6-naphthyridine-3-carboxamide;
6-benzyl-N-(2-fluorobenzyl)-2-methyl-5-oxo-5,6-dihydro-1,6-naphthyridine-3-carboxamide;
N,6-dibenzyl-2-methyl-5-oxo-5,6-dihydro-1,6-naphthyridine-3-carboxamide;
6-benzyl-2-methyl-N-((2-methyloxazol-4-yl)methyl)-5-oxo-5,6-dihydro-1,6-naphthyridine-3-carboxamide;
6-benzyl-2-methyl-N-((5-methyl-1,2,4-oxadiazol-3-yl)methyl)-5-oxo-5,6-dihydro-1,6-naphthyridine-3-carboxamide;
N-(4-chlorobenzyl)-6-(2-methoxyethyl)-2-methyl-5-oxo-5,6-dihydro-1,6-naphthyridine-3-carboxamide;
6-(2-methoxyethyl)-2-methyl-N-((2-methyloxazol-5-yl)methyl)-5-oxo-5,6-dihydro-1,6-naphthyridine-3-carboxamide;
6-benzyl-N-(4-chlorobenzyl)-2-methyl-5-oxo-5,6-dihydro-1,6-naphthyridine-3-carboxamide;
6-benzyl-2-methyl-5-oxo-N-(pyridin-3-ylmethyl)-5,6-dihydro-1,6-naphthyridine-3-carboxamide;
6-benzyl-N-(4-methoxybenzyl)-2-methyl-5-oxo-5,6-dihydro-1,6-naphthyridine-3-carboxamide;
6-benzyl-2-methyl-5-oxo-N-(4-(trifluoromethyl)benzyl)-5,6-dihydro-1,6-naphthyridine-3-carboxamide;
N-(4-chlorobenzyl)-2-methyl-5-oxo-6-((tetrahydro-2H-pyran-2-yl)methyl)-5,6-dihydro-1,6-naphthyridine-3-carboxamide;
N-(4-chlorobenzyl)-2-methyl-6-(oxetan-2-ylmethyl)-5-oxo-5,6-dihydro-1,6-naphthyridine-3-carboxamide;
6-benzyl-2-methyl-5-oxo-N-(1-(pyridin-2-yl)ethyl)-5,6-dihydro-1,6-naphthyridine-3-carboxamide;
6-benzyl-2-methyl-5-oxo-N-(pyrimidin-2-ylmethyl)-5,6-dihydro-1,6-naphthyridine-3-carboxamide;
6-(2-methoxyethyl)-2-methyl-5-oxo-N-(pyridin-2-ylmethyl)-5,6-dihydro-1,6-naphthyridine-3-carboxamide;
6-(4-chlorobenzyl)-2-methyl-5-oxo-N-(pyridin-2-ylmethyl)-5,6-dihydro-1,6-naphthyridine-3-carboxamide;
6-benzyl-2-methyl-5-oxo-N-(pyrazin-2-ylmethyl)-5,6-dihydro-1,6-naphthyridine-3-carboxamide;
6-benzyl-2-methyl-5-oxo-N-(pyridazin-3-ylmethyl)-5,6-dihydro-1,6-naphthyridine-3-carboxamide;
6-benzyl-2-methyl-5-oxo-N-(pyrimidin-5-ylmethyl)-5,6-dihydro-1,6-naphthyridine-3-carboxamide;
6-benzyl-N-((5-fluoropyridin-2-yl)methyl)-2-methyl-5-oxo-5,6-dihydro-1,6-naphthyridine-3-carboxamide;
6-benzyl-N-((3-fluoropyridin-2-yl)methyl)-2-methyl-5-oxo-5,6-dihydro-1,6-naphthyridine-3-carboxamide;
2-methyl-5-oxo-6-(1-phenylethyl)-N-(pyridin-2-ylmethyl)-5,6-dihydro-1,6-naphthyridine-3-carboxamide;
6-benzyl-N-((5-chloropyridin-2-yl)methyl)-2-methyl-5-oxo-5,6-dihydro-1,6-naphthyridine-3-carboxamide;
6-benzyl-N-((4-fluoropyridin-2-yl)methyl)-2-methyl-5-oxo-5,6-dihydro-1,6-naphthyridine-3-carboxamide;
6-(cyclohexylmethyl)-2-methyl-5-oxo-N-(pyridin-2-ylmethyl)-5,6-dihydro-1,6-naphthyridine-3-carboxamide;
2-methyl-5-oxo-N,6-bis(pyridin-2-ylmethyl)-5,6-dihydro-1,6-naphthyridine-3-carboxamide;
6-benzyl-N,2-dimethyl-5-oxo-5,6-dihydro-1,6-naphthyridine-3-carboxamide;
6-benzyl-N-(4-carbamoylbenzyl)-2-methyl-5-oxo-5,6-dihydro-1,6-naphthyridine-3-carboxamide;
6-((5-fluoropyridin-2-yl)methyl)-2-methyl-5-oxo-N-(pyridin-2-ylmethyl)-5,6-dihydro-1,6-naphthyridine-3-carboxamide;
6-isopropyl-2-methyl-5-oxo-N-(pyridin-2-ylmethyl)-5,6-dihydro-1,6-naphthyridine-3-carboxamide;
2-methyl-5-oxo-6-phenyl-N-(pyridin-2-ylmethyl)-5,6-dihydro-1,6-naphthyridine-3-carboxamide;
6-(4-fluorobenzyl)-2-methyl-5-oxo-N-(pyridin-2-ylmethyl)-5,6-dihydro-1,6-naphthyridine-3-carboxamide;
2-methyl-6-((5-methyloxazol-2-yl)methyl)-5-oxo-N-(pyridin-2-ylmethyl)-5,6-dihydro-1,6-naphthyridine-3-carboxamide;
2-methyl-6-((1-methyl-1H-pyrazol-4-yl)methyl)-5-oxo-N-(pyridin-2-ylmethyl)-5,6-dihydro-1,6-naphthyridine-3-carboxamide;
6-benzyl-N-((4,5-difluoropyridin-2-yl)methyl)-2-methyl-5-oxo-5,6-dihydro-1,6-naphthyridine-3-carboxamide;
6-(4-chlorobenzyl)-N-((5-fluoropyridin-2-yl)methyl)-2-methyl-5-oxo-5,6-dihydro-1,6-naphthyridine-3-carboxamide;
2-methyl-5-oxo-N-(pyridin-2-ylmethyl)-6-(pyridin-4-ylmethyl)-5,6-dihydro-1,6-naphthyridine-3-carboxamide;
2,6-dimethyl-5-oxo-N-(pyridin-2-ylmethyl)-5,6-dihydro-1,6-naphthyridine-3-carboxamide;
2-methyl-6-(4-methylbenzyl)-5-oxo-N-(pyridin-2-ylmethyl)-5,6-dihydro-1,6-naphthyridine-3-carboxamide;
6-(3-fluorobenzyl)-2-methyl-5-oxo-N-(pyridin-2-ylmethyl)-5,6-dihydro-1,6-naphthyridine-3-carboxamide;
6-(2-fluorobenzyl)-2-methyl-5-oxo-N-(pyridin-2-ylmethyl)-5,6-dihydro-1,6-naphthyridine-3-carboxamide;
6-benzyl-N-((5-chloro-4-fluoropyridin-2-yl)methyl)-2-methyl-5-oxo-5,6-dihydro-1,6-naphthyridine-3-carboxamide;
2-methyl-5-oxo-N-(pyridin-2-ylmethyl)-6-(pyridin-3-ylmethyl)-5,6-dihydro-1,6-naphthyridine-3-carboxamide;
N-((5-chloropyridin-2-yl)methyl)-6-(2-(2-(2-methoxyethoxy)ethoxy)ethyl)-2-methyl-5-oxo-5,6-dihydro-1,6-naphthyridine-3-carboxamide;
6-(bicyclo[1.1.1]pentan-1-ylmethyl)-2-methyl-5-oxo-N-(pyridin-2-ylmethyl)-5,6-dihydro-1,6-naphthyridine-3-carboxamide;
6-(3,4-difluorobenzyl)-2-methyl-5-oxo-N-(pyridin-2-ylmethyl)-5,6-dihydro-1,6-naphthyridine-3-carboxamide;
6-(3,4-difluorobenzyl)-N-((4,5-difluoropyridin-2-yl)methyl)-2-methyl-5-oxo-5,6-dihydro-1,6-naphthyridine-3-carboxamide;
6-(3,4-difluorobenzyl)-N-((5-fluoropyridin-2-yl)methyl)-2-methyl-5-oxo-5,6-dihydro-1,6-naphthyridine-3-carboxamide;
N-((5-chloropyridin-2-yl)methyl)-6-(4-(2-(2-methoxyethoxy)ethoxy)benzyl)-2-methyl-5-oxo-5,6-dihydro-1,6-naphthyridine-3-carboxamide;
6-benzyl-5-oxo-N-(pyridin-2-ylmethyl)-5,6-dihydro-1,6-naphthyridine-3-carboxamide;
6-benzyl-2-methyl-N-((4-methyloxazol-2-yl)methyl)-5-oxo-5,6-dihydro-1,6-naphthyridine-3-carboxamide;
6-benzyl-N-(4-isopropoxybenzyl)-2-methyl-5-oxo-5,6-dihydro-1,6-naphthyridine-3-carboxamide;
6-benzyl-2-methyl-N-(4-methylbenzyl)-5-oxo-5,6-dihydro-1,6-naphthyridine-3-carboxamide;
N-(benzo[d][1,3]dioxol-5-ylmethyl)-6-benzyl-2-methyl-5-oxo-5,6-dihydro-1,6-naphthyridine-3-carboxamide;
2-methyl-N-((5-methylfuran-2-yl)methyl)-5-oxo-6-(thiophen-2-ylmethyl)-5,6-dihydro-1,6-naphthyridine-3-carboxamide;
6-benzyl-2-methyl-5-oxo-N-(thiophen-2-ylmethyl)-5,6-dihydro-1,6-naphthyridine-3-carboxamide;
6-(2-methoxyethyl)-2-methyl-N-((5-methylfuran-2-yl)methyl)-5-oxo-5,6-dihydro-1,6-naphthyridine-3-carboxamide;
6-benzyl-N-(2-(furan-2-yl)ethyl)-2-methyl-5-oxo-5,6-dihydro-1,6-naphthyridine-3-carboxamide;
2-amino-6-benzyl-5-oxo-N-(pyridin-2-ylmethyl)-5,6-dihydro-1,6-naphthyridine-3-carboxamide;
6-(4-chloro-3-fluorobenzyl)-2-methyl-5-oxo-N-(pyridin-2-ylmethyl)-5,6-dihydro-1,6-naphthyridine-3-carboxamide;
6-(4-chlorobenzyl)-N-((5-(2-(2-methoxyethoxy)ethoxy)pyridin-2-yl)methyl)-2-methyl-5-oxo-5,6-dihydro-1,6-naphthyridine-3-carboxamide;
6-(3,4-difluorobenzyl)-2-methyl-N-((2-methyloxazol-4-yl)methyl)-5-oxo-5,6-dihydro-1,6-naphthyridine-3-carboxamide;
6-(4-fluorobenzyl)-2-methyl-N-((2-methyloxazol-4-yl)methyl)-5-oxo-5,6-dihydro-1,6-naphthyridine-3-carboxamide;
6-(3-fluorobenzyl)-2-methyl-N-((2-methyloxazol-4-yl)methyl)-5-oxo-5,6-dihydro-1,6-naphthyridine-3-carboxamide;
6-(4-chlorobenzyl)-2-methyl-N-((2-methyloxazol-4-yl)methyl)-5-oxo-5,6-dihydro-1,6-naphthyridine-3-carboxamide; and
6-(3,5-difluorobenzyl)-2-methyl-N-((2-methyloxazol-4-yl)methyl)-5-oxo-5,6-dihydro-1,6-naphthyridine-3-carboxamide;
or a pharmaceutically acceptable salt thereof.

In a twentieth embodiment, the present disclosure provides a compound represented by Formula (II):

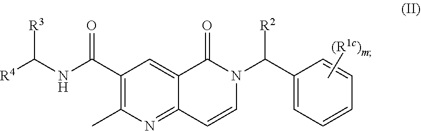

or a pharmaceutically acceptable salt thereof, wherein:
R² is H or C₁₋₆alkyl;
R⁴ is H or C₁₋₆alkyl; R^{1c} is independently halo or C₁₋₆alkyl;
m is 0 to 2;
when m is 0, R³ is a 5-membered monocyclic heteroaryl selected from the group consisting of oxazoyl, oxadiazoyl, pyrazoyl, and triazoyl or a 6-membered monocyclic heteroaryl, each of which is optionally substituted with one or two R^{3c}; or
when m is 1 or 2, R³ is 5 or 6-membered monocyclic heteroaryl optionally substituted with one or two R^{3c};
R^{3c} is halo or C₁₋₆alkyl.

In a twenty-first embodiment, the present disclosure provides a compound according to the twentieth embodiment, or a pharmaceutically acceptable salt thereof, wherein R² and R⁴ are both H; and the remaining variables are as described in the twentieth embodiment.

In a twenty-second embodiment, the present disclosure provides a compound according to the twentieth or twenty-first embodiment, or a pharmaceutically acceptable salt thereof, wherein R³ is pyridyl, pyrimidinyl, pyridazinyl, pyrazinyl, oxazoyl, oxadiazoyl, pyrazoyl, or triazoyl, each of which is optionally substituted with one to two R^{3c}; and the remaining variables are as described in the twentieth or twenty-first embodiment.

In a twenty-third embodiment, the present disclosure provides a compound according to the twentieth, twenty-first, or twenty-second embodiment, or a pharmaceutically acceptable salt thereof, wherein R³ is pyridyl; and the remaining variables are as described in the twentieth, twenty-first, or twenty-second embodiment.

In a twenty-fourth embodiment, the present disclosure provides a compound according to the twentieth, twenty-first, twenty-second, or twenty-third embodiment, or a pharmaceutically acceptable salt thereof, wherein each R^{1c} is independently H, —Cl, —F, or —CH₃, and wherein each R^{3c} is independently —F, —Cl, or —CH₃; and the remaining variables are as described in the twentieth, twenty-first, twenty-second, or twenty-third embodiment.

In a twenty-fifth embodiment, the present disclosure provides a compound according to the twentieth, twenty-first, twenty-second, twenty-third, or twenty-fourth embodiment, or a pharmaceutically acceptable salt thereof, wherein R^{1c} is H, —Cl, or —CH₃, and wherein R^{3c} is —F; and the remaining variables are as described in the twentieth, twenty-first, twenty-second, twenty-third, or twenty-fourth embodiment.

In a twenty-sixth embodiment, the present disclosure provides a compound selected from the group consisting of:
6-((5-fluoropyridin-2-yl)methyl)-2-methyl-N-((5-methylfuran-2-yl)methyl)-5-oxo-5,6-dihydro-1,6-naphthyridine-3-carboxamide;
6-benzyl-2-methyl-N-((5-methyloxazol-2-yl)methyl)-5-oxo-5,6-dihydro-1,6-naphthyridine-3-carboxamide;
6-benzyl-2-methyl-N-((2-methyloxazol-5-yl)methyl)-5-oxo-5,6-dihydro-1,6-naphthyridine-3-carboxamide;
6-benzyl-2-methyl-N-(3-methylbenzyl)-5-oxo-5,6-dihydro-1,6-naphthyridine-3-carboxamide;
6-benzyl-2-methyl-N-((6-methylpyridin-2-yl)methyl)-5-oxo-5,6-dihydro-1,6-naphthyridine-3-carboxamide;
6-benzyl-2-methyl-5-oxo-N-(pyridin-2-ylmethyl)-5,6-dihydro-1,6-naphthyridine-3-carboxamide;
6-benzyl-2-methyl-N-((1-methyl-1H-1,2,3-triazol-4-yl)methyl)-5-oxo-5,6-dihydro-1,6-naphthyridine-3-carboxamide;
6-benzyl-2-methyl-N-((2-methyl-2H-1,2,3-triazol-4-yl)methyl)-5-oxo-5,6-dihydro-1,6-naphthyridine-3-carboxamide;
6-benzyl-2-methyl-N-((3-methyl-1,2,4-oxadiazol-5-yl)methyl)-5-oxo-5,6-dihydro-1,6-naphthyridine-3-carboxamide;
N-(4-chlorobenzyl)-2-methyl-5-oxo-6-((tetrahydrofuran-2-yl)methyl)-5,6-dihydro-1,6-naphthyridine-3-carboxamide;
6-benzyl-2-methyl-5-oxo-N-(pyridin-4-ylmethyl)-5,6-dihydro-1,6-naphthyridine-3-carboxamide;
6-benzyl-2-methyl-N-((5-methyl-1,3,4-oxadiazol-2-yl)methyl)-5-oxo-5,6-dihydro-1,6-naphthyridine-3-carboxamide;
6-benzyl-2-methyl-N-((1-methyl-1H-pyrazol-3-yl)methyl)-5-oxo-5,6-dihydro-1,6-naphthyridine-3-carboxamide;
6-benzyl-2-methyl-N-((1-methyl-1H-pyrazol-4-yl)methyl)-5-oxo-5,6-dihydro-1,6-naphthyridine-3-carboxamide;
6-benzyl-2-methyl-N-((2-methyloxazol-4-yl)methyl)-5-oxo-5,6-dihydro-1,6-naphthyridine-3-carboxamide;
6-benzyl-2-methyl-N-((5-methyl-1,2,4-oxadiazol-3-yl)methyl)-5-oxo-5,6-dihydro-1,6-naphthyridine-3-carboxamide;
6-(2-methoxyethyl)-2-methyl-N-((2-methyloxazol-5-yl)methyl)-5-oxo-5,6-dihydro-1,6-naphthyridine-3-carboxamide;
6-benzyl-2-methyl-5-oxo-N-(pyridin-3-ylmethyl)-5,6-dihydro-1,6-naphthyridine-3-carboxamide;
6-benzyl-N-(4-methoxybenzyl)-2-methyl-5-oxo-5,6-dihydro-1,6-naphthyridine-3-carboxamide;
6-benzyl-2-methyl-5-oxo-N-(4-(trifluoromethyl)benzyl)-5,6-dihydro-1,6-naphthyridine-3-carboxamide;
N-(4-chlorobenzyl)-2-methyl-5-oxo-6-((tetrahydro-2H-pyran-2-yl)methyl)-5,6-dihydro-1,6-naphthyridine-3-carboxamide;
N-(4-chlorobenzyl)-2-methyl-6-(oxetan-2-ylmethyl)-5-oxo-5,6-dihydro-1,6-naphthyridine-3-carboxamide;
6-benzyl-2-methyl-5-oxo-N-(1-(pyridin-2-yl)ethyl)-5,6-dihydro-1,6-naphthyridine-3-carboxamide;
6-benzyl-2-methyl-5-oxo-N-(pyrimidin-2-ylmethyl)-5,6-dihydro-1,6-naphthyridine-3-carboxamide;
6-(2-methoxyethyl)-2-methyl-5-oxo-N-(pyridin-2-ylmethyl)-5,6-dihydro-1,6-naphthyridine-3-carboxamide;
6-(4-chlorobenzyl)-2-methyl-5-oxo-N-(pyridin-2-ylmethyl)-5,6-dihydro-1,6-naphthyridine-3-carboxamide;
6-benzyl-2-methyl-5-oxo-N-(pyrazin-2-ylmethyl)-5,6-dihydro-1,6-naphthyridine-3-carboxamide;
6-benzyl-2-methyl-5-oxo-N-(pyridazin-3-ylmethyl)-5,6-dihydro-1,6-naphthyridine-3-carboxamide;
6-benzyl-2-methyl-5-oxo-N-(pyrimidin-5-ylmethyl)-5,6-dihydro-1,6-naphthyridine-3-carboxamide;
6-benzyl-N-((5-fluoropyridin-2-yl)methyl)-2-methyl-5-oxo-5,6-dihydro-1,6-naphthyridine-3-carboxamide;
6-benzyl-N-((3-fluoropyridin-2-yl)methyl)-2-methyl-5-oxo-5,6-dihydro-1,6-naphthyridine-3-carboxamide;
2-methyl-5-oxo-6-(1-phenylethyl)-N-(pyridin-2-ylmethyl)-5,6-dihydro-1,6-naphthyridine-3-carboxamide;
6-benzyl-N-((5-chloropyridin-2-yl)methyl)-2-methyl-5-oxo-5,6-dihydro-1,6-naphthyridine-3-carboxamide;
6-benzyl-N-((4-fluoropyridin-2-yl)methyl)-2-methyl-5-oxo-5,6-dihydro-1,6-naphthyridine-3-carboxamide;
6-(cyclohexylmethyl)-2-methyl-5-oxo-N-(pyridin-2-ylmethyl)-5,6-dihydro-1,6-naphthyridine-3-carboxamide;
2-methyl-5-oxo-N,6-bis(pyridin-2-ylmethyl)-5,6-dihydro-1,6-naphthyridine-3-carboxamide;
6-benzyl-N,2-dimethyl-5-oxo-5,6-dihydro-1,6-naphthyridine-3-carboxamide;
6-benzyl-N-(4-carbamoylbenzyl)-2-methyl-5-oxo-5,6-dihydro-1,6-naphthyridine-3-carboxamide;
6-((5-fluoropyridin-2-yl)methyl)-2-methyl-5-oxo-N-(pyridin-2-ylmethyl)-5,6-dihydro-1,6-naphthyridine-3-carboxamide;
6-isopropyl-2-methyl-5-oxo-N-(pyridin-2-ylmethyl)-5,6-dihydro-1,6-naphthyridine-3-carboxamide;
2-methyl-5-oxo-6-phenyl-N-(pyridin-2-ylmethyl)-5,6-dihydro-1,6-naphthyridine-3-carboxamide;
6-(4-fluorobenzyl)-2-methyl-5-oxo-N-(pyridin-2-ylmethyl)-5,6-dihydro-1,6-naphthyridine-3-carboxamide;
2-methyl-6-((5-methyloxazol-2-yl)methyl)-5-oxo-N-(pyridin-2-ylmethyl)-5,6-dihydro-1,6-naphthyridine-3-carboxamide;
2-methyl-6-((1-methyl-1H-pyrazol-4-yl)methyl)-5-oxo-N-(pyridin-2-ylmethyl)-5,6-dihydro-1,6-naphthyridine-3-carboxamide;
6-benzyl-N-((4,5-difluoropyridin-2-yl)methyl)-2-methyl-5-oxo-5,6-dihydro-1,6-naphthyridine-3-carboxamide;
6-(4-chlorobenzyl)-N-((5-fluoropyridin-2-yl)methyl)-2-methyl-5-oxo-5,6-dihydro-1,6-naphthyridine-3-carboxamide;
2-methyl-5-oxo-N-(pyridin-2-ylmethyl)-6-(pyridin-4-ylmethyl)-5,6-dihydro-1,6-naphthyridine-3-carboxamide;
2,6-dimethyl-5-oxo-N-(pyridin-2-ylmethyl)-5,6-dihydro-1,6-naphthyridine-3-carboxamide;
2-methyl-6-(4-methylbenzyl)-5-oxo-N-(pyridin-2-ylmethyl)-5,6-dihydro-1,6-naphthyridine-3-carboxamide;
6-(3-fluorobenzyl)-2-methyl-5-oxo-N-(pyridin-2-ylmethyl)-5,6-dihydro-1,6-naphthyridine-3-carboxamide;
6-(2-fluorobenzyl)-2-methyl-5-oxo-N-(pyridin-2-ylmethyl)-5,6-dihydro-1,6-naphthyridine-3-carboxamide;
6-benzyl-N-((5-chloro-4-fluoropyridin-2-yl)methyl)-2-methyl-5-oxo-5,6-dihydro-1,6-naphthyridine-3-carboxamide;
2-methyl-5-oxo-N-(pyridin-2-ylmethyl)-6-(pyridin-3-ylmethyl)-5,6-dihydro-1,6-naphthyridine-3-carboxamide;
N-((5-chloropyridin-2-yl)methyl)-6-(2-(2-(2-methoxyethoxy)ethoxy)ethyl)-2-methyl-5-oxo-5,6-dihydro-1,6-naphthyridine-3-carboxamide;
6-(bicyclo[1.1.1]pentan-1-ylmethyl)-2-methyl-5-oxo-N-(pyridin-2-ylmethyl)-5,6-dihydro-1,6-naphthyridine-3-carboxamide;
6-(3,4-difluorobenzyl)-2-methyl-5-oxo-N-(pyridin-2-ylmethyl)-5,6-dihydro-1,6-naphthyridine-3-carboxamide;
6-(3,4-difluorobenzyl)-N-((4,5-difluoropyridin-2-yl)methyl)-2-methyl-5-oxo-5,6-dihydro-1,6-naphthyridine-3-carboxamide;
6-(3,4-difluorobenzyl)-N-((5-fluoropyridin-2-yl)methyl)-2-methyl-5-oxo-5,6-dihydro-1,6-naphthyridine-3-carboxamide;
N-((5-chloropyridin-2-yl)methyl)-6-(4-(2-(2-methoxyethoxy)ethoxy)benzyl)-2-methyl-5-oxo-5,6-dihydro-1,6-naphthyridine-3-carboxamide;
6-benzyl-5-oxo-N-(pyridin-2-ylmethyl)-5,6-dihydro-1,6-naphthyridine-3-carboxamide;
6-benzyl-2-methyl-N-((4-methyloxazol-2-yl)methyl)-5-oxo-5,6-dihydro-1,6-naphthyridine-3-carboxamide;
2-amino-6-benzyl-5-oxo-N-(pyridin-2-ylmethyl)-5,6-dihydro-1,6-naphthyridine-3-carboxamide;
6-(4-chloro-3-fluorobenzyl)-2-methyl-5-oxo-N-(pyridin-2-ylmethyl)-5,6-dihydro-1,6-naphthyridine-3-carboxamide;
6-(4-chlorobenzyl)-N-((5-(2-(2-methoxyethoxy)ethoxy)pyridin-2-yl)methyl)-2-methyl-5-oxo-5,6-dihydro-1,6-naphthyridine-3-carboxamide;
6-(3,4-difluorobenzyl)-2-methyl-N-((2-methyloxazol-4-yl)methyl)-5-oxo-5,6-dihydro-1,6-naphthyridine-3-carboxamide;
6-(4-fluorobenzyl)-2-methyl-N-((2-methyloxazol-4-yl)methyl)-5-oxo-5,6-dihydro-1,6-naphthyridine-3-carboxamide;
6-(3-fluorobenzyl)-2-methyl-N-((2-methyloxazol-4-yl)methyl)-5-oxo-5,6-dihydro-1,6-naphthyridine-3-carboxamide;
6-(4-chlorobenzyl)-2-methyl-N-((2-methyloxazol-4-yl)methyl)-5-oxo-5,6-dihydro-1,6-naphthyridine-3-carboxamide; and
6-(3,5-difluorobenzyl)-2-methyl-N-((2-methyloxazol-4-yl)methyl)-5-oxo-5,6-dihydro-1,6-naphthyridine-3-carboxamide;
or a pharmaceutically acceptable salt thereof.

In a twenty-seventh embodiment, the present disclosure provides a pharmaceutical composition comprising a compound according to the twentieth, twenty-first, twenty-second, twenty-third, twenty-fourth, twenty-fifth, or twenty-sixth embodiment, or a pharmaceutically acceptable salt thereof, and a pharmaceutically acceptable carrier or diluent.

In a twenty-eighth embodiment, the present disclosure provides a method of treating a lysosomal storage disorder, infection, metabolic disease, muscle disease, neurodegenerative disease, renal disease, hematologic disease, or optical disease, comprising administering to a subject an effective amount of a compound according to the twentieth, twenty-first, twenty-second, twenty-third, twenty-fourth, twenty-fifth, or twenty-sixth embodiment, or a pharmaceutically acceptable salt thereof, or the pharmaceutical composition according to the first, second, third, fourth, fifth, sixth, seventh, eighth, ninth, tenth, eleventh, twelfth, thirteenth, fourteenth, fifteenth, sixteenth, seventeenth, eighteenth, nineteenth, or twenty-seventh embodiment.

In a twenty-ninth embodiment, the present disclosure provides a method according to the twenty-eighth embodiment, wherein:
the lysosomal storage disorder is selected from Gaucher disease, Pompe disease, NPC, cystinosis, Krabbe disease, Sanfilippo syndrome, multiple sulfatase deficiency, alpha-mannosidosis, Fabry disease, Hunter syndrome, Scheie syndrome, Maroteaux-Lamy syndrome, hyaluronidase deficiency, sialidosis, mucolipidin 1 deficiency, neuronal ceroid lipofuscinoses (Batten Disease), mucopolysaccharidoses Type I, II, III, IV, VI, VII, and IX, Hurler-Scheie syndrome, Morquio syndrome, gly coproteinosis, glycogen storage disease, metachromatic Leukodystrophy, Sly syndrome, I-cell disease, Danon disease, Niemann-Pick disease type A, B, C1 and C2, Sandhoff disease, lysosomal acid lipase deficiency, GM2 gangliosidoses, Tay-Sachs disease, Gaucher disease, Salla disease, cholesteryl ester storage disease, aspartylglucosaminuria, cystinosis, mucolipidosis type I-IV, Schindler disease type I and II, Wolman disease, fucosidosis, pycnodysostosis, and free sialic acid storage disease;
the infection is selected from a bacterial infection, viral infection, and eukaryotic parasites;
the metabolic and muscle disease is selected from al antitrypsin deficiency, polymyositis, and DMD;
the neurodegenerative disease is selected from Parkinson's, Huntington's, Alzheimer's, and Lewy Body Dementia;
the renal disease is selected from PKD, AKI, Kidney Interstitial Fibrosis, and Diabetic Kidney Disease;
the hematologic disease is β-thalassemia; and
the optical disease is selected from Macular Degeneration and Retinitis Pigmentosa.

In a thirtieth embodiment, the present disclosure provides a method of treating a disease or disorder mediated by TFEB, comprising administering to a subject an effective amount of a pharmaceutical composition disclosed herein or a compound disclosed herein.

The compounds and intermediates described herein may be isolated and used as the compound per se, i.e., the neutral form. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two.

Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

### Methods of Use

The compounds disclosed herein have TEFB activation or stimulation activity. As used herein “TFEB activation activity” or “TFEB stimulation activity” refers to the ability of a compound or composition to induce a detectable increase in TFEB activity in vivo or in vitro (e.g., at least 10% increase in TFEB activity as measured by a given assay such as the bioassay described in the examples and known in the art).

In certain embodiments, the present disclosure provides a method of treating a disease or disorder responsive to the activation of TFEB activity (referred to herein as TFEB mediated disease or disorder) in a subject in need of the treatment. The method comprises administering to the subject a compound described herein (e.g., the pharmaceutical composition of any one of the first to nineteenth or twenty-seventh embodiments or the compound of any one of the twentieth to twenty-sixth embodiments) or a pharmaceutically acceptable salt thereof or a pharmaceutical composition thereof.

In certain embodiments, the present disclosure provides the use of a compound described herein (e.g. the pharmaceutical composition of any one of the first to nineteenth or twenty-seventh embodiments or the compound of any one of the twentieth to twenty-sixth embodiments) or a pharmaceutically acceptable salt thereof or a pharmaceutical composition comprising a compound described herein or a pharmaceutically acceptable salt thereof for the manufacture of a medicament for the treatment of a TFEB mediated disease or disorder in a subject in need of the treatment.

In certain embodiments, the present disclosure provides a compound described herein (e.g., the pharmaceutical composition of any one of the first to nineteenth or twenty-seventh embodiments or the compound of any one of the twentieth to twenty-sixth embodiments) or a pharmaceutically acceptable salt thereof or a pharmaceutical composition comprising a compound described herein or a pharmaceutically acceptable salt thereof for use in the treatment of a TFEB mediated disease or disorder in a subject in need of the treatment.

In certain embodiments, the present disclosure provides a method of treating a lysosomal storage disorder, infection, metabolic disease, muscle disease, neurodegenerative disease, renal disease, hematologic disease, or optical disease, in a subject in need of the treatment. The method comprises administering to the subject a compound described herein (e.g., the pharmaceutical composition of any one of the first to nineteenth or twenty-seventh embodiments or the compound of any one of the twentieth to twenty-sixth embodiments) or a pharmaceutically acceptable salt thereof or a pharmaceutical composition thereof.

In certain embodiments, the present disclosure provides the use of a compound described herein (e.g., the pharmaceutical composition of any one of the first to nineteenth or twenty-seventh embodiments or the compound of any one of the twentieth to twenty-sixth embodiments) or a pharmaceutically acceptable salt thereof or a pharmaceutical composition comprising a compound described herein or a pharmaceutically acceptable salt thereof for the manufacture of a medicament for the treatment of a lysosomal storage disorder, infection, metabolic disease, muscle disease, neurodegenerative disease, renal disease, hematologic disease, or optical disease, in a subject in need of the treatment.

In certain embodiments, the present disclosure provides a compound described herein (e.g., the pharmaceutical composition of any one of the first to nineteenth or twenty-seventh embodiments or the compound of any one of the twentieth to twenty-sixth embodiments) or a pharmaceutically acceptable salt thereof or a pharmaceutical composition comprising a compound described herein or a pharmaceutically acceptable salt thereof for use in the treatment of a lysosomal storage disorder, infection, metabolic disease, muscle disease, neurodegenerative disease, renal disease, hematologic disease, or optical disease, in a subject in need of the treatment.

In some embodiments, the lysosomal storage disorder is selected from Gaucher disease, Pompe disease, NPC, cystinosis, Krabbe disease, Sanfilippo syndrome, multiple sulfatase deficiency, alpha-mannosidosis, Fabry disease, Hunter syndrome, Scheie syndrome, Maroteaux-Lamy syndrome, hyaluronidase deficiency, sialidosis, mucolipidin 1 deficiency, neuronal ceroid lipofuscinoses (Batten Disease), mucopolysaccharidoses Type I, II, III, IV, VI, VII, and IX, Hurler-Scheie syndrome, Morquio syndrome, glycoproteinosis, glycogen storage disease, metachromatic Leukodystrophy, Sly syndrome, I-cell disease, Danon disease, Niemann-Pick disease type A, B, C1 and C2, Sandhoff disease, lysosomal acid lipase deficiency, GM2 gangliosidoses, Tay-Sachs disease, Gaucher disease, Salla disease, cholesteryl ester storage disease, aspartylglucosaminuria, cystinosis, mucolipidosis type I-IV, Schindler disease type I and II, Wolman disease, fucosidosis, pycnodysostosis, and free sialic acid storage disease.

In some embodiments, the infection is selected from a bacterial infection, viral infection, and eukaryotic parasites.

In some embodiments, the metabolic and muscle disease is selected from al antitrypsin deficiency, polymyositis, and Duchenne Muscular Dystrophy (DMD).

In some embodiments, the neurodegenerative disease is selected from Parkinson's, Huntington's, Alzheimer's, and Lewy Body Dementia.

In some embodiments, the renal disease is selected from Polycystic Kidney Disease (PKD), Acute Kidney Injury (AKI), Kidney Interstitial Fibrosis, and Diabetic Kidney Disease.

In some embodiments, the hematologic disease is β-thalassemia.

In some embodiments, the optical disease is selected from Macular Degeneration and Retinitis Pigmentosa.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier or diluent). As used herein, the term “pharmaceutically acceptable carrier or diluent” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Definitions

As used herein, a “patient,” “subject” or “individual” are used interchangeably and refer to either a human or non-human animal. The term includes mammals such as humans. Typically, the animal is a mammal. A subject also refers to, for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “stimulate”, “stimulation”, “activate”, or “activation” refers to a significant increase in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtain desired pharmacological and/or physiological effect. The treatment includes therapeutic treatment and prophylactic treatment. The therapeutic treatment includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder. The prophylactic treatment is reducing the likelihood of developing the disease in a subject with known risk factors.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general, the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of perhalogenation, such as perchloro or perfluoroalkyls).

As used herein, the term “alkoxy” refers to a fully saturated branched or unbranched alkyl moiety attached through an oxygen bridge (i.e., a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄alkyl group as defined herein, wherein at least one of the hydrogen atoms is replaced by an C₁₋₄alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or a bicyclic system with two aromatic carbocyclic rings containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrazolyl, imidazolyl, oxazolyl, pyridyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “non-aromatic carbocyclic ring” or “non-aromatic carbocyclyl” refers to a 3- to 12-membered saturated or partially unsaturated hydrocarbon ring system and may exist as a monocylic ring or a polycylic ring (e.g., a bicyclic ring (including fused, spiro or bridged carbocyclic rings) or a tricyclic ring). Bi-cyclic non-aromatic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the non-aromatic carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, benzo[d][1,3]dioxoly, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6-membered monocyclic heterocyclyl group. In some embodiments, the heterocyclyl group is a 4 to 6-membered monocyclic saturated heterocyclyl group. In some embodiments, the heterocyclyl group is an 8 to 10-membered bicyclic heterocyclyl group. In some embodiments, the heterocyclyl group is an 8 to 10-membered bicyclic saturated heterocyclyl group.

As used herein the term “spiro” ring means a two-ring system wherein both rings share one common ring atom. Examples of spiro rings include, 2-oxa-6-azaspiro[3.3]heptanyl and the like.

The term “fused” ring refers to two ring systems sharing two adjacent ring atoms. Fused heterocycles have at least one ring system containing a ring atom that is a heteroatom selected from O, N and S (e.g., 3-oxabicyclo[3.1.0]hexane).

As used herein, the term “bridged” refers to two-ring systems containing 5 to 10 ring atoms and sharing three or more ring atoms, with the two bridgehead ring atoms separated by a bridge containing at least one atom (e.g., bicyclo[1.1.1]pentane or 5-oxabicyclo[2.1.1]hexane).

The phrase “pharmaceutically acceptable” indicates that the substance, composition or dosage form must be chemically compatible with the other ingredients in the formulation.

Alternatively, or in addition, the phrase “pharmaceutically acceptable” indicates that the substance, composition or dosage form must be toxicologically compatible with the mammal being treated therewith.

Unless specified otherwise, the term “compounds of the present disclosure” refers to compounds of Formula (I), as well as all stereoisomers (including diastereoisomers and enantiomers), rotamers, and tautomers. When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form.

In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### EXEMPLIFICATION

### Abbreviations

DCM=dichloromethane
DIPEA=DIEA=diisopropylethyl amine
DMF=dimethylformamide
DMSO=dimethylsulfoxide
EDC=1-Ethyl-3-(3-dimethylaminopropyl)carbodiimide
ESI=electrospray ionisation
EtOAc=EA=ethyl acetate
EtOH=ethanol
FA=formic acid
H₂O=water
HCl=hydrochloric acid
HPLC=high pressure liquid chromatography
HOBT=1-Hydroxybenzotriazole
K₂CO₃=potassium carbonate
LCMS=liquid chromatography mass spectrometry
MeCN=ACN=acetonitrile
MeOH=methanol
N₂=Nitrogen
Na₂SO₄=sodium sulfate
NaBH₄=sodium borohydride
NaOH=Sodium Hydroxide
NH₃=ammonia
NH₄HCO₃=Ammonium Bicarbonate
NMR=Nuclear Magnetic Resonance Spectroscopy
Pd(PCy₃)₂Cl₂=Dichlorobis(tricyclohexylphosphine)palladium(II)
THF=tetrahydrofuran

### Example 1: 6-benzyl-2-methyl-N-((5-methylfuran-2-yl)methyl)-5-oxo-5,6-dihydro-1,6-naphthyridine-3-carboxamide

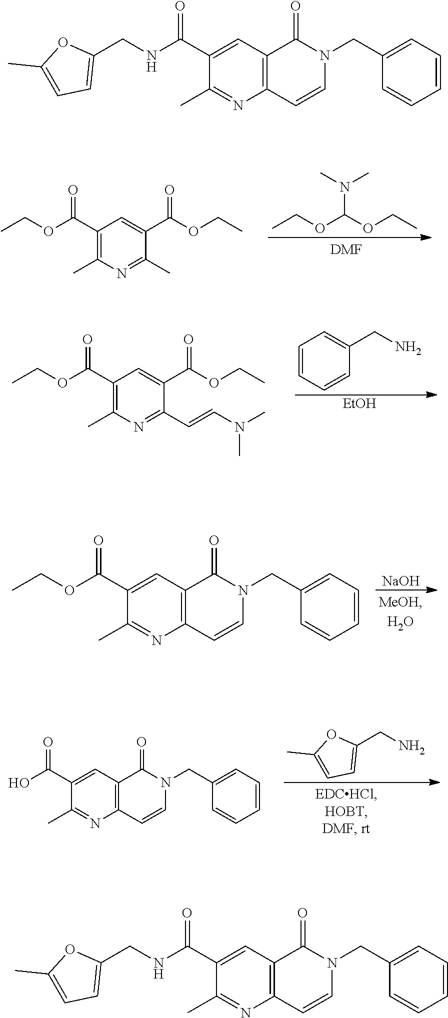

### 1. Synthesis of diethyl (E)-2-(2-(dimethylamino)vinyl)-6-methylpyridine-3,5-dicarboxylate

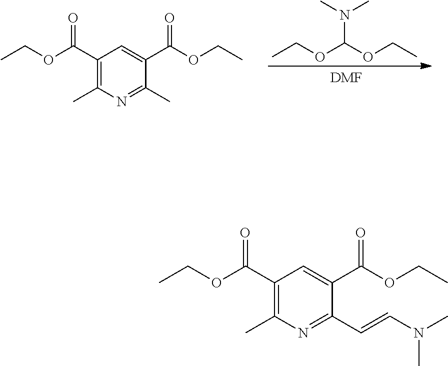

To a solution of diethyl 2,6-dimethylpyridine-3,5-dicarboxylate (520 mg, 2.069 mmol) in DMF (5 mL) was added (diethoxymethyl)dimethylamine (365.5 mg, 2.483 mmol) and the resulting solution was stirred at 100° C. for 8 h. Desired product could be detected by LCMS. Without any treatment, proceed to the next step. LCMS (ESI+): m/z calcd. for C₁₆H₂₃N₂O₄ [M+H]⁺, 307; found, 307.

### 2. Synthesis of ethyl 6-benzyl-2-methyl-5-oxo-5,6-dihydro-1,6-naphthyridine-3-carboxylate

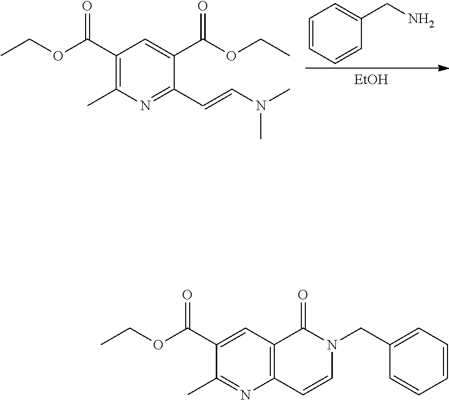

A solution of diethyl (E)-2-(2-(dimethylamino)vinyl)-6-methylpyridine-3,5-dicarboxylate (300 mg, 0.979 mmol) and benzylamine (125.9 mg, 1.175 mmol) in EtOH (3 mL) was stirred at 80° C. for 8 h. The precipitated solids were collected by filtration and washed with water (2×5 mL) to afford ethyl 6-benzyl-2-methyl-5-oxo-5,6-dihydro-1,6-naphthyridine-3-carboxylate as yellow solid (300 mg, yield: 90.2%). LCMS (ESI+): m/z calcd. for C₁₉H₁₈N₂O₃ [M+H]⁺, 323; found, 323.

### 3. Synthesis of 6-benzyl-2-methyl-5-oxo-5,6-dihydro-1,6-naphthyridine-3-carboxylic acid

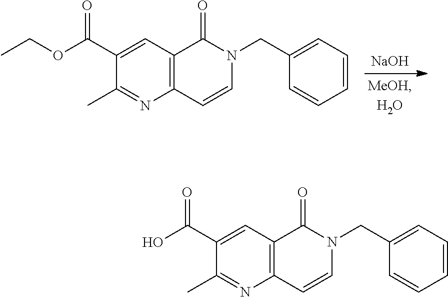

A solution of ethyl 6-benzyl-2-methyl-5-oxo-5,6-dihydro-1,6-naphthyridine-3-carboxylate (322 mg, 0.999 mmol) and NaOH (47.9 mg, 1.199 mmol) in MeOH (3 mL) and H₂O (1 mL) was stirred for 3 h at 40° C. The residue was acidified to pH 4 with conc. HCl, and the aqueous layer was extracted with EtOAc. The combined organic layers were dried over Na₂SO₄ and concentrated in vacuo to afford 6-benzyl-2-methyl-5-oxo-5,6-dihydro-1,6-naphthyridine-3-carboxylic acid as a white solid (250 mg, yield: 67.4%). LCMS (ESI+): m/z calcd. for C₁₇H₁₅N₂O₃ [M+H]⁺, 295; found, 295.

### 4. Synthesis of 6-benzyl-2-methyl-N-((5-methylfuran-2-yl)methyl)-5-oxo-5,6-dihydro-1,6-naphthyridine-3-carboxamide

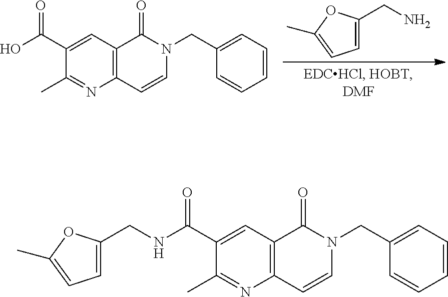

To a solution of 6-benzyl-2-methyl-5-oxo-1,6-naphthyridine-3-carboxylic acid (350 mg, 1.189 mmol) in DMF (5 mL) at 0° C. was added EDC·HCl (276.9 mg, 1.784 mmol) and HOBT (241.0 mg, 1.784 mmol), followed by addition of 1-(5-methylfuran-2-yl)methanamine (198.3 mg, 1.784 mmol). The reaction mixture was stirred at room temperature for 2 h. The crude product was purified by Prep-HPLC with the following conditions (Column: XBridge Prep OBD C₁₈ Column, 30*150 mm, 5 m; Mobile Phase A: Water (10 mmol/L NH₄HCO₃), Mobile Phase B: ACN; Flow rate: 60 mL/min; Gradient: 33% B to 43% B in 8 min, 43%) to afford 6-benzyl-2-methyl-N-((5-methylfuran-2-yl)methyl)-5-oxo-5,6-dihydro-1,6-naphthyridine-3-carboxamide as a white solid (136 mg, yield: 29.4%). LCMS (ESI+): m/z calcd. for C₂₃H₂₂N₃O₃ [M+H]⁺, 388; found, 388. ¹HNMR (400 MHz, DMSO-d₆, δ): 9.06 (t, J=5.6 Hz, 1H), 8.43 (s, 1H), 7.91 (d, J=7.6 Hz, 1H), 7.36-7.27 (m, 5H), 6.72 (d, J=7.6 Hz, 1H), 6.20 (d, J=2.8 Hz, 1H), 6.02 (d, J=1.6 Hz, 1H), 5.22 (s, 2H), 4.43 (d, J=5.6 Hz, 2H), 2.65 (s, 3H), 2.26 (s, 3H).

### Example 2: 6-((5-fluoropyridin-2-yl)methyl)-2-methyl-N-((5-methylfuran-2-yl)methyl)-5-oxo-5,6-dihydro-1,6-naphthyridine-3-carboxamide

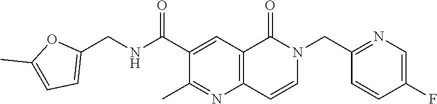 [SMILES: Cc1ccc(CNC(=O)c2cc3c(=O)n(Cc4ccc(F)cn4)ccc3nc2C)o1]

### 1. Synthesis of ethyl 6-((5-fluoropyridin-2-yl)methyl)-2-methyl-5-oxo-5,6-dihydro-1,6-naphthyridine-3-carboxylate

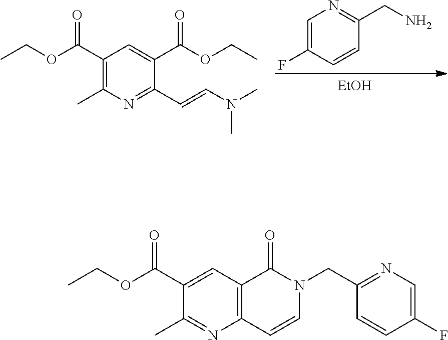

A solution of diethyl (E)-2-(2-(dimethylamino)vinyl)-6-methylpyridine-3,5-dicarboxylate (200 mg, 0.653 mmol) and 1-(5-fluoropyridin-2-yl)methanamine (98.8 mg, 0.784 mmol) in EtOH (2 mL) was stirred for overnight at 80° C. under nitrogen atmosphere. The mixture was allowed to cool down to 0° C., the precipitated solids were collected by filtration and washed with H₂O (3×10 mL) to afford ethyl 6-((5-fluoropyridin-2-yl)methyl)-2-methyl-5-oxo-5,6-dihydro-1,6-naphthyridine-3-carboxylate as a yellow solid (100 mg, yield: 44.8%). LCMS (ESI+): m/z calcd. for C₁₈H₁₇FN₃O₃[M+H]⁺, 342.1; found, 342.1.

### 2. Synthesis of 6-((5-fluoropyridin-2-yl)methyl)-2-methyl-5-oxo-5,6-dihydro-1,6-naphthyridine-3-carboxylic acid

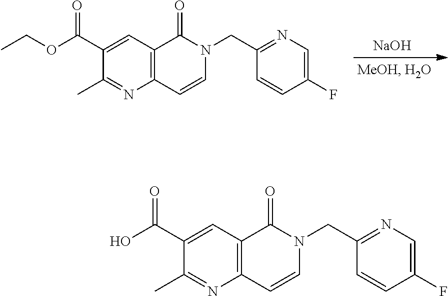 [SMILES: CCOC(=O)c1cc2c(=O)n(Cc3ccc(F)cn3)ccc2nc1C ; "NaOH" ; Cc1nc2ccn(Cc3ccc(F)cn3)c(=O)c2cc1C(=O)O]

A solution of ethyl 6-((5-fluoropyridin-2-yl)methyl)-2-methyl-5-oxo-5,6-dihydro-1,6-naphthyridine-3-carboxylate (90 mg, 0.264 mmol) and NaOH (15.8 mg, 0.396 mmol) in MeOH (1 mL) and H₂O (0.3 mL) was stirred for 2 h at 40° C. The residue was acidified to pH 7 with conc. HCl, and the aqueous layer was extracted with EtOAc. The combined organic layers were dried over Na₂SO₄ and concentrated in vacuo to afford 6-((5-fluoropyridin-2-yl)methyl)-2-methyl-5-oxo-5,6-dihydro-1,6-naphthyridine-3-carboxylic acid as a yellow solid (80 mg, yield: 92.0%). LCMS (ESI+): m/z calcd. for C₁₆H₁₃FN₃O₃[M+H]⁺, 314.1; found, 314.1.

### 3. Synthesis of 6-((5-fluoropyridin-2-yl)methyl)-2-methyl-N-((5-methylfuran-2-yl)methyl)-5-oxo-5,6-dihydro-1,6-naphthyridine-3-carboxamide

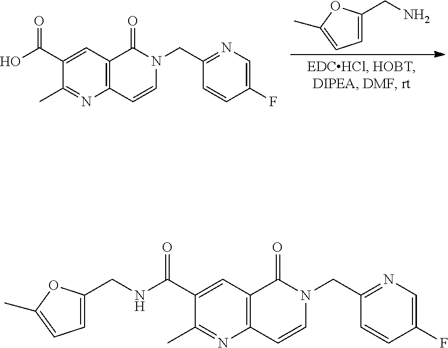

To a solution of 6-((5-fluoropyridin-2-yl)methyl)-2-methyl-5-oxo-5,6-dihydro-1,6-naphthyridine-3-carboxylic acid (42.5 mg, 0.136 mmol) and DIPEA (52.6 mg, 0.407 mmol) in DMF (1 mL) at 0° C. was added EDC·HCl (38.9 mg, 0.203 mmol) and HOBT (27.5 mg, 0.204 mmol), followed by addition of 1-(5-methylfuran-2-yl)methanamine (22.6 mg, 0.203 mmol). The reaction mixture was stirred at room temperature for 3 h. The crude product was purified by Prep-HPLC with the following conditions (Column: XBridge Prep Phenyl OBD Column, 19*250 mm, 5 m; Mobile Phase A: Water (10 mmol/L NH₄HCO₃), Mobile Phase B: MeOH-HPLC; Flow rate: 25 mL/min; Gradient: 60% B to 60% B in 10 min, 60%) to afford 6-((5-fluoropyridin-2-yl)methyl)-2-methyl-N-((5-methylfuran-2-yl)methyl)-5-oxo-5,6-dihydro-1,6-naphthyridine-3-carboxamide as a white solid (10 mg, yield: 18.1%). LCMS (ESI+): m/z calcd. for C₂₂H₂₀FN₄O₃[M+H]⁺, 407.1; found, 407.1. ¹HNMR (400 MHz, DMSO-d₆, δ): 9.05 (t, J=5.6 Hz, 1H), 8.48 (d, J=2.8 Hz, 1H), 8.38 (s, 1H), 7.89 (d, J=7.6 Hz, 1H), 7.74-7.69 (m, 1H), 7.44-7.41 (m, 1H), 6.72 (d, J=7.6 Hz, 1H), 6.18 (d, J=2.8 Hz, 1H), 6.02-6.01 (m, 1H), 5.31 (s, 2H), 4.41 (d, J=5.6 Hz, 2H), 2.66 (s, 3H), 2.25 (s, 3H).

### Example 3: 6-benzyl-2-methyl-N-((5-methyloxazol-2-yl)methyl)-5-oxo-5,6-dihydro-1,6-naphthyridine-3-carboxamide

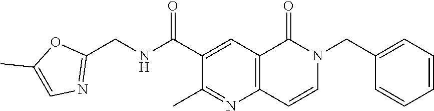 [SMILES: Cc1cnc(CNC(=O)c2cc3c(=O)n(Cc4ccccc4)ccc3nc2C)o1]

### 1. Synthesis of 6-benzyl-2-methyl-N-((5-methyloxazol-2-yl)methyl)-5-oxo-5,6-dihydro-1,6-naphthyridine-3-carboxamide

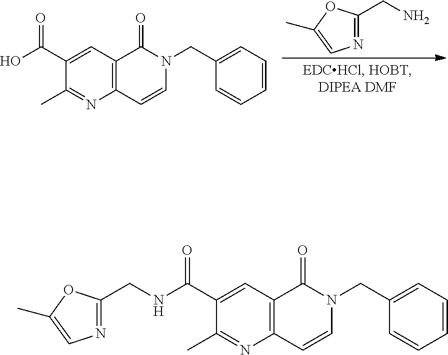

To a solution of 6-benzyl-2-methyl-5-oxo-5,6-dihydro-1,6-naphthyridine-3-carboxylic acid (200 mg, 0.680 mmol) and DIPEA (263.4 mg, 2.040 mmol) in DMF (2 mL) at 0° C. was added EDC·HCl (158.2 mg, 1.020 mmol) and HOBT (137.7 mg, 1.020 mmol), followed by addition of (5-methyloxazol-2-yl)methanamine (114.3 mg, 1.020 mmol). The reaction mixture was stirred at room temperature for 1 h. The crude product was purified by Prep-HPLC with the following conditions (Column: XBridge Prep OBD C₁₈ Column, 30*150 mm, 5 μm; Mobile Phase A: Water (10 mmol/L NH₄HCO₃), Mobile Phase B: ACN; Flow rate: 60 mL/min; Gradient: 23% B to 33% B in 8 min, 33%) to afford 6-benzyl-2-methyl-N-((5-methyloxazol-2-yl)methyl)-5-oxo-5,6-dihydro-1,6-naphthyridine-3-carboxamide as a white solid (22.8 mg, yield: 8.58%). LCMS (ESI+): m/z calcd. for C₂₂H₂₁N₄O₃ [M+H]⁺, 389.2; found, 389.2. ¹HNMR (400 MHz, DMSO-d₆, δ): 9.25 (t, J=5.8 Hz, 1H), 8.49 (s, 1H), 7.91 (d, J=7.6 Hz, 1H), 7.35-7.29 (m, 5H), 6.80-6.79 (m, 1H), 6.73-6.71 (m, 1H), 5.22 (s, 2H), 4.53 (d, J=5.6 Hz, 2H), 2.68 (s, 3H), 2.30-2.29 (m, 3H).

### Example 4: 6-benzyl-2-methyl-N-((2-methyloxazol-5-yl)methyl)-5-oxo-5,6-dihydro-1,6-naphthyridine-3-carboxamide

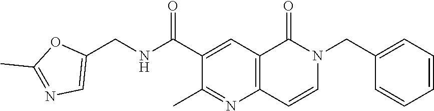 [SMILES: Cc1ncc(CNC(=O)c2cc3c(=O)n(Cc4ccccc4)ccc3nc2C)o1]

### 1. Synthesis of 6-benzyl-2-methyl-N-((2-methyloxazol-5-yl)methyl)-5-oxo-5,6-dihydro-1,6-naphthyridine-3-carboxamide

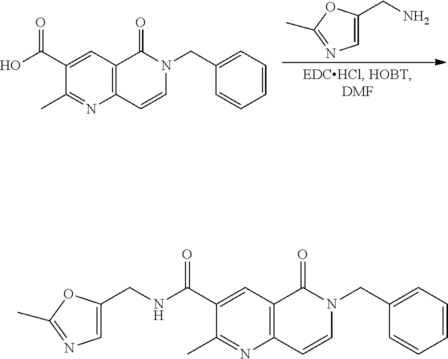

To a solution of 6-benzyl-2-methyl-5-oxo-5,6-dihydro-1,6-naphthyridine-3-carboxylic acid (200 mg, 0.680 mmol) in DMF (2 mL) at 0° C. was added EDC·HCl (195.4 mg, 1.020 mmol) and HOBT (137.7 mg, 1.020 mmol), followed by addition of (2-methyloxazol-5-yl)methanamine (114.3 mg, 1.020 mmol). The reaction mixture was stirred at room temperature for 2 h. The crude product was purified by Prep-HPLC with the following conditions (Column: XBridge Prep OBD C₁₈ Column, 30*150 mm, 5 m; Mobile Phase A: Water (10 mmol/L NH₄HCO₃), Mobile Phase B: ACN; Flow rate: 60 mL/min; Gradient: 22% B to 32% B in 8 min, 32%) to afford 6-benzyl-2-methyl-N-((2-methyloxazol-5-yl)methyl)-5-oxo-5,6-dihydro-1,6-naphthyridine-3-carboxamide as a white solid (86.6 mg, yield: 32.7%). LCMS (ESI+): m/z calcd. for C₂₂H₂₁N₄O₃ [M+H]⁺, 389.2; found, 389.1. ¹HNMR (400 MHz, DMSO-d₆, δ): 9.13 (t, J=5.4 Hz, 1H), 8.44 (s, 1H), 7.91 (d, J=7.6 Hz, 1H), 7.36-7.28 (m, 5H), 6.92 (s, 1H), 6.72-6.70 (m, 1H), 5.21 (s, 2H), 4.49 (d, J=5.2 Hz, 2H), 2.65 (s, 3H), 2.39 (s, 3H).

### Example 5: 6-benzyl-2-methyl-N-(3-methylbenzyl)-5-oxo-5,6-dihydro-1,6-naphthyridine-3-carboxamide

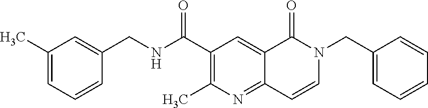 [SMILES: Cc1cccc(CNC(=O)c2cc3c(=O)n(Cc4ccccc4)ccc3nc2C)c1]

### 1. Synthesis of 6-benzyl-2-methyl-N-(3-methylbenzyl)-5-oxo-5,6-dihydro-1,6-naphthyridine-3-carboxamide

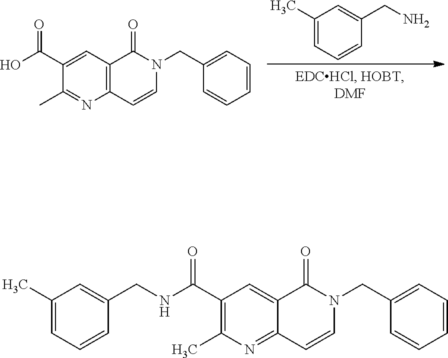

To a solution of 6-benzyl-2-methyl-5-oxo-5,6-dihydro-1,6-naphthyridine-3-carboxylic acid (200 mg, 0.680 mmol) in DMF (4 mL) at 0° C. was added EDC·HCl (195.4 mg, 1.020 mmol) and HOBT (137.7 mg, 1.020 mmol), followed by addition of m-tolylmethanamine (56 mg, 0.518 mmol). The reaction mixture was stirred at room temperature for 1 h. The crude product was purified by Prep-HPLC with the following conditions (Column: XBridge Shield RP18 OBD Column, 30*150 mm, 5 m; Mobile Phase A: Water (10 mmol/L NH₄HCO₃), Mobile Phase B: ACN; Flow rate: 60 mL/min; Gradient: 40% B to 55% B in 8 min, 55% B) to afford 6-benzyl-2-methyl-N-(3-methylbenzyl)-5-oxo-5,6-dihydro-1,6-naphthyridine-3-carboxamide as a white solid (125.7 mg, yield: 46.1%). LCMS (ESI+): m/z calcd. for C₂₅H₂₄N₃O₂ [M+H]⁺, 398.2; found, 398.2. ¹HNMR (300 MHz, DMSO-d₆, δ): 9.12 (t, J=5.9 Hz, 1H), 8.46 (s, 1H), 7.91-7.89 (m, 1H), 7.36-7.23 (m, 6H), 7.17-7.14 (m, 2H), 7.10-7.07 (s, 1H), 6.71 (d, J=7.5 Hz, 1H), 5.21 (s, 2H), 4.45 (d, J=6.0 Hz, 2H), 2.66 (s, 3H), 2.31 (s, 3H).

### Example 6: 6-benzyl-2-methyl-N-((6-methylpyridin-2-yl)methyl)-5-oxo-5,6-dihydro-1,6-naphthyridine-3-carboxamide

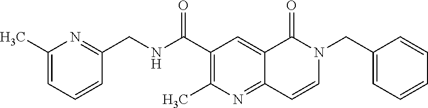 [SMILES: Cc1cccc(CNC(=O)c2cc3c(=O)n(Cc4ccccc4)ccc3nc2C)n1]

### 1. Synthesis of 6-benzyl-2-methyl-N-((6-methylpyridin-2-yl)methyl)-5-oxo-5,6-dihydro-1,6-naphthyridine-3-carboxamide

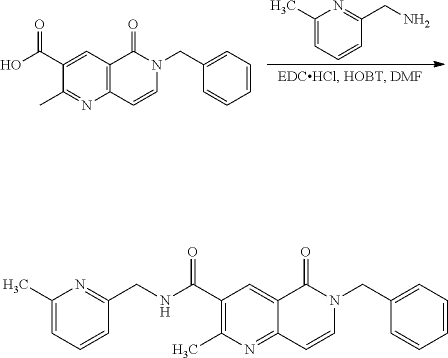

To a solution of 6-benzyl-2-methyl-5-oxo-5,6-dihydro-1,6-naphthyridine-3-carboxylic acid (250 mg, 0.849 mmol) in DMF (2.5 mL) at 0° C. was added EDC·HCl (197.8 mg, 1.273 mmol) and HOBT (172.1 mg, 1.273 mmol), followed by addition of (6-methylpyridin-2-yl)methanamine (155.6 mg, 1.273 mmol). The reaction mixture was stirred at room temperature for 2 h. The crude product was purified by Prep-HPLC with the following conditions (Column: XBridge Prep OBD C₁₈ Column, 30*150 mm, 5 m; Mobile Phase A: Water (10 mmol/L NH₄HCO₃), Mobile Phase B: ACN; Flow rate: 60 mL/min; Gradient: 25% B to 35% B in 8 min, 35% B) to afford 6-benzyl-2-methyl-N-((6-methylpyridin-2-yl)methyl)-5-oxo-5,6-dihydro-1,6-naphthyridine-3-carboxamide as a light yellow solid (136.4 mg, yield: 40.2%). LCMS (ESI+): m/z calcd. for C₂₄H₂₃N₄O₂ [M+H]⁺, 399.2; found, 399.2. ¹HNMR (400 MHz, DMSO-d₆, δ): 9.20 (t, J=6.0 Hz, 1H), 8.55 (s, 1H), 7.91 (d, J=7.6 Hz, 1H), 7.69 (t, J=7.7 Hz, 1H), 7.40-7.24 (m, 5H), 7.17 (dd, J=15.4, 7.7 Hz, 2H), 6.73 (d, J=7.6 Hz, 1H), 5.23 (s, 2H), 4.54 (d, J=6.0 Hz, 2H), 2.69 (s, 3H), 2.48 (s, 3H).

### Example 7: 6-benzyl-2-methyl-5-oxo-N-(pyridin-2-ylmethyl)-5,6-dihydro-1,6-naphthyridine-3-carboxamide

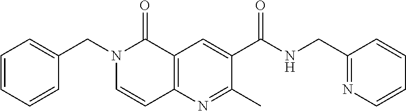 [SMILES: Cc1nc2ccn(Cc3ccccc3)c(=O)c2cc1C(=O)NCc1ccccn1]

### 1. Synthesis of 6-benzyl-2-methyl-5-oxo-N-(pyridin-2-ylmethyl)-5,6-dihydro-1,6-naphthyridine-3-carboxamide

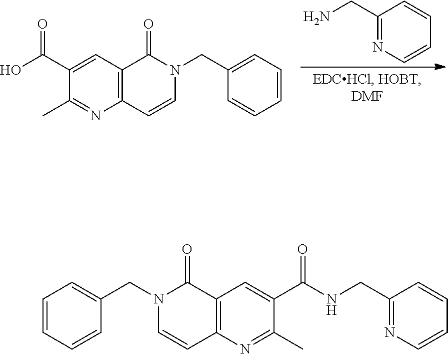 [SMILES: Cc1nc2ccn(Cc3ccccc3)c(=O)c2cc1C(=O)O ; NCc1ccccn1 ; Cc1nc2ccn(Cc3ccccc3)c(=O)c2cc1C(=O)NCc1ccccn1]

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (97.7 mg, 0.510 mmol) and HOBT (68.87 mg, 0.510 mmol), followed by addition of 2-pyridinemethaneamine (55.1 mg, 0.510 mmol). The reaction mixture was stirred at room temperature for 1 h. Then crude was purified by reverse phase column (gradient: 0˜50% ACN/H₂O (0.5% NH₄HCO₃)) to afford 6-benzyl-2-methyl-5-oxo-N-(pyridin-2-ylmethyl)-5,6-dihydro-1,6-naphthyridine-3-carboxamide as a white solid (52.1 mg, yield: 39.8%). LCMS (ESI+): m/z calcd. for C₂₃H₂₁N₄O₂ [M+H]⁺, 385.2; found, 385.1. ¹HNMR (400 MHz, DMSO-d₆, δ): 9.23 (t, J=6.0 Hz, 1H), 8.55-8.54 (m, 2H), 7.92 (d, J=7.6 Hz, 1H), 7.83-7.79 (m, 1H), 7.42-7.28 (m, 7H), 6.73 (d, J=7.6 Hz, 1H), 5.22 (s, 2H), 4.59 (d, J=5.6 Hz, 2H), 2.69 (s, 3H).

### Example 8: 6-benzyl-2-methyl-N-((1-methyl-11H-1,2,3-triazol-4-yl)methyl)-5-oxo-5,6-dihydro-1,6-naphthyridine-3-carboxamide

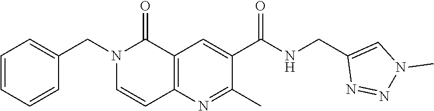 [SMILES: Cc1nc2ccn(Cc3ccccc3)c(=O)c2cc1C(=O)NCc1cn(C)nn1]

### 1. Synthesis of 6-benzyl-2-methyl-N-((1-methyl-1H-1,2,3-triazol-4-yl)methyl)-5-oxo-5,6-dihydro-1,6-naphthyridine-3-carboxamide

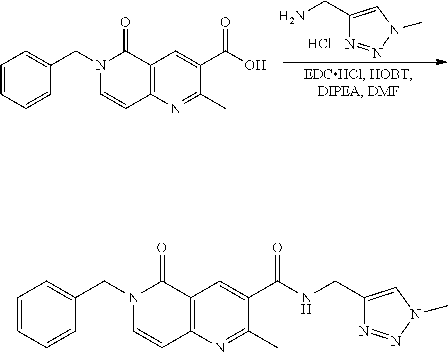

To a solution of 6-benzyl-2-methyl-5-oxo-5,6-dihydro-1,6-naphthyridine-3-carboxylic acid (100 mg, 0.340 mmol) and DIEA (131.7 mg, 1.020 mmol) in DMF (1 mL) at 0° C. was added EDC·HCl (97.7 mg, 0.510 mmol) and HOBT (68.8 mg, 0.510 mmol), followed by addition of a (1-methyl-1H-1,2,3-triazol-4-yl)methanamine hydrochloride (76.2 mg, 0.515 mmol). The reaction mixture was stirred at room temperature for 1 h. Then crude was purified by reverse phase column (gradient: 0˜50% MeOH/H₂O (0.5% TFA)) to afford 6-benzyl-2-methyl-N-((1-methyl-1H-1,2,3-triazol-4-yl)methyl)-5-oxo-5,6-dihydro-1,6-naphthyridine-3-carboxamide as white solid (5.0 mg, yield: 3.8%). LCMS (ESI+): m/z calcd. for C₂₁H₂₁N₆O₂ [M+H]⁺, 389; found, 389. ¹HNMR (400 MHz, MeOH-d₄, δ): 8.67-8.63 (m, 1H), 7.96 (s, 1H), 7.80-7.56 (m, 1H), 7.57-7.31 (m, 5H), 6.79 (d, J=7.6 Hz, 1H), 5.27 (s, 2H), 4.18-4.13 (m, 2H), 2.68 (s, 3H), 2.76 (s, 3H).

### Example 9: 6-benzyl-2-methyl-N-((2-methyl-2H-1,2,3-triazol-4-yl)methyl)-5-oxo-5,6-dihydro-1,6-naphthyridine-3-carboxamide

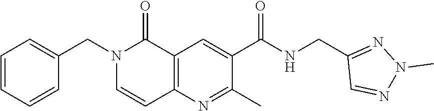 [SMILES: Cc1nc2ccn(Cc3ccccc3)c(=O)c2cc1C(=O)NCc1cnn(C)n1]

### 1. Synthesis of 6-benzyl-2-methyl-N-((2-methyl-2H-1,2,3-triazol-4-yl)methyl)-5-oxo-5,6-dihydro-1,6-naphthyridine-3-carboxamide

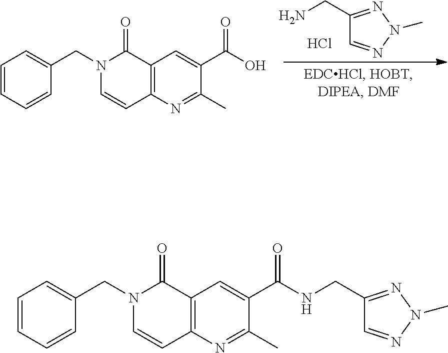

To a solution of 6-benzyl-2-methyl-5-oxo-5,6-dihydro-1,6-naphthyridine-3-carboxylic acid (100 mg, 0.340 mmol) and DIEA (142.2 mg, 1.102 mmol) in DMF (1 mL) at 0° C. was added EDC·HCl (98.2 mg, 0.514 mmol) and HOBT (70.1 mg, 0.519 mmol), followed by addition of a (2-methyl-2H-1,2,3-triazol-4-yl)methanamine hydrochloride (78.5 mg, 0.528 mmol). The reaction mixture was stirred at room temperature for 1 h. Then crude was purified by reverse phase column (gradient: 0˜50% MeOH/H₂O (0.5% FA)) to afford 6-benzyl-2-methyl-N-((2-methyl-2H-1,2,3-triazol-4-yl)methyl)-5-oxo-5,6-dihydro-1,6-naphthyridine-3-carboxamide as white solid (6.6 mg, yield: 3.8%). LCMS (ESI+): m/z calcd. for C₂₁H₂₁N₆O₂ [M+H]⁺, 389; found, 389. ¹HNMR (400 MHz, DMSO-d₆, δ): 9.17-9.11 (m, 1H), 8.46 (m, 1H), 7.91 (d, J=7.6 Hz, 1H), 7.67 (s, 1H), 7.33-7.26 (m, 5H), 6.71 (d, J=7.6 Hz, 1H), 5.22 (s, 2H), 4.52-4.51 (m, 2H), 4.13 (s, 3H), 2.66 (s, 3H).

### Example 10: 6-benzyl-2-methyl-N-((3-methyl-1,2,4-oxadiazol-5-yl)methyl)-5-oxo-5,6-dihydro-1,6-naphthyridine-3-carboxamide

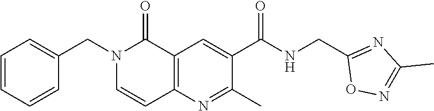 [SMILES: Cc1noc(CNC(=O)c2cc3c(=O)n(Cc4ccccc4)ccc3nc2C)n1]

### 1. Synthesis of 6-benzyl-2-methyl-N-((3-methyl-1,2,4-oxadiazol-5-yl)methyl)-5-oxo-5,6-dihydro-1,6-naphthyridine-3-carboxamide

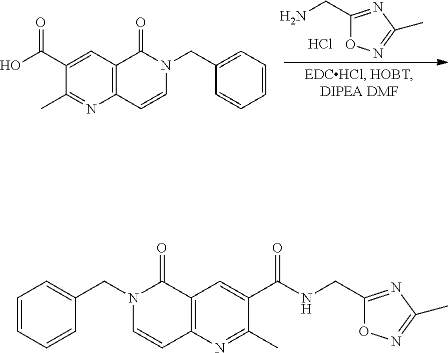

To a solution of 6-benzyl-2-methyl-5-oxo-5,6-dihydro-1,6-naphthyridine-3-carboxylic acid (100 mg, 0.340 mmol) and DIPEA (131.7 mg, 1.020 mmol) in DMF (2 mL) at 0° C. was added EDC·HCl (97.7 mg, 0.510 mmol) and HOBT (68.8 mg, 0.510 mmol), followed by addition of a (3-methyl-1,2,4-oxadiazol-5-yl)methanamine hydrochloride (70.1 mg, 0.469 mmol). The reaction mixture was stirred at room temperature for 1 h. Then crude was purified by reverse phase column (gradient: 0˜50% MeCN/H₂O (0.5% FA)) to afford 6-benzyl-2-methyl-N-((3-methyl-1,2,4-oxadiazol-5-yl)methyl)-5-oxo-5,6-dihydro-1,6-naphthyridine-3-carboxamide as white solid (11.8 mg, yield: 8.8%). LCMS (ESI+): m/z calcd. for C₂₁H₂₀N₅O₃ [M+H]⁺, 390.2, found, 390.2. ¹HNMR (400 MHz, DMSO-d₆, δ): 9.47 (t, J=5.6 Hz, 1H), 8.54 (s, 1H), 7.93 (d, J=7.6 Hz, 1H), 7.37-7.27 (m, 5H), 6.73 (d, J=7.6 Hz, 1H), 5.23 (s, 2H), 4.74 (d, J=5.6 Hz, 2H), 2.68 (s, 3H), 2.36 (s, 3H).

### Example 11: N-(4-chlorobenzyl)-2-methyl-5-oxo-6-((tetrahydrofuran-2-yl)methyl)-5,6-dihydro-1,6-naphthyridine-3-carboxamide

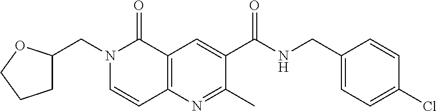 [SMILES: Cc1nc2ccn(CC3CCCO3)c(=O)c2cc1C(=O)NCc1ccc(Cl)cc1]

### 1. Synthesis of ethyl 2-methyl-5-oxo-6-((tetrahydrofuran-2-yl)methyl)-5,6-dihydro-1,6-naphthyridine-3-carboxylate

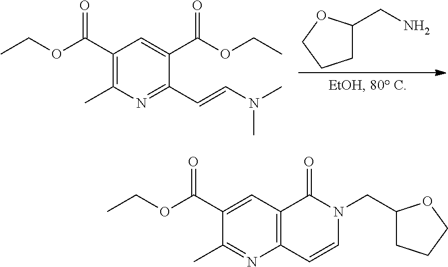

A solution of diethyl (E)-2-(2-(dimethylamino)vinyl)-6-methylpyridine-3,5-dicarboxylate (1.0 g, 3.267 mmol) and 1-(oxolan-2-yl) methanamine (396.5 mg, 3.9204 mmol) in EtOH (5 mL) was stirred for overnight at 80° C. under nitrogen atmosphere. The mixture was cooled to 0° C., the precipitated solids were collected by filtration and washed with H₂O (3×10 mL) to afford ethyl 2-methyl-5-oxo-6-((tetrahydrofuran-2-yl)methyl)-5,6-dihydro-1,6-naphthyridine-3-carboxylate as a yellow solid (457.0 mg, yield 44.1%). LCMS (ESI+): m/z calcd. for C₁₇H₂₁N₂O₄ [M+H]⁺, 317, found, 317.

### 2. Synthesis of 2-methyl-5-oxo-6-((tetrahydrofuran-2-yl)methyl)-5,6-dihydro-1,6-naphthyridine-3-carboxylic acid

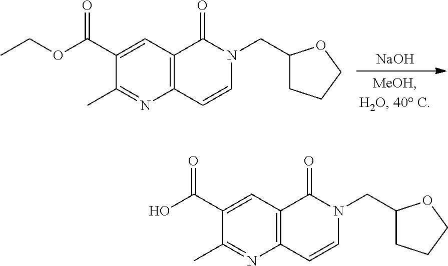

A solution of ethyl 2-methyl-5-oxo-6-((tetrahydrofuran-2-yl)methyl)-5,6-dihydro-1,6-naphthyridine-3-carboxylate (200 mg, 0.632 mmol) and NaOH (76 mg, 1.900 mmol) in MeOH (3 mL) and H₂O (1 mL) was stirred for 2 h at 40° C. The residue was acidified to pH 7 with conc. HCl, and the aqueous layer was extracted with EtOAc. The combined organic layers were dried over Na₂SO₄ and concentrated in vacuo to afford 2-methyl-5-oxo-6-((tetrahydrofuran-2-yl)methyl)-5,6-dihydro-1,6-naphthyridine-3-carboxylic acid as a white solid (120 mg, yield: 65.8%). LCMS (ESI+): m/z calcd. for C₁₅H₁₆N₂O₄ [M+H]⁺, 289, found, 289.

### 3. Synthesis of N-(4-chlorobenzyl)-2-methyl-5-oxo-6-((tetrahydrofuran-2-yl)methyl)-5,6-dihydro-1,6-naphthyridine-3-carboxamide

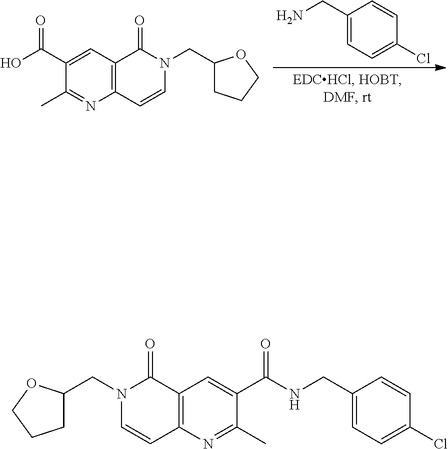 [SMILES: Cc1nc2ccn(CC3CCCO3)c(=O)c2cc1C(=O)O ; NCc1ccc(Cl)cc1 ; Cc1nc2ccn(CC3CCCO3)c(=O)c2cc1C(=O)NCc1ccc(Cl)cc1]

To a solution of 2-methyl-5-oxo-6-((tetrahydrofuran-2-yl)methyl)-5,6-dihydro-1,6-naphthyridine-3-carboxylic acid (120 mg, 0.416 mmol) in DMF (2 mL) at 0° C. was added EDC·HCl (120 mg, 0.626 mmol) and HOBT (84 mg, 0.622 mmol), followed by addition of 1-(4-chlorophenyl)methanamine (88 mg, 0.621 mmol). The reaction mixture was stirred at room temperature for 1 h. Then crude was purified by reverse phase column (gradient: 0˜50% ACN/H₂O (0.5% NH₄HCO₃)) to afford N-(4-chlorobenzyl)-2-methyl-5-oxo-6-((tetrahydrofuran-2-yl)methyl)-5,6-dihydro-1,6-naphthyridine-3-carboxamide as a white solid (96.9 mg, yield: 56.3%). LCMS (ESI+): m/z calcd. for C₂₂H₂₃ClN₃O₃[M+H]⁺, 412, found, 412. ¹HNMR (400 MHz, DMSO-d₆, δ): 9.19 (t, J=5.9 Hz, 1H), 8.47 (s, 1H), 7.73 (d, J=7.6 Hz, 1H), 7.45-7.39 (m, 4H), 6.66 (d, J=7.6 Hz, 1H), 4.48 (d, J=6.0 Hz, 2H), 4.18-4.12 (m, 2H), 3.97-3.92 (m, 1H), 3.80-3.76 (m, 1H), 3.66-3.61 (m, 1H), 2.66 (s, 3H), 1.98-1.88 (m, 1H), 1.86-1.74 (m, 2H), 1.64-1.52 (m, 1H).

### Example 12: 6-benzyl-2-methyl-5-oxo-N-(pyridin-4-ylmethyl)-5,6-dihydro-1,6-naphthyridine-3-carboxamide

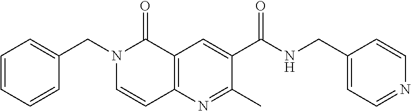 [SMILES: Cc1nc2ccn(Cc3ccccc3)c(=O)c2cc1C(=O)NCc1ccncc1]

### 1. Synthesis of 6-benzyl-2-methyl-5-oxo-N-(pyridin-4-ylmethyl)-5,6-dihydro-1,6-naphthyridine-3-carboxamide

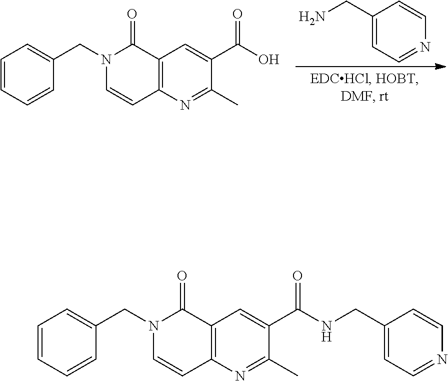 [SMILES: Cc1nc2ccn(Cc3ccccc3)c(=O)c2cc1C(=O)O ; NCc1ccncc1 ; Cc1nc2ccn(Cc3ccccc3)c(=O)c2cc1C(=O)NCc1ccncc1]

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (98 mg, 0.511 mmol) and HOBT (69 mg, 0.511 mmol), followed by addition of 4-pyridinemethaneamine (56 mg, 0.518 mmol). The reaction mixture was stirred at room temperature for 1 h. Then crude was purified by reverse phase column (gradient: 0˜50% ACN/H₂O (0.5% NH₄HCO₃)) to afford 6-benzyl-2-methyl-5-oxo-N-(pyridin-4-ylmethyl)-5,6-dihydro-1,6-naphthyridine-3-carboxamide as a white solid (43.4 mg, yield: 33.1%). LCMS (ESI+): m/z calcd. for C₂₃H₂₁N₄O₂ [M+H]⁺, 385.2, found, 385.2. T HNMR (400 MHz, DMSO-d₆, δ): 9.26 (t, J=5.8 Hz, 1H), 8.55 (d, J=4.0 Hz, 3H), 7.92 (d, J=7.6 Hz, 1H), 7.37-7.27 (m, 7H), 6.72 (d, J=7.6 Hz, 1H), 5.23 (s, 2H), 4.52 (d, J=6.0 Hz, 2H), 2.68 (s, 3H).

### Example 13: 6-benzyl-2-methyl-N-((5-methyl-1,3,4-oxadiazol-2-yl)methyl)-5-oxo-5,6-dihydro-1,6-naphthyridine-3-carboxamide

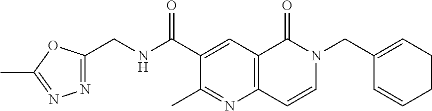 [SMILES: Cc1nnc(CNC(=O)c2cc3c(=O)n(CC4=CCCC=C4)ccc3nc2C)o1]

### 1. Synthesis of 6-benzyl-2-methyl-N-((5-methyl-1,3,4-oxadiazol-2-yl)methyl)-5-oxo-5,6-dihydro-1,6-naphthyridine-3-carboxamide

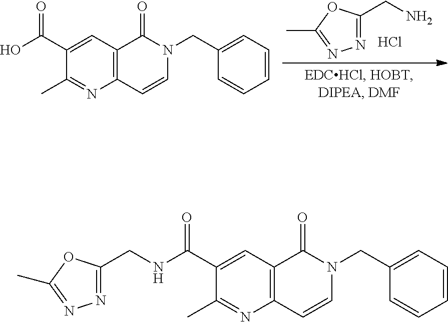

To a solution of 6-benzyl-2-methyl-5-oxo-5,6-dihydro-1,6-naphthyridine-3-carboxylic acid (200 mg, 0.680 mmol) and DIPEA (131.7 mg, 1.020 mmol) in DMF (2 mL) at 0° C. was added EDC·HCl (195.4 mg, 1.020 mmol) and HOBT (137.7 mg, 1.020 mmol), followed by addition of a (5-methyl-1,3,4-oxadiazol-2-yl)methanamine hydrochloride (115.3 mg, 0.774 mmol). The reaction mixture was stirred at room temperature for 2 h. The crude product was purified by Prep-HPLC with the following conditions (Column: XBridge Shield RP18 OBD Column, 30*150 mm, 5 m; Mobile Phase A: Water (10 mmol/L NH₄HCO₃), Mobile Phase B: ACN; Flow rate: 60 mL/min; Gradient: 20% B to 35% B in 80 min, 35%) to afford 6-benzyl-2-methyl-N-((5-methyl-1,3,4-oxadiazol-2-yl)methyl)-5-oxo-5,6-dihydro-1,6-naphthyridine-3-carboxamide as a white solid (28.3 mg, yield: 10.7%). LCMS (ESI+): m/z calcd. for C₂₁H₂₀N₅O₃ [M+H]⁺, 390, found, 390. ¹HNMR (300 MHz, DMSO-d₆, δ): 9.37 (t, J=6.2 Hz, 1H), 8.51 (s, 1H), 7.94 (d, J=7.8 Hz, 1H), 7.34-7.28 (m, 5H), 6.72 (d, J=7.5 Hz, 1H), 5.22 (s, 2H), 4.67 (d, J=5.7 Hz, 2H), 2.87 (s, 3H), 2.51 (s, 3H).

### Example 14: 6-benzyl-2-methyl-N-((1-methyl-1H-pyrazol-3-yl)methyl)-5-oxo-5,6-dihydro-1,6-naphthyridine-3-carboxamide

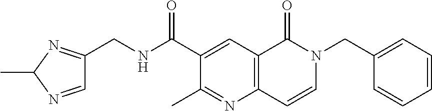 [SMILES: Cc1nc2ccn(Cc3ccccc3)c(=O)c2cc1C(=O)NCC1=NC(C)N=C1]

### 1. Synthesis of 6-benzyl-2-methyl-N-((1-methyl-1H-pyrazol-3-yl)methyl)-5-oxo-5,6-dihydro-1,6-naphthyridine-3-carboxamide

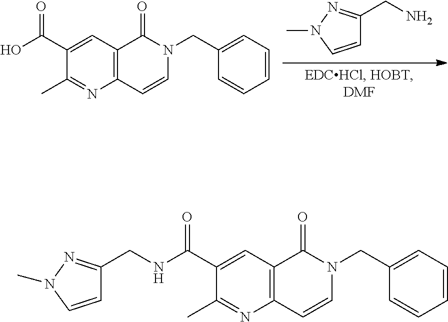

To a solution of 6-benzyl-2-methyl-5-oxo-5,6-dihydro-1,6-naphthyridine-3-carboxylic acid acid (200 mg, 0.680 mmol) in DMF (2 mL) at 0° C. was added EDC·HCl (195.4 mg, 1.020 mmol) and HOBT (137.74 mg, 1.020 mmol), followed by addition of 1-(1-methylpyrazol-3-yl)methanamine (113.3 mg, 1.020 mmol). The reaction mixture was stirred at room temperature for 2 h. The crude product was purified by Prep-HPLC with the following conditions (Column: Xselect CSH C₁₈ OBD Column 30*150 mm 5 m, n; Mobile Phase A: Water (0.1% FA), Mobile Phase B: ACN; Flow rate: 60 mL/min; Gradient: 20% B to 40% B in 12 min, 40%) to afford 6-benzyl-2-methyl-N-((1-methyl-1H-pyrazol-3-yl)methyl)-5-oxo-5,6-dihydro-1,6-naphthyridine-3-carboxamide as a white solid (45 mg, yield: 16.8%). LCMS (ESI+): m/z calcd. for C₂₂H₂₂N₅O₂ [M+H]⁺, 388.2, found, 388.2. ¹HNMR (400 MHz, DMSO-d₆, δ): 9.02-9.01 (m, 1H), 8.41 (s, 1H), 7.91-7.89 (m, 1H), 7.62-7.61 (m, 1H), 7.40-7.26 (m, 5H), 6.71-6.69 (m, 1H), 6.21-6.17 (m, 1H), 5.21 (s, 2H), 4.40 (d, J=5.6 Hz, 2H), 3.80 (s, 3H), 2.65 (s, 3H).

### Example 15: 6-benzyl-2-methyl-N-((1-methyl-1H-pyrazol-4-yl)methyl)-5-oxo-5,6-dihydro-1,6-naphthyridine-3-carboxamide

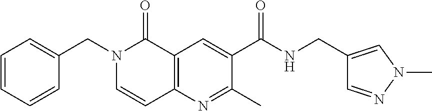 [SMILES: Cc1nc2ccn(Cc3ccccc3)c(=O)c2cc1C(=O)NCc1cnn(C)c1]

### 1. Synthesis of 6-benzyl-2-methyl-N-((1-methyl-1H-pyrazol-4-yl)methyl)-5-oxo-5,6-dihydro-1,6-naphthyridine-3-carboxamide

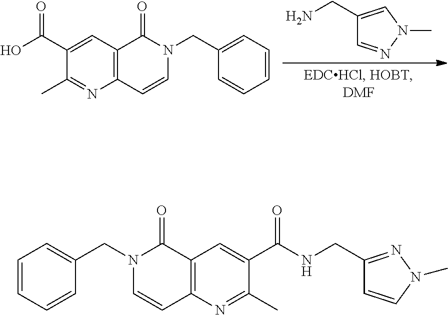

To a solution of 6-benzyl-2-methyl-5-oxo-5,6-dihydro-1,6-naphthyridine-3-carboxylic acid (100 mg, 0.340 mmol) in DMF (1 mL) at 0° C. was added EDC·HCl (97.7 mg, 0.510 mmol) and HOBT (68.8 mg, 0.510 mmol), followed by addition of 1-(1-methylpyrazol-4-yl)methanamine (56.6 mg, 0.510 mmol). The reaction mixture was stirred at room temperature for 1 h. Then crude was purified by reverse phase column (gradient: 0˜50% ACN/H₂O (0.5% FA)) to afford 6-benzyl-2-methyl-N-((1-methyl-1H-pyrazol-4-yl)methyl)-5-oxo-5,6-dihydro-1,6-naphthyridine-3-carboxamide as a white solid (31.4 mg, yield: 23.7%). LCMS (ESI+): m/z calcd. for C₂₂H₂₂N₅O₂ [M+H]⁺, 388.2, found, 388.2. ¹HNMR (400 MHz, DMSO-d₆, δ): 8.94 (t, J=5.6 Hz, 1H), 8.41 (s, 1H), 7.90 (d, J=7.6 Hz, 1H), 7.65 (s, 1H), 7.40-7.26 (m, 6H), 6.70 (d, J=7.6 Hz, 1H), 5.21 (s, 2H), 4.31 (d, J=5.6 Hz, 2H), 3.81 (s, 3H), 2.65 (s, 3H).

### Example 16: 6-benzyl-N-(2-fluorobenzyl)-2-methyl-5-oxo-5,6-dihydro-1,6-naphthyridine-3-carboxamide

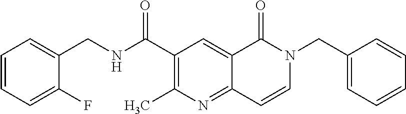 [SMILES: Cc1nc2ccn(Cc3ccccc3)c(=O)c2cc1C(=O)NCc1ccccc1F]

### 1. Synthesis of 6-benzyl-N-(2-fluorobenzyl)-2-methyl-5-oxo-5,6-dihydro-1,6-naphthyridine-3-carboxamide

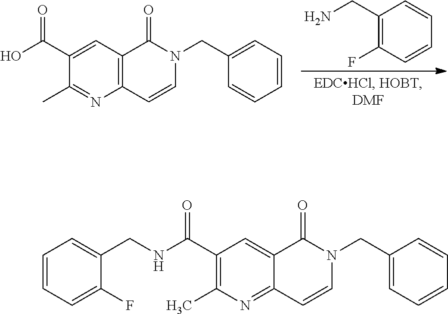 [SMILES: Cc1nc2ccn(Cc3ccccc3)c(=O)c2cc1C(=O)O ; NCc1ccccc1F ; Cc1nc2ccn(Cc3ccccc3)c(=O)c2cc1C(=O)NCc1ccccc1F]

To a solution of 6-benzyl-2-methyl-5-oxo-5,6-dihydro-1,6-naphthyridine-3-carboxylic acid (100 mg, 0.340 mmol) in DMF (1.5 mL) at 0° C. was added EDC·HCl (79.1 mg, 0.510 mmol) and HOBT (68.8 mg, 0.510 mmol), followed by addition of 1-(2-fluorophenyl)methanamine (63.7 mg, 0.510 mmol). The reaction mixture was stirred at room temperature for 1 h. Then crude was purified by reverse phase column (gradient: 0˜50% ACN/H₂O (0.5% NH₄HCO₃)) to afford 6-benzyl-N-(2-fluorobenzyl)-2-methyl-5-oxo-5,6-dihydro-1,6-naphthyridine-3-carboxamide as a white solid (73.4 mg, yield: 52.3%). LCMS (ESI+): m/z calcd. for C₂₄H₂₁FN₃O₂[M+H]⁺, 402.2, found, 402.2. ¹HNMR (400 MHz, DMSO-d₆, δ): 9.15 (t, J=5.8 Hz, 1H), 8.47 (s, 1H), 7.90 (d, J=7.6 Hz, 1H), 7.50-7.41 (m, 1H), 7.39-7.27 (m, 6H), 7.29-7.18 (m, 2H), 6.72 (d, J=7.6 Hz, 1H), 5.21 (s, 2H), 4.53 (d, J=5.7 Hz, 2H), 2.65 (s, 3H).

### Example 17: N,6-dibenzyl-2-methyl-5-oxo-5,6-dihydro-1,6-naphthyridine-3-carboxamide

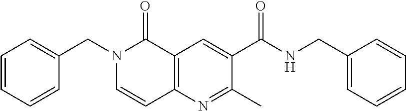 [SMILES: Cc1nc2ccn(Cc3ccccc3)c(=O)c2cc1C(=O)NCc1ccccc1]

### 1. Synthesis of N,6-dibenzyl-2-methyl-5-oxo-5,6-dihydro-1,6-naphthyridine-3-carboxamide

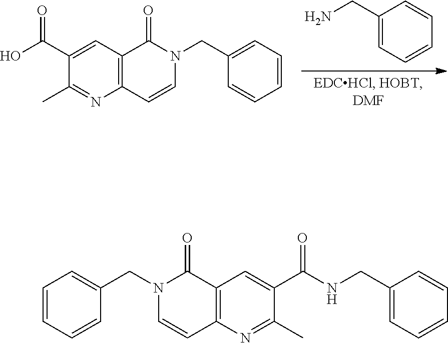 [SMILES: Cc1nc2ccn(Cc3ccccc3)c(=O)c2cc1C(=O)O ; NCc1ccccc1 ; Cc1nc2ccn(Cc3ccccc3)c(=O)c2cc1C(=O)NCc1ccccc1]

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (97.7 mg, 0.510 mmol) and HOBT (68.8 mg, 0.510 mmol), followed by addition of benzylamine (54.6 mg, 0.510 mmol). The reaction mixture was stirred at room temperature for 1 h. Then crude was purified by reverse phase column (gradient: 0˜50% ACN/H₂O (0.5% NH₄HCO₃)) to afford N,6-dibenzyl-2-methyl-5-oxo-5,6-dihydro-1,6-naphthyridine-3-carboxamide as a white solid (32.7 mg, yield: 25%). LCMS (ESI+): m/z calcd. for C₂₄H₂₂N₃O₂ [M+H]⁺, 384.2, found, 384.2. ¹HNMR (400 MHz, DMSO-d₆, δ): 9.16 (t, J=5.8 Hz, 1H), 8.48 (s, 1H), 7.91 (d, J=7.2 Hz, 1H), 7.40-7.26 (m, 10H), 6.72-6.71 (m, 1H), 5.22 (s, 2H), 4.50 (d, J=6.0 Hz, 2H), 2.67 (s, 3H).

### Example 18: 6-benzyl-2-methyl-N-((2-methyloxazol-4-yl)methyl)-5-oxo-5,6-dihydro-1,6-naphthyridine-3-carboxamide

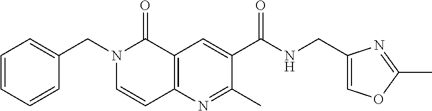 [SMILES: Cc1nc(CNC(=O)c2cc3c(=O)n(Cc4ccccc4)ccc3nc2C)co1]

### 1. Synthesis of 6-benzyl-2-methyl-N-((2-methyloxazol-4-yl)methyl)-5-oxo-5,6-dihydro-1,6-naphthyridine-3-carboxamide

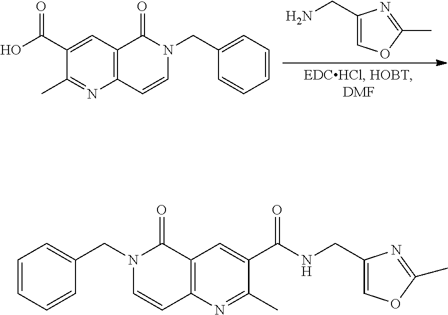

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (79.1 mg, 0.510 mmol) and HOBT (70 mg, 0.518 mmol), followed by addition of (2-methyloxazol-4-yl)methanamine e (57.1 mg, 0.510 mmol). The reaction mixture was stirred at room temperature for 1 h. Then crude was purified by reverse phase column (gradient: 0-50% ACN/H₂O (0.5% FA)) to afford 6-benzyl-2-methyl-N-((2-methyloxazol-4-yl)methyl)-5-oxo-5,6-dihydro-1,6-naphthyridine-3-carboxamide as a yellow solid (12.2 mg, yield: 9.2%). LCMS (ESI+): m/z calcd. for C₂₂H₂₁N₄O₃ [M+H]⁺, 389, found, 389. ¹HNMR (400 MHz, DMSO-d₆, δ): 9.02 (t, J=5.6 Hz, 1H), 8.45 (s, 1H), 7.90 (d, J=7.6 Hz, 1H), 7.85 (s, 1H), 7.35-7.28 (m, 5H), 6.71 (d, J=7.6 Hz, 1H), 5.22 (s, 2H), 4.33-4.31 (m, 2H), 2.66 (s, 3H), 2.40 (s, 3H).

### Example 19: 6-benzyl-2-methyl-N-((5-methyl-1,2,4-oxadiazol-3-yl)methyl)-5-oxo-5,6-dihydro-1,6-naphthyridine-3-carboxamide

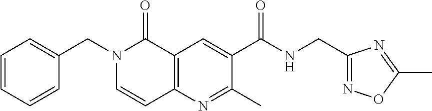 [SMILES: Cc1nc(CNC(=O)c2cc3c(=O)n(Cc4ccccc4)ccc3nc2C)no1]

### 1. Synthesis of 6-benzyl-2-methyl-N-((5-methyl-1,2,4-oxadiazol-3-yl)methyl)-5-oxo-5,6-dihydro-1,6-naphthyridine-3-carboxamide

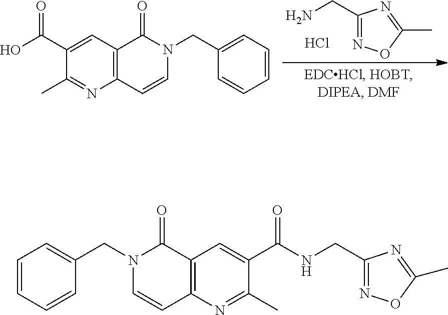

To a solution of 6-benzyl-2-methyl-5-oxo-5,6-dihydro-1,6-naphthyridine-3-carboxylic acid (100 mg, 0.340 mmol) and DIPEA (131.7 mg, 1.020 mmol) in DMF (2 mL) at 0° C. was added EDC·HCl (97.7 mg, 0.510 mmol) and HOBT (68.8 mg, 0.510 mmol), followed by addition of a (5-methyl-1,2,4-oxadiazol-3-yl)methanamine hydrochloride (71 mg, 0.476 mmol). The reaction mixture was stirred at room temperature for 1 h. Then crude was purified by reverse phase column (gradient: 0˜50% MeOH/H₂O (0.5% FA)) to afford 6-benzyl-2-methyl-N-((5-methyl-1,2,4-oxadiazol-3-yl)methyl)-5-oxo-5,6-dihydro-1,6-naphthyridine-3-carboxamide as white solid (15.8 mg, yield: 11.8%). LCMS (ESI+): m/z calcd. for C₂₁H₂₀N₅O₃ [M+H]⁺, 390.2, found, 390.1. ¹HNMR (400 MHz, DMSO-d₆, δ): 9.29 (t, J=5.8 Hz, 1H), 8.50 (s, 1H), 7.92 (d, J=7.6 Hz, 1H), 7.34-7.26 (m, 5H), 6.72 (d, J=7.6 Hz, 1H), 5.22 (s, 2H), 4.57 (d, J=5.6 Hz, 2H), 2.68 (s, 3H), 2.51 (s, 3H).

### Example 20: N-(4-chlorobenzyl)-6-(2-methoxyethyl)-2-methyl-5-oxo-5,6-dihydro-1,6-naphthyridine-3-carboxamide

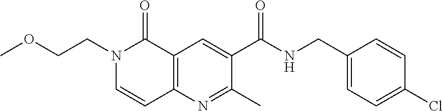 [SMILES: COCCn1ccc2nc(C)c(C(=O)NCc3ccc(Cl)cc3)cc2c1=O]

### 1. Synthesis of ethyl 6-(2-methoxyethyl)-2-methyl-5-oxo-5,6-dihydro-1,6-naphthyridine-3-carboxylate

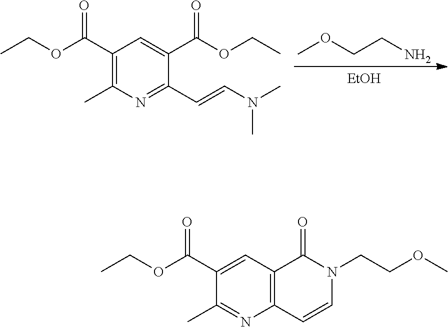

A solution of diethyl (E)-2-(2-(dimethylamino)vinyl)-6-methylpyridine-3,5-dicarboxylate (900 mg, 2.938 mmol) and 2-methoxyethan-1-amine (264.7 mg, 3.526 mmol) in EtOH (9 mL) was stirred at 80° C. for 8 h. The precipitated solids were collected by filtration and washed with water (2×5 mL) to afford ethyl 6-(2-methoxyethyl)-2-methyl-5-oxo-5,6-dihydro-1,6-naphthyridine-3-carboxylate as yellow solid (850 mg, yield: 96.7%). LCMS (ESI+): m/z calcd. for C₁₅H₁₉N₂O₄ [M+H]⁺, 291.1, found, 291.0.

### 2. Synthesis of 6-(2-methoxyethyl)-2-methyl-5-oxo-5,6-dihydro-1,6-naphthyridine-3-carboxylic acid

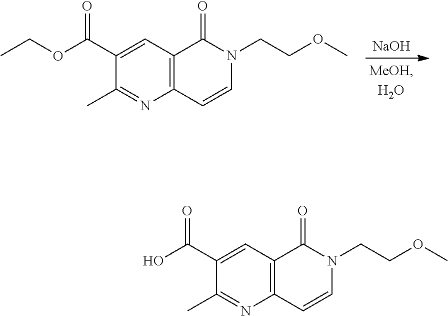

A solution of ethyl 6-(2-methoxyethyl)-2-methyl-5-oxo-5,6-dihydro-1,6-naphthyridine-3-carboxylate (700 mg, 2.411 mmol) and NaOH (144.6 mg, 3.617 mmol) in MeOH (7 mL) and H₂O (2 mL) at room temperature. The resulting mixture was stirred for 3 h at 40° C. The residue was acidified to pH 4 with conc. HCl, and the aqueous layer was extracted with EtOAc. The combined organic layers were dried over Na₂SO₄ and concentrated in vacuo to afford 6-(2-methoxyethyl)-2-methyl-5-oxo-5,6-dihydro-1,6-naphthyridine-3-carboxylic acid as a yellow solid (600 mg, yield: 90.1%). LCMS (ESI+): m/z calcd. for C₁₃H₁₅N₂O₄ [M+H]⁺, 263, found, 263.

### 3. Synthesis of N-(4-chlorobenzyl)-6-(2-methoxyethyl)-2-methyl-5-oxo-5,6-dihydro-1,6-naphthyridine-3-carboxamide

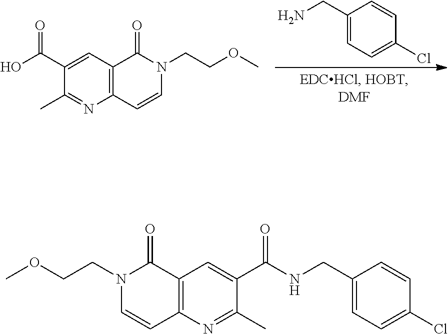 [SMILES: COCCn1ccc2nc(C)c(C(=O)O)cc2c1=O ; NCc1ccc(Cl)cc1 ; COCCn1ccc2nc(C)c(C(=O)NCc3ccc(Cl)cc3)cc2c1=O]

To a solution of 6-(2-methoxyethyl)-2-methyl-5-oxo-5,6-dihydro-1,6-naphthyridine-3-carboxylic acid (200 mg, 0.763 mmol) in DMF (2 mL) at 0° C. was added EDC·HCl (219.2 mg, 1.145 mmol) and HOBT (154.5 mg, 1.145 mmol), followed by addition of 1-(4-chlorophenyl)methanamine (161.9 mg, 1.145 mmol). The reaction mixture was stirred at room temperature for 1 h. Then crude was purified by reverse phase column (gradient: 0-50% ACN/H₂O (0.5% NH₄HCO₃)) to afford N-(4-chlorobenzyl)-6-(2-methoxyethyl)-2-methyl-5-oxo-5,6-dihydro-1,6-naphthyridine-3-carboxamide as a white solid (121.2 mg, yield: 40.8%). LCMS (ESI+): m/z calcd. for C₂₀H₂₁ClN₃O₃[M+H]⁺, 386, found, 386. ¹HNMR (400 MHz, DMSO-d₆, δ): 9.18 (t, J=5.8 Hz, 1H), 8.47 (s, 1H), 7.73 (d, J=7.6 Hz, 1H), 7.45-7.39 (m, 4H), 6.66 (d, J=7.6 Hz, 1H), 4.48 (d, J=6.0 Hz, 2H), 4.18-4.15 (m, 2H), 3.64-3.62 (m, 2H), 3.25 (s, 3H), 2.66 (s, 3H).

### Example 21: 6-(2-methoxyethyl)-2-methyl-N-((2-methyloxazol-5-yl)methyl)-5-oxo-5,6-dihydro-1,6-naphthyridine-3-carboxamide

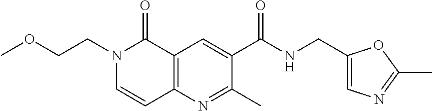

### 1. Synthesis of 6-(2-methoxyethyl)-2-methyl-N-((2-methyloxazol-5-yl)methyl)-5-oxo-5,6-dihydro-1,6-naphthyridine-3-carboxamide

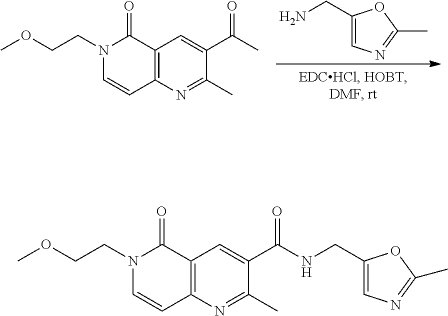

To a solution of 6-(2-methoxyethyl)-2-methyl-5-oxo-5,6-dihydro-1,6-naphthyridine-3-carboxylic acid (150 mg, 0.573 mmol) in DMF (1 mL) at 0° C. was added EDC·HCl (140.5 mg, 0.736 mmol) and HOBT (119.2 mg, 0.883 mmol), followed by addition of a (2-methyloxazol-5-yl)methanamine (76.1 mg, 0.679 mmol). The reaction mixture was stirred at room temperature for 1 h. Then crude was purified by reverse phase column (gradient: 0-50% MeOH/H₂O (0.5% FA)) to afford 6-(2-methoxyethyl)-2-methyl-N-((2-methyloxazol-5-yl)methyl)-5-oxo-5,6-dihydro-1,6-naphthyridine-3-carboxamide as white solid (80.3 mg, yield: 39.3%). LCMS (ESI+): m/z calcd. for C₁₈H₂₁N₄O₄ [M+H]⁺, 357, found, 357. ¹HNMR (400 MHz, DMSO-d₆, δ): 9.12 (t, J=5.6 Hz, 1H), 8.44 (s, 1H), 7.73 (d, J=7.6 Hz, 1H), 6.93 (s, 1H), 6.67-6.65 (m, 1H), 4.51 (d, J=5.6 Hz, 2H), 4.17 (t, J=5.2 Hz, 2H), 3.63 (t, J=5.2 Hz, 2H), 3.25 (s, 3H), 2.66 (s, 3H), 2.40 (s, 3H).

### Example 22: 6-benzyl-N-(4-chlorobenzyl)-2-methyl-5-oxo-5,6-dihydro-1,6-naphthyridine-3-carboxamide

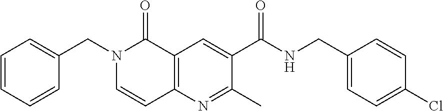 [SMILES: Cc1nc2ccn(Cc3ccccc3)c(=O)c2cc1C(=O)NCc1ccc(Cl)cc1]

### 1. Synthesis of 6-benzyl-N-(4-chlorobenzyl)-2-methyl-5-oxo-5,6-dihydro-1,6-naphthyridine-3-carboxamide

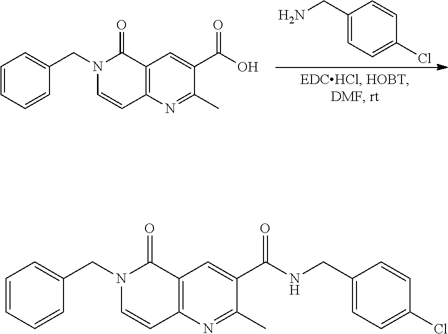 [SMILES: Cc1nc2ccn(Cc3ccccc3)c(=O)c2cc1C(=O)O ; NCc1ccc(Cl)cc1 ; Cc1nc2ccn(Cc3ccccc3)c(=O)c2cc1C(=O)NCc1ccc(Cl)cc1]

To a solution of 6-benzyl-2-methyl-5-oxo-5,6-dihydro-1,6-naphthyridine-3-carboxylic acid (150 mg, 0.510 mmol) in DMF (2 mL) at 0° C. was added EDC·HCl (147 mg, 0.767 mmol) and HOBT (104 mg, 0.770 mmol), followed by addition of 1-(4-chlorophenyl) methanamine (108 mg, 0.763 mmol). The reaction mixture was stirred at room temperature for 1 h. Then crude was purified by reverse phase column (gradient: 0˜50% ACN/H₂O (0.5% NH₄HCO₃)) to afford 6-benzyl-N-(4-chlorobenzyl)-2-methyl-5-oxo-5,6-dihydro-1,6-naphthyridine-3-carboxamide as a white solid (47.2 mg, yield: 22.09%). LCMS (ESI+): m/z calcd. for C₂₄H₂₁ClN₃O₂[M+H]⁺, 418, found, 418. ¹HNMR (400 MHz, DMSO-d₆, δ): 9.18-9.16 (m, 1H), 8.49 (s, 1H), 7.92-7.90 (m, 1H), 7.45-7.29 (m, 9H), 6.73-6.68 (m, 1H), 5.22 (s, 2H), 4.48-4.47 (m, 2H), 2.66 (s, 3H).

### Example 23: 6-benzyl-2-methyl-5-oxo-N-(pyridin-3-ylmethyl)-5,6-dihydro-1,6-naphthyridine-3-carboxamide

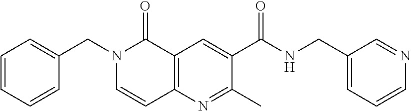 [SMILES: Cc1nc2ccn(Cc3ccccc3)c(=O)c2cc1C(=O)NCc1cccnc1]

1. Synthesis of 6-benzyl-2-methyl-5-oxo-N-(pyridin-3-ylmethyl)-5,6-dihydro-1,6-naphthyridine-3-carbonxmide

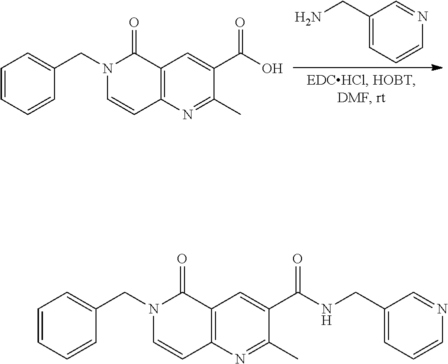 [SMILES: Cc1nc2ccn(Cc3ccccc3)c(=O)c2cc1C(=O)O ; NCc1cccnc1 ; Cc1nc2ccn(Cc3ccccc3)c(=O)c2cc1C(=O)NCc1cccnc1]

To a solution of 6-benzyl-2-methyl-5-oxo-5,6-dihydro-1,6-naphthyridine-3-carboxylic acid (100 mg, 0.340 mmol) and in DMF (2 mL) at 0° C. was added EDC·HCl (98 mg, 0.511 mmol) and HOBT (69 mg, 0.511 mmol), followed by addition of 3-pyridinemethaneamine (56 mg, 0.518 mmol). The reaction mixture was stirred at room temperature for 1 h. Then crude was purified by reverse phase column (gradient: 0˜50% ACN/H₂O (0.5% FA) to afford 6-benzyl-2-methyl-5-oxo-N-(pyridin-3-ylmethyl)-5,6-dihydro-1,6-naphthyridine-3-carboxamide as a white solid (58.9 mg, yield: 44.9%). LCMS (ESI+): m/z calcd. for C₂₃H₂₁N₄O₂ [M+H]⁺, 385.2, found, 385.2. ¹HNMR (400 MHz, DMSO-d₆, δ): 9.22 (t, J=5.8 Hz, 1H), 8.59 (d, J=1.2 Hz, 1H), 8.49 (d, J=3.2 Hz, 2H), 7.91 (d, J=7.6 Hz, 1H), 7.78 (d, J=8.0 Hz, 1H), 7.42-7.39 (m, 1H), 7.36-7.26 (m, 5H), 6.71 (d, J=7.6 Hz, 1H), 5.22 (s, 2H), 4.51 (d, J=5.6 Hz, 2H), 2.66 (s, 3H).

### Example 24: 6-benzyl-N-(4-methoxybenzyl)-2-methyl-5-oxo-5,6-dihydro-1,6-naphthyridine-3-carboxamide

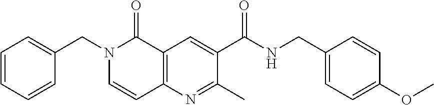 [SMILES: COc1ccc(CNC(=O)c2cc3c(=O)n(Cc4ccccc4)ccc3nc2C)cc1]

### 1. Synthesis of 6-benzyl-N-(4-methoxybenzyl)-2-methyl-5-oxo-5,6-dihydro-1,6-naphthyridine-3-carboxamide

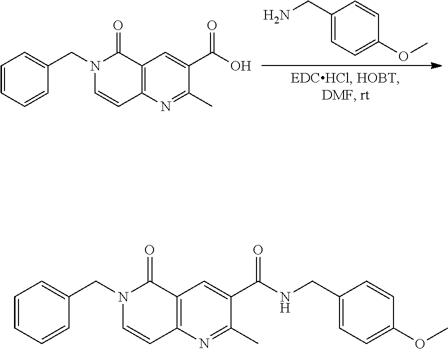

To a solution of 6-benzyl-2-methyl-5-oxo-5,6-dihydro-1,6-naphthyridine-3-carboxylic acid (100 mg, 0.340 mmol) and in DMF (2 mL) at 0° C. was added EDC·HCl (98 mg, 0.631 mmol) and HOBT (69 mg, 0.511 mmol), followed by addition of (4-methoxyphenyl)methanamine (70 mg, 0.510 mmol). The reaction mixture was stirred at room temperature for 1 h. Then crude was purified by reverse phase column (gradient: 0˜50% ACN/H₂O (0.5% FA)) to afford 6-benzyl-N-(4-methoxybenzyl)-2-methyl-5-oxo-5,6-dihydro-1,6-naphthyridine-3-carboxamide as a white solid (47.2 mg, yield: 33.5%). LCMS (ESI+): m/z calcd. for C₂₅H₂₄N₃O₃ [M+H]⁺, 414.2, found, 414.2. ¹HNMR (400 MHz, DMSO-d₆, 6): 9.10 (t, J=5.8 Hz, 1H), 8.44 (s, 1H), 7.91 (d, J=7.6 Hz, 1H), 7.36-7.26 (m, 7H), 6.93 (d, J=8.8 Hz, 2H), 6.71 (d, J=7.6 Hz, 1H), 5.21 (s, 2H), 4.42 (d, J=6.0 Hz, 2H), 3.73 (s, 3H), 2.66 (s, 3H).

### Example 25: 6-benzyl-2-methyl-5-oxo-N-(4-(trifluoromethyl)benzyl)-5,6-dihydro-1,6-naphthyridine-3-carboxamide

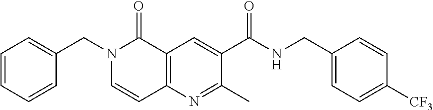 [SMILES: Cc1nc2ccn(Cc3ccccc3)c(=O)c2cc1C(=O)NCc1ccc(C(F)(F)F)cc1]

### 1. Synthesis of 6-benzyl-2-methyl-5-oxo-N-(4-(trifluoromethyl)benzyl)-5,6-dihydro-1,6-naphthyridine-3-carboxamide

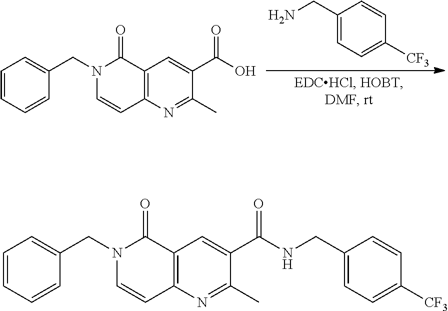 [SMILES: Cc1nc2ccn(Cc3ccccc3)c(=O)c2cc1C(=O)O ; NCc1ccc(C(F)(F)F)cc1 ; Cc1nc2ccn(Cc3ccccc3)c(=O)c2cc1C(=O)NCc1ccc(C(F)(F)F)cc1]

To a solution of 6-benzyl-2-methyl-5-oxo-5,6-dihydro-1,6-naphthyridine-3-carboxylic acid (100 mg, 0.340 mmol) and in DMF (2 mL) at 0° C. was added EDC·HCl (98 mg, 0.511 mmol) and HOBT (69 mg, 0.511 mmol), followed by addition of 1-[4-(trifluoromethyl)phenyl]methanamine (89 mg, 0.508 mmol). The reaction mixture was stirred at room temperature for 1 h. Then crude was purified by reverse phase column (gradient: 0˜50% ACN/H₂O (0.5% FA) to afford 6-benzyl-2-methyl-5-oxo-N-(4-(trifluoromethyl)benzyl)-5,6-dihydro-1,6-naphthyridine-3-carboxamide as a white solid (72.5 mg, yield: 47.1%). LCMS (ESI+): m/z calcd. for C₂₅H₂₁F₃N₃O₂[M+H]⁺, 452.2, found, 452.2. ¹HNMR (400 MHz, DMSO-d₆, δ): 9.28 (t, J=5.8 Hz, 1H), 8.53 (s, 1H), 7.93 (d, J=7.6 Hz, 1H), 7.75 (d, J=8.0 Hz, 2H), 7.59 (d, J=8.0 Hz, 2H), 7.34-7.26 (m, 5H), 6.72 (d, J=7.2 Hz, 1H), 5.22 (s, 2H), 4.58 (d, J=5.6 Hz, 2H), 2.68 (s, 3H).

### Example 26: N-(4-chlorobenzyl)-2-methyl-5-oxo-6-((tetrahydro-2H-pyran-2-yl)methyl)-5,6-dihydro-1,6-naphthyridine-3-carboxamide

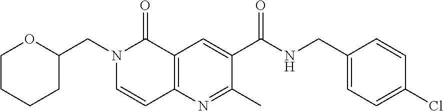 [SMILES: Cc1nc2ccn(CC3CCCCO3)c(=O)c2cc1C(=O)NCc1ccc(Cl)cc1]

### 1. Synthesis of ethyl 2-methyl-5-oxo-6-((tetrahydro-2H-pyran-2-yl)methyl)-5,6-dihydro-1,6-naphthyridine-3-carboxylate

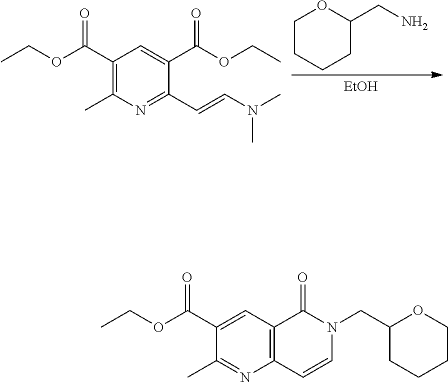

A solution of diethyl (E)-2-(2-(dimethylamino)vinyl)-6-methylpyridine-3,5-dicarboxylate (500 mg, 1.710 mmol) and (tetrahydro-2H-pyran-2-yl)methanamine (230 mg, 1.997 mmol) in EtOH (5 mL) was stirred at 80° C. for 8 h. The precipitated solids were collected by filtration and washed with water (2×5 mL) to afford ethyl 2-methyl-5-oxo-6-((tetrahydro-2H-pyran-2-yl)methyl)-5,6-dihydro-1,6-naphthyridine-3-carboxylate as yellow solid (392 mg, yield: 69.4%). LCMS (ESI+): m/z calcd. for C₁₈H₂₃N₂O₄ [M+H]⁺, 331, found, 331.

### 2. Synthesis of 2-methyl-5-oxo-6-((tetrahydro-2H-pyran-2-yl)methyl)-5,6-dihydro-1,6-naphthyridine-3-carboxylic acid

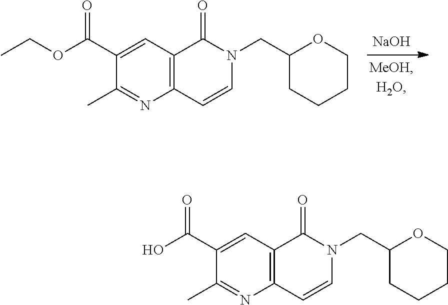 [SMILES: CCOC(=O)c1cc2c(=O)n(CC3CCCCO3)ccc2nc1C ; Cc1nc2ccn(CC3CCCCO3)c(=O)c2cc1C(=O)O]

A solution of ethyl 2-methyl-5-oxo-6-((tetrahydro-2H-pyran-2-yl)methyl)-5,6-dihydro-1,6-naphthyridine-3-carboxylate (350 mg, 1.059 mmol) and sodium hydroxide (127 mg, 3.175 mmol) in MeOH (3 mL) and H₂O (1 mL) was stirred for 3 h at 40° C. The residue was acidified to pH 4 with conc. HCl, and the aqueous layer was extracted with EtOAc. The combined organic layers were dried over Na₂SO₄ and concentrated in vacuum to afford 2-methyl-5-oxo-6-((tetrahydro-2H-pyran-2-yl)methyl)-5,6-dihydro-1,6-naphthyridine-3-carboxylic acid as a white solid (129 mg, yield: 40.8%). LCMS (ESI+): m/z calcd. for C₁₆H₁₉N₂O₄ [M+H]⁺, 303, found, 303.

### 3. Synthesis of N-(4-chlorobenzyl)-2-methyl-5-oxo-6-((tetrahydro-2H-pyran-2-yl)methyl)-5,6-dihydro-1,6-naphthyridine-3-carboxamide

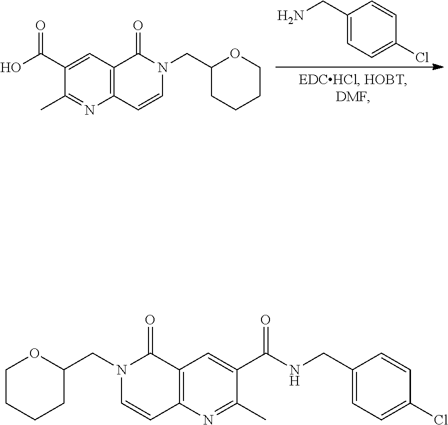 [SMILES: Cc1nc2ccn(CC3CCCCO3)c(=O)c2cc1C(=O)O ; NCc1ccc(Cl)cc1 ; Cc1nc2ccn(CC3CCCCO3)c(=O)c2cc1C(=O)NCc1ccc(Cl)cc1]

To a solution of 2-methyl-5-oxo-6-((tetrahydro-2H-pyran-2-yl)methyl)-5,6-dihydro-1,6-naphthyridine-3-carboxylic acid (120 mg, 0.397 mmol) and in DMF (1.2 mL) at 0° C. was added EDC·HCl (185 mg, 0.965 mmol) and HOBT (86 mg, 0.636 mmol), followed by addition of 1-(4-chlorophenyl)methanamine (88 mg, 0.621 mmol). The reaction mixture was stirred at room temperature for 2 h. Then crude was purified by reverse phase column (gradient: 0˜50% ACN/H₂O (0.5% NH₄HCO₃)) to afford N-(4-chlorobenzyl)-2-methyl-5-oxo-6-((tetrahydro-2H-pyran-2-yl)methyl)-5,6-dihydro-1,6-naphthyridine-3-carboxamide as a white solid (56.8 mg, yield: 33.60%). LCMS (ESI+): m/z calcd. for C₂₃H₂₅ClN₃O₃[M+H]⁺, 426, found, 426. T HNMR (400 MHz, DMSO-d₆, δ): 9.21-9.18 (m, 1H), 8.46 (s, 1H), 7.69 (d, J=7.6 Hz, 1H), 7.45-7.39 (m, 4H), 6.64 (d, J=7.6 Hz, 1H), 4.49 (d, J=6 Hz, 2H), 4.13-4.09 (m, 1H), 3.93-3.84 (m, 2H), 3.63-3.60 (m, 1H), 3.29-3.20 (m, 1H), 2.65 (s, 3H), 1.77 (s, 1H), 1.61 (s, 1H), 1.53-1.50 (m, 3H), 1.21-1.10 (m, 1H).

### Example 27: N-(4-chlorobenzyl)-2-methyl-6-(oxetan-2-ylmethyl)-5-oxo-5,6-dihydro-1,6-naphthyridine-3-carboxamide

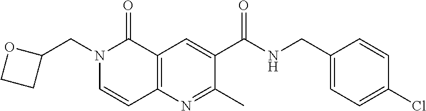 [SMILES: Cc1nc2ccn(CC3CCO3)c(=O)c2cc1C(=O)NCc1ccc(Cl)cc1]

### 1. Synthesis of ethyl 2-methyl-6-(oxetan-2-ylmethyl)-5-oxo-5,6-dihydro-1,6-naphthyridine-3-carboxylate

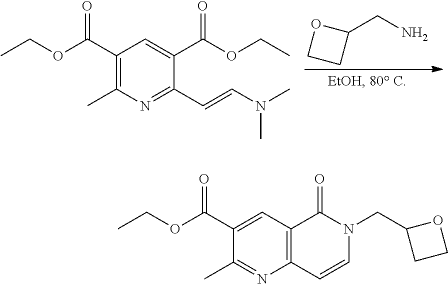

A solution of diethyl (E)-2-(2-(dimethylamino)vinyl)-6-methylpyridine-3,5-dicarboxylate (400 mg, 1.306 mmol) and 1-(oxetan-2-yl)methanamine (208 mg, 2.387 mmol) in EtOH (6 mL) was stirred for overnight at 80° C. under nitrogen atmosphere. The mixture was allowed to cool down to 0° C., the precipitated solids were collected by filtration and washed with H₂O (3×10 mL) to afford ethyl 2-methyl-6-(oxetan-2-ylmethyl)-5-oxo-5,6-dihydro-1,6-naphthyridine-3-carboxylate as an orange solid (350 mg, yield: 88.6%). LCMS (ESI+): m/z calcd. for C₁₆H₁₉N₂O₄ [M+H]⁺, 303.1, found, 303.1.

### 2. Synthesis of 2-methyl-6-(oxetan-2-ylmethyl)-5-oxo-5,6-dihydro-1,6-naphthyridine-3-carboxylic acid

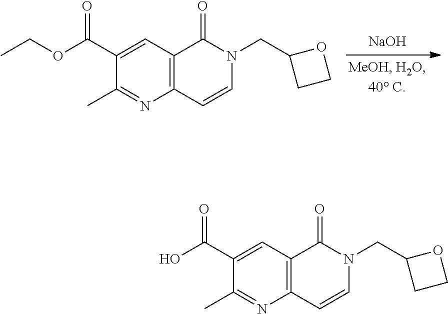 [SMILES: CCOC(=O)c1cc2c(=O)n(CC3CCO3)ccc2nc1C ; Cc1nc2ccn(CC3CCO3)c(=O)c2cc1C(=O)O]

A solution of ethyl 2-methyl-6-(oxetan-2-ylmethyl)-5-oxo-5,6-dihydro-1,6-naphthyridine-3-carboxylate (350 mg, 1.158 mmol) and NaOH (187 mg, 4.675 mmol) in MeOH (3 mL) and H₂O (1 mL) was stirred for overnight at 40° C. The residue was acidified to pH 7 with conc. HCl, and the aqueous layer was extracted with EtOAc. The combined organic layers were dried over Na₂SO₄ and concentrated in vacuo to afford 2-methyl-6-(oxetan-2-ylmethyl)-5-oxo-5,6-dihydro-1,6-naphthyridine-3-carboxylic acid as an orange oil (200 mg, yield: 62.9%). LCMS (ESI+): m/z calcd. for C₁₄H₁₅N₂O₄ [M+H]⁺, 275.1, found, 275.1.

### 3. Synthesis of N-(4-chlorobenzyl)-2-methyl-6-(oxetan-2-ylmethyl)-5-oxo-5,6-dihydro-1,6-naphthyridine-3-carboxamide

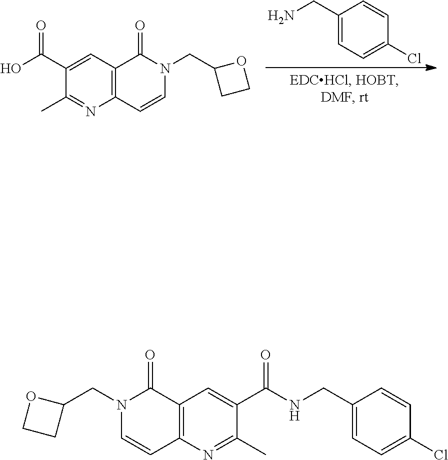 [SMILES: Cc1nc2ccn(CC3CCO3)c(=O)c2cc1C(=O)O ; NCc1ccc(Cl)cc1 ; Cc1nc2ccn(CC3CCO3)c(=O)c2cc1C(=O)NCc1ccc(Cl)cc1]

To a solution of 2-methyl-6-(oxetan-2-ylmethyl)-5-oxo-5,6-dihydro-1,6-naphthyridine-3-carboxylic acid (200 mg, 0.729 mmol) in DMF (2 mL) at 0° C. was added EDC·HCl (211 mg, 1.101 mmol) and HOBT (148 mg, 1.095 mmol), followed by addition of 1-(4-chlorophenyl)methanamine (155 mg, 1.095 mmol). The reaction mixture was stirred at room temperature for 1 h. Then crude was purified by reverse phase column (gradient: 0˜50% ACN/H₂O (0.5% NH₄HCO₃)) to afford N-(4-chlorobenzyl)-2-methyl-6-(oxetan-2-ylmethyl)-5-oxo-5,6-dihydro-1,6-naphthyridine-3-carboxamide as a white solid (188.1 mg, yield: 63.4%). LCMS (ESI+): m/z calcd. for C₂₁H₂₁ClN₃O₃[M+H]⁺, 398.1, found, 398.1. ¹HNMR (400 MHz, DMSO-d₆, δ): 9.19 (d, J=5.6 Hz, 1H), 8.48 (s, 1H), 7.78 (d, J=7.6 Hz, 1H), 7.45-7.38 (m, 4H), 6.68 (d, J=7.6 Hz, 1H), 5.00 (s, 1H), 4.52-4.47 (m, 3H), 4.38-4.31 (m, 2H), 4.23-4.18 (m, 1H), 2.69-2.62 (m, 4H), 2.40-2.35 (m, 1H).

### Example 28: 6-benzyl-2-methyl-5-oxo-N-(1-(pyridin-2-yl)ethyl)-5,6-dihydro-1,6-naphthyridine-3-carboxamide

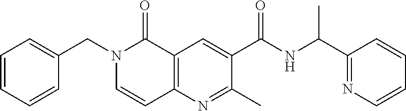 [SMILES: Cc1nc2ccn(Cc3ccccc3)c(=O)c2cc1C(=O)NC(C)c1ccccn1]

### 1. Synthesis of 6-benzyl-2-methyl-5-oxo-N-(1-(pyridin-2-yl)ethyl)-5,6-dihydro-1,6-naphthyridine-3-carboxamide

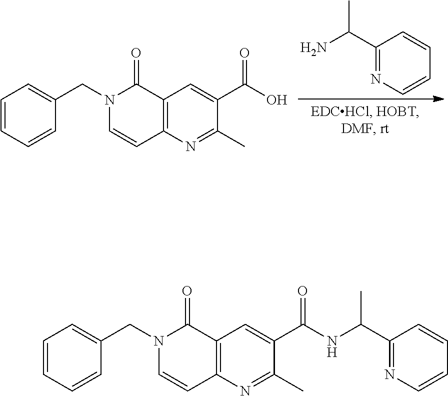

To a solution of 6-benzyl-2-methyl-5-oxo-5,6-dihydro-1,6-naphthyridine-3-carboxylic acid (100 mg, 0.340 mmol) and in DMF (2 mL) at 0° C. was added EDC·HCl (98 mg, 0.511 mmol) and HOBT (69 mg, 0.511 mmol), followed by addition of 1-(pyridin-2-yl)ethanamine (63 mg, 0.516 mmol). The reaction mixture was stirred at room temperature for 1 h. Then crude was purified by reverse phase column (gradient: 0˜50% ACN/H₂O (0.5% NH₄HCO₃) to afford 6-benzyl-2-methyl-5-oxo-N-(1-(pyridin-2-yl)ethyl)-5,6-dihydro-1,6-naphthyridine-3-carboxamide as a white solid (50.2 mg, yield: 37.0%). LCMS (ESI+): m/z calcd. for C₂₄H₂₃N₄O₂ [M+H]⁺, 399.2, found, 399.2. ¹HNMR (400 MHz, DMSO-d6, δ): 9.08 (d, J=8.0 Hz, 1H), 8.56-8.49 (m, 2H), 7.91 (d, J=7.6 Hz, 1H), 7.82-7.78 (m, 1H), 7.45 (d, J=8.0 Hz, 1H), 7.37-7.27 (m, 6H), 6.71 (d, J=7.6 Hz, 1H), 5.22-5.15 (m, 3H), 2.63 (s, 3H), 1.50 (d, J=6.8 Hz, 3H).

### Example 29: 6-benzyl-2-methyl-5-oxo-N-(pyrimidin-2-ylmethyl)-5,6-dihydro-1,6-naphthyridine-3-carboxamide

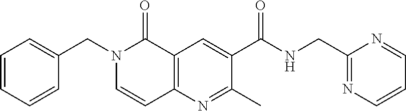 [SMILES: Cc1nc2ccn(Cc3ccccc3)c(=O)c2cc1C(=O)NCc1ncccn1]

### 1. Synthesis of 6-benzyl-2-methyl-5-oxo-N-(pyrimidin-2-ylmethyl)-5,6-dihydro-1,6-naphthyridine-3-carboxamide

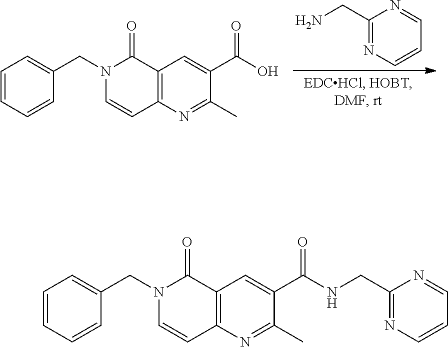 [SMILES: Cc1nc2ccn(Cc3ccccc3)c(=O)c2cc1C(=O)O ; NCc1ncccn1 ; Cc1nc2ccn(Cc3ccccc3)c(=O)c2cc1C(=O)NCc1ncccn1]

To a solution of 6-benzyl-2-methyl-5-oxo-5,6-dihydro-1,6-naphthyridine-3-carboxylic acid (100 mg, 0.340 mmol) and in DMF (2 mL) at 0° C. was added EDC·HCl (98 mg, 0.511 mmol) and HOBT (69 mg, 0.511 mmol), followed by addition of 1-(pyrimidin-2-yl)methanamine (56 mg, 0.513 mmol). The reaction mixture was stirred at room temperature for 1 h. Then crude was purified by reverse phase column (gradient: 0˜50% ACN/H₂O (0.5% NH₄HCO₃) to afford 6-benzyl-2-methyl-5-oxo-N-(pyrimidin-2-ylmethyl)-5,6-dihydro-1,6-naphthyridine-3-carboxamide as a white solid (52.0 mg, yield: 39.2%). LCMS (ESI+): m/z calcd. for C₂₂H₂₀N₅O₂ [M+H]⁺, 386.2, found, 386.2. ¹HNMR (400 MHz, DMSO-d₆, 6): 9.19 (t, J=6.0 Hz, 1H), 8.82 (d, J=4.8 Hz, 2H), 8.60 (d, J=14.8 Hz, 1H), 7.91 (d, J=7.6 Hz, 1H), 7.43-7.26 (m, 6H), 6.72 (d, J=7.6 Hz, 1H), 5.23 (s, 2H), 4.67 (d, J=6.0 Hz, 2H), 2.70 (s, 3H).

### Example 30: 6-(2-methoxyethyl)-2-methyl-5-oxo-N-(pyridin-2-ylmethyl)-5,6-dihydro-1,6-naphthyridine-3-carboxamide

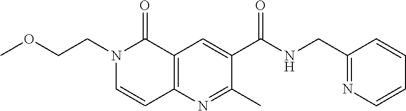 [SMILES: COCCn1ccc2nc(C)c(C(=O)NCc3ccccn3)cc2c1=O]

### 1. Synthesis of ethyl 6-(2-methoxyethyl)-2-methyl-5-oxo-5,6-dihydro-1,6-naphthyridine-3-carboxylate

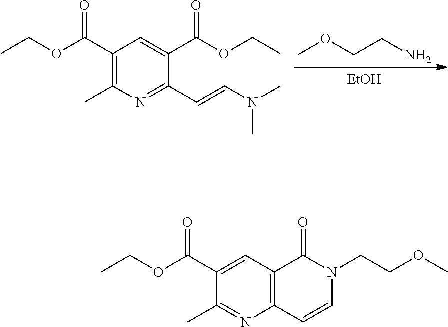

A solution of diethyl (E)-2-(2-(dimethylamino)vinyl)-6-methylpyridine-3,5-dicarboxylate (500 mg, 1.632 mmol) and ethanamine, 2-methoxy- (160 mg, 2.130 mmol) in EtOH (5 mL) was stirred at 80° C. for 8 h. The precipitated solids were collected by filtration and washed with water (2×5 mL) afford ethyl 6-(2-methoxyethyl)-2-methyl-5-oxo-5,6-dihydro-1,6-naphthyridine-3-carboxylate as yellow solid (450 mg, yield: 94.9%). LCMS (ESI+): m/z calcd. for C₁₅H₁₉N₂O₄ [M+H]⁺, 291, found, 291.

### 2. Synthesis of 6-(2-methoxyethyl)-2-methyl-5-oxo-5,6-dihydro-1,6-naphthyridine-3-carboxylic acid

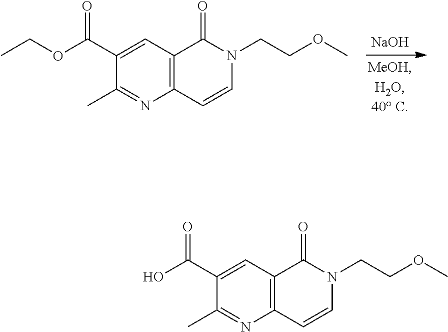

A solution of ethyl 6-(2-methoxyethyl)-2-methyl-5-oxo-5,6-dihydro-1,6-naphthyridine-3-carboxylate (500 mg, 1.722 mmol) and sodium hydroxide (210 mg, 5.250 mmol) in MeOH (4 mL) and H₂O (1.5 mL) was stirred for 8 h at 40° C. The residue was acidified to pH 4 with conc. HCl, and the aqueous layer was extracted with EtOAc. The combined organic layers were dried over Na₂SO₄ and concentrated in vacuo to afford 6-(2-methoxyethyl)-2-methyl-5-oxo-5,6-dihydro-1,6-naphthyridine-3-carboxylic acid as a white solid (340 mg, yield: 5.2%). LCMS (ESI+): m/z calcd. for C₁₃H₁₅N₂O₄ [M+H]⁺, 263, found, 263.

### 3. Synthesis of 6-(2-methoxyethyl)-2-methyl-5-oxo-N-(pyridin-2-ylmethyl)-5,6-dihydro-1,6-naphthyridine-3-carboxamide

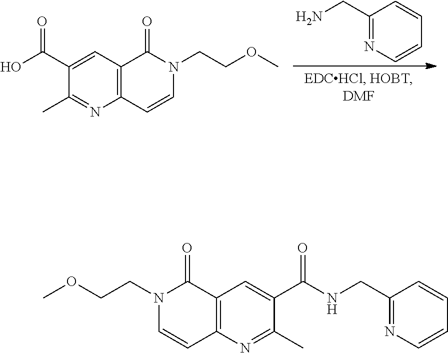

To a solution of 6-(2-methoxyethyl)-2-methyl-5-oxo-5,6-dihydro-1,6-naphthyridine-3-carboxylic acid (115 mg, 0.483 mmol) and in DMF (2 mL) at 0° C. was added EDC·HCl (124 mg, 0.674 mmol) and HOBT (88.0 mg, 0.651 mmol), followed by addition of 2-pyridinemethaneamine (72 mg, 0.666 mmol). The reaction mixture was stirred at room temperature for 2 h. The crude was purified by reverse phase column (gradient: 0˜50% ACN/H₂O (0.5% NH₄HCO₃)) to afford 6-(2-methoxyethyl)-2-methyl-5-oxo-N-(pyridin-2-ylmethyl)-5,6-dihydro-1,6-naphthyridine-3-carboxamide as a white solid (27 mg, yield: 17.47%). LCMS (ESI+): m/z calcd. for C₁₉H₂₁N₄O₃ [M+H]⁺, 353, found, 353. ¹HNMR (400 MHz, DMSO-d₆, δ): 9.25-9.22 (m, 1H), 8.56-8.53 (m, 2H), 7.84-7.80 (m, 1H), 7.74 (d, J=7.6 Hz, 1H), 7.42 (d, J=7.6 Hz, 1H), 7.32-7.29 (m, 1H), 6.68 (d, J=7.6 Hz, 1H), 4.60 (d, J=5.6 Hz, 2H), 4.19-4.16 (m, 2H), 3.65-3.62 (m, 2H), 3.35 (s, 3H), 2.68 (s, 3H).

### Example 31: 6-(4-chlorobenzyl)-2-methyl-5-oxo-N-(pyridin-2-ylmethyl)-5,6-dihydro-1,6-naphthyridine-3-carboxamide

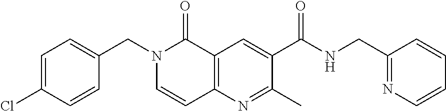 [SMILES: Cc1nc2ccn(Cc3ccc(Cl)cc3)c(=O)c2cc1C(=O)NCc1ccccn1]

### 1. Synthesis of ethyl 6-(4-chlorobenzyl)-2-methyl-5-oxo-5,6-dihydro-1,6-naphthyridine-3-carboxylate

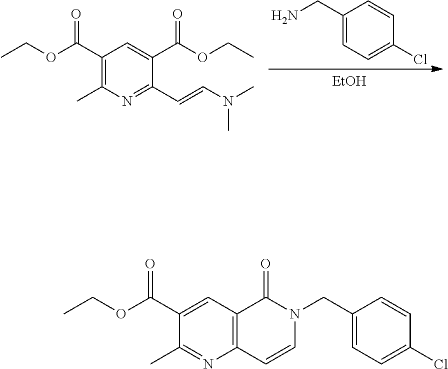 [SMILES: "EtOH" ; CCOC(=O)c1cc(C(=O)OCC)c(/C=C/N(C)C)nc1C ; NCc1ccc(Cl)cc1 ; CCOC(=O)c1cc2c(=O)n(Cc3ccc(Cl)cc3)ccc2nc1C]

A solution of diethyl (E)-2-(2-(dimethylamino)vinyl)-6-methylpyridine-3,5-dicarboxylate (500 mg, 1.632 mmol) and 1-(4-chlorophenyl)methanamine (268 mg, 1.893 mmol) in EtOH (5 mL) was stirred for overnight at 80° C. under nitrogen atmosphere. The mixture was allowed to cool down to 0° C., the precipitated solids were collected by filtration and washed with H₂O (3×10 mL) to afford ethyl 6-(4-chlorobenzyl)-2-methyl-5-oxo-5,6-dihydro-1,6-naphthyridine-3-carboxylate as a yellow solid (270.0 mg, yield: 46.3%). LCMS (ESI+): m/z calcd. for C₁₉H₁₈ClN₂O₃[M+H]⁺, 357, found, 357.

### 2. Synthesis of 6-(4-chlorobenzyl)-2-methyl-5-oxo-5,6-dihydro-1,6-naphthyridine-3-carboxylic acid

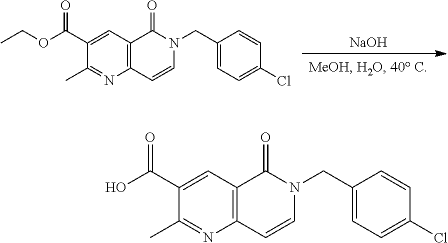 [SMILES: CCOC(=O)c1cc2c(=O)n(Cc3ccc(Cl)cc3)ccc2nc1C ; "NaOH" ; Cc1nc2ccn(Cc3ccc(Cl)cc3)c(=O)c2cc1C(=O)O]

A solution of ethyl 6-(4-chlorobenzyl)-2-methyl-5-oxo-5,6-dihydro-1,6-naphthyridine-3-carboxylate (330 mg, 0.925 mmol) and sodium hydroxide (114 mg, 2.850 mmol) in MeOH (3 mL) and H₂O (1 mL) was stirred for 8 h at 40° C. The residue was acidified to pH 7 with conc. HCl, and the aqueous layer was extracted with EtOAc. The combined organic layers were dried over Na₂SO₄ and concentrated in vacuo to afford 6-(4-chlorobenzyl)-2-methyl-5-oxo-5,6-dihydro-1,6-naphthyridine-3-carboxylic acid as a white solid (240 mg, yield: 78.9%). LCMS (ESI+): m/z calcd. for C₁₇H₁₄ClN₂O₃[M+H]⁺, 329, found, 329.

### 3. Synthesis of 6-(4-chlorobenzyl)-2-methyl-5-oxo-N-(pyridin-2-ylmethyl)-5,6-dihydro-1,6-naphthyridine-3-carboxamide

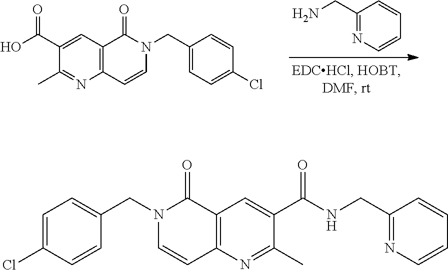 [SMILES: Cc1nc2ccn(Cc3ccc(Cl)cc3)c(=O)c2cc1C(=O)O ; NCc1ccccn1 ; Cc1nc2ccn(Cc3ccc(Cl)cc3)c(=O)c2cc1C(=O)NCc1ccccn1]

To a solution of 6-(4-chlorobenzyl)-2-methyl-5-oxo-5,6-dihydro-1,6-naphthyridine-3-carboxylic acid (122 mg, 0.371 mmol) in DMF (2 mL) at 0° C. was added EDC·HCl (107 mg, 0.558 mmol) and HOBT (76 mg, 0.562 mmol), followed by addition of 2-pyridinemethaneamine (60 mg, 0.555 mmol). The reaction mixture was stirred at room temperature for 1 h. Then crude was purified by reverse phase column (gradient: 0˜50% ACN/H₂O (0.5% NH₄HCO₃)) to afford 6-(4-chlorobenzyl)-2-methyl-5-oxo-N-(pyridin-2-ylmethyl)-5,6-dihydro-1,6-naphthyridine-3-carboxamide as a white solid (75.3 mg, yield: 48.4%). LCMS (ESI+): m/z calcd. for C₂₃H₂₀ClN₄O₂[M+H]⁺, 419.1, found, 419.1. ¹HNMR (400 MHz, DMSO-d₆, δ): 9.24-9.22 (m, 1H), 8.55-8.53 (m, 2H), 7.94 (d, J=7.6 Hz, 1H), 7.83-7.79 (m, 1H), 7.42-7.40 (m, 3H), 7.39-7.28 (m, 2H), 7.33-7.26 (m, 1H), 6.74 (d, J=7.2 Hz, 1H), 5.21 (s, 2H), 4.59 (d, J=6.0 Hz, 2H), 2.69 (s, 3H).

### Example 32: 6-benzyl-2-methyl-5-oxo-N-(pyrazin-2-ylmethyl)-5,6-dihydro-1,6-naphthyridine-3-carboxamide

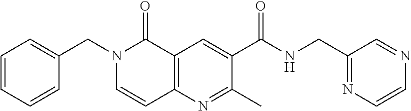 [SMILES: Cc1nc2ccn(Cc3ccccc3)c(=O)c2cc1C(=O)NCc1cnccn1]

### 1. Synthesis of 6-benzyl-2-methyl-5-oxo-N-(pyrazin-2-ylmethyl)-5,6-dihydro-1,6-naphthyridine-3-carboxamide

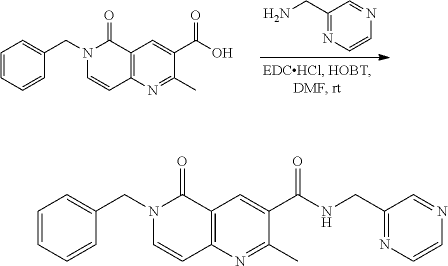 [SMILES: Cc1nc2ccn(Cc3ccccc3)c(=O)c2cc1C(=O)O ; NCc1cnccn1 ; Cc1nc2ccn(Cc3ccccc3)c(=O)c2cc1C(=O)NCc1cnccn1]

To a solution of 6-benzyl-2-methyl-5-oxo-5,6-dihydro-1,6-naphthyridine-3-carboxylic acid (100 mg, 0.340 mmol) and in DMF (4 mL) at 0° C. was added EDC·HCl (98 mg, 0.631 mmol) and HOBT (69 mg, 0.511 mmol), followed by addition of 1-(pyrazin-2-yl)methanamine (56 mg, 0.513 mmol). The reaction mixture was stirred at room temperature for 1 h. Then crude was purified by reverse phase column (gradient: 0˜50% ACN/H₂O (0.5% NH₄HCO₃) to afford 6-benzyl-2-methyl-5-oxo-N-(pyrazin-2-ylmethyl)-5,6-dihydro-1,6-naphthyridine-3-carboxamide as a white solid (67.2 mg, yield: 51.1%). LCMS (ESI+): m/z calcd. for C₂₂H₂₀N₅O₂ [M+H]⁺, 386.2, found, 386.2. ¹HNMR (400 MHz, DMSO-d₆, δ): 9.30 (t, J=5.4 Hz, 1H), 8.71 (s, 1H), 8.63 (s, 1H), 8.56 (d, J=13.2 Hz, 2H), 7.92 (d, J=7.2 Hz, 1H), 7.36-7.28 (m, 5H), 6.72 (d, J=7.6 Hz, 1H), 5.22 (s, 2H), 4.64 (d, J=5.6 Hz, 2H), 2.63 (s, 3H).

### Example 33: 6-benzyl-2-methyl-5-oxo-N-(pyridazin-3-ylmethyl)-5,6-dihydro-1,6-naphthyridine-3-carboxamide

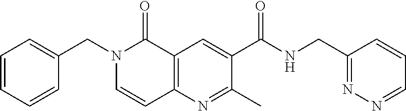 [SMILES: Cc1nc2ccn(Cc3ccccc3)c(=O)c2cc1C(=O)NCc1cccnn1]

### 1. Synthesis of 6-benzyl-2-methyl-5-oxo-N-(pyridazin-3-ylmethyl)-5,6-dihydro-1,6-naphthyridine-3-carboxamide

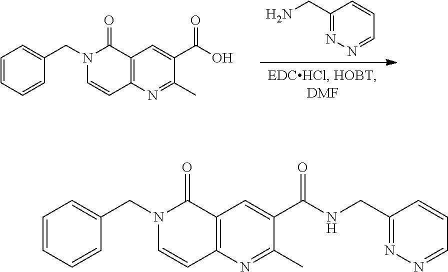 [SMILES: Cc1nc2ccn(Cc3ccccc3)c(=O)c2cc1C(=O)O ; NCc1cccnn1 ; Cc1nc2ccn(Cc3ccccc3)c(=O)c2cc1C(=O)NCc1cccnn1]

To a solution of 6-benzyl-2-methyl-5-oxo-5,6-dihydro-1,6-naphthyridine-3-carboxylic acid (52 mg, 0.177 mmol) in DMF (2 mL) at 0° C. was added EDC·HCl (51 mg, 0.266 mmol) and HOBT (35 mg, 0.259 mmol), followed by addition of 1-(pyridazin-3-yl)methanamine (30 mg, 0.275 mmol). The reaction mixture was stirred at room temperature for 1 h. Then crude was purified by reverse phase column (gradient: 0˜50% ACN/H₂O (0.5% NH₄HCO₃)) to afford 6-benzyl-2-methyl-5-oxo-N-(pyridazin-3-ylmethyl)-5,6-dihydro-1,6-naphthyridine-3-carboxamide as a white solid (8.9 mg, yield: 13.0%). LCMS (ESI+): m/z calcd. for C₂₂H₂₀N₅O₂ [M+H]⁺, 386.2, found, 386.2. ¹HNMR (400 MHz, DMSO-d₆, δ): 9.37-9.25 (m, 1H), 9.17 (s, 1H), 8.56 (s, 1H), 7.93 (d, J=7.6 Hz, 1H), 7.72-7.71 (m, 2H), 7.36-7.28 (m, 5H), 6.73 (d, J=6.4 Hz, 1H), 5.22 (s, 2H), 4.78 (d, J=6.0 Hz, 2H), 2.67 (s, 3H).

### Example 34: 6-benzyl-2-methyl-5-oxo-N-(pyrimidin-5-ylmethyl)-5,6-dihydro-1,6-naphthyridine-3-carboxamide

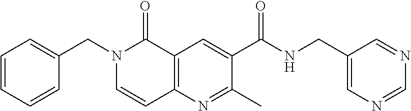 [SMILES: Cc1nc2ccn(Cc3ccccc3)c(=O)c2cc1C(=O)NCc1cncnc1]

### 1. Synthesis of 6-benzyl-2-methyl-5-oxo-N-(pyrimidin-5-ylmethyl)-5,6-dihydro-1,6-naphthyridine-3-carboxamide

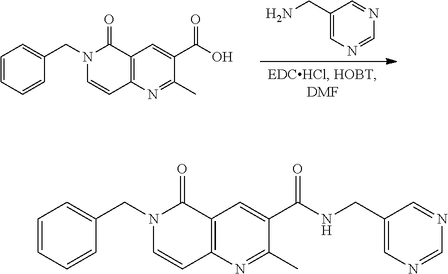 [SMILES: Cc1nc2ccn(Cc3ccccc3)c(=O)c2cc1C(=O)O ; NCc1cncnc1 ; Cc1nc2ccn(Cc3ccccc3)c(=O)c2cc1C(=O)NCc1cncnc1]

To a solution of 6-benzyl-2-methyl-5-oxo-5,6-dihydro-1,6-naphthyridine-3-carboxylic acid (51 mg, 0.173 mmol) in DMF (1.5 mL) at 0° C. was added EDC·HCl (48 mg, 0.250 mmol) and HOBT (34 mg, 0.252 mmol), followed by addition of 4-pyridinemethaneamine (56 mg, 0.518 mmol). The reaction mixture was stirred at room temperature for 1 h. Then crude was purified by reverse phase column (gradient: 0˜50% ACN/H₂O (0.5% NH₄HCO₃)) to afford 6-benzyl-2-methyl-5-oxo-N-(pyrimidin-5-ylmethyl)-5,6-dihydro-1,6-naphthyridine-3-carboxamide as a white solid (16.7 mg, yield: 25.0%). LCMS (ESI+): m/z calcd. for C₂₂H₂₀N₅O₂ [M+H]⁺, 386.2, found, 386.2. ¹HNMR (400 MHz, DMSO-d₆, δ): 9.24-9.23 (m, 1H), 9.12 (s, 1H), 8.83 (s, 2H), 8.52 (s, 1H), 7.93 (d, J=7.6 Hz, 1H), 7.36-7.30 (m, 5H), 6.73 (d, J=7.6 Hz, 1H), 5.22 (s, 2H), 4.53 (d, J=5.6 Hz, 2H), 2.72 (s, 3H).

### Example 35: 6-benzyl-N-((5-fluoropyridin-2-yl)methyl)-2-methyl-5-oxo-5,6-dihydro-1,6-naphthyridine-3-carboxamide

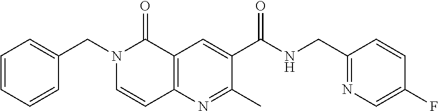 [SMILES: Cc1nc2ccn(Cc3ccccc3)c(=O)c2cc1C(=O)NCc1ccc(F)cn1]

### 1. Synthesis of 6-benzyl-N-((5-fluoropyridin-2-yl)methyl)-2-methyl-5-oxo-5,6-dihydro-1,6-naphthyridine-3-carboxamide

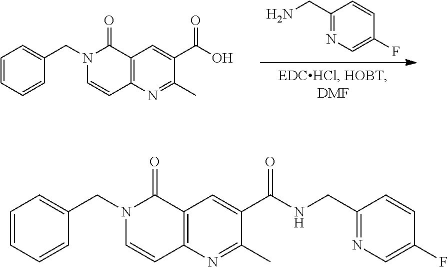 [SMILES: Cc1nc2ccn(Cc3ccccc3)c(=O)c2cc1C(=O)O ; NCc1ccc(F)cn1 ; Cc1nc2ccn(Cc3ccccc3)c(=O)c2cc1C(=O)NCc1ccc(F)cn1]

To a solution of 6-benzyl-2-methyl-5-oxo-5,6-dihydro-1,6-naphthyridine-3-carboxylic acid (51 mg, 0.173 mmol) in DMF (2 mL) at 0° C. was added EDC·HCl (48 mg, 0.250 mmol) and HOBT (35 mg, 0.259 mmol), followed by addition of 1-(5-fluoropyridin-2-yl)methanamine (32 mg, 0.254 mmol). The reaction mixture was stirred at room temperature for 1 h. Then crude was purified by reverse phase column (gradient: 0˜50% ACN/H₂O (0.5% NH₄HCO₃)) to afford 6-benzyl-N-((5-fluoropyridin-2-yl)methyl)-2-methyl-5-oxo-5,6-dihydro-1,6-naphthyridine-3-carboxamide as a white solid (13.5 mg, yield: 19.4%). LCMS (ESI+): m/z calcd. for C₂₃H₂₀FN₄O₂[M+H]⁺, 403.2, found, 403.2. ¹HNMR (400 MHz, DMSO-d₆, δ): 9.26-9.24 (m, 1H), 8.55-8.53 (m, 2H), 7.92 (d, J=7.6, 1H), 7.72-7.70 (m, 1H), 7.50 (d, J=2.4, 1H), 7.49-7.23 (m, 5H), 6.73 (d, J=7.6 Hz, 1H), 5.22 (s, 2H), 4.58 (d, J=5.6 Hz, 2H), 2.68 (s, 3H).

### Example 36: 6-benzyl-N-((3-fluoropyridin-2-yl)methyl)-2-methyl-5-oxo-5,6-dihydro-1,6-naphthyridine-3-carboxamide

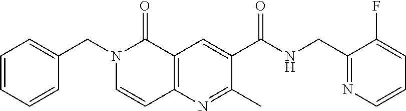 [SMILES: Cc1nc2ccn(Cc3ccccc3)c(=O)c2cc1C(=O)NCc1ncccc1F]

### 1. Synthesis of 6-benzyl-N-((3-fluoropyridin-2-yl)methyl)-2-methyl-5-oxo-5,6-dihydro-1,6-naphthyridine-3-carboxamide

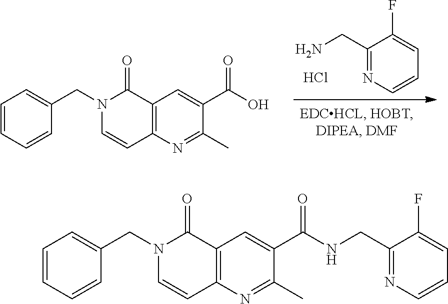 [SMILES: Cc1nc2ccn(Cc3ccccc3)c(=O)c2cc1C(=O)O ; Cl ; NCc1ncccc1F ; Cc1nc2ccn(Cc3ccccc3)c(=O)c2cc1C(=O)NCc1ncccc1F]

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (102 mg, 0.532 mmol) and HOBT (70 mg, 0.518 mmol) and DIEA (78 mg, 0.603 mmol), followed by addition of 1-(3-fluoropyridin-2-yl)methanamine (83 mg, 0.658 mmol). The reaction mixture was stirred at room temperature for 1 h. Then crude was purified by reverse phase column (gradient: 0-50% ACN/H₂O (0.5% NH₄HCO₃)) to afford 6-benzyl-N-((3-fluoropyridin-2-yl)methyl)-2-methyl-5-oxo-5,6-dihydro-1,6-naphthyridine-3-carboxamide as a white solid (52.6 mg, yield: 38.47%). LCMS (ESI+): m/z calcd. for C₂₃H₂₀FN₄O₂[M+H]⁺, 403.2, found, 403.2. ¹HNMR (400 MHz, DMSO-d₆, δ): 9.17-9.14 (m, 1H), 8.50 (s, 1H), 8.43 (d, J=3.6 Hz, 1H), 7.92 (s, 1H), 7.76-7.71 (m, 1H), 7.45-7.41 (m, 1H), 7.36-7.27 (m, 5H), 6.73 (d, J=7.2 Hz, 1H), 5.22 (s, 2H), 4.67 (d, J=5.6 Hz, 2H), 2.66 (s, 3H).

### Example 37: 2-methyl-5-oxo-6-(1-phenylethyl)-N-(pyridin-2-ylmethyl)-5,6-dihydro-1,6-naphthyridine-3-carboxamide

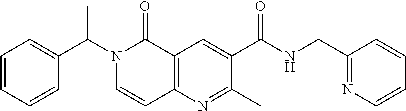 [SMILES: Cc1nc2ccn(C(C)c3ccccc3)c(=O)c2cc1C(=O)NCc1ccccn1]

### 1. Synthesis of ethyl 2-methyl-5-oxo-6-(1-phenylethyl)-5,6-dihydro-1,6-naphthyridine-3-carboxylate

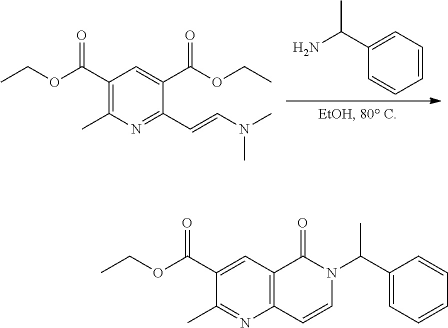

A solution of diethyl (E)-2-(2-(dimethylamino)vinyl)-6-methylpyridine-3,5-dicarboxylate (250 mg, 0.816 mmol) and (+/−)-α-methylbenzylamine (145 mg, 1.197 mmol) in EtOH (3 mL) was stirred for overnight at 80° C. under nitrogen atmosphere. The mixture was allowed to cool down to 0° C., the precipitated solids were collected by filtration and washed with H₂O (3×10 mL) to afford ethyl 2-methyl-5-oxo-6-(1-phenylethyl)-5,6-dihydro-1,6-naphthyridine-3-carboxylate as a brown solid (200 mg, yield: 72.8%). LCMS (ESI+): m/z calcd. for C₂₀H₂₁N₂O₃ [M+H]⁺, 337.2, found, 337.1.

### 2. Synthesis of 2-methyl-5-oxo-6-(1-phenylethyl)-5,6-dihydro-1,6-naphthyridine-3-carboxylic acid

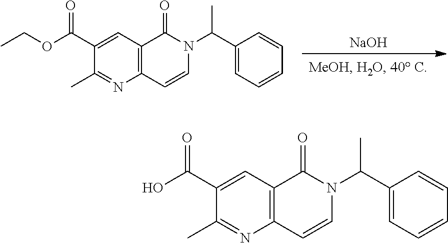 [SMILES: CCOC(=O)c1cc2c(=O)n(C(C)c3ccccc3)ccc2nc1C ; "NaOH" ; Cc1nc2ccn(C(C)c3ccccc3)c(=O)c2cc1C(=O)O]

A solution of ethyl 2-methyl-5-oxo-6-(1-phenylethyl)-5,6-dihydro-1,6-naphthyridine-3-carboxylate (200 mg, 0.595 mmol) and NaOH (90 mg, 2.250 mmol) in MeOH (3 mL) and H₂O (1 mL) was stirred for overnight at 40° C. The residue was acidified to pH 7 with conc. HCl, and the aqueous layer was extracted with EtOAc. The combined organic layers were dried over Na₂SO₄ and concentrated in vacuo to afford 2-methyl-5-oxo-6-(1-phenylethyl)-5,6-dihydro-1,6-naphthyridine-3-carboxylic acid as a brown oil (138 mg, yield: 75.2%). LCMS (ESI+): m/z calcd. for C₁₈H₁₇N₂O₃ [M+H]⁺, 309.1, found, 309.1.

### 3. Synthesis of 2-methyl-5-oxo-6-(I-phenylethyl)-N-(pyridin-2-ylmethyl)-5,6-dihydro-1,6-naphthyridine-3-carboxamide

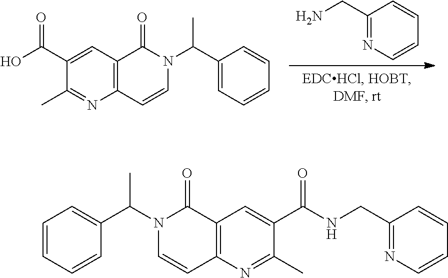 [SMILES: Cc1nc2ccn(C(C)c3ccccc3)c(=O)c2cc1C(=O)O ; NCc1ccccn1 ; Cc1nc2ccn(C(C)c3ccccc3)c(=O)c2cc1C(=O)NCc1ccccn1]

To a solution of 2-methyl-5-oxo-6-(1-phenylethyl)-5,6-dihydro-1,6-naphthyridine-3-carboxylic acid (140 mg, 0.454 mmol) in DMF (2 mL) at 0° C. was added EDC·HCl (131 mg, 0.683 mmol) and HOBT (92 mg, 0.681 mmol), followed by addition of 2-pyridinemethaneamine (74 mg, 0.684 mmol). The reaction mixture was stirred at room temperature for 1 h. Then crude was purified by reverse phase column (gradient: 0˜50% ACN/H₂O (0.5% NH₄HCO₃)) to afford 2-methyl-5-oxo-6-(1-phenylethyl)-N-(pyridin-2-ylmethyl)-5,6-dihydro-1,6-naphthyridine-3-carboxamide as a white solid (59.4 mg, yield: 32.8%). LCMS (ESI+): m/z calcd. for C₂₄H₂₃N₄O₂ [M+H]⁺, 399.2, found, 399.2. ¹HNMR (400 MHz, DMSO-d₆, δ): 9.24 (t, J=5.8 Hz, 1H), 8.55 (d, J=5.6 Hz, 2H), 7.82 (t, J=7.8 Hz, 1H), 7.73 (d, J=8.0 Hz, 1H), 7.42-7.31 (m, 5H), 7.29 (d, J=6.4 Hz, 2H), 6.70 (d, J=7.6 Hz, 1H), 6.29-6.23 (m, 1H), 4.59 (d, J=5.6 Hz, 2H), 2.68 (s, 3H), 1.78 (d, J=7.2 Hz, 3H).

### Example 38: 6-benzyl-N-((5-chloropyridin-2-yl)methyl)-2-methyl-5-oxo-5,6-dihydro-1,6-naphthyridine-3-carboxamide

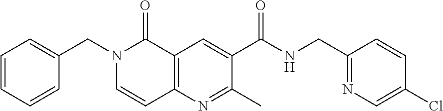 [SMILES: Cc1nc2ccn(Cc3ccccc3)c(=O)c2cc1C(=O)NCc1ccc(Cl)cn1]

### 1. Synthesis of 6-benzyl-N-((5-chloropyridin-2-yl)methyl)-2-methyl-5-oxo-5,6-dihydro-1,6-naphthyridine-3-carboxamide

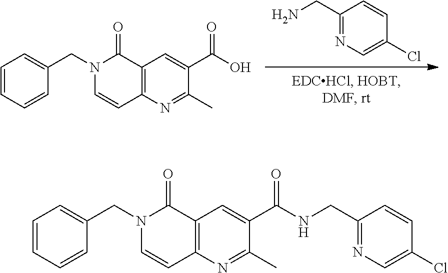 [SMILES: Cc1nc2ccn(Cc3ccccc3)c(=O)c2cc1C(=O)O ; NCc1ccc(Cl)cn1 ; Cc1nc2ccn(Cc3ccccc3)c(=O)c2cc1C(=O)NCc1ccc(Cl)cn1]

To a solution of 6-benzyl-2-methyl-5-oxo-5,6-dihydro-1,6-naphthyridine-3-carboxylic acid (50 mg, 0.170 mmol) and in DMF (4 mL) at 0° C. was added EDC·HCl (49 mg, 0.316 mmol) and HOBT (35 mg, 0.259 mmol), followed by addition of 1-(5-chloropyridin-2-yl)methanamine (37 mg, 0.259 mmol). The reaction mixture was stirred at room temperature for 1 h. Then crude was purified by reverse phase column (gradient: 0˜50% ACN/H₂O (0.5% NH₄HCO₃) to afford 6-benzyl-N-((5-chloropyridin-2-yl)methyl)-2-methyl-5-oxo-5,6-dihydro-1,6-naphthyridine-3-carboxamide as a white solid (34.3 mg, yield: 47.7%). LCMS (ESI+): m/z calcd. for C₂₃H₂₀ClN₄O₂[M+H]⁺, 419.1, found, 419.1. ¹HNMR (400 MHz, DMSO-d₆, δ): 9.27 (s, 1H), 8.59 (s, 1H), 8.54 (s, 1H), 7.96-7.91 (m, 2H), 7.46 (d, J=8.4 Hz, 1H), 7.35-7.28 (m, 5H), 6.72 (d, J=7.6 Hz, 1H), 5.22 (s, 2H), 4.57 (d, J=6.0 Hz, 2H), 2.67 (s, 3H).

### Example 39: 6-benzyl-N-((4-fluoropyridin-2-yl)methyl)-2-methyl-5-oxo-5,6-dihydro-1,6-naphthyridine-3-carboxamide

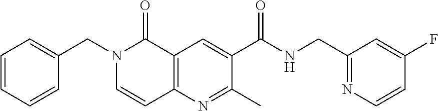 [SMILES: Cc1nc2ccn(Cc3ccccc3)c(=O)c2cc1C(=O)NCc1cc(F)ccn1]

### 1. Synthesis of 6-benzyl-N-((4-fluoropyridin-2-yl)methyl)-2-methyl-5-oxo-5,6-dihydro-1,6-naphthyridine-3-carboxamide

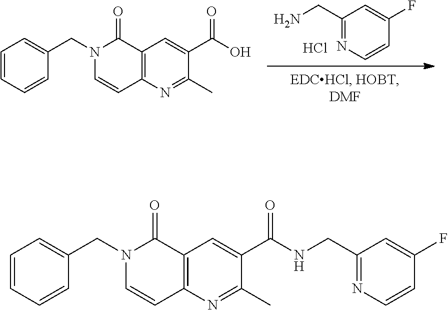 [SMILES: Cc1nc2ccn(Cc3ccccc3)c(=O)c2cc1C(=O)O ; NCc1cc(F)ccn1 ; Cl ; Cc1nc2ccn(Cc3ccccc3)c(=O)c2cc1C(=O)NCc1cc(F)ccn1]

To a solution of 6-benzyl-2-methyl-5-oxo-5,6-dihydro-1,6-naphthyridine-3-carboxylic acid (65 mg, 0.221 mmol) in DMF (1.8 mL) at 0° C. was added EDC·HCl (60 mg, 0.313 mmol) and HOBT (42 mg, 0.311 mmol) and DIPEA (45 mg, 0.348 mmol), followed by addition of 1-(4-fluoropyridin-2-yl)methanamine (52 mg, 0.412 mmol). The reaction mixture was stirred at room temperature for 1 h. Then crude was purified by reverse phase column (gradient: 0-50% ACN/H₂O (0.5% NH₄HCO₃)) to afford 6-benzyl-N-((4-fluoropyridin-2-yl)methyl)-2-methyl-5-oxo-5,6-dihydro-1,6-naphthyridine-3-carboxamide as a white solid (18.0 mg, yield: 20.2%). LCMS (ESI+): m/z calcd. for C₂₃H₂₀FN₄O₂[M+H]⁺, 403.2, found, 403.2. ¹HNMR (400 MHz, DMSO-d₆, δ): 9.25 (m, 1H), 8.65-8.57 (m, 2H), 7.94 (s, 1H), 7.29-7.23 (m, 7H), 6.75 (s, 1H), 5.22 (s, 2H), 4.61 (d, J=6.0 Hz, 2H), 2.80 (s, 3H).

### Example 40: 6-(cyclohexylmethyl)-2-methyl-5-oxo-N-(pyridin-2-ylmethyl)-5,6-dihydro-1,6-naphthyridine-3-carboxamide

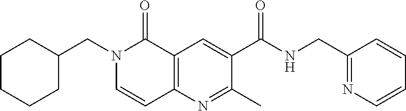 [SMILES: Cc1nc2ccn(CC3CCCCC3)c(=O)c2cc1C(=O)NCc1ccccn1]

### 1. Synthesis of ethyl 6-(cyclohexylmethyl)-2-methyl-5-oxo-5,6-dihydro-1,6-naphthyridine-3-carboxylate

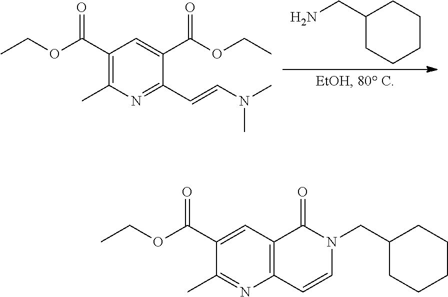

A solution of diethyl (E)-2-(2-(dimethylamino)vinyl)-6-methylpyridine-3,5-dicarboxylate (200 mg, 0.653 mmol) and (aminomethyl)cyclohexane (135 mg, 1.193 mmol) in EtOH (3 mL) was stirred for overnight at 80° C. under nitrogen atmosphere. The mixture was allowed to cool down to 0° C., the precipitated solids were collected by filtration and washed with H₂O (3×10 mL) to afford ethyl 6-(cyclohexylmethyl)-2-methyl-5-oxo-5,6-dihydro-1,6-naphthyridine-3-carboxylate as a brown solid (190 mg, yield: 88.6%). LCMS (ESI+): m/z calcd. for C₁₉H₂₅N₂O₃ [M+H]⁺, 329.2, found, 329.2.

### 2. Synthesis of 6-(cyclohexylmethyl)-2-methyl-5-oxo-5,6-dihydro-1,6-naphthyridine-3-carboxylic acid

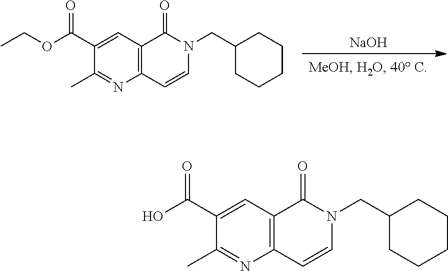 [SMILES: CCOC(=O)c1cc2c(=O)n(CC3CCCCC3)ccc2nc1C ; "NaOH" ; Cc1nc2ccn(CC3CCCCC3)c(=O)c2cc1C(=O)O]

A solution of ethyl 6-(cyclohexylmethyl)-2-methyl-5-oxo-5,6-dihydro-1,6-naphthyridine-3-carboxylate (190 mg, 0.579 mmol) and NaOH (70 mg, 1.750 mmol) in MeOH (3 mL) and H₂O (1 mL) was stirred for overnight at 40° C. The residue was acidified to pH 7 with conc. HCl, and the aqueous layer was extracted with EtOAc. The combined organic layers were dried over Na₂SO₄ and concentrated in vacuo to afford 6-(cyclohexylmethyl)-2-methyl-5-oxo-5,6-dihydro-1,6-naphthyridine-3-carboxylic acid as a white solid (170 mg, yield: 97.8%). LCMS (ESI+): m/z calcd. for C₁₇H₂₁N₂O₃ [M+H]⁺, 301.2, found, 301.1.

### 3. Synthesis of 6-(cyclohexylmethyl)-2-methyl-5-oxo-N-(pyridin-2-ylmethyl)-5,6-dihydro-1,6-naphthyridine-3-carboxamide

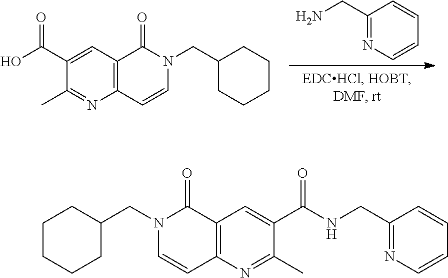 [SMILES: Cc1nc2ccn(CC3CCCCC3)c(=O)c2cc1C(=O)O ; NCc1ccccn1 ; Cc1nc2ccn(CC3CCCCC3)c(=O)c2cc1C(=O)NCc1ccccn1]

To a solution of 6-(cyclohexylmethyl)-2-methyl-5-oxo-5,6-dihydro-1,6-naphthyridine-3-carboxylic acid (130 mg, 0.433 mmol) in DMF (2 mL) at 0° C. was added EDC·HCl (125 mg, 0.652 mmol) and HOBT (88 mg, 0.651 mmol), followed by addition of 2-pyridinemethaneamine (71 mg, 0.657 mmol). The reaction mixture was stirred at room temperature for 1 h. Then crude was purified by reverse phase column (gradient: 0˜50% ACN/H₂O (0.5% NH₄HCO₃)) to afford 6-(cyclohexylmethyl)-2-methyl-5-oxo-N-(pyridin-2-ylmethyl)-5,6-dihydro-1,6-naphthyridine-3-carboxamide as a white solid (59.8 mg, yield: 35.2%). LCMS (ESI+): m/z calcd. for C₂₃H₂₇N₄O₂ [M+H]⁺, 391.2, found, 391.2. ¹HNMR (400 MHz, DMSO-d₆, δ): 9.22 (d, J=6.0 Hz, 1H), 8.55-8.52 (t, J=7.0, 2H), 7.83-7.81 (m, 1H), 7.79-7.73 (m, 1H), 7.41 (d, J=7.6 Hz, 1H), 7.31-7.28 (m, 1H), 6.65 (d, J=7.2 Hz, 1H), 4.58 (d, J=6.0 Hz, 2H), 3.84 (d, J=7.2 Hz, 2H), 2.51-2.49 (m, 3H), 1.84-1.75 (m, 1H), 1.67-1.60 (m, 2H), 1.57-1.54 (m, 3H), 1.15 (s, 3H), 1.04-0.96 (m, 2H).

### Example 41: 2-methyl-5-oxo-N,6-bis(pyridin-2-ylmethyl)-5,6-dihydro-1,6-naphthyridine-3-carboxamide

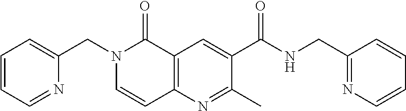 [SMILES: Cc1nc2ccn(Cc3ccccn3)c(=O)c2cc1C(=O)NCc1ccccn1]

### 1. Synthesis of ethyl 2-methyl-5-oxo-6-(pyridin-2-ylmethyl)-5,6-dihydro-1,6-naphthyridine-3-carboxylate

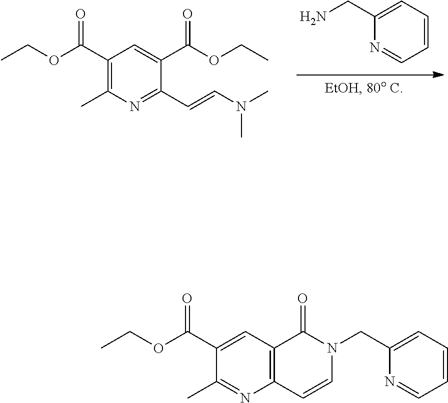 [SMILES: CCOC(=O)c1cc(C(=O)OCC)c(/C=C/N(C)C)nc1C ; NCc1ccccn1 ; "EtOH, 80° C." ; CCOC(=O)c1cc2c(=O)n(Cc3ccccn3)ccc2nc1C]

A solution of diethyl (E)-2-(2-(dimethylamino)Vinyl)-6-methylpyridine-3,5-dicarboxylate (250 mg, 0.816 mmol) and 2-pyridinemethaneamine (106 mg, 0.980 mmol) in EtOH (2 mL) was stirred for overnight at 80 0C under nitrogen atmosphere. The mixture was allowed to cool down to 0° C., the precipitated solids were collected by filtration and washed with H₂O (3×10 mL) to afford ethyl 2-methyl-5-oxo-6-(pyridin-2-ylmethyl)-5,6-dihydro-1,6-naphthyridine-3-carboxylate as a yellow solid (200 mg, yield: 75.8%). LCMS (ESI+): m/z calcd. for C₁₈H₁₈N₃O₃ [M+H]⁺, 324.1, found, 324.1.

### 2. Synthesis of 2-methyl-5-oxo-6-(pyridin-2-ylmethyl)-5,6-dihydro-1,6-naphthyridine-3-carboxylic acid

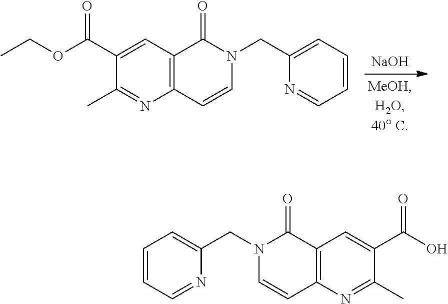

A solution of ethyl 2-methyl-5-oxo-6-(pyridin-2-ylmethyl)-5,6-dihydro-1,6-naphthyridine-3-carboxylate (400 mg, 1.237 mmol) and NaOH (150 mg, 3.750 mmol) in MeOH (3 mL) and H₂O (1 mL) was stirred for overnight at 40° C. The residue was acidified to pH 7 with conc. HCl, and the aqueous layer was extracted with EtOAc. The combined organic layers were dried over Na₂SO₄ and concentrated in vacuo to afford 2-methyl-5-oxo-6-(pyridin-2-ylmethyl)-5,6-dihydro-1,6-naphthyridine-3-carboxylic acid as a yellow solid (300 mg, yield: 82.1%). LCMS (ESI+): m/z calcd. for C₁₆H₁₃N₃O₃ [M+H]⁺, 296.1, found, 296.1.

### 3. Synthesis of 2-methyl-5-oxo-N, 6-bis(pyridin-2-ylmethyl)-5,6-dihydro-1,6-naphthyridine-3-carboxamide

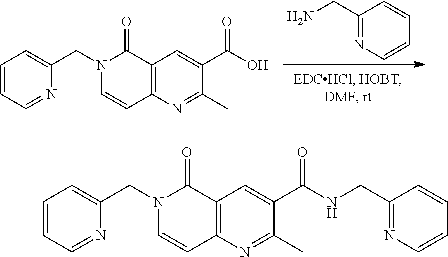 [SMILES: Cc1nc2ccn(Cc3ccccn3)c(=O)c2cc1C(=O)O ; NCc1ccccn1 ; Cc1nc2ccn(Cc3ccccn3)c(=O)c2cc1C(=O)NCc1ccccn1]

To a solution of 2-methyl-5-oxo-6-(pyridin-2-ylmethyl)-5,6-dihydro-1,6-naphthyridine-3-carboxylic acid (150 mg, 0.508 mmol) in DMF (2 mL) at 0° C. was added EDC·HCl (147 mg, 0.767 mmol) and HOBT (103 mg, 0.762 mmol), followed by addition of 2-pyridinemethaneamine (83 mg, 0.767 mmol). The reaction mixture was stirred at room temperature for 1 h. Then crude was purified by reverse phase column (gradient: 0˜50% ACN/H₂O (0.5% NH₄HCO₃)) to afford 2-methyl-5-oxo-N,6-bis(pyridin-2-ylmethyl)-5,6-dihydro-1,6-naphthyridine-3-carboxamide as a white solid (17.9 mg, yield: 8.9%). LCMS (ESI+): m/z calcd. for C₂₂H₂₀N₅O₂ [M+H]⁺, 386.2, found, 386.2. ¹HNMR (400 MHz, DMSO-d₆, δ): 9.22 (d, J=6.0 Hz, 1H), 8.54-8.47 (m, 3H), 7.90 (d, J=7.6 Hz, 1H), 7.82-7.75 (m, 2H), 7.39 (d, J=7.6 Hz, 1H), 7.30-7.27 (m, 3H), 6.73 (d, J=7.6 Hz, 1H), 5.31 (s, 2H), 4.58 (d, J=6.0 Hz, 2H), 2.69 (s, 3H).

### Example 42: 6-benzyl-N,2-dimethyl-5-oxo-5,6-dihydro-1,6-naphthyridine-3-carboxamide

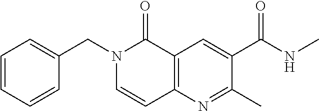 [SMILES: CNC(=O)c1cc2c(=O)n(Cc3ccccc3)ccc2nc1C]

### 1. Synthesis of 6-benzyl-N,2-dimethyl-5-oxo-5,6-dihydro-1,6-naphthyridine-3-carboxamide

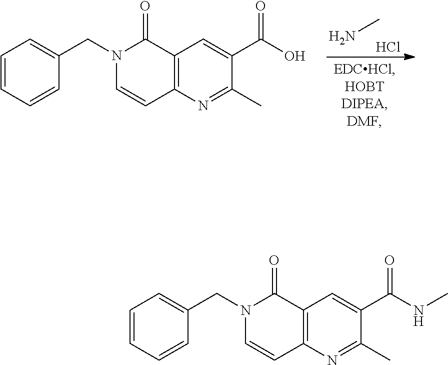

To a solution of 6-benzyl-2-methyl-5-oxo-5,6-dihydro-1,6-naphthyridine-3-carboxylic acid (57 mg, 0.194 mmol) in DMF (2.5 mL) at 0° C. was added EDC·HCl (57 mg, 0.297 mmol) and HOBT (45 mg, 0.333 mmol) and DIEA (58 mg, 0.449 mmol), followed by addition of CH3NH2HCl (21 mg, 0.311 mmol). The reaction mixture was stirred at room temperature for 1 h. Then crude was purified by reverse phase column (gradient: 0˜50% ACN/H₂O (0.5% NH₄HCO₃)) to afford 6-benzyl-N,2-dimethyl-5-oxo-5,6-dihydro-1,6-naphthyridine-3-carboxamide as a white solid (28.7 mg, yield: 48.2%). LCMS (ESI+): m/z calcd. for C₁₈H₁₈N₃O₂[M+H]⁺, 308.1, found, 308.1. ¹HNMR (400 MHz, DMSO-d₆, δ): 8.54-8.53 (m, 1H), 8.43 (s, 1H), 7.90 (d, J=7.6 Hz, 1H), 7.37-7.27 (m, 5H), 6.72 (d, J=7.6 Hz, 1H), 5.22 (s, 2H), 2.81 (d, J=4.4 Hz, 3H), 2.68 (s, 3H).

### Example 43: 6-benzyl-N-(4-carbamoylbenzyl)-2-methyl-5-oxo-5,6-dihydro-1,6-naphthyridine-3-carboxamide

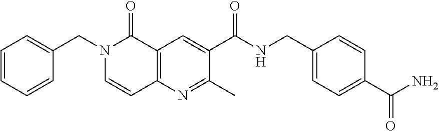 [SMILES: Cc1nc2ccn(Cc3ccccc3)c(=O)c2cc1C(=O)NCc1ccc(C(N)=O)cc1]

### 1. Synthesis of 6-benzyl-N-(4-carbamoylbenzyl)-2-methyl-5-oxo-5,6-dihydro-1,6-naphthyridine-3-carboxamide

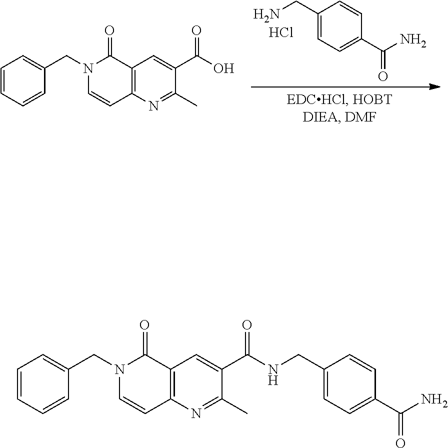 [SMILES: Cc1nc2ccn(Cc3ccccc3)c(=O)c2cc1C(=O)O ; NCc1ccc(C(N)=O)cc1 ; Cl ; Cc1nc2ccn(Cc3ccccc3)c(=O)c2cc1C(=O)NCc1ccc(C(N)=O)cc1]

To a solution of 6-benzyl-2-methyl-5-oxo-5,6-dihydro-1,6-naphthyridine-3-carboxylic acid (52 mg, 0.177 mmol) in DMF (1.5 mL) at 0° C. was added EDC·HCl (50 mg, 0.261 mmol) and HOBT (35 mg, 0.259 mmol) and DIEA (45 mg, 0.348 mmol), followed by addition of 4-(aminomethyl)benzamide hydrochloride (48 mg, 0.257 mmol). The reaction mixture was stirred at room temperature for 2 h. Then crude was purified by reverse phase column (gradient: 0˜50% ACN/H₂O (0.5% NH₄HCO₃)) to afford 6-benzyl-N-(4-carbamoylbenzyl)-2-methyl-5-oxo-5,6-dihydro-1,6-naphthyridine-3-carboxamide as a white solid (35.4 mg, 46.9%). LCMS (ESI+): m/z calcd. for C₂₅H₂₃N₄O₃ [M+H]⁺, 427.2, found, 427.2. ¹HNMR (400 MHz, DMSO-d₆, δ): 9.22 (m, 1H), 8.50 (s, 1H), 7.92-7.86 (m, 4H), 7.54-7.44 (m, 2H), 7.39-7.26 (m, 6H), 6.73 (d, J=7.6 Hz, 1H), 5.22 (s, 2H), 4.54 (d, J=6.0 Hz, 2H), 2.67 (s, 3H).

### Example 44: 6-((5-fluoropyridin-2-yl)methyl)-2-methyl-5-oxo-N-(pyridin-2-ylmethyl)-5,6-dihydro-1,6-naphthyridine-3-carboxamide

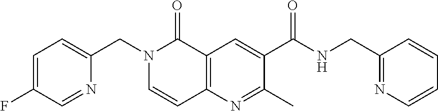 [SMILES: Cc1nc2ccn(Cc3ccc(F)cn3)c(=O)c2cc1C(=O)NCc1ccccn1]

### 1. Synthesis of 6-((5-fluoropyridin-2-yl)methyl)-2-methyl-5-oxo-N-(pyridin-2-ylmethyl)-5,6-dihydro-1,6-naphthyridine-3-carboxamide

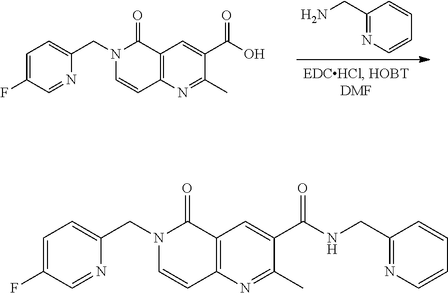 [SMILES: Cc1nc2ccn(Cc3ccc(F)cn3)c(=O)c2cc1C(=O)O ; NCc1ccccn1 ; Cc1nc2ccn(Cc3ccc(F)cn3)c(=O)c2cc1C(=O)NCc1ccccn1]

To a solution of 6-((5-fluoropyridin-2-yl)methyl)-2-methyl-5-oxo-5,6-dihydro-1,6-naphthyridine-3-carboxylic acid (90.0 mg, 0.28 mmol) and in DMF (2 mL) at 0° C. was added EDC·HCl (83.0 mg, 0.44 mmol) and HOBT (57.0 mg, 0.44 mmol), followed by addition of 2-pyridinemethaneamine (50.7 mg, 0.46 mmol). The reaction mixture was stirred at room temperature for 2 h. The crude was purified by reverse phase column (gradient: 0˜50% ACN/H₂O (0.5% NH₄HCO₃)) to afford 6-((5-fluoropyridin-2-yl)methyl)-2-methyl-5-oxo-N-(pyridin-2-ylmethyl)-5,6-dihydro-1,6-naphthyridine-3-carboxamide as a white solid (50.5 mg, yield: 43.58%). LCMS (ESI+): m/z calcd. for C₂₂H₁₉FN₅O₂[M+H]⁺, 404.1, found, 404.2. ¹HNMR (400 MHz, DMSO-d₆, δ): 9.24-9.21 (m, 1H), 8.54-8.48 (m, 3H), 7.91 (d, J=7.6 Hz, 1H), 7.82-7.80 (m, 1H), 7.72-7.70 (m, 1H), 7.45-7.41 (m, 2H), 7.31-7.28 (m, 1H), 6.74 (d, J=7.6 Hz, 1H), 5.37 (s, 2H), 4.58 (d, J=6.0 Hz, 2H), 2.85 (s, 3H).

### Example 45: 6-isopropyl-2-methyl-5-oxo-N-(pyridin-2-ylmethyl)-5,6-dihydro-1,6-naphthyridine-3-carboxamide

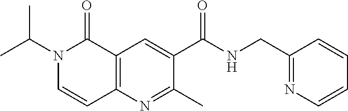 [SMILES: Cc1nc2ccn(C(C)C)c(=O)c2cc1C(=O)NCc1ccccn1]

### 1. Synthesis of ethyl 6-isopropyl-2-methyl-5-oxo-5,6-dihydro-1,6-naphthyridine-3-carboxylate

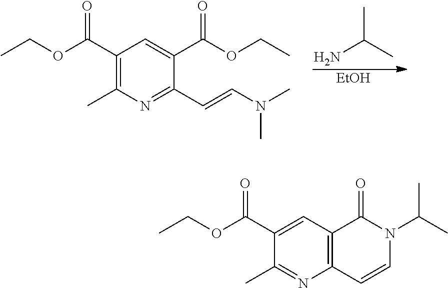

Under nitrogen, a solution of diethyl (E)-2-(2-(dimethylamino)vinyl)-6-methylpyridine-3,5-dicarboxylate (250 mg, 0.82 mmol) and isopropylamine (102 mg, 1.726 mmol) in EtOH (2.5 mL) was stirred overnight at 80° C. The mixture was allowed to cool down to 0° C., solids were collected by filtration and washed with H₂O (3×10 mL) to afford ethyl 6-isopropyl-2-methyl-5-oxo-5,6-dihydro-1,6-naphthyridine-3-carboxylate as a yellow solid (200.0 mg, yield: 89.3%). LCMS (ESI+): m/z calcd. for C₁₅H₁₉N₂O₃ [M+H]⁺, 275, found, 275.

### 2. Synthesis of 6-isopropyl-2-methyl-5-oxo-5,6-dihydro-1,6-naphthyridine-3-carboxylic acid

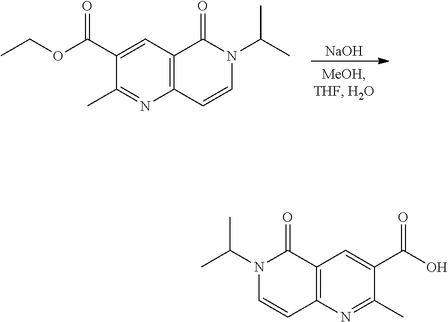

A solution of ethyl 6-isopropyl-2-methyl-5-oxo-5,6-dihydro-1,6-naphthyridine-3-carboxylate (200 mg, 0.729 mmol) and NaOH (90 mg, 2.250 mmol) in MeOH (0.9 mL) and THF (0.9 mL) H₂O (0.3 mL) was stirred for 4 h at 40° C. The residue was acidified to pH 4 with conc. HCl, and the aqueous layer was extracted with EtOAc. The combined organic layers were dried over Na₂SO₄ and concentrated in vacuo to afford 6-isopropyl-2-methyl-5-oxo-5,6-dihydro-1,6-naphthyridine-3-carboxylic acid as a yellow solid (60 mg, yield: 33.4%). LCMS (ESI+): m/z calcd. for C₁₃H₁₅N₂O₃ [M+H]⁺, 247, found, 247.

### 3. Synthesis of 6-isopropyl-2-methyl-5-oxo-N-(pyridin-2-ylmethyl)-5,6-dihydro-1,6-naphthyridine-3-carboxamide

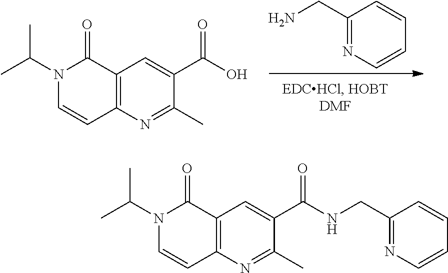

To a solution of 6-isopropyl-2-methyl-5-oxo-5,6-dihydro-1,6-naphthyridine-3-carboxylic acid (60 mg, 0.258 mmol) in DMF (2 mL) at 0° C. was added EDC·HCl (66 mg, 0.344 mmol) and HOBT (51 mg, 0.377 mmol), followed by addition of 2-pyridinemethaneamine (42 mg, 0.388 mmol). The reaction mixture was stirred at room temperature for 2 h. Then crude was purified by reverse phase column (gradient: 0˜50% ACN/H₂O (0.5% NH₄HCO₃)) to afford 6-isopropyl-2-methyl-5-oxo-N-(pyridin-2-ylmethyl)-5,6-dihydro-1,6-naphthyridine-3-carboxamide as a white solid (19.4 mg, yield: 22.3%). LCMS (ESI+): m/z calcd. for C₁₉H₂₁N₄O₂ [M+H]⁺, 337.2, found, 337.3. ¹HNMR (400 MHz, DMSO-d₆, δ): 9.24-9.21 (m, 1H), 8.56-8.52 (m, 2H), 7.88-7.80 (m, 2H), 7.42 (d, J=8.0 Hz, 1H), 7.32-7.30 (m, 1H), 6.71 (s, 1H), 5.18-5.12 (m, 1H), 4.60 (d, J=6.0 Hz, 2H), 2.68 (s, 3H), 1.37-1.35 (m, 6H).

### Example 46: 2-methyl-5-oxo-6-phenyl-N-(pyridin-2-ylmethyl)-5,6-dihydro-1,6-naphthyridine-3-carboxamide

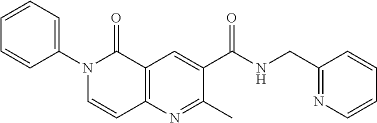 [SMILES: Cc1nc2ccn(-c3ccccc3)c(=O)c2cc1C(=O)NCc1ccccn1]

### 1. Synthesis of ethyl 2-methyl-S-oxo-6-phenyl-S, 6-dihydro-1,6-naphthyridine-3-carboxylate

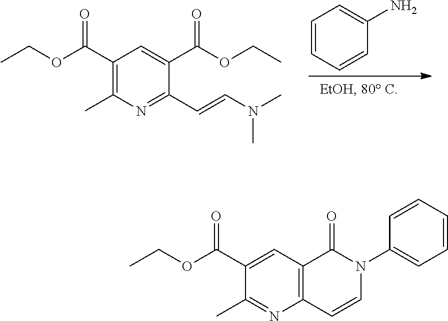

A solution of diethyl (E)-2-(2-(dimethylamino)vinyl)-6-methylpyridine-3,5-dicarboxylate (100 mg, 0.32 mmol) and aniline (129 mg, 1.38 mmol) in EtOH (1.5 mL) was stirred for 3 days at 80° C. under nitrogen atmosphere. The reaction mixture purified by reverse phase column (gradient: 0˜50% ACN/H₂O (0.5% NH₄HCO₃)) to afford ethyl 2-methyl-5-oxo-6-phenyl-5,6-dihydro-1,6-naphthyridine-3-carboxylate as a white solid (50 mg, yield: 49.68%). LCMS (ESI+): m/z calcd. for C₁₈H₁₇N₂O₃ [M+H]⁺, 309, found, 309.

### 2. Synthesis of 2-methyl-5-oxo-6-phenyl-5,6-dihydro-1,6-naphthyridine-3-carboxylic acid

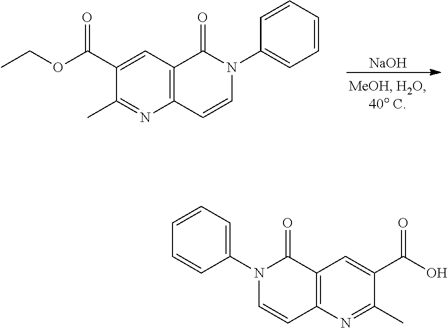

A solution of ethyl 2-methyl-5-oxo-6-phenyl-5,6-dihydro-1,6-naphthyridine-3-carboxylate (50 mg, 0.16 mmol) and NaOH (20 mg, 0.50 mmol) in MeOH (3 mL) and H₂O (1 m) was stirred for overnight at 40° C. The residue was acidified to pH 7 with conc. HCl, and the aqueous layer was extracted with EtOAc. The combined organic layers were dried over Na₂SO₄ and concentrated in vacuo to afford 2-methyl-5-oxo-6-phenyl-5,6-dihydro-1,6-naphthyridine-3-carboxylic acid as a white solid (30 mg, yield: 66.0%). LCMS (ESI+): m/z calcd. for C₁₆H₁₃N₂O₃ [M+H]⁺, 281.1, found, 281.0.

### 3. Synthesis of 2-methyl-5-oxo-6-phenyl-N-(pyridin-2-ylmethyl)-5,6-dihydro-1,6-naphthyridine-3-carboxamide

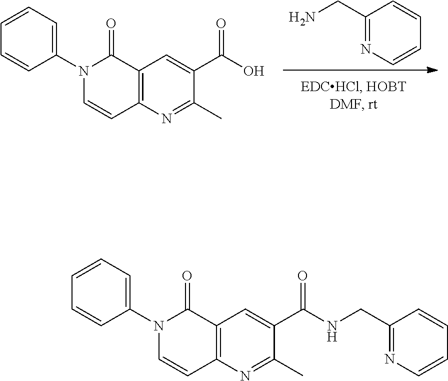 [SMILES: Cc1nc2ccn(-c3ccccc3)c(=O)c2cc1C(=O)O ; NCc1ccccn1 ; Cc1nc2ccn(-c3ccccc3)c(=O)c2cc1C(=O)NCc1ccccn1]

To a solution of 2-methyl-5-oxo-6-phenyl-5,6-dihydro-1,6-naphthyridine-3-carboxylic acid (30 mg, 0.10 mmol) in DMF (1 mL) at 0° C. was added EDC·HCl (31 mg, 0.16 mmol) and HOBT (22 mg, 0.16 mmol), followed by addition of 2-pyridinemethaneamine (18 mg, 0.16 mmol). The reaction mixture was stirred at room temperature for 0.5 h. Then crude was purified by reverse phase column (gradient: 0˜50% ACN/H₂O (0.5% NH₄HCO₃)) to afford 2-methyl-5-oxo-6-phenyl-N-(pyridin-2-ylmethyl)-5,6-dihydro-1,6-naphthyridine-3-carboxamide as a white solid (8.5 mg, yield: 21.35%). LCMS (ESI+): m/z calcd. for C₂₂H₁₉N₄O₂ [M+H]⁺, 371.1, found, 371.1. ¹HNMR (400 MHz, DMSO-d₆, δ): 9.25 (t, J=5.9 Hz, 1H), 8.55 (d, J=4.8 Hz, 2H), 7.85-7.77 (m, 2H), 7.57 (m, 2H), 7.53-7.47 (m, 3H), 7.42 (d, J=7.8 Hz, 1H), 7.30 (m, 1H), 6.76 (d, J=7.6 Hz, 1H), 4.60 (d, J=5.9 Hz, 2H), 2.72 (s, 3H).

### Example 47: 6-(4-fluorobenzyl)-2-methyl-5-oxo-N-(pyridin-2-ylmethyl)-5,6-dihydro-1,6-naphthyridine-3-carboxamide

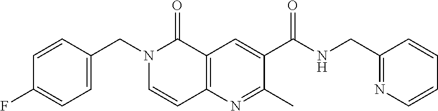 [SMILES: Cc1nc2ccn(Cc3ccc(F)cc3)c(=O)c2cc1C(=O)NCc1ccccn1]

### 1. Synthesis of ethyl 6-(4-fluorobenzyl)-2-methyl-5-oxo-5,6-dihydro-1,6-naphthyridine-3-carboxylate

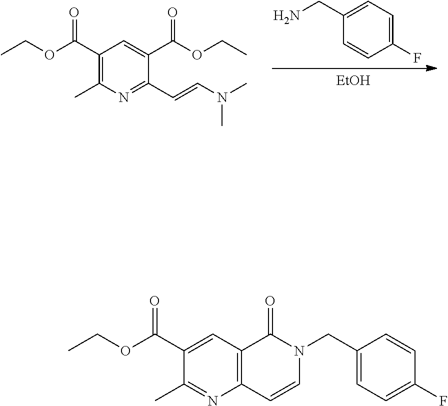 [SMILES: "EtOH" ; CCOC(=O)c1cc(C(=O)OCC)c(/C=C/N(C)C)nc1C ; NCc1ccc(F)cc1 ; CCOC(=O)c1cc2c(=O)n(Cc3ccc(F)cc3)ccc2nc1C]

A solution of diethyl (E)-2-(2-(dimethylamino)vinyl)-6-methylpyridine-3,5-dicarboxylate (250 mg, 0.816 mmol) and 4-fluorobenzylamine (154 mg, 1.231 mmol) in EtOH (3 mL) was stirred for overnight at 80° C. under nitrogen atmosphere. The mixture was allowed to cool down to 0° C., the precipitated solids were collected by filtration and washed with H₂O (3×10 mL) to afford ethyl 6-(4-fluorobenzyl)-2-methyl-5-oxo-5,6-dihydro-1,6-naphthyridine-3-carboxylate as a yellow solid (180.0 mg, yield: 64.8%). LCMS (ESI+): m/z calcd. for C₁₉H₁₈FN₂O₃[M+H]⁺, 341, found, 341.

### 2. Synthesis of 6-(4-fluorobenzyl)-2-methyl-5-oxo-5,6-dihydro-1,6-naphthyridine-3-carboxylic acid

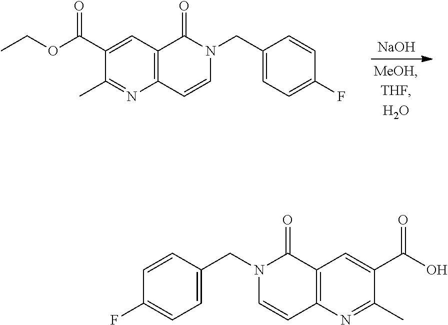 [SMILES: CCOC(=O)c1cc2c(=O)n(Cc3ccc(F)cc3)ccc2nc1C ; "NaOH" ; Cc1nc2ccn(Cc3ccc(F)cc3)c(=O)c2cc1C(=O)O]

A solution of ethyl 6-(4-fluorobenzyl)-2-methyl-5-oxo-5,6-dihydro-1,6-naphthyridine-3-carboxylate (180 mg, 0.529 mmol) and NaOH (65.57 mg, 1.640 mmol) in MeOH (1.5 mL) and THF (1.5 mL) and H₂O (0.5 mL) was stirred for 4 h at 40° C. The residue was acidified to pH 7 with conc. HCl, and the aqueous layer was extracted with EtOAc. The combined organic layers were dried over Na₂SO₄ and concentrated in vacuo to afford 6-(4-fluorobenzyl)-2-methyl-5-oxo-5,6-dihydro-1,6-naphthyridine-3-carboxylic acid as a yellow solid (100 mg, yield: 60.5%). LCMS (ESI+): m/z calcd. for C₁₇H₁₄FN₂O₃[M+H]⁺, 313, found, 313.

### 3. Synthesis of 6-(4-fluorobenzyl)-2-methyl-5-oxo-N-(pyridin-2-ylmethyl)-5,6-dihydro-1,6-naphthyridine-3-carboxamide

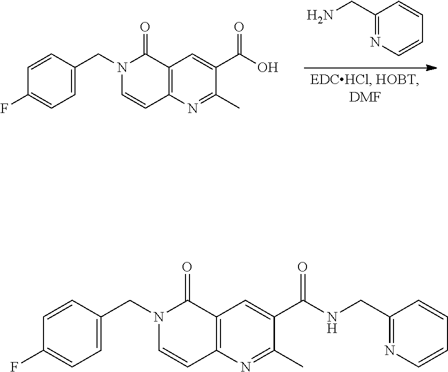 [SMILES: Cc1nc2ccn(Cc3ccc(F)cc3)c(=O)c2cc1C(=O)O ; NCc1ccccn1 ; Cc1nc2ccn(Cc3ccc(F)cc3)c(=O)c2cc1C(=O)NCc1ccccn1]

To a solution of 6-(4-fluorobenzyl)-2-methyl-5-oxo-5,6-dihydro-1,6-naphthyridine-3-carboxylic acid (100 mg, 0.320 mmol) in DMF (2.5 mL) at 0° C. was added EDC·HCl (111 mg, 0.579 mmol) and HOBT (71 mg, 0.525 mmol), followed by addition of 2-pyridinemethaneamine (55 mg, 0.509 mmol). The reaction mixture was stirred at room temperature for 1 h. Then crude was purified by reverse phase column (gradient: 0˜50% ACN/H₂O (0.5% NH₄HCO₃)) to afford 6-(4-fluorobenzyl)-2-methyl-5-oxo-N-(pyridin-2-ylmethyl)-5,6-dihydro-1,6-naphthyridine-3-carboxamide as a white solid (57.1 mg, yield: 44.3%). LCMS (ESI+): m/z calcd. for C₂₃H₂₀FN₄O₂[M+H]⁺, 403.2, found, 403.3. ¹HNMR (400 MHz, DMSO-d₆, δ): 9.25-9.21 (m, 1H), 8.56-8.54 (m, 2H), 7.95 (d, J=7.6 Hz, 1H), 7.83-7.79 (m, 1H), 7.43-7.40 (m, 3H), 7.39-7.32 (m, 1H), 7.29-7.16 (m, 2H), 6.74 (d, J=7.6 Hz, 1H), 5.20 (s, 2H), 4.59 (d, J=6.0 Hz, 2H), 2.69 (s, 3H).

### Example 48: 2-methyl-6-((5-methyloxazol-2-yl)methyl)-5-oxo-N-(pyridin-2-ylmethyl)-5,6-dihydro-1,6-naphthyridine-3-carboxamide

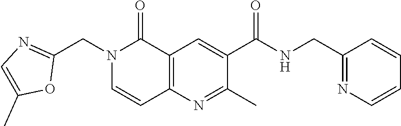 [SMILES: Cc1cnc(Cn2ccc3nc(C)c(C(=O)NCc4ccccn4)cc3c2=O)o1]

### 1. Synthesis of ethyl 2-methyl-6-((5-methyloxazol-2-yl)methyl)-5-oxo-5,6-dihydro-1,6-naphthyridine-3-carboxylate

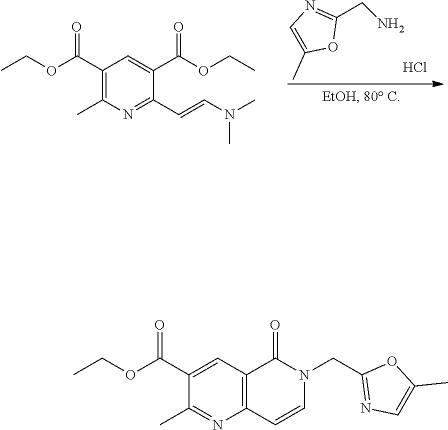

A solution of diethyl (E)-2-(2-(dimethylamino)vinyl)-6-methylpyridine-3,5-dicarboxylate (50 mg, 0.16 mmol) and (5-methyloxazol-2-yl)methanaminehydrochloride (29 mg, 0.19 mmol) in EtOH (1 mL) was stirred for overnight at 80° C. under nitrogen atmosphere. The mixture was allowed to cool down to 0° C., the precipitated solids were collected by filtration and washed with H₂O (3×10 mL) to afford ethyl 2-methyl-6-((5-methyloxazol-2-yl)methyl)-5-oxo-5,6-dihydro-1,6-naphthyridine-3-carboxylate as a yellow solid (50 mg, yield: 93.59%). LCMS (ESI+): m/z calcd. for C₁₇H₁₈N₃O₄ [M+H]⁺, 328, found, 328.

### 2. Synthesis of 2-methyl-6-((5-methyloxazol-2-yl)methyl)-5-oxo-5,6-dihydro-1,6-naphthyridine-3-carboxylic acid

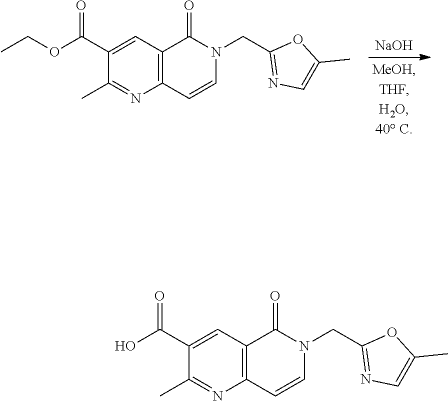

A solution of ethyl 2-methyl-6-((5-methyloxazol-2-yl)methyl)-5-oxo-5,6-dihydro-1,6-naphthyridine-3-carboxylate (52 mg, 0.15 mmol) and NaOH (20 mg, 0.50 mmol) in MeOH (2 mL), THF (2 mL) and H₂O (1 mL) was stirred for 4 h at 40° C. The residue was acidified to pH 7 with conc. HCl, and the aqueous layer was extracted with EtOAc. The combined organic layers were dried over Na₂SO₄ and concentrated in vacuo to afford 2-methyl-6-((5-methyloxazol-2-yl)methyl)-5-oxo-5,6-dihydro-1,6-naphthyridine-3-carboxylic acid as a white solid (10 mg, yield: 21.03%). LCMS (ESI+): m/z calcd. for C₁₅H₁₄N₃O₄ [M+H]⁺, 300.1, found, 300.1.

### 3. Synthesis of 2-methyl-6-((5-methyloxazol-2-yl)methyl)-5-oxo-N-(pyridin-2-ylmethyl)-5,6-dihydro-1,6-naphthyridine-3-carboxamide

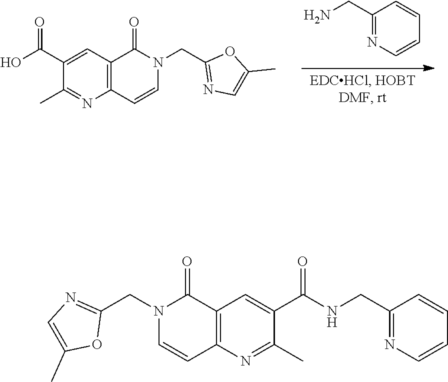 [SMILES: Cc1cnc(Cn2ccc3nc(C)c(C(=O)O)cc3c2=O)o1 ; NCc1ccccn1 ; Cc1cnc(Cn2ccc3nc(C)c(C(=O)NCc4ccccn4)cc3c2=O)o1]

To a solution of 2-methyl-6-((5-methyloxazol-2-yl)methyl)-5-oxo-5,6-dihydro-1,6-naphthyridine-3-carboxylic acid (10 mg, 0.03 mmol) in DMF (1 mL) at 0° C. was added EDC·HCl (10 mg, 0.05 mmol) and HOBT (7 mg, 0.05 mmol), followed by addition of 2-pyridinemethaneamine (6 mg, 0.05 mmol). The reaction mixture was stirred at room temperature for 0.5 h. Then crude was purified by reverse phase column (gradient: 0˜50% ACN/H₂O (0.5% NH₄HCO₃)) to afford 2-methyl-6-((5-methyloxazol-2-yl)methyl)-5-oxo-N-(pyridin-2-ylmethyl)-5,6-dihydro-1,6-naphthyridine-3-carboxamide as a white solid (2.5 mg, yield: 19.08%). LCMS (ESI+): m/z calcd. for C₂₁H₂₀N₅O₃ [M+H]⁺, 390.2, found, 390.1. ¹HNMR (400 MHz, DMSO-d₆, δ): 9.23 (t, J=6.0 Hz, 1H), 8.63-8.34 (m, 2H), 7.93-7.74 (m, 2H), 7.41 (d, J=7.8 Hz, 1H), 7.34-7.25 (m, 1H), 6.75 (d, J=8.4 Hz, 2H), 5.30 (s, 2H), 4.59 (d, J=5.9 Hz, 2H), 2.70 (s, 3H), 2.26 (s, 3H).

### Example 49: 2-methyl-6-((1-methyl-1H-pyrazol-4-yl)methyl)-5-oxo-N-(pyridin-2-ylmethyl)-5,6-dihydro-1,6-naphthyridine-3-carboxamide

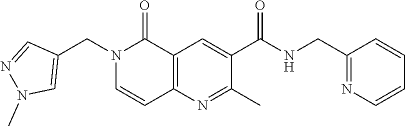 [SMILES: Cc1nc2ccn(Cc3cnn(C)c3)c(=O)c2cc1C(=O)NCc1ccccn1]

### 1. Synthesis of ethyl 2-methyl-6-((1-methyl-1H-pyrazol-4-yl)methyl)-5-oxo-5,6-dihydro-1,6-naphthyridine-3-carboxylate

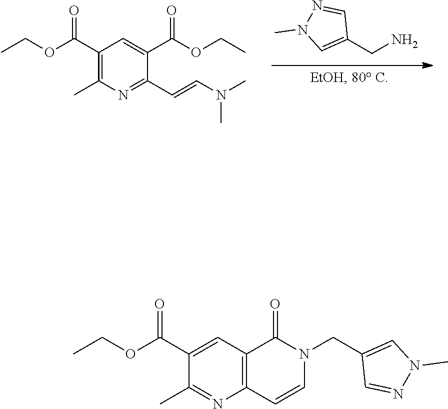

A solution of diethyl (E)-2-(2-(dimethylamino)vinyl)-6-methylpyridine-3,5-dicarboxylate (250 mg, 0.81 mmol) and 1-(1-methylpyrazol-4-yl)methanamine (109 mg, 0.98 mmol) in EtOH (3 mL) was stirred for overnight at 80° C. under nitrogen atmosphere. The mixture was allowed to cool down to 0° C., the precipitated solids were collected by filtration and washed with H₂O (3×10 mL) to afford ethyl 2-methyl-6-((1-methyl-1H-pyrazol-4-yl)methyl)-5-oxo-5,6-dihydro-1,6-naphthyridine-3-carboxylate as a yellow solid (73 mg, yield: 27.41%). LCMS (ESI+): m/z calcd. for C₁₇H₁₉N₄O₃ [M+H]⁺, 327.1, found, 327.1.

### 2. Synthesis of 2-methyl-6-((1-methyl-1H-pyrazol-4-yl)methyl)-5-oxo-5,6-dihydro-1,6-naphthyridine-3-carboxylic acid

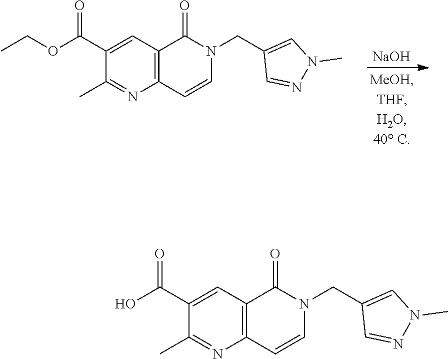

A solution of ethyl 2-methyl-6-((1-methyl-1H-pyrazol-4-yl)methyl)-5-oxo-5,6-dihydro-1,6-naphthyridine-3-carboxylate (304 mg, 0.93 mmol) and NaOH (112 mg, 2.80 mmol) in MeOH (4 mL), THF (4 mL) and H₂O (2 mL) was stirred for 4 h at 40° C. The residue was acidified to pH 7 with conc. HCl, and the aqueous layer was extracted with EtOAc. The combined organic layers were dried over Na₂SO₄ and concentrated in vacuo to afford 2-methyl-6-((1-methyl-1H-pyrazol-4-yl)methyl)-5-oxo-5,6-dihydro-1,6-naphthyridine-3-carboxylic acid as a white solid (90 mg, yield: 32.39%). LCMS (ESI+): m/z calcd. for C₁₅H₁₅N₄O₃ [M+H]⁺, 299.1, found, 299.1.

### 3. Synthesis of 2-methyl-6-((1-methyl-1H-pyrazol-4-yl)methyl)-5-oxo-N-(pyridin-2-ylmethyl)-5,6-dihydro-1,6-naphthyridine-3-carboxamide

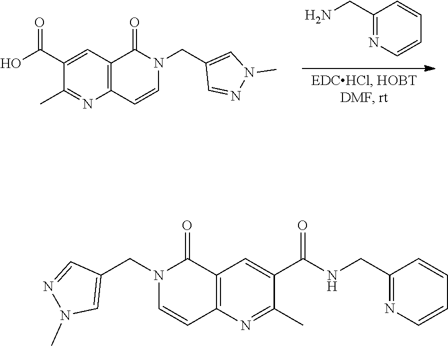 [SMILES: Cc1nc2ccn(Cc3cnn(C)c3)c(=O)c2cc1C(=O)O ; NCc1ccccn1 ; Cc1nc2ccn(Cc3cnn(C)c3)c(=O)c2cc1C(=O)NCc1ccccn1]

To a solution of 2-methyl-6-((1-methyl-1H-pyrazol-4-yl)methyl)-5-oxo-5,6-dihydro-1,6-naphthyridine-3-carboxylic acid (110 mg, 0.36 mmol) in DMF (4 mL, 51.68 mmol) at 0° C. was added EDC·HCl (107 mg, 0.55 mmol) and HOBT (75 mg, 0.55 mmol), followed by addition of 2-pyridinemethaneamine (60 mg, 0.55 mmol). The reaction mixture was stirred at room temperature for 0.5 h. Then crude was purified by reverse phase column (gradient: 0˜50% ACN/H₂O (0.5% NH₄HCO₃)) to afford 2-methyl-6-((1-methyl-1H-pyrazol-4-yl)methyl)-5-oxo-N-(pyridin-2-ylmethyl)-5,6-dihydro-1,6-naphthyridine-3-carboxamide as a white solid (33.2 mg, yield: 22.30%). LCMS (ESI+): m/z calcd. for C₂₁H₂₁N₆O₂ [M+H]⁺, 389.2, found, 389.1. ¹HNMR (400 MHz, DMSO-d₆, δ): 9.23 (t, J=6.0 Hz, 1H), 8.58-8.47 (m, 2H), 7.89 (d, J=7.6 Hz, 1H), 7.82 (m, 1H), 7.72 (s, 1H), 7.47-7.39 (m, 2H), 7.34-7.27 (m, 1H), 6.68 (d, J=7.6 Hz, 1H), 5.02 (s, 2H), 4.59 (d, J=5.9 Hz, 2H), 3.78 (s, 3H), 2.67 (s, 3H).

### Example 50: 6-benzyl-N-((4,5-difluoropyridin-2-yl)methyl)-2-methyl-5-oxo-5,6-dihydro-1,6-naphthyridine-3-carboxamide

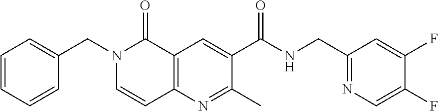 [SMILES: Cc1nc2ccn(Cc3ccccc3)c(=O)c2cc1C(=O)NCc1cc(F)c(F)cn1]

### 1. Synthesis of tert-butyl ((4,5-difluoropyridin-2-yl)methyl)carbamate

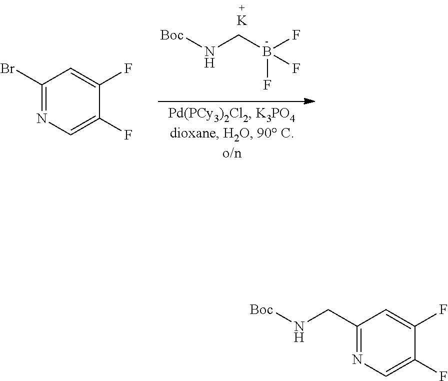

A solution of 2-bromo-4,5-difluoropyridine (200 mg, 1.03 mmol), tert-butyl N-[(trifluoro-lambda4-boranyl)methyl]carbamate potassium (270 mg, 1.13 mmol), Pd(PCy₃)₂Cl₂ (114 mg, 0.15 mmol) and K₃PO₄ (660 mg, 3.10 mmol) in dioxane (10 mL) and H₂O (2 mL) was stirred overnight at 90° C. under nitrogen atmosphere. The resulting mixture was filtered, extracted with EtOAc, and dried over anhydrous Na₂SO₄. The filtrate was concentrated under reduced pressure and purified by silica gel column chromatography, eluted with EtOAc in petroleum ether (0-50%) to afford tert-butyl ((4,5-difluoropyridin-2-yl)methyl)carbamate as a colorless oil (28 mg, yield: 11.12%). LCMS (ESI+): m/z calcd. for C₁₁H₁₅F₂N₂O₂[M+H]⁺, 245.1, found, 245.1.

### 2. Synthesis of (4,5-difluoropyridin-2-yl) methanamine hydrochloride

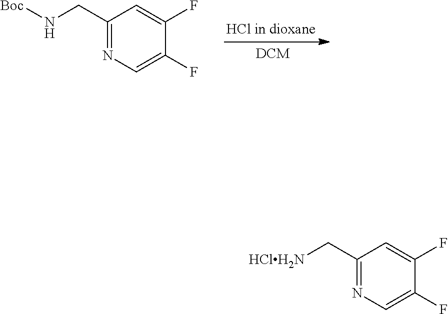

To a solution of tert-butyl ((4,5-difluoropyridin-2-yl)methyl)carbamate (28 mg, 0.11 mmol) in HCl (gas) in 1,4-dioxane (1 mL) and DCM (1 mL) was stirred for 1 h at room temperature. The resulting mixture was concentrated under reduced pressure to afford crude product (4,5-difluoropyridin-2-yl) methanamine hydrochloride (15 mg, yield: 90.7%) was used in the next step directly without further purification. LCMS (ESI+): m/z calcd. for C₆H₇F₂N₂[M+H]⁺, 145.1, found, 145.0.

### 3. Synthesis of 6-benzyl-N-((4,5-difluoropyridin-2-yl)methyl)-2-methyl-5-oxo-5,6-dihydro-1,6-naphthyridine-3-carboxamide

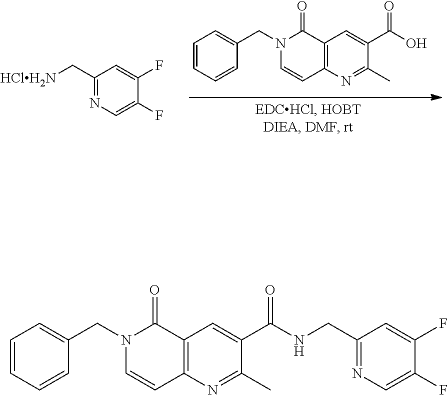

To a solution of 6-benzyl-2-methyl-5-oxo-5,6-dihydro-1,6-naphthyridine-3-carboxylic acid (25 mg, 0.08 mmol) in DMF (1 mL) at 0° C. was added EDC·HCl (24 mg, 0.12 mmol) and HOBT (17 mg, 0.12 mmol), followed by addition of (4,5-difluoropyridin-2-yl) methanamine hydrochloride (15 mg, 0.10 mmol) and DIEA (33 mg, 0.25 mmol). The reaction mixture was stirred at room temperature for 1 h. Then crude was purified by reverse phase column (gradient: 0-50% ACN/H₂O (0.5% NH₄HCO₃)) to afford 6-benzyl-N-((4,5-difluoropyridin-2-yl)methyl)-2-methyl-5-oxo-5,6-dihydro-1,6-naphthyridine-3-carboxamide as a white solid (16.5 mg, yield: 46%). LCMS (ESI+): m/z calcd. for C₂₃H₁₉F₂N₄O₂[M+H]⁺, 421.1, found, 421.1. T HNMR (400 MHz, DMSO-d₆, δ): 9.24 (t, J=6.0 Hz, 1H), 8.73 (m, 1H), 8.57 (s, 1H), 7.92 (d, J=7.5 Hz, 1H), 7.59 (m, 1H), 7.39-7.23 (m, 5H), 6.72 (d, J=7.6 Hz, 1H), 5.23 (s, 2H), 4.57 (d, J=5.9 Hz, 2H), 2.68 (s, 3H).

### Example 51: 6-(4-chlorobenzyl)-N-((5-fluoropyridin-2-yl)methyl)-2-methyl-5-oxo-5,6-dihydro-1,6-naphthyridine-3-carboxamide

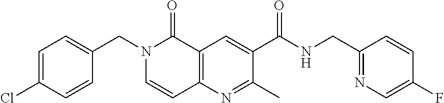 [SMILES: Cc1nc2ccn(Cc3ccc(Cl)cc3)c(=O)c2cc1C(=O)NCc1ccc(F)cn1]

### 1. Synthesis of ethyl 6-(4-chlorobenzyl)-2-methyl-5-oxo-5,6-dihydro-1,6-naphthyridine-3-carboxylate

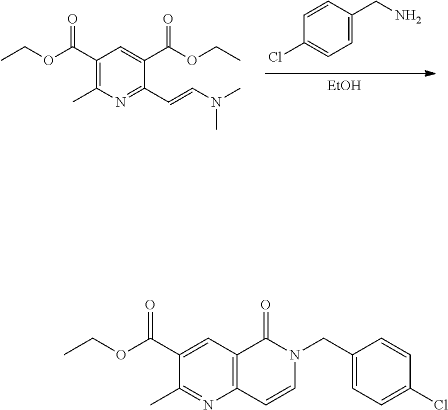 [SMILES: "EtOH" ; CCOC(=O)c1cc(C(=O)OCC)c(/C=C/N(C)C)nc1C ; NCc1ccc(Cl)cc1 ; CCOC(=O)c1cc2c(=O)n(Cc3ccc(Cl)cc3)ccc2nc1C]

A solution of diethyl (E)-2-(2-(dimethylamino)vinyl)-6-methylpyridine-3,5-dicarboxylate (250 mg, 0.816 mmol) and 1-(4-chlorophenyl)methanamine (165 mg, 1.165 mmol) in EtOH (4 mL) was stirred for overnight at 80° C. under nitrogen atmosphere. The mixture was allowed to cool down to 0° C., the precipitated solids were collected by filtration and washed with H₂O (3×10 mL) to afford ethyl 6-(4-chlorobenzyl)-2-methyl-5-oxo-5,6-dihydro-1,6-naphthyridine-3-carboxylate as a yellow solid (150.0 mg, yield: 51.2%). LCMS (ESI+): m/z calcd. for C₁₉H₁₈ClN₂O₃[M+H]⁺, 357, found, 357.

### 2. Synthesis of 6-(4-chlorobenzyl)-2-methyl-5-oxo-5,6-dihydro-1,6-naphthyridine-3-carboxylic acid

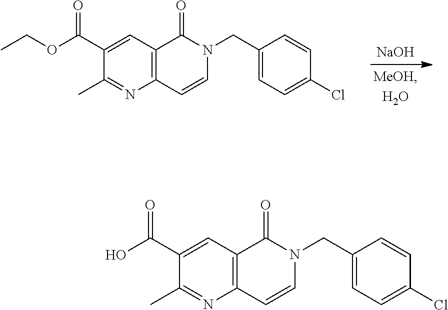 [SMILES: CCOC(=O)c1cc2c(=O)n(Cc3ccc(Cl)cc3)ccc2nc1C ; "NaOH" ; Cc1nc2ccn(Cc3ccc(Cl)cc3)c(=O)c2cc1C(=O)O]

A solution of ethyl 6-(4-chlorobenzyl)-2-methyl-5-oxo-5,6-dihydro-1,6-naphthyridine-3-carboxylate (150 mg, 0.420 mmol) and NaOH (33 mg, 0.825 mmol) in MeOH (3 mL) and H₂O (1 mL) was stirred for 8 h at 40° C. The residue was acidified to pH 4 with conc. HCl, and the aqueous layer was extracted with EtOAc. The combined organic layers were dried over Na₂SO₄ and concentrated in vacuo to afford 6-(4-chlorobenzyl)-2-methyl-5-oxo-5,6-dihydro-1,6-naphthyridine-3-carboxylic acid as a white solid (100 mg, yield: 72.3%). LCMS (ESI+): m/z calcd. for C₁₇H₁₄Cl N₂O₃[M+H]⁺, 329, found, 329.

### 3. Synthesis of 6-(4-chlorobenzyl)-N-((5-fluoropyridin-2-yl)methyl)-2-methyl-5-oxo-5,6-dihydro-1,6-naphthyridine-3-carboxamide

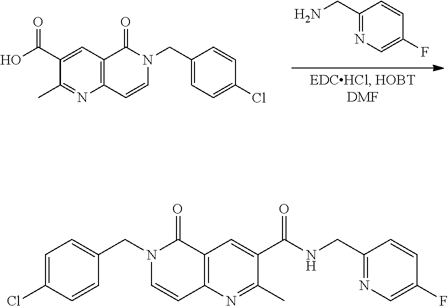 [SMILES: Cc1nc2ccn(Cc3ccc(Cl)cc3)c(=O)c2cc1C(=O)O ; NCc1ccc(F)cn1 ; Cc1nc2ccn(Cc3ccc(Cl)cc3)c(=O)c2cc1C(=O)NCc1ccc(F)cn1]

To a solution of 6-(4-chlorobenzyl)-2-methyl-5-oxo-5,6-dihydro-1,6-naphthyridine-3-carboxylic acid (150 mg, 0.456 mmol) in DMF (2 mL) at 0° C. was added EDC·HCl (57 mg, 0.396 mmol) and HOBT (87 mg, 0.455 mmol), followed by addition of 1-(5-fluoropyridin-2-yl)methanamine (57 mg, 0.452 mmol). The reaction mixture was stirred at room temperature for 1 h. Then crude was purified by reverse phase column (gradient: 0˜50% ACN/H₂O (0.5% NH₄HCO₃)) to afford 6-(4-chlorobenzyl)-N-((5-fluoropyridin-2-yl)methyl)-2-methyl-5-oxo-5,6-dihydro-1,6-naphthyridine-3-carboxamide as a white solid (82.6 mg, yield: 41.4%). LCMS (ESI+): m/z calcd. for C₂₃H₁₉ClFN₄O₂[M+H]⁺, 437.1, found, 437.1. ¹HNMR (400 MHz, DMSO-d₆, δ): 9.26-9.26 (m, 1H), 8.55-8.53 (m, 2H), 7.94 (d, J=7.6 Hz, 1H), 7.77-7.72 (m, 1H), 7.51 (d, J=4.8 Hz, 1H), 7.49-7.48 (m, 2H), 7.43-7.41 (m, 2H), 6.74 (d, J=7.6 Hz, 1H), 5.21 (s, 2H), 4.59 (d, J=5.6 Hz, 2H), 2.68 (s, 3H).

### Example 52: 2-methyl-5-oxo-N-(pyridin-2-ylmethyl)-6-(pyridin-4-ylmethyl)-5,6-dihydro-1,6-naphthyridine-3-carboxamide

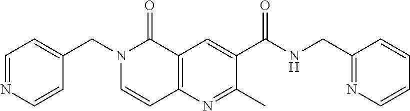 [SMILES: Cc1nc2ccn(Cc3ccncc3)c(=O)c2cc1C(=O)NCc1ccccn1]

### 1. Synthesis of ethyl 2-methyl-5-oxo-6-(pyridin-4-ylmethyl)-5,6-dihydro-1,6-naphthyridine-3-carboxylate

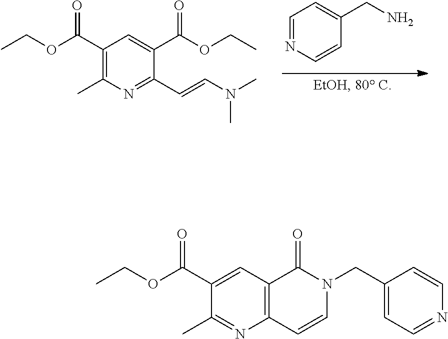

To a solution of diethyl (E)-2-(2-(dimethylamino)vinyl)-6-methylpyridine-3,5-dicarboxylate (300.5 mg, 0.979 mmol) and pyridin-4-ylmethanamine (211.5 mg, 1.958 mmol) in EtOH (3 mL) was stirred at 80° C. for 8 h. The mixture was added water (10 mL) at 0° C. The solids were collected by filtration to afford ethyl 2-methyl-5-oxo-6-(pyridin-4-ylmethyl)-5,6-dihydro-1,6-naphthyridine-3-carboxylate as yellow solid (300.6 mg, yield: 94.8%). LCMS (ESI+): m/z calcd. for C₁₈H₁₈N₃O₃[M+H]⁺, 324, found, 324.

### 2. Synthesis of 2-methyl-5-oxo-6-(pyridin-4-ylmethyl)-5,6-dihydro-1,6-naphthyridine-3-carboxylic acid

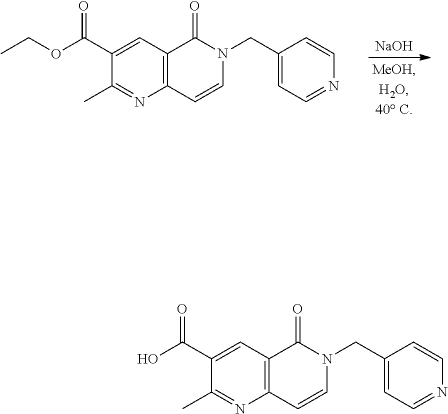

To a solution of ethyl 2-methyl-5-oxo-6-(pyridin-4-ylmethyl)-5,6-dihydro-1,6-naphthyridine-3-carboxylate (200.5 mg, 0.62 mmol) and NaOH (74.4 mg, 1.86 mmol) in MeOH (3 mL) and H₂O (1 mL) was stirred for 3 h at 40° C. The residue was acidified to pH 4 with conc. HCl, and the aqueous layer was extracted with EtOAc. The combined organic layers were dried over Na₂SO₄ and concentrated in vacuo to afford 2-methyl-5-oxo-6-(pyridin-4-ylmethyl)-5,6-dihydro-1,6-naphthyridine-3-carboxylic acid as a white solid (123.5 mg, yield: 67.4%). LCMS (ESI+): m/z calcd. for C₁₆H₁₄N₃O₃ [M+H]⁺, 296, found, 296.

### 3. Synthesis of 2-methyl-5-oxo-N-(pyridin-2-ylmethyl)-6-(pyridin-4-ylmethyl)-5,6-dihydro-1,6-naphthyridine-3-carboxamide

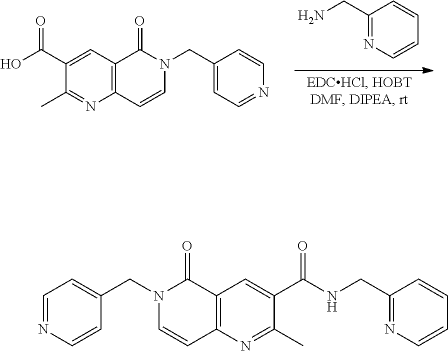 [SMILES: Cc1nc2ccn(Cc3ccncc3)c(=O)c2cc1C(=O)O ; NCc1ccccn1 ; Cc1nc2ccn(Cc3ccncc3)c(=O)c2cc1C(=O)NCc1ccccn1]

To a solution of 2-methyl-5-oxo-6-(pyridin-4-ylmethyl)-5,6-dihydro-1,6-naphthyridine-3-carboxylic acid (80.5 mg, 0.27 mmol) and in DMF (5 mL) at 0° C. was added EDC·HCl (78.2 mg, 0.405 mmol) and HOBT (55.3 mg, 0.405 mmol), DIPEA (70.5 mg, 0.54 mmol) followed by addition of pyridin-2-ylmethanamine (43.7 mg, 0.405 mmol). The reaction mixture was stirred at room temperature for 2 h. The crude product was purified by Prep-HPLC with the following conditions (Column: XBridge Prep OBD C₁₈ Column, 30*150 mm, 5 μm; Mobile Phase A: Water (10 mmol/L NH₄HCO₃), Mobile Phase B: ACN; Flow rate: 60 mL/min; Gradient: 33% B to 43% B in 8 min, 43%) to afford 2-methyl-5-oxo-N-(pyridin-2-ylmethyl)-6-(pyridin-4-ylmethyl)-5,6-dihydro-1,6-naphthyridine-3-carboxamide as a white solid (8.4 mg, yield: 8%). LCMS (ESI+): m/z calcd. for C₂₂H₂₀N₅O₂ [M+H]⁺, 386.2, found, 386.2. ¹HNMR (400 MHz, DMSO-d₆, δ): 9.24 (t, J=6.0 Hz, 1H), 8.58-8.50 (m, 4H), 7.93 (d, J=7.6 Hz, 1H), 7.86-7.76 (m, 1H), 7.41 (d, J=7.8 Hz, 1H), 7.33-7.27 (m, 1H), 7.26-7.21 (m, 2H), 6.77 (d, J=7.6 Hz, 1H), 5.26 (s, 2H), 4.58 (d, J=5.9 Hz, 2H), 2.70 (s, 3H).

### Example 53: 2,6-dimethyl-5-oxo-N-(pyridin-2-ylmethyl)-5,6-dihydro-1,6-naphthyridine-3-carboxamide

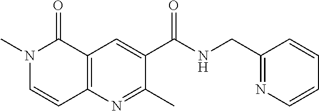 [SMILES: Cc1nc2ccn(C)c(=O)c2cc1C(=O)NCc1ccccn1]

### 1. Synthesis of ethyl 2,6-dimethyl-5-oxo-5,6-dihydro-1,6-naphthyridine-3-carboxylate

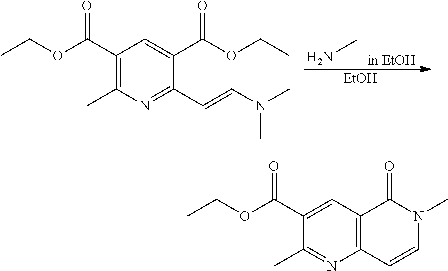

A solution of diethyl (E)-2-(2-(dimethylamino)vinyl)-6-methylpyridine-3,5-dicarboxylate (250 mg, 0.816 mmol) and methylamine (151 mg, 4.862 mmol) in EtOH (3 mL) was stirred at 80° C. for 8 h. The precipitated solids were collected by filtration and washed with water (2×5 mL). The solids were collected by filtration to afford ethyl 2,6-dimethyl-5-oxo-5,6-dihydro-1,6-naphthyridine-3-carboxylate as yellow solid (240 mg, yield: 74.64%). LCMS (ESI+): m/z calcd. for C₁₃H₁₅N₂O₃ [M+H]⁺, 247, found, 247.

### 2. Synthesis of 2,6-dimethyl-5-oxo-5,6-dihydro-1,6-naphthyridine-3-carboxylic acid

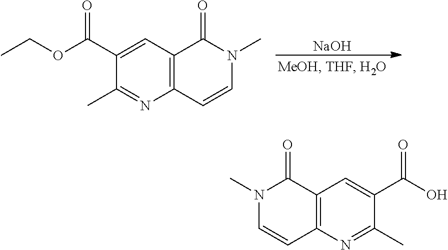

A solution of ethyl 2,6-dimethyl-5-oxo-5,6-dihydro-1,6-naphthyridine-3-carboxylate (150 mg, 0.61 mmol) and NaOH (75.0 mg, 1.88 mmol) in MeOH (0.9 mL) and THF (0.9) and H₂O (0.3 mL) was stirred for 4 h at 40° C. The reaction was acidified to pH 4 with conc. HCl, and was extracted with EtOAc. The combined organic layers were dried over Na₂SO₄ and concentrated in vacuo to afford 2,6-dimethyl-5-oxo-5,6-dihydro-1,6-naphthyridine-3-carboxylic acid as a yellow solid (80 mg, yield: 60.2%). LCMS (ESI+): m/z calcd. for C₁₁H₁₁N₂O₃[M+H]⁺, 219, found, 219.

### 3. Synthesis of 2,6-dimethyl-5-oxo-N-(pyridin-2-ylmethyl)-5,6-dihydro-1,6-naphthyridine-3-carboxamide

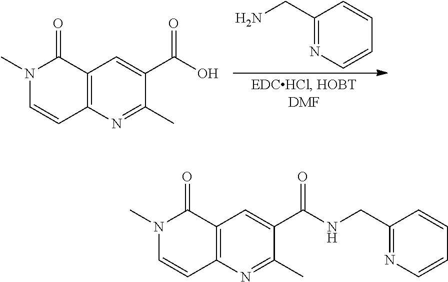 [SMILES: Cc1nc2ccn(C)c(=O)c2cc1C(=O)O ; NCc1ccccn1 ; Cc1nc2ccn(C)c(=O)c2cc1C(=O)NCc1ccccn1]

To a solution of 2,6-dimethyl-5-oxo-5,6-dihydro-1,6-naphthyridine-3-carboxylic acid (80 mg, 0.367 mmol) and in DMF (2 mL) at 0° C. was added EDC·HCl (100 mg, 0.522 mmol) and HOBT (73 mg, 0.540 mmol), followed by addition of 2-pyridinemethaneamine (62 mg, 0.573 mmol)). The reaction mixture was stirred at room temperature for 2 h. Then mixture was purified by reverse phase column (gradient: 0˜50% ACN/H₂O (0.5% NH₄HCO₃)) to afford 2,6-dimethyl-5-oxo-N-(pyridin-2-ylmethyl)-5,6-dihydro-1,6-naphthyridine-3-carboxamide as a white solid (18.2 mg, yield: 16.1%). LCMS (ESI+): m/z calcd. for C₁₇H₁₇N₄O₂ [M+H]⁺, 309.1, found, 309.2. ¹HNMR (400 MHz, DMSO-d₆, δ): 9.26-9.25 (m, 1H), 8.56-8.52 (m, 2H), 7.84-7.80 (m, 2H), 7.42 (d, J=7.6 Hz, 1H), 7.32-7.29 (m, 1H), 6.68 (d, J=7.2 Hz, 1H), 4.60 (d, J=6.0 Hz, 2H), 3.54 (s, 3H), 2.68 (s, 3H).

### Example 54: 2-methyl-6-(4-methylbenzyl)-5-oxo-N-(pyridin-2-ylmethyl)-5,6-dihydro-1,6-naphthyridine-3-carboxamide

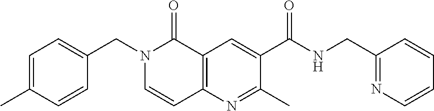 [SMILES: Cc1ccc(Cn2ccc3nc(C)c(C(=O)NCc4ccccn4)cc3c2=O)cc1]

### 1. Synthesis of ethyl 2-methyl-6-(4-methylbenzyl)-5-oxo-5,6-dihydro-1,6-naphthyridine-3-carboxylate

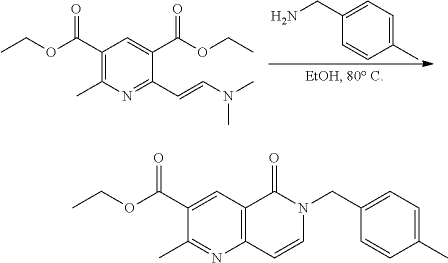

A solution of diethyl (E)-2-(2-(dimethylamino)vinyl)-6-methylpyridine-3,5-dicarboxylate (250 mg, 0.81 mmol) and p-tolylmethanamine (119 mg, 0.98 mmol) in EtOH (3 mL) was stirred for overnight at 80° C. under nitrogen atmosphere. The mixture was allowed to cool down to 0° C., the solids were collected by filtration and washed with H₂O (3×5 mL) to afford crude product ethyl 2-methyl-6-(4-methylbenzyl)-5-oxo-5,6-dihydro-1,6-naphthyridine-3-carboxylate as a yellow solid (290 mg, yield: 88.1%). LCMS (ESI+): m/z calcd. for C₂₀H₂₁N₂O₃ [M+H]⁺, 337, found, 337.

### 2. Synthesis of 2-methyl-6-(4-methylbenzyl)-5-oxo-5,6-dihydro-1,6-naphthyridine-3-carboxylic acid

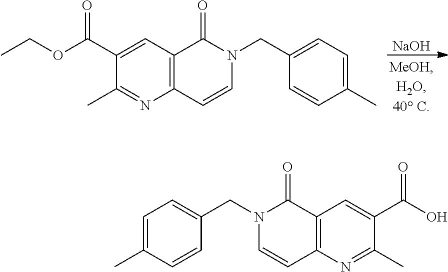

A solution of ethyl 2-methyl-6-(4-methylbenzyl)-5-oxo-5,6-dihydro-1,6-naphthyridine-3-carboxylate (290 mg, 0.86 mmol) and NaOH (104 mg, 2.60 mmol) in MeOH (6 mL) and H₂O (2 mL) was stirred for overnight at 40° C. The mixture was acidified to pH 7 with conc. HCl, and the aqueous layer was extracted with EtOAc. The combined organic layers were dried over Na₂SO₄ and concentrated in vacuo to afford 2-methyl-6-(4-methylbenzyl)-5-oxo-5,6-dihydro-1,6-naphthyridine-3-carboxylic acid as a yellow solid (150 mg, yield: 56.43%). LCMS (ESI+): m/z calcd. for C₁₈H₁₇N₂O₃ [M+H]⁺, 309, found, 309.

### 3. Synthesis of 2-methyl-6-(4-methylbenzyl)-5-oxo-N-(pyridin-2-ylmethyl)-5,6-dihydro-1,6-naphthyridine-3-carboxamide

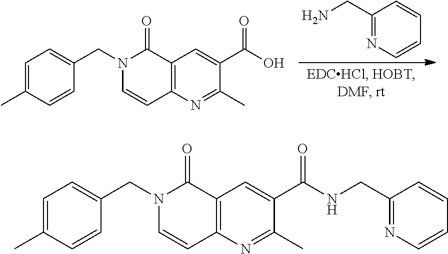 [SMILES: Cc1ccc(Cn2ccc3nc(C)c(C(=O)O)cc3c2=O)cc1 ; NCc1ccccn1 ; Cc1ccc(Cn2ccc3nc(C)c(C(=O)NCc4ccccn4)cc3c2=O)cc1]

To a solution of 2-methyl-6-(4-methylbenzyl)-5-oxo-5,6-dihydro-1,6-naphthyridine-3-carboxylic acid (100 mg, 0.32 mmol) in DMF (4 mL) at 0° C. was added EDC·HCl (94.1 mg, 0.49 mmol) and HOBT (66.5 mg, 0.48 mmol), followed by addition of 2-pyridinemethaneamine (53.8 mg, 0.49 mmol). The reaction mixture was stirred at room temperature for 0.5 h. Then crude was purified by reverse phase column (gradient: 0˜50% ACN/H₂O (0.5% NH₄HCO₃)) to afford 2-methyl-6-(4-methylbenzyl)-5-oxo-N-(pyridin-2-ylmethyl)-5,6-dihydro-1,6-naphthyridine-3-carboxamide as a white solid (54 mg, yield: 41.74%). LCMS (ESI+): m/z calcd. for C₂₄H₂₃N₄O₂ [M+H]⁺, 399.2, found, 399.1. ¹HNMR (400 MHz, DMSO-d₆, δ): 9.23 (t, J=6.0 Hz, 1H), 8.59-8.47 (m, 2H), 7.89 (d, J=7.6 Hz, 1H), 7.83-7.80 (m, 1H), 7.41 (m, 1H), 7.30 (m, 1H), 7.23 (d, J=8.1 Hz, 2H), 7.15 (d, J=7.8 Hz, 2H), 6.71 (m, 1H), 5.17 (s, 2H), 4.58 (d, J=5.9 Hz, 2H), 2.68 (s, 3H), 2.27 (s, 3H).

### Example 55: 6-(3-fluorobenzyl)-2-methyl-5-oxo-N-(pyridin-2-ylmethyl)-5,6-dihydro-1,6-naphthyridine-3-carboxamide

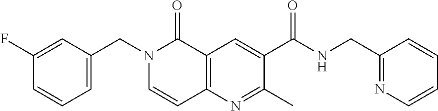 [SMILES: Cc1nc2ccn(Cc3cccc(F)c3)c(=O)c2cc1C(=O)NCc1ccccn1]

### 1. Synthesis of ethyl 6-(3-fluorobenzyl)-2-methyl-5-oxo-5,6-dihydro-1,6-naphthyridine-3-carboxylate

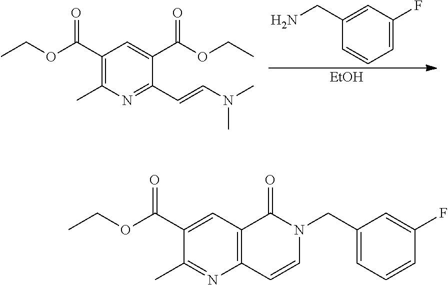 [SMILES: "EtOH" ; CCOC(=O)c1cc(C(=O)OCC)c(/C=C/N(C)C)nc1C ; NCc1cccc(F)c1 ; CCOC(=O)c1cc2c(=O)n(Cc3cccc(F)c3)ccc2nc1C]

A solution of diethyl (E)-2-(2-(dimethylamino)vinyl)-6-methylpyridine-3,5-dicarboxylate (500 mg, 1.63 mmol) and 1-(3-fluorophenyl)methanamine (310 mg, 2.47 mmol) in EtOH (5 mL) was stirred at 80° C. for 8 h. The precipitated solids were collected by filtration and washed with water (2×5 mL) to afford ethyl 6-(3-fluorobenzyl)-2-methyl-5-oxo-5,6-dihydro-1,6-naphthyridine-3-carboxylate as yellow solid (400 mg, yield: 72.0%). LCMS (ESI+): m/z calcd. for C₁₉H₁₈FN₂O₃[M+H]⁺, 341, found, 341.

### 2. Synthesis of 6-(3-fluorobenzyl)-2-methyl-5-oxo-5,6-dihydro-1,6-naphthyridine-3-carboxylic acid

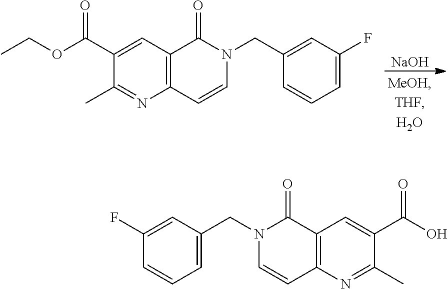

A solution of ethyl 6-(3-fluorobenzyl)-2-methyl-5-oxo-5,6-dihydro-1,6-naphthyridine-3-carboxylate (400 mg, 1.226 mmol) and NaOH (141 mg, 3.52 mmol) in MeOH (3 mL) and THF (3 mL) and H₂O (1 mL) was stirred for 4 h at 40° C. The residue was acidified to pH 4 with conc. HCl, and the aqueous layer was extracted with EtOAc. The combined organic layers were dried over Na₂SO₄ and concentrated in vacuo to afford 6-(3-fluorobenzyl)-2-methyl-5-oxo-5,6-dihydro-1,6-naphthyridine-3-carboxylic acid as a yellow solid (200 mg, yield: 52.2%). LCMS (ESI+): m/z calcd. for C₁₇H₁₄FN₂O₃[M+H]⁺, 313, found, 313.

### 3. Synthesis of 6-(3-fluorobenzyl)-2-methyl-5-oxo-N-(pyridin-2-ylmethyl)-5,6-dihydro-1,6-naphthyridine-3-carboxamide

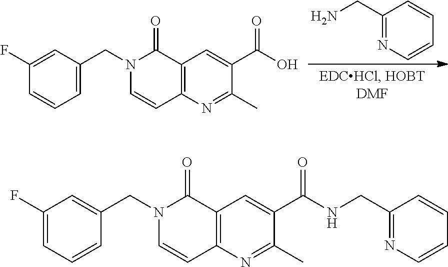 [SMILES: Cc1nc2ccn(Cc3cccc(F)c3)c(=O)c2cc1C(=O)O ; NCc1ccccn1 ; Cc1nc2ccn(Cc3cccc(F)c3)c(=O)c2cc1C(=O)NCc1ccccn1]

To a solution of 6-(3-fluorobenzyl)-2-methyl-5-oxo-5,6-dihydro-1,6-naphthyridine-3-carboxylic acid (200 mg, 0.640 mmol) and in DMF (3.5 mL) at 0° C. was added EDC·HCl (194 mg, 1.012 mmol) and HOBT (130 mg, 0.962 mmol), followed by addition of 2-pyridinemethaneamine (120 mg, 1.110 mmol). The reaction mixture was stirred at room temperature for 2 h. Then crude was purified by reverse phase column (gradient: 0˜50% ACN/H₂O (0.5% NH₄HCO₃)) to afford 6-(3-fluorobenzyl)-2-methyl-5-oxo-N-(pyridin-2-ylmethyl)-5,6-dihydro-1,6-naphthyridine-3-carboxamide as a white solid (78.3 mg, yield: 30.3%). LCMS (ESI+): m/z calcd. for C₂₃H₂₀FN₄O₂[M+H]⁺, 403.2, found, 403.3. ¹HNMR (400 MHz, DMSO-d₆, δ): 9.25 (d, J=6.0 Hz, 1H), 8.55 (d, J=5.2 Hz, 2H), 7.95 (d, J=7.6 Hz, 1H), 7.83-7.80 (m, 1H), 7.41-7.37 (m, 2H), 7.31 (d, J=5.2 Hz, 1H), 7.28-7.21 (m, 3H), 6.75 (d, J=7.6 Hz, 1H), 5.23 (s, 2H), 4.59 (d, J=5.6 Hz, 2H), 2.69 (s, 3H).

### Example 56: 6-(2-fluorobenzyl)-2-methyl-5-oxo-N-(pyridin-2-ylmethyl)-5,6-dihydro-1,6-naphthyridine-3-carboxamide

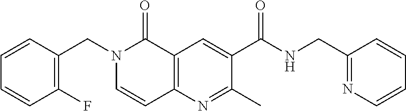 [SMILES: Cc1nc2ccn(Cc3ccccc3F)c(=O)c2cc1C(=O)NCc1ccccn1]

### 1. Synthesis of ethyl 6-(2-fluorobenzyl)-2-methyl-5-oxo-5,6-dihydro-1,6-naphthyridine-3-carboxylate

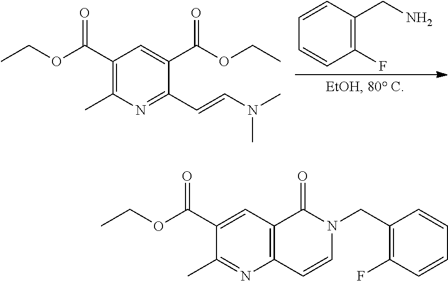

A solution of diethyl (E)-2-(2-(dimethylamino)vinyl)-6-methylpyridine-3,5-dicarboxylate (250 mg, 0.81 mmol) and 1-(2-fluorophenyl)methanamine (123 mg, 0.98 mmol) in EtOH (3 mL) was stirred for overnight at 80° C. under nitrogen atmosphere. The mixture was allowed to cool down to 0° C., the precipitated solids were collected by filtration and washed with H₂O (3×10 mL) to afford ethyl 6-(2-fluorobenzyl)-2-methyl-5-oxo-5,6-dihydro-1,6-naphthyridine-3-carboxylate as a yellow solid (223 mg, yield: 80.29%). LCMS (ESI+): m/z calcd. for C₁₉H₁₈FN₂O₃[M+H]⁺, 341, found, 341.

### 2. Synthesis of 6-(2-fluorobenzyl)-2-methyl-5-oxo-5,6-dihydro-1,6-naphthyridine-3-carboxylic acid

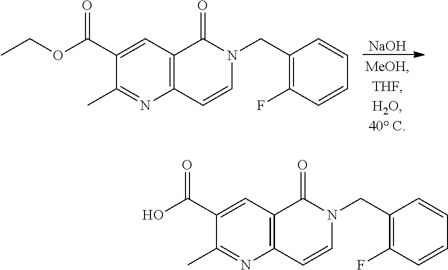

A solution of ethyl 6-(2-fluorobenzyl)-2-methyl-5-oxo-5,6-dihydro-1,6-naphthyridine-3-carboxylate (385 mg, 1.13 mmol) and NaOH (136 mg, 3.40 mmol) in MeOH (6 mL), THF (6 mL) and H₂O (2 mL) was stirred for 4 h at 40° C. The residue was acidified to pH 7 with conc. HCl, and the aqueous layer was extracted with EtOAc. The combined organic layers were dried over Na₂SO₄ and concentrated in vacuo to afford 6-(2-fluorobenzyl)-2-methyl-5-oxo-5,6-dihydro-1,6-naphthyridine-3-carboxylic acid as a white solid (196 mg, yield: 55%). LCMS (ESI+): m/z calcd. for C₁₇H₁₃FN₂O₃[M+H]⁺, 313, found, 313.

### 3. Synthesis of 6-(2-fluorobenzyl)-2-methyl-5-oxo-N-(pyridin-2-ylmethyl)-5,6-dihydro-1,6-naphthyridine-3-carboxamide

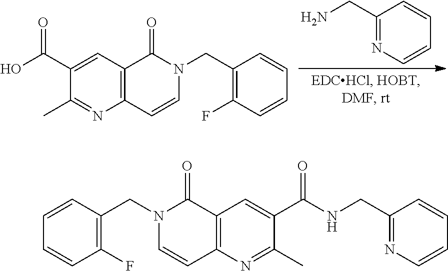 [SMILES: Cc1nc2ccn(Cc3ccccc3F)c(=O)c2cc1C(=O)O ; NCc1ccccn1 ; Cc1nc2ccn(Cc3ccccc3F)c(=O)c2cc1C(=O)NCc1ccccn1]

To a solution of 6-(2-fluorobenzyl)-2-methyl-5-oxo-5,6-dihydro-1,6-naphthyridine-3-carboxylic acid (190 mg, 0.60 mmol) in DMF (6 mL) at 0° C. was added EDC·HCl (240 mg, 1.25 mmol) and HOBT (124 mg, 0.91 mmol), followed by addition of 2-pyridinemethaneamine (99 mg, 0.91 mmol). The reaction mixture was stirred at room temperature for 0.5 h. Then crude was purified by reverse phase column (gradient: 0˜50% ACN/H₂O (0.5% NH₄HCO₃)) to afford 6-(2-fluorobenzyl)-2-methyl-5-oxo-N-(pyridin-2-ylmethyl)-5,6-dihydro-1,6-naphthyridine-3-carboxamide as a white solid (126.3 mg, yield: 51.38%). LCMS (ESI+): m/z calcd. for C₂₃H₂₀FN₄O₂[M+H]⁺, 403.2, found, 403.1. ¹HNMR (400 MHz, DMSO-d₆, δ): 9.23 (t, J=6.0 Hz, 1H), 8.60-8.45 (m, 2H), 7.94-7.75 (m, 2H), 7.44-7.09 (m, 6H), 6.75 (d, J=7.6 Hz, 1H), 5.27 (s, 2H), 4.58 (d, J=5.9 Hz, 2H), 2.69 (s, 3H).

### Example 57: 6-benzyl-N-((5-chloro-4-fluoropyridin-2-yl)methyl)-2-methyl-5-oxo-5,6-dihydro-1,6-naphthyridine-3-carboxamide

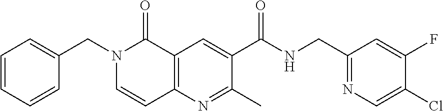 [SMILES: Cc1nc2ccn(Cc3ccccc3)c(=O)c2cc1C(=O)NCc1cc(F)c(Cl)cn1]

### 1. Synthesis of tert-butyl ((5-chloro-4-fluoropyridin-2-yl)methyl)carbamate

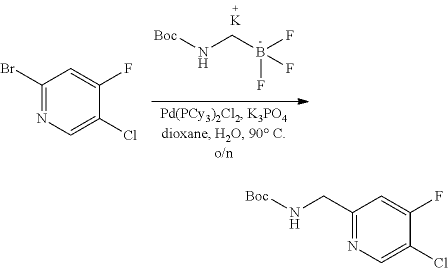

A solution of 2-bromo-5-chloro-4-fluoropyridine (50 mg, 0.23 mmol), tert-butyl N-[(trifluoro-lambda4-boranyl)methyl]carbamate potassium (63 mg, 0.26 mmol), Pd(PCy₃)₂Cl₂ (27 mg, 0.03 mmol) and K₃PO₄ (153 mg, 0.72 mmol) in dioxane (2 mL) and H₂O (0.4 mL) was stirred for overnight at 90° C. under nitrogen atmosphere. The resulting mixture was filtered, extracted with EtOAc, and dried over anhydrous Na₂SO₄. The filtrate was concentrated under reduced pressure and purified by silica gel column chromatography, eluted with EtOAc in petroleum ether (0-50%) to afford tert-butyl ((5-chloro-4-fluoropyridin-2-yl)methyl)carbamate as a colorless oil (10 mg, yield: 16.14%). LCMS (ESI+): m/z calcd. for C₁₁H₁₅ClFN₂O₂[M+H]⁺, 261.1, found, 261.0.

### 2. Synthesis of (5-chloro-4-fluoropyridin-2-yl)methanamine hydrochloride

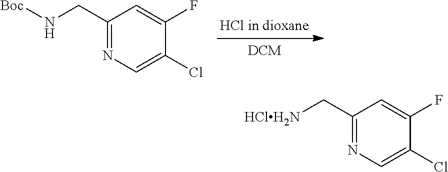

To a solution of tert-butyl ((5-chloro-4-fluoropyridin-2-yl)methyl)carbamate (10 mg, 0.03 mmol) in HCl(gas) in 1,4-dioxane (1 mL) and DCM (1 mL) was stirred for 1 h at room temperature. The resulting mixture was concentrated under reduced pressure to afford crude product (5-chloro-4-fluoropyridin-2-yl)methanamine hydrochloride (5 mg, yield: 89%) was used in the next step directly without further purification. LCMS (ESI+): m/z calcd. for C₆H₇ClFN₂ [M+H]⁺, 161.0, found, 161.0.

### 3. Synthesis of 6-benzyl-N-((5-chloro-4-fluoropyridin-2-yl)methyl)-2-methyl-5-oxo-5,6-dihydro-1,6-naphthyridine-3-carboxamide

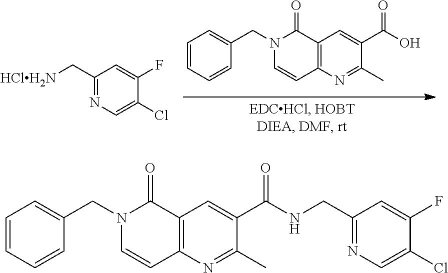

To a solution of 6-benzyl-2-methyl-5-oxo-5,6-dihydro-1,6-naphthyridine-3-carboxylic acid (14 mg, 0.04 mmol) in DMF (1 mL, 12.92 mmol) at 0° C. was added EDC·HCl (19 mg, 0.09 mmol) and HOBT (13 mg, 0.09 mmol), followed by addition of (5-chloro-4-fluoropyridin-2-yl)methanamine hydrochloride (5 mg, 0.03 mmol) and DIEA (19 mg, 0.14 mmol). The reaction mixture was stirred at room temperature for 1 h. Then crude was purified by reverse phase column (gradient: 0˜50% ACN/H₂O (0.5% NH₄HCO₃)) to afford 6-benzyl-N-((5-chloro-4-fluoropyridin-2-yl)methyl)-2-methyl-5-oxo-5,6-dihydro-1,6-naphthyridine-3-carboxamide as a white solid (9.6 mg, yield: 46%). LCMS (ESI+): m/z calcd. for C₂₃H₁₉ClFN₂O₂[M+H]⁺, 437.1, found, 437.1. ¹HNMR (400 MHz, DMSO-d₆, δ): 9.26 (t, J=5.8 Hz, 1H), 8.74 (d, J=9.7 Hz, 1H), 8.58 (s, 1H), 7.92 (m, 1H), 7.55 (d, J=10.2 Hz, 1H), 7.40-7.25 (m, 5H), 6.72 (d, J=7.5 Hz, 1H), 5.23 (s, 2H), 4.59 (d, J=5.8 Hz, 2H), 2.68 (s, 3H).

### Example 58: 2-methyl-5-oxo-N-(pyridin-2-ylmethyl)-6-(pyridin-3-ylmethyl)-5,6-dihydro-1,6-naphthyridine-3-carboxamide

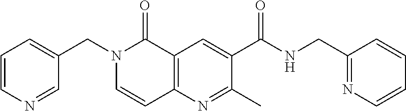 [SMILES: Cc1nc2ccn(Cc3cccnc3)c(=O)c2cc1C(=O)NCc1ccccn1]

### 1. Synthesis of ethyl 2-methyl-5-oxo-6-(pyridin-3-ylmethyl)-5,6-dihydro-1,6-naphthyridine-3-carboxylate

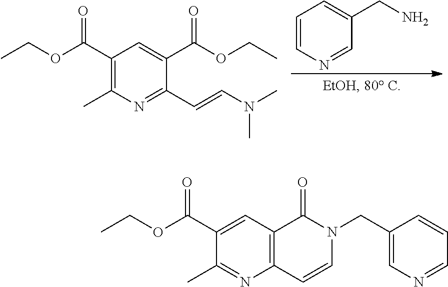

A solution of diethyl (E)-2-(2-(dimethylamino)vinyl)-6-methylpyridine-3,5-dicarboxylate (300.3 mg, 0.979 mmol) and pyridin-3-ylmethanamine (211.5 mg, 1.958 mmol) in EtOH (3 mL) was stirred at 80° C. for 8 h. Water (10 mL) was added at 0° C. and the solids were collected by filtration to afford ethyl 2-methyl-5-oxo-6-(pyridin-3-ylmethyl)-5,6-dihydro-1,6-naphthyridine-3-carboxylate as yellow solid (300.5 mg, yield: 94.8%). LCMS (ESI+): m/z calcd. for C₁₈H₁₈N₃O₃ [M+H]⁺, 324, found, 324.

### 2. Synthesis of 2-methyl-5-oxo-6-(pyridin-3-ylmethyl)-5,6-dihydro-1,6-naphthyridine-3-carboxylic acid

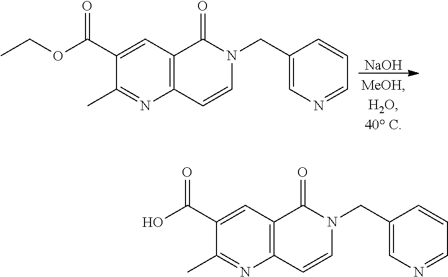

A solution of ethyl 2-methyl-5-oxo-6-(pyridin-3-ylmethyl)-5,6-dihydro-1,6-naphthyridine-3-carboxylate (200.2 mg, 0.62 mmol) and NaOH (74.4 mg, 1.86 mmol) in MeOH (3 mL) and H₂O (1 mL) was stirred for 3 h at 40° C. The residue was acidified to pH 4 with conc. HCl, and the aqueous layer was extracted with EtOAc. The combined organic layers were dried over Na₂SO₄ and concentrated in vacuo to afford 2-methyl-5-oxo-6-(pyridin-3-ylmethyl)-5,6-dihydro-1,6-naphthyridine-3-carboxylic acid as a white solid (123.3 mg, yield: 67.4%). LCMS (ESI+): m/z calcd. for C₁₆H₁₄N₃O₃ [M+H]⁺, 296, found, 296.

### 3. Synthesis of 2-methyl-5-oxo-N-(pyridin-2-ylmethyl)-6-(pyridin-3-ylmethyl)-5,6-dihydro-1,6-naphthyridine-3-carboxamide

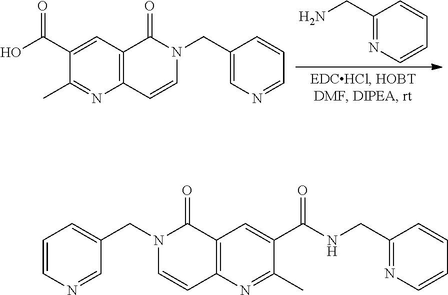 [SMILES: Cc1nc2ccn(Cc3cccnc3)c(=O)c2cc1C(=O)O ; NCc1ccccn1 ; Cc1nc2ccn(Cc3cccnc3)c(=O)c2cc1C(=O)NCc1ccccn1]

To a solution of 2-methyl-5-oxo-6-(pyridin-3-ylmethyl)-5,6-dihydro-1,6-naphthyridine-3-carboxylic acid (80.5 mg, 0.27 mmol) and in DMF (5 mL) at 0° C. was added EDC·HCl (78.3 mg, 0.405 mmol) and HOBT (55.6 mg, 0.405 mmol), DIPEA (70.1 mg, 0.54 mmol) followed by addition of pyridin-2-ylmethanamine (43.7 mg, 0.405 mmol). The reaction mixture was stirred at room temperature for 2 h. The crude product was purified by Prep-HPLC with the following conditions (Column: XBridge Prep OBD C₁₈ Column, 30*150 mm, 5 μm; Mobile Phase A: Water (10 mmol/L NH₄HCO₃), Mobile Phase B: ACN; Flow rate: 60 mL/min; Gradient: 33% B to 43% B in 8 min, 43%) to afford 2-methyl-5-oxo-N-(pyridin-2-ylmethyl)-6-(pyridin-3-ylmethyl)-5,6-dihydro-1,6-naphthyridine-3-carboxamide as a white solid (20.3 mg, yield: 19%). LCMS (ESI+): m/z calcd. for C₂₂H₂₀N₅O₂ [M+H]⁺, 386.2, found, 386.2. ¹HNMR (400 MHz, DMSO-d₆, δ): 9.22 (t, J=6.0 Hz, 1H), 8.63 (d, J=2.3 Hz, 1H), 8.54 (d, J=8.6 Hz, 2H), 8.51-8.49 (m, 1H), 8.00 (d, J=7.6 Hz, 1H), 7.84-7.78 (m, 1H), 7.76-7.72 (m, 1H), 7.43-7.35 (m, 2H), 7.32-7.28 (m, 1H), 6.75 (d, J=7.6 Hz, 1H), 5.25 (s, 2H), 4.58 (d, J=6.0 Hz, 2H), 2.69 (s, 3H).

### Example 59: N-((5-chloropyridin-2-yl)methyl)-6-(2-(2-(2-methoxyethoxy)ethoxy)ethyl)-2-methyl-5-oxo-5,6-dihydro-1,6-naphthyridine-3-carboxamide

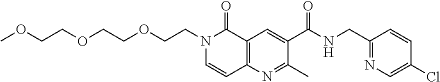 [SMILES: COCCOCCOCCn1ccc2nc(C)c(C(=O)NCc3ccc(Cl)cn3)cc2c1=O]

### 1. Synthesis of ethyl 6-(2-(2-(2-methoxyethoxy)ethoxy)ethyl)-2-methyl-5-oxo-5,6-dihydro-1,6-naphthyridine-3-carboxylate

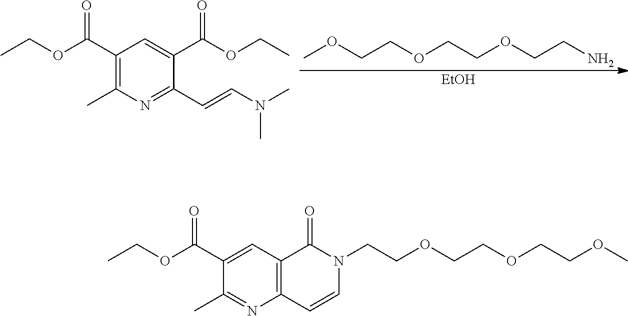

A solution of diethyl (E)-2-(2-(dimethylamino)vinyl)-6-methylpyridine-3,5-dicarboxylate (250 mg, 0.816 mmol) and 1-[2-(2-aminoethoxy)ethoxy]-2-methoxyethane (265 mg, 1.624 mmol) in EtOH (2.5 mL) was stirred for overnight at 80° C. under nitrogen atmosphere. The mixture was allowed to cool down to 0° C., the precipitated solids were collected by filtration and washed with H₂O (3×10 mL) to afford ethyl 6-(2-(2-(2-methoxyethoxy)ethoxy)ethyl)-2-methyl-5-oxo-5,6-dihydro-1,6-naphthyridine-3-carboxylate as a yellow solid (200.0 mg, yield: 64.7%). LCMS (ESI+): m/z calcd. for C₁₉H₂₇N₂06 [M+H]⁺, 379, found, 379.

### 2. Synthesis of 6-(2-(2-(2-methoxyethoxy)ethoxy)ethyl)-2-methyl-5-oxo-5,6-dihydro-1,6-naphthyridine-3-carboxylic acid

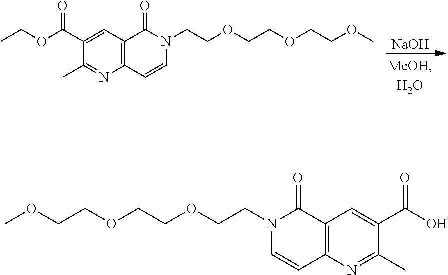

A solution of ethyl 6-(2-(2-(2-methoxyethoxy)ethoxy)ethyl)-2-methyl-5-oxo-5,6-dihydro-1,6-naphthyridine-3-carboxylate (200 mg, 0.549 mmol) and NaOH (63 mg, 1.575 mmol) in MeOH (3 mL) and H₂O (1 mL) was stirred for 4 h at 40° C. The residue was acidified to pH 4 with conc. HCl, and the aqueous layer was extracted with EtOAc. The combined organic layers were dried over Na₂SO₄ and concentrated in vacuo to afford 6-(2-(2-(2-methoxyethoxy)ethoxy)ethyl)-2-methyl-5-oxo-5,6-dihydro-1,6-naphthyridine-3-carboxylic acid as a white solid (70 mg, yield: 36.4%). LCMS (ESI+): m/z calcd. for C₁₇H₂₃N₂O₆ [M+H]⁺, 351, found, 351.

### 3. Synthesis of N-((5-chloropyridin-2-yl)methyl)-6-(2-(2-(2-methoxyethoxy)ethoxy)ethyl)-2-methyl-5-oxo-5,6-dihydro-1,6-naphthyridine-3-carboxamide

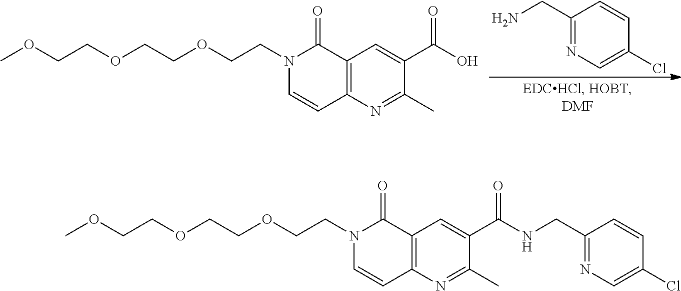 [SMILES: COCCOCCOCCn1ccc2nc(C)c(C(=O)O)cc2c1=O ; NCc1ccc(Cl)cn1 ; COCCOCCOCCn1ccc2nc(C)c(C(=O)NCc3ccc(Cl)cn3)cc2c1=O]

To a solution of 6-(2-(2-(2-methoxyethoxy)ethoxy)ethyl)-2-methyl-5-oxo-5,6-dihydro-1,6-naphthyridine-3-carboxylic acid (70 mg, 0.20 mmol) in DMF (2 mL) at 0° C. was added EDC·HCl (57 mg, 0.29 mmol) and HOBT (40 mg, 0.29 mmol), followed by addition of 1-(5-chloropyridin-2-yl)methanamine (42 mg, 0.295 mmol). The reaction mixture was stirred at room temperature for 2 h. Then crude was purified by reverse phase column (gradient: 0-50% ACN/H₂O (0.5% NH₄HCO₃)) to afford N-((5-chloropyridin-2-yl)methyl)-6-(2-(2-(2-methoxyethoxy)ethoxy)ethyl)-2-methyl-5-oxo-5,6-dihydro-1,6-naphthyridine-3-carboxamide as a white solid (36.3 mg, yield: 38.2%). LCMS (ESI+): m/z calcd. for C₂₃H₂₈ClN₄O₅ [M+H]⁺, 475.2, found, 475.2. ¹HNMR (400 MHz, DMSO-d₆, δ): 9.28-9.25 (m, 1H), 8.61 (d, J=2.4 Hz, 1H), 8.53 (s, 1H), 7.97-7.94 (m, 1H), 7.76 (d, J=7.6 Hz, 1H), 7.48 (d, J=8.4 Hz, 1H), 6.67 (d, J=7.5 Hz, 1H), 4.59 (d, J=6.0 Hz, 2H), 4.19-4.15 (m, 2H), 3.72-3.70 (m, 2H), 3.54-3.52 (m, 2H), 3.49-3.44 (m, 4H), 3.37 (s, 2H), 3.20 (s, 3H), 2.68 (s, 3H).

### Example 60: 6-(bicyclo[1.1.1]pentan-1-ylmethyl)-2-methyl-5-oxo-N-(pyridin-2-ylmethyl)-5,6-dihydro-1,6-naphthyridine-3-carboxamide

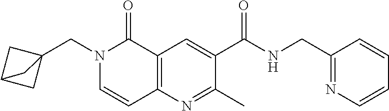 [SMILES: Cc1nc2ccn(CC34CC(C3)C4)c(=O)c2cc1C(=O)NCc1ccccn1]

### 1. Synthesis of ethyl 6-(bicyclo[1.1.1]pentan-1-ylmethyl)-2-methyl-5-oxo-5,6-dihydro-1,6-naphthyridine-3-carboxylate

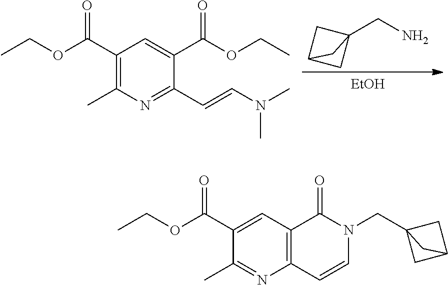

A solution of diethyl (E)-2-(2-(dimethylamino)vinyl)-6-methylpyridine-3,5-dicarboxylate (345 mg, 1.12 mmol) and 1-{bicyclo[1.1.1]pentan-1-yl}methanamine (150 mg, 1.54 mmol) in EtOH (4.0 mL) was stirred for overnight at 80° C. under nitrogen atmosphere. Then crude was purified by reverse phase column (gradient: 0˜50% ACN/H₂O (0.5% NH₄HCO₃)) to afford ethyl 6-(bicyclo[1.1.1]pentan-1-ylmethyl)-2-methyl-5-oxo-5,6-dihydro-1,6-naphthyridine-3-carboxylate (300 mg, yield: 85.2%) as a yellow solid. LCMS (ESI+): m/z calcd. for C₁₈H₂₁N₂O₃ [M+H]⁺, 313, found, 313.

### 2. Synthesis of 6-(bicyclo[1.1.1]pentan-1-ylmethyl)-2-methyl-5-oxo-5,6-dihydro-1,6-naphthyridine-3-carboxylic acid

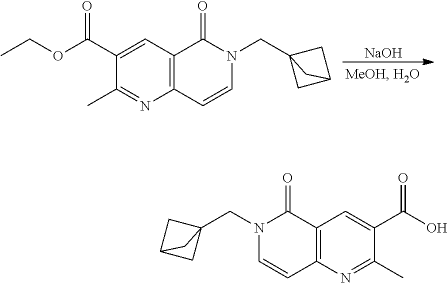

A solution of ethyl 6-(bicyclo[1.1.1]pentan-1-ylmethyl)-2-methyl-5-oxo-5,6-dihydro-1,6-naphthyridine-3-carboxylate (300 mg, 0.960 mmol) and NaOH (125 mg, 3.12 mmol) in MeOH (3 mL) and H₂O (1 mL) was stirred for 4 h at 40° C. The residue was acidified to pH 4 with conc. HCl, and the aqueous layer was extracted with EtOAc. The combined organic layers were dried over Na₂SO₄ and concentrated in vacuo to afford 6-(bicyclo[1.1.1]pentan-1-ylmethyl)-2-methyl-5-oxo-5,6-dihydro-1,6-naphthyridine-3-carboxylic acid (150 mg, yield: 54.9%) as a white solid. LCMS (ESI+): m/z calcd. for C₁₆H₁₇N₂O₃ [M+H]⁺, 285, found, 285.

### 3. Synthesis of 6-(bicyclo[1.1.1]pentan-1-ylmethyl)-2-methyl-5-oxo-N-(pyridin-2-ylmethyl)-5,6-dihydro-1,6-naphthyridine-3-carboxamide

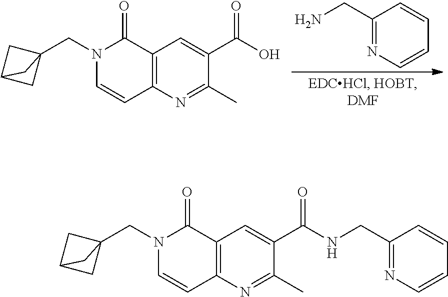 [SMILES: Cc1nc2ccn(CC34CC(C3)C4)c(=O)c2cc1C(=O)O ; NCc1ccccn1 ; Cc1nc2ccn(CC34CC(C3)C4)c(=O)c2cc1C(=O)NCc1ccccn1]

To a solution of 6-(bicyclo[1.1.1]pentan-1-ylmethyl)-2-methyl-5-oxo-5,6-dihydro-1,6-naphthyridine-3-carboxylic acid (50 mg, 0.176 mmol) in DMF (2 mL) at 0° C. was added EDC·HCl (50 mg, 0.261 mmol) and HOBT (35 mg, 0.259 mmol), followed by addition of 2-pyridinemethaneamine (28 mg, 0.259 mmol). The reaction mixture was stirred at room temperature for 2 h. Then crude was purified by reverse phase column (gradient: 0-50% ACN/H₂O (0.5% NH₄HCO₃)) to afford 6-(bicyclo[1.1.1]pentan-1-ylmethyl)-2-methyl-5-oxo-N-(pyridin-2-ylmethyl)-5,6-dihydro-1,6-naphthyridine-3-carboxamide (17.5 mg, yield: 26.2%) as a white solid. LCMS (ESI+): m/z calcd. for C₂₂H₂₃N₄O₂ [M+H]⁺, 375.1, found, 375.1. ¹HNMR (400 MHz, DMSO-d₆, δ): 9.26-9.24 (m, 1H), 8.56-8.53 (m, 2H), 7.84-7.80 (m, 1H), 7.68 (d, J=7.5 Hz, 1H), 7.42 (d, J=7.9 Hz, 1H), 7.31 (d, J=2.6 Hz, 1H), 6.68 (d, J=7.5 Hz, 1H), 4.59 (d, J=5.9 Hz, 2H), 4.08 (s, 2H), 2.69 (s, 3H), 2.47 (s, 1H), 1.69 (s, 6H).

### Example 61: 6-(3,4-difluorobenzyl)-2-methyl-5-oxo-N-(pyridin-2-ylmethyl)-5,6-dihydro-1,6-naphthyridine-3-carboxamide

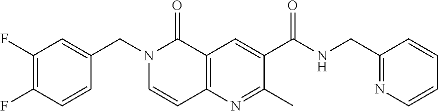 [SMILES: Cc1nc2ccn(Cc3ccc(F)c(F)c3)c(=O)c2cc1C(=O)NCc1ccccn1]

### 1. Synthesis of ethyl 6-(3,4-difluorobenzyl)-2-methyl-5-oxo-5,6-dihydro-1,6-naphthyridine-3-carboxylate

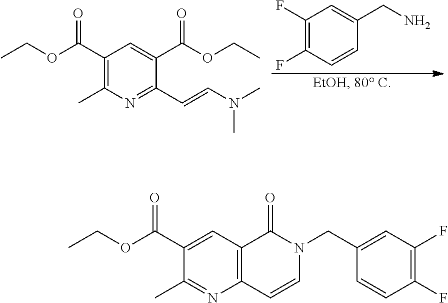

A solution of diethyl (E)-2-(2-(dimethylamino)vinyl)-6-methylpyridine-3,5-dicarboxylate (500 mg, 1.63 mmol) and 1-(3,4-difluorophenyl) methanamine (282 mg, 1.97 mmol) in EtOH (5 mL) was stirred for overnight at 80° C. under nitrogen atmosphere. The mixture was cooled down to 0° C., the precipitated solids were collected by filtration and washed with H₂O (3×10 mL) to afford crude ethyl 6-(3,4-difluorobenzyl)-2-methyl-5-oxo-5,6-dihydro-1,6-naphthyridine-3-carboxylate (510 mg) as an orange solid. LCMS (ESI+): m/z calcd. for C₁₉H₁₇F₂N₂O₃[M+H]⁺, 359, found, 359.

### 2. Synthesis of 6-(3,4-difluorobenzyl)-2-methyl-5-oxo-5,6-dihydro-1,6-naphthyridine-3-carboxylic acid

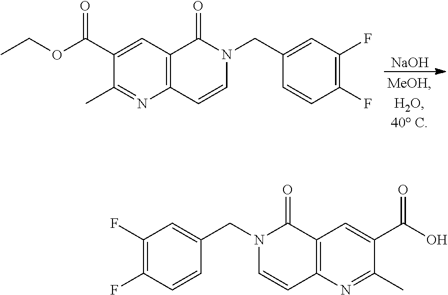

A solution of ethyl 6-(3,4-difluorobenzyl)-2-methyl-5-oxo-5,6-dihydro-1,6-naphthyridine-3-carboxylate (500 mg, 1.39 mmol) and NaOH (158 mg, 3.95 mmol) in MeOH (10 mL) and H₂O (3 mL) was stirred for overnight at 40° C. The residue was acidified to pH 4 with conc. HCl, and the aqueous layer was extracted with EtOAc. The combined organic layers were dried over Na₂SO₄ and concentrated in vacuo to afford 6-(3,4-difluorobenzyl)-2-methyl-5-oxo-5,6-dihydro-1,6-naphthyridine-3-carboxylic acid (410 mg, yield: 88.9%) as a yellow solid. LCMS (ESI+): m/z calcd. for C₁₇H₁₃F₂N₂O₃[M+H]⁺, 331, found, 331.

### 3. Synthesis of 6-(3,4-difluorobenzyl)-2-methyl-5-oxo-N-(pyridin-2-ylmethyl)-5,6-dihydro-1,6-naphthyridine-3-carboxamide

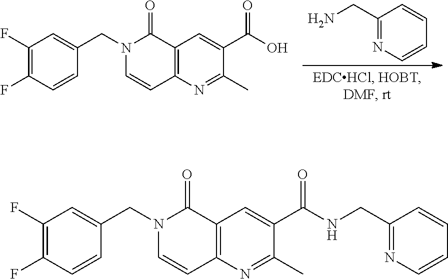 [SMILES: Cc1nc2ccn(Cc3ccc(F)c(F)c3)c(=O)c2cc1C(=O)O ; NCc1ccccn1 ; Cc1nc2ccn(Cc3ccc(F)c(F)c3)c(=O)c2cc1C(=O)NCc1ccccn1]

A solution of 6-(3,4-difluorobenzyl)-2-methyl-5-oxo-5,6-dihydro-1,6-naphthyridine-3-carboxylic acid (100 mg, 0.30 mmol) in DMF (3 mL) at 0° C. was added EDC·HCl (88 mg, 0.45 mmol) and HOBT (62 mg, 0.45 mmol), followed by addition of 2-pyridinemethaneamine (49 mg, 0.45 mmol). The reaction mixture was stirred at room temperature for 0.5 h. Then crude was purified by reverse phase column: ACN/H₂O (0.5% NH₄HCO₃) to afford 6-(3,4-difluorobenzyl)-2-methyl-5-oxo-N-(pyridin-2-ylmethyl)-5,6-dihydro-1,6-naphthyridine-3-carboxamide (67.2 mg, yield: 52.7%) as a white solid. LCMS (ESI+): m/z calcd. for C₂₃H₁₉F₂N₄O₂[M+H]⁺, 421.1, found, 421.1. ¹HNMR (400 MHz, DMSO-d₆, δ): 9.22 (t, J=6.0 Hz, 1H), 8.58-8.46 (m, 2H), 7.95 (d, J=7.6 Hz, 1H), 7.81 (m, 1H), 7.55-7.34 (m, 3H), 7.31-7.28 (m, 1H), 7.20 (m, 1H), 6.74 (d, J=7.6 Hz, 1H), 5.20 (s, 2H), 4.59 (d, J=5.9 Hz, 2H), 2.69 (s, 3H).

### Example 62: 6-(3,4-difluorobenzyl)-N-((4,5-difluoropyridin-2-yl)methyl)-2-methyl-5-oxo-5,6-dihydro-1,6-naphthyridine-3-carboxamide

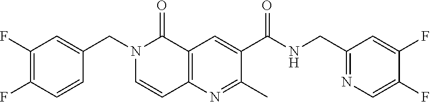 [SMILES: Cc1nc2ccn(Cc3ccc(F)c(F)c3)c(=O)c2cc1C(=O)NCc1cc(F)c(F)cn1]

### 1. Synthesis of 6-(3,4-difluorobenzyl)-N-((4,5-difluoropyridin-2-yl)methyl)-2-methyl-5-oxo-5,6-dihydro-1,6-naphthyridine-3-carboxamide

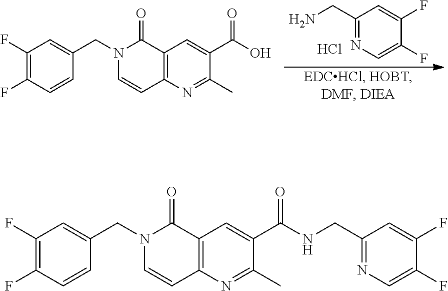

A solution of 6-(3,4-difluorobenzyl)-2-methyl-5-oxo-5,6-dihydro-1,6-naphthyridine-3-carboxylic acid (28 mg, 0.08 mmol) in DMF (1 mL) at 0° C. was added EDC·HCl (22 mg, 0.11 mmol), HOBT (15 mg, 0.11 mmol) and DIEA (22 mg, 0.17 mmol), followed by addition of 1-(4,5-difluoropyridin-2-yl) methanamine hydrochloride (10 mg, 0.05 mmol). The reaction mixture was stirred at room temperature for 0.5 h. Then crude was purified by reverse phase column (gradient: 0˜50% ACN/H₂O (0.50% NH₄HCO₃)) to afford 6-(3,4-difluorobenzyl)-N-((4,5-difluoropyridin-2-yl)methyl)-2-methyl-5-oxo-5,6-dihydro-1,6-naphthyridine-3-carboxamide (14 mg, yield: 35.7%) as a white solid. LCMS (ESI+): m/z calcd. for C₂₃H₁₇F₄N₄O₂[M+H]⁺, 457.1, found, 457.2. ¹HNMR (400 MHz, DMSO-d₆, δ): 9.23 (t, J=5.9 Hz, 1H), 8.74-8.71 (m, 1H), 8.56 (s, 1H), 7.94 (d, J=7.6 Hz, 1H), 7.60-7.56 (m, 1H), 7.49-7.36 (m, 2H), 7.23-7.15 (m, 1H), 6.73 (d, J=7.6 Hz, 1H), 5.19 (s, 2H), 4.56 (d, J=5.8 Hz, 2H), 2.67 (s, 3H).

### Example 63: 6-(3,4-difluorobenzyl)-N-((5-fluoropyridin-2-yl)methyl)-2-methyl-5-oxo-5,6-dihydro-1,6-naphthyridine-3-carboxamide

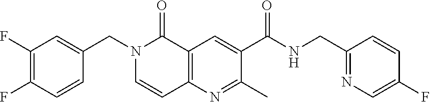 [SMILES: Cc1nc2ccn(Cc3ccc(F)c(F)c3)c(=O)c2cc1C(=O)NCc1ccc(F)cn1]

### 1. Synthesis of 6-(3,4-difluorobenzyl)-N-((5-fluoropyridin-2-yl)methyl)-2-methyl-5-oxo-5,6-dihydro-1,6-naphthyridine-3-carboxamide

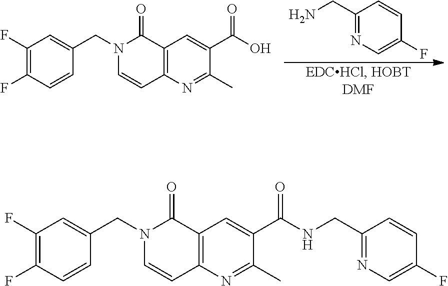

A solution of 6-(3,4-difluorobenzyl)-2-methyl-5-oxo-5,6-dihydro-1,6-naphthyridine-3-carboxylic acid (100 mg, 0.30 mmol) in DMF (2 mL) at 0° C. was added EDC·HCl (88 mg, 0.45 mmol) and HOBT (62 mg, 0.45 mmol), followed by addition of 1-(5-fluoropyridin-2-yl) methanamine (58 mg, 0.46 mmol). The reaction mixture was stirred at room temperature for 0.5 h. Then crude was purified by reverse phase column: ACN/H₂O (0.1% NH₄HCO₃) to afford 6-(3,4-difluorobenzyl)-N-((5-fluoropyridin-2-yl)methyl)-2-methyl-5-oxo-5,6-dihydro-1,6-naphthyridine-3-carboxamide (74.5 mg, yield: 55.0%) as a white solid. LCMS (ESI+): m/z calcd. for C₂₃H₁₈F₃N₄O₂ [M+H]⁺, 439.1, found, 439.1. ¹HNMR (400 MHz, DMSO-d₆, δ): 9.25 (t, J=6.0 Hz, 1H), 8.61-8.46 (m, 2H), 7.95 (d, J=7.6 Hz, 1H), 7.77-7.72 (m, 1H), 7.57-7.33 (m, 3H), 7.21-7.18 (m, 1H), 6.74 (d, J=7.5 Hz, 1H), 5.20 (s, 2H), 4.58 (d, J=5.9 Hz, 2H), 2.68 (s, 3H).

### Example 64: N-((5-chloropyridin-2-yl)methyl)-6-(4-(2-(2-methoxyethoxy)ethoxy)benzyl)-2-methyl-5-oxo-5,6-dihydro-1,6-naphthyridine-3-carboxamide

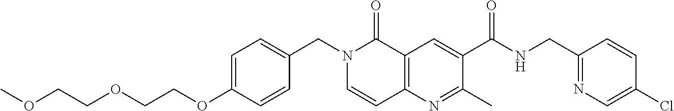 [SMILES: COCCOCCOc1ccc(Cn2ccc3nc(C)c(C(=O)NCc4ccc(Cl)cn4)cc3c2=O)cc1]

### 1. Synthesis of tert-butyl (4-(2-(2-methoxyethoxy)ethoxy)benzyl)carbamate

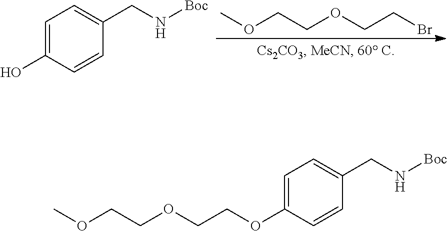

A solution of tert-butyl (4-hydroxybenzyl)carbamate (300 mg, 1.34 mmol), 1-bromo-2-(2-methoxyethoxy)ethane (245 mg, 1.34 mmol) and Cs₂CO₃ (1.31 g, 4.03 mmol) in MeCN (3 mL) was stirred for 2 h at 60° C. under an nitrogen atmosphere. The reaction was quenched with water (3 mL). The mixture was extracted with EtOAc (3×5 mL) to afford tert-butyl (4-(2-(2-methoxyethoxy)ethoxy)benzyl)carbamate (324 mg, yield: 74.1%) as a white solid. LCMS (ESI+): m/z calcd. for C₁₇H₂₈NO₅ [M+H]⁺, 326, found, 326.

### 2. Synthesis of (4-(2-(2-methoxyethoxy)ethoxy)phenyl)methanamine hydrochloride

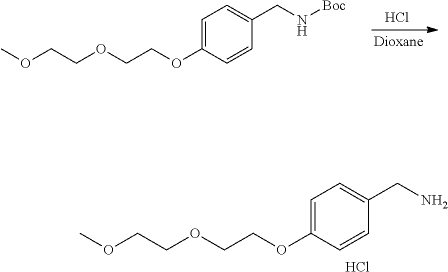 [SMILES: COCCOCCOc1ccc(CNC(=O)OC(C)(C)C)cc1 ; Cl ; COCCOCCOc1ccc(CN)cc1 ; Cl]

A solution of tert-butyl (4-(2-(2-methoxyethoxy)ethoxy)benzyl)carbamate (324 mg, 0.996 mmol) in HCl (gas) in 1,4-dioxane (3 mL) was stirred for 30 min at room temperature. The precipitated solids were collected by filtration to afford (4-(2-(2-methoxyethoxy)ethoxy)phenyl)methanamine hydrochloride (227 mg, yield: 87.1%) as a grey solid. LCMS (ESI+): m/z calcd. for C₁₂H₂₀NO₃ [M+H]⁺, 226, found, 226.

### 3. Synthesis of ethyl 6-(4-(2-(2-methoxyethoxy)ethoxy)benzyl)-2-methyl-5-oxo-5,6-dihydro-1,6-naphthyridine-3-carboxylate

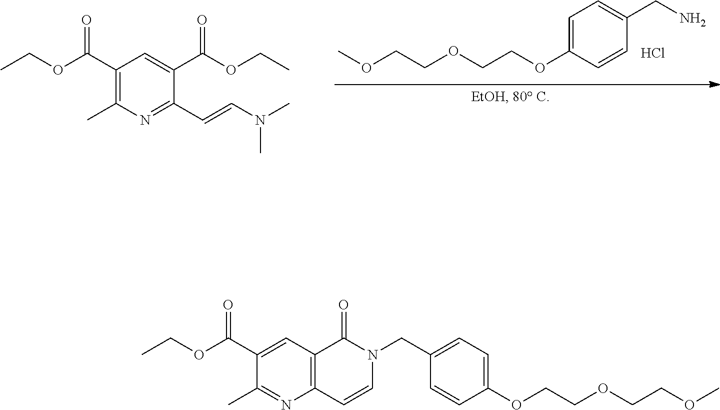 [SMILES: CCOC(=O)c1cc(C(=O)OCC)c(/C=C/N(C)C)nc1C ; COCCOCCOc1ccc(CN)cc1 ; Cl ; CCOC(=O)c1cc2c(=O)n(Cc3ccc(OCCOCCOC)cc3)ccc2nc1C]

To a stirred solution of diethyl (E)-2-(2-(dimethylamino)vinyl)-6-methylpyridine-3,5-dicarboxylate (150 mg, 0.490 mmol) in EtOH (1.6 mL) was added (4-(2-(2-methoxyethoxy)ethoxy)phenyl)methanamine hydrochloride (220 mg, 0.980 mmol) under an nitrogen atmosphere, at room temperature. The resulting mixture was stirred overnight at 80° C. The reaction was quenched by the addition of water (2 mL) at 0° C. The mixture was extracted with EtOAc (3×5 mL). The organic layers were concentrated under reduced pressure to afford ethyl 6-(4-(2-(2-methoxyethoxy)ethoxy)benzyl)-2-methyl-5-oxo-5,6-dihydro-1,6-naphthyridine-3-carboxylate (180 mg, yield: 83.4%) as a yellow solid. LCMS (ESI+): m/z calcd. for C₂₄H₂₉N₂O₆ [M+H]⁺, 441, found, 441.

### 4. Synthesis of 6-(4-(2-(2-methoxyethoxy)ethoxy)benzyl)-2-methyl-5-oxo-5,6-dihydro-1,6-naphthyridine-3-carboxylic acid

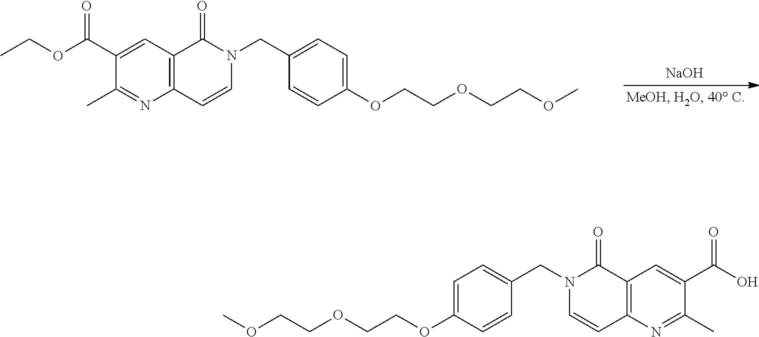

A solution of ethyl 6-(4-(2-(2-methoxyethoxy)ethoxy)benzyl)-2-methyl-5-oxo-5,6-dihydro-1,6-naphthyridine-3-carboxylate (120 mg, 0.272 mmol) and NaOH (32.7 mg, 0.816 mmol) in MeOH:H₂O=3:1 (2 mL) was stirred for overnight at 40° C. The mixture was acidified to pH 5 with HCl (aq.). The residue was purified by reverse flash chromatography with MeOH in water (0.1% HCl), 15% to 45% in 20 min. to afford 6-(4-(2-(2-methoxyethoxy)ethoxy)benzyl)-2-methyl-5-oxo-5,6-dihydro-1,6-naphthyridine-3-carboxylic acid (100 mg, yield: 89.0%) as a yellow solid. LCMS (ESI+): m/z calcd. for C₂₂H₂₅N₂O₆ [M+H]⁺, 413, found, 413.

### 5. Synthesis of N-((5-chloropyridin-2-yl)methyl)-6-(4-(2-(2-methoxyethoxy)ethoxy)benzyl)-2-methyl-5-oxo-5,6-dihydro-1,6-naphthyridine-3-carboxamide

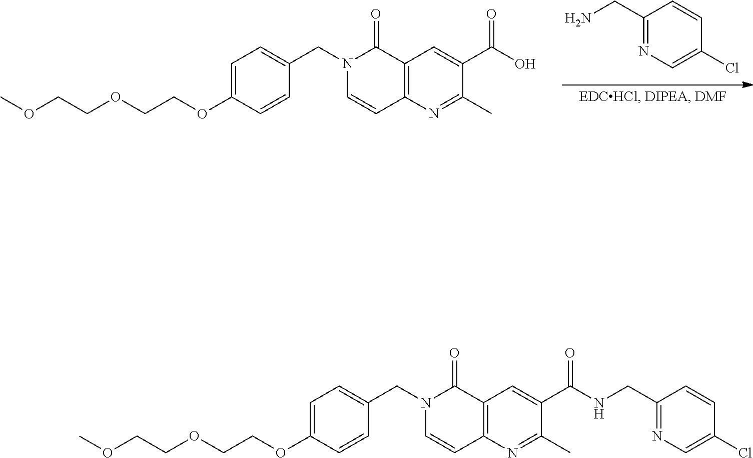 [SMILES: COCCOCCOc1ccc(Cn2ccc3nc(C)c(C(=O)O)cc3c2=O)cc1 ; NCc1ccc(Cl)cn1 ; COCCOCCOc1ccc(Cn2ccc3nc(C)c(C(=O)NCc4ccc(Cl)cn4)cc3c2=O)cc1]

To a solution of 6-(4-(2-(2-methoxyethoxy)ethoxy)benzyl)-2-methyl-5-oxo-5,6-dihydro-1,6-naphthyridine-3-carboxylic acid (100 mg, 0.242 mmol) in DMF under an nitrogen atmosphere was added EDC·HCl (69.7 mg, 0.363 mmol), DIPEA (94.0 mg, 0.726 mmol) and 1-(5-chloropyridin-2-yl)methanamine (51.8 mg, 0.363 mmol) at room temperature. The reaction was stirred for 1 h at room temperature. The reaction was purified by reverse flash chromatography with MeOH in Water (10 mmol/L NH₄HCO₃), 30% to 60% in 30 min to afford N-((5-chloropyridin-2-yl)methyl)-6-(4-(2-(2-methoxyethoxy)ethoxy)benzyl)-2-methyl-5-oxo-5,6-dihydro-1,6-naphthyridine-3-carboxamide (27 mg, yield: 20.5%) as a white solid. LCMS (ESI+): m/z calcd. for C₂₈H₃₀ClN₄O₅[M+H]⁺, 537.1, found, 537.1. ¹HNMR (400 MHz, DMSO-d₆, δ): 9.30-9.24 (m, 1H), 8.60 (d, J=2.5 Hz, 1H), 8.54 (s, 1H), 7.97-7.88 (m, 2H), 7.47 (d, J=8.4 Hz, 1H), 7.31 (d, J=8.4 Hz, 2H), 6.91 (d, J=8.5 Hz, 2H), 6.70 (d, J=7.6 Hz, 1H), 5.14 (s, 2H), 4.58 (d, J=5.9 Hz, 2H), 4.08-4.02 (m, 2H), 3.75-3.68 (m, 2H), 3.60-3.54 (m, 2H), 3.47-3.42 (m, 2H), 3.23 (s, 3H), 2.67 (s, 3H).

### Example 65: 6-benzyl-5-oxo-N-(pyridin-2-ylmethyl)-5,6-dihydro-1,6-naphthyridine-3-carboxamide

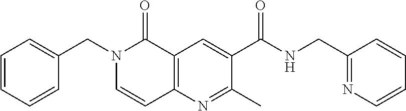 [SMILES: Cc1nc2ccn(Cc3ccccc3)c(=O)c2cc1C(=O)NCc1ccccn1]

### 1. Synthesis of diethyl 2-methylpyridine-3,5-dicarboxylate

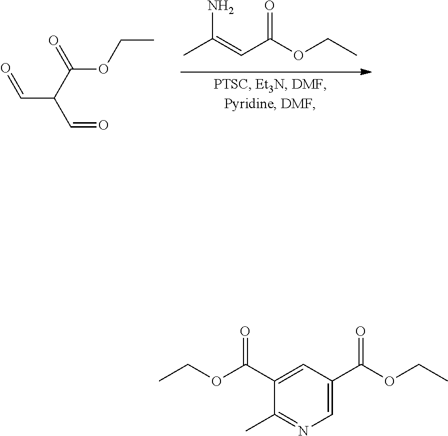

Under nitrogen atmosphere, to a mixture of ethyl 2-formyl-3-oxopropanoate (1 g, 6.93 mmol) in Et₂O (7 mL) was added Et₃N (0.77 g, 7.61 mmol) dropwise at 0° C. The reaction mixture was stirred at 25° C. for 2 h, then the solvent was evaporated under reduced pressure. The residue was diluted with DMF (5 mL) and methylbenzenesulfonyl (1.45 g, 7.606 mmol) in DMF (6 mL) was added at 0° C. The reaction mixture was stirred for 4 h at room temperature. To the mixture was added a solution of ethyl (2Z)-3-aminobut-2-enoate (0.95 g, 7.35 mmol) and pyridine (2.2 g, 27.8 mmol) in DMF (5 mL) at 25° C. The reaction mixture was stirred at 80° C. for 16 h. The reaction mixture was diluted with H₂O (10 mL), and extracted with ethyl acetate (2×20 mL). The combined organic layer was washed with saturated brine (2×10 mL), dried over anhydrous sodium sulfate, and concentrated under reduced pressure. The residue was purified by flash column chromatography on silica gel (gradient, 10%-30% EtOAc/petroleum ether) to afford diethyl 2-methylpyridine-3,5-dicarboxylate (950 mg, yield: 57.7%) as a yellow solid. LCMS (ESI+): m/z calcd. for C₁₂H₁₆NO₄ [M+H]⁺, 238, found, 238.

### 2. Synthesis of diethyl (E)-2-(2-(dimethylamino)vinyl)pyridine-3,5-dicarboxylate

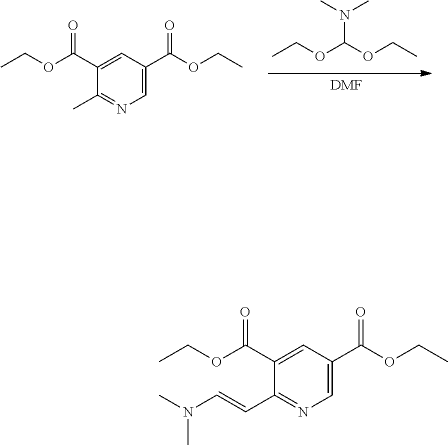

To a solution of diethyl 2-methylpyridine-3,5-dicarboxylate (950 mg, 4.00 mmol) in DMF (10 mL) was added (diethoxymethyl)dimethylamine (884 mg, 6.00 mmol) and the resulting solution was stirred at 100° C. for 8 h. The resulting solution was taken directly to the next step. LCMS (ESI+): m/z calcd. for C₁₅H₂₁N₂O₄ [M+H]⁺, 293, found, 293.

### 3. Synthesis of ethyl 6-benzyl-5-oxo-5,6-dihydro-1,6-naphthyridine-3-carboxylate

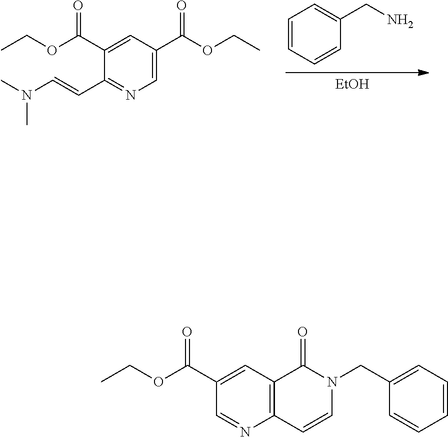

A solution of diethyl (E)-2-(2-(dimethylamino)vinyl)pyridine-3,5-dicarboxylate (950 mg, 3.25 mmol) and benzylamine (522 mg, 4.87 mmol) in EtOH (10 mL) was stirred at 80° C. for 8 h. The precipitated solids were collected by filtration and washed with water (2×5 mL) to afford ethyl 6-benzyl-5-oxo-5,6-dihydro-1,6-naphthyridine-3-carboxylate (400 mg, yield: 39.9%) as yellow solid. LCMS (ESI+): m/z calcd. for C₁₈H₁₇N₂O₃ [M+H]⁺, 309, found, 309.

### 4. Synthesis of 6-benzyl-5-oxo-5,6-dihydro-1,6-naphthyridine-3-carboxylic acid

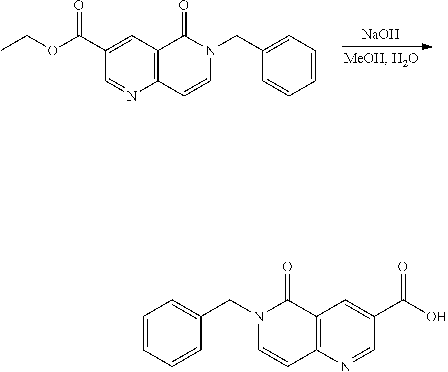

A solution of ethyl 6-benzyl-5-oxo-5,6-dihydro-1,6-naphthyridine-3-carboxylate (100 mg, 0.324 mmol) and NaOH (38 mg, 0.950 mmol) in MeOH (3 mL) and H₂O (1 mL) was stirred for 3 h at 40° C. The residue was acidified to pH 4 with conc. HCl. The aqueous layer was extracted with EtOAc (2×5 mL). The combined organic layers were dried over Na₂SO₄ and concentrated in vacuo to afford 6-benzyl-5-oxo-5,6-dihydro-1,6-naphthyridine-3-carboxylic acid (250 mg, 60.6%) as a white solid. LCMS (ESI+): m/z calcd. for C₁₆H₁₃N₂O₃ [M+H]⁺, 281, found, 281.

### 5. Synthesis of 6-benzyl-5-oxo-N-(pyridin-2-ylmethyl)-5,6-dihydro-1,6-naphthyridine-3-carboxamide

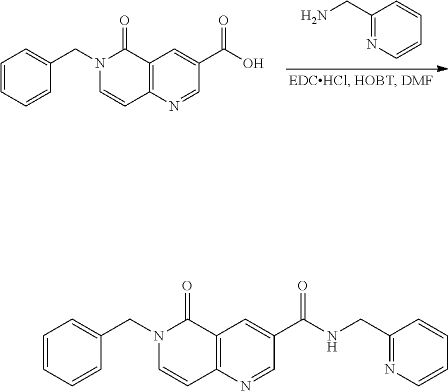 [SMILES: O=C(O)c1cnc2ccn(Cc3ccccc3)c(=O)c2c1 ; NCc1ccccn1 ; O=C(NCc1ccccn1)c1cnc2ccn(Cc3ccccc3)c(=O)c2c1]

To a solution of 6-benzyl-5-oxo-5,6-dihydro-1,6-naphthyridine-3-carboxylic acid (60 mg, 0.214 mmol) in DMF (2 mL) at 0° C. was added EDC·HCl (61.5 mg, 0.321 mmol) and HOBT (43.4 mg, 0.321 mmol), followed by addition of 2-pyridinemethaneamine (34.7 mg, 0.321 mmol). The reaction mixture was stirred at room temperature for 2 h. Then crude was purified by reverse phase column: ACN/H₂O (0.1% NH₄HCO₃) to afford 6-benzyl-5-oxo-N-(pyridin-2-ylmethyl)-5,6-dihydro-1,6-naphthyridine-3-carboxamide (24.6 mg, yield: 31.0%) as a white solid. LCMS (ESI+): m/z calcd. for C₂₂H₁₉N₄O₂ [M+H]⁺, 371.1, found, 371.1. ¹HNMR (400 MHz, DMSO-d₆, δ): 9.63-9.49 (m, 1H), 9.37 (d, J=2.3 Hz, 1H), 9.09 (d, J=2.3 Hz, 1H), 8.58-8.48 (m, 1H), 7.99 (d, J=7.6 Hz, 1H), 7.80-7.73 (m, 1H), 7.41-7.24 (m, 7H), 6.83 (d, J=7.6 Hz, 1H), 5.25 (s, 2H), 4.62 (d, J=5.9 Hz, 2H).

### Example 66: 6-benzyl-2-methyl-N-((4-methyloxazol-2-yl)methyl)-5-oxo-5,6-dihydro-1,6-naphthyridine-3-carboxamide

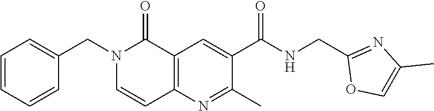 [SMILES: Cc1coc(CNC(=O)c2cc3c(=O)n(Cc4ccccc4)ccc3nc2C)n1]

### 1. Synthesis of (4-methyloxazol-2-yl)methanamine hydrochloride

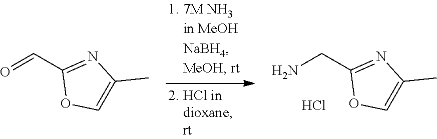

A solution of 4-methyloxazole-2-carbaldehyde (20.0 mg, 0.181 mmol) in MeOH (0.5 mL) was added NH₃ ammonia (g) in MeOH (0.5 mL, 3.50 mmol) was stirred for 3 h at 25° C. NaBH₄ (13.2 mg, 0.349 mmol) was added and the resulting mixture was stirred for 1 h at 25° C. The reaction was quenched with water/ice and extracted with EtOAc. The combined organic layers were dried over anhydrous Na₂SO₄ and concentrated under reduced pressure. followed by addition of HCl (gas) in 1,4-dioxane (1 mL, 4.0 mmol) and stirred for 5 min at 0° C. The resulting mixture was concentrated under reduced pressure to afford (4-methyloxazol-2-yl)methanamine hydrochloride (22 mg, yield: 82.1%) as a yellow oil. LCMS (ESI+): m/z calcd. for C₅H₉N₂O [M+H]⁺, 113, found, 113.

### 2. Synthesis of 6-benzyl-2-methyl-N-((4-methyloxazol-2-yl)methyl)-5-oxo-5,6-dihydro-1,6-naphthyridine-3-carboxamide

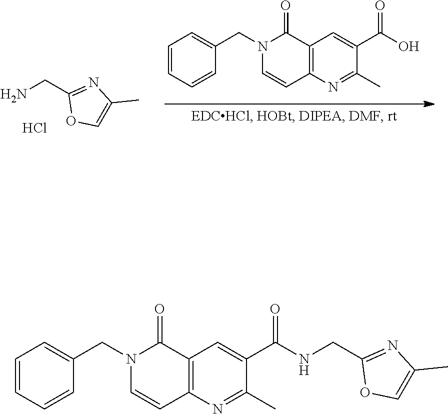

To a solution of 6-benzyl-2-methyl-5-oxo-5,6-dihydro-1,6-naphthyridine-3-carboxylic acid (43.6 mg, 0.148 mmol) in DMF (2 mL) at 0° C. was added EDC·HCl (40.6 mg, 0.212 mmol) and HOBT (29.5 mg, 0.218 mmol), followed by addition of (4-methyloxazol-2-yl)methanamine hydrochloride (20.5 mg, 0.138 mmol). The residue was purified by reverse flash chromatography eluted with MeCN in water (0.5% NH₄HCO₃), 10% to 100% gradient in 30 min to afford 6-benzyl-2-methyl-N-((4-methyloxazol-2-yl)methyl)-5-oxo-5,6-dihydro-1,6-naphthyridine-3-carboxamide (3.6 mg, yield: 6.7%) as a white solid. LCMS (ESI+): m/z calcd. for C₂₂H₂₁N₄O₃ [M+H]⁺, 389.2, found, 389.2. ¹HNMR (400 MHz, DMSO-d₆, δ): 9.39 (t, J=6.0 Hz, 1H), 8.50 (s, 1H), 7.95-7.90 (m, 1H), 7.77-7.71 (m, 1H), 7.43-7.33 (m, 5H), 6.73-6.71 (m, 1H), 5.22 (s, 2H), 4.55 (d, J=5.6 Hz, 2H), 2.67 (s, 3H), 2.20 (s, 3H).

### Example 67: 6-benzyl-N-(4-isopropoxybenzyl)-2-methyl-5-oxo-5,6-dihydro-1,6-naphthyridine-3-carboxamide

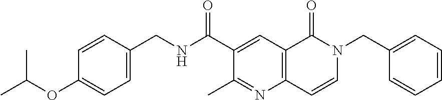 [SMILES: Cc1nc2ccn(Cc3ccccc3)c(=O)c2cc1C(=O)NCc1ccc(OC(C)C)cc1]

### 1. Synthesis of 6-benzyl-N-(4-isopropoxybenzyl)-2-methyl-5-oxo-5,6-dihydro-1,6-naphthyridine-3-carboxamide

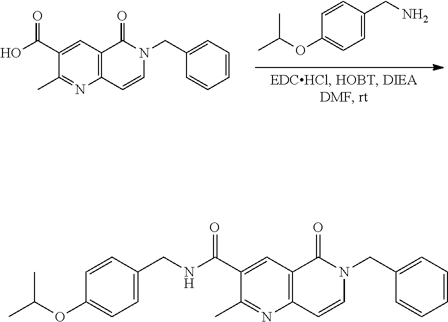

To a solution of 6-benzyl-2-methyl-5-oxo-5,6-dihydro-1,6-naphthyridine-3-carboxylic acid (100 mg, 0.34 mmol) in DMF (3 mL) at 0° C. was added EDC·HCl (98 mg, 0.51 mmol), HOBT (69 mg, 0.51 mmol) and DIEA (88 mg, 0.68 mmol), followed by addition of 1-(4-isopropoxyphenyl) methanamine hydrochloride (103 mg, 0.51 mmol). The reaction mixture was stirred at room temperature for 0.5 h. Then crude was purified by reverse phase column (gradient: 0˜50% ACN/H₂O (0.5% NH₄HCO₃)) to afford 6-benzyl-N-(4-isopropoxybenzyl)-2-methyl-5-oxo-5,6-dihydro-1,6-naphthyridine-3-carboxamide (81.3 mg, yield: 54.03%) as a white solid. LCMS (ESI+): m/z calcd. for C₂₇H₂₈N₃O₃ [M+H]⁺, 442.2, found, 442.2. ¹HNMR (400 MHz, DMSO-d₆, δ): 9.07 (t, J=6.0 Hz, 1H), 8.44 (s, 1H), 7.90 (m, 1H), 7.41-7.15 (m, 7H), 6.98-6.82 (m, 2H), 6.71 (d, J=7.5 Hz, 1H), 5.21 (s, 2H), 4.62-4.56 (m, 1H), 4.40 (d, J=5.8 Hz, 2H), 2.65 (d, J=1.7 Hz, 3H), 1.41-1.24 (m, 6H).

### Example 68: 6-benzyl-2-methyl-N-(4-methylbenzyl)-5-oxo-5,6-dihydro-1,6-naphthyridine-3-carboxamide

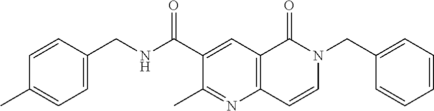 [SMILES: Cc1ccc(CNC(=O)c2cc3c(=O)n(Cc4ccccc4)ccc3nc2C)cc1]

### 1. Synthesis of 6-benzyl-2-methyl-N-(4-methylbenzyl)-5-oxo-5,6-dihydro-1,6-naphthyridine-3-carboxamide

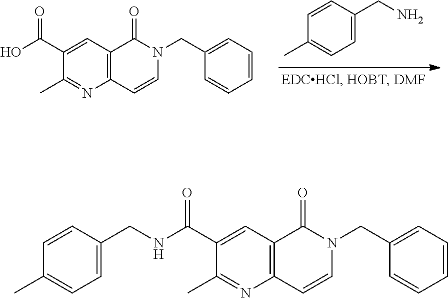

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (65.1 mg, 0.340 mmol) and HOBT (68.8 mg, 0.510 mmol), followed by addition of benzenemethanamine, 4-methyl- (61.7 mg, 0.510 mmol). The reaction mixture was stirred at room temperature for 2 h. The mixture was purified by reverse phase column 0˜50% ACN/H₂O (0.5% NH₄HCO₃)) to afford 6-benzyl-2-methyl-N-(4-methylbenzyl)-5-oxo-5,6-dihydro-1,6-naphthyridine-3-carboxamide (58.8 mg, yield: 42.7%) as a white solid. LCMS (ESI+): m/z calcd. for C₂₅H₂₄N₃O₂ [M+H]⁺, 398.2, found, 398.1. ¹HNMR (400 MHz, DMSO-d₆, δ): 9.12-9.10 (m, 1H), 8.45 (s, 1H), 7.91 (d, J=7.6 Hz, 1H), 7.36-7.32 (m, 7H), 7.18 (d, J=8.0 Hz, 2H), 6.72 (d, J=7.6 Hz, 1H), 5.22 (s, 2H), 4.45 (d, J=6.0 Hz, 2H), 2.67 (s, 3H), 2.30 (s, 3H).

### Example 69: N-(benzo[d][1,3]dioxol-5-ylmethyl)-6-benzyl-2-methyl-5-oxo-5,6-dihydro-1,6-naphthyridine-3-carboxamide

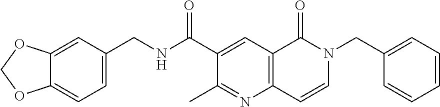 [SMILES: Cc1nc2ccn(Cc3ccccc3)c(=O)c2cc1C(=O)NCc1ccc2c(c1)OCO2]

### 1. Synthesis of N-(benzo[d][1,3]dioxol-5-ylmethyl)-6-benzyl-2-methyl-5-oxo-5,6-dihydro-1,6-naphthyridine-3-carboxamide

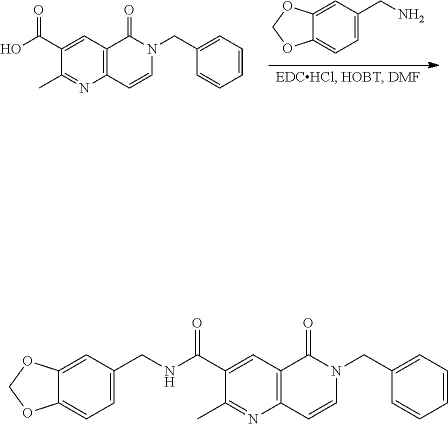

To a solution of 6-benzyl-2-methyl-5-oxo-5,6-dihydro-1,6-naphthyridine-3-carboxylic acid (100 mg, 0.340 mmol) in DMF (2 mL) at 0° C. was added EDC·HCl (97.7 mg, 0.510 mmol) and HOBT (68.9 mg, 0.510 mmol), followed by addition of 1-(2H-1,3-benzodioxol-5-yl)methanamine (77.0 mg, 0.510 mmol). The reaction mixture was stirred at room temperature for 2 h. The crude was purified by reverse phase column (gradient: 0˜50% ACN/H₂O (0.5% NH₄HCO₃)) to afford N-(benzo[d][1,3]dioxol-5-ylmethyl)-6-benzyl-2-methyl-5-oxo-5,6-dihydro-1,6-naphthyridine-3-carboxamide (60.9 mg, yield: 41.1%) as a white solid. LCMS (ESI+): m/z calcd. for C₂₅H₂₂N₃O₄ [M+H]⁺, 428.2, found, 428.1. ¹HNMR (400 MHz, DMSO-d₆, δ): 9.10-9.07 (m, 1H), 8.44 (s, 1H), 7.99 (d, J=7.6 Hz, 1H), 7.36-7.32 (m, 5H), 7.30-7.29 (m, 3H), 6.88-6.85 (m, 1H), 6.01 (s, 2H), 5.98 (s, 2H), 4.39 (d, J=5.6 Hz, 2H), 2.66 (s, 3H).

### Example 70: 2-methyl-N-((5-methylfuran-2-yl)methyl)-5-oxo-6-(thiophen-2-ylmethyl)-5,6-dihydro-1,6-naphthyridine-3-carboxamide

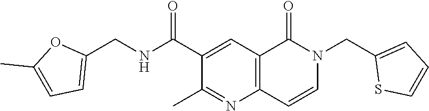 [SMILES: Cc1ccc(CNC(=O)c2cc3c(=O)n(Cc4cccs4)ccc3nc2C)o1]

### 1. Synthesis of 2-methyl-N-((5-methylfuran-2-yl)methyl)-5-oxo-6-(thiophen-2-ylmethyl)-5,6-dihydro-1,6-naphthyridine-3-carboxamide

2-methyl-N-((5-methylfuran-2-yl)methyl)-5-oxo-6-(thiophen-2-ylmethyl)-5,6-dihydro-1,6-naphthyridine-3-carboxamide was synthesized in a similar way as Example 1 as a white solid (13.7 mg). LCMS (ESI+): m/z calcd. for C₂₁H₂₀N₃O₃S [M+H]⁺, 394.1, found, 394.1. ¹HNMR (400 MHz, DMSO-d₆, δ): 9.10-9.07 (m, 1H), 8.45-8.44 (m, 1H), 7.94 (d, J=7.6 Hz, 1H), 7.47-7.44 (m, 1H), 7.22-7.21 (m, 1H), 7.01-6.98 (m, 1H), 6.72 (d, J=7.6 Hz, 1H), 6.20 (d, J=2.8 Hz, 1H), 6.03-6.02 (m, 1H), 5.37 (s, 2H), 4.43 (d, J=5.6 Hz, 2H), 2.66 (s, 3H), 2.26 (s, 3H).

### Example 71: 6-benzyl-2-methyl-5-oxo-N-(thiophen-2-ylmethyl)-5,6-dihydro-1,6-naphthyridine-3-carboxamide

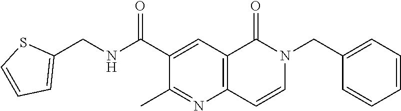 [SMILES: Cc1nc2ccn(Cc3ccccc3)c(=O)c2cc1C(=O)NCc1cccs1]

### 1. Synthesis of 6-benzyl-2-methyl-5-oxo-N-(thiophen-2-ylmethyl)-5,6-dihydro-1,6-naphthyridine-3-carboxamide

6-benzyl-2-methyl-5-oxo-N-(thiophen-2-ylmethyl)-5,6-dihydro-1,6-naphthyridine-3-carboxamide was synthesized in a similar way as Example 1 as a white solid (74.1 mg). LCMS (ESI+): m/z calcd. for C₂₂H₂₀N₃O₂S [M+H]⁺, 390.1, found, 390.1. ¹HNMR (400 MHz, DMSO-d₆, δ): 9.28-9.26 (m, 1H), 8.45 (s, 1H), 7.92 (d, J=7.6 Hz, 1H), 7.44-7.43 (m, 1H), 7.37-7.27 (m, 5H), 7.06-7.01 (m, 1H), 7.00-6.99 (m, 1H), 6.72 (d, J=7.6 Hz, 1H), 5.22 (s, 2H), 4.66 (d, J=6.0 Hz, 2H), 2.67 (s, 3H).

### Example 72: 6-(2-methoxyethyl)-2-methyl-N-((5-methylfuran-2-yl)methyl)-5-oxo-5,6-dihydro-1,6-naphthyridine-3-carboxamide

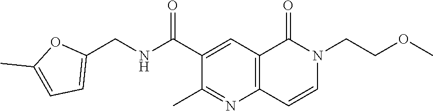

### 1. Synthesis of 6-(2-methoxyethyl)-2-methyl-N-((5-methylfuran-2-yl)methyl)-5-oxo-5,6-dihydro-1,6-naphthyridine-3-carboxamide

6-(2-methoxyethyl)-2-methyl-N-((5-methylfuran-2-yl)methyl)-5-oxo-5,6-dihydro-1,6-naphthyridine-3-carboxamide was synthesized in a similar way as Example 20 as a white solid (14.9 mg). LCMS (ESI+): m/z calcd. for C₁₉H₂₂N₃O₄ [M+H]⁺, 356.1, found, 356.1. ¹HNMR (400 MHz, DMSO-d₆, δ): 9.06-9.05 (m, 1H), 8.42 (s, 1H), 7.73 (d, J=7.6 Hz, 1H), 6.66 (d, J=7.6 Hz, 1H), 6.20 (d, J=2.4 Hz, 1H), 6.03 (s, 1H), 4.43 (d, J=5.2 Hz, 2H), 4.18-4.15 (m, 2H), 3.64-3.61 (m, 2H), 3.25-3.24 (m, 3H), 2.65 (s, 3H), 2.26 (s, 3H).

### Example 73: 6-benzyl-N-(2-(furan-2-yl)ethyl)-2-methyl-5-oxo-5,6-dihydro-1,6-naphthyridine-3-carboxamide

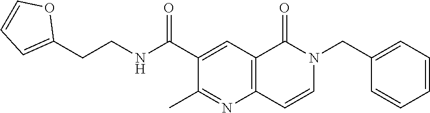 [SMILES: Cc1nc2ccn(Cc3ccccc3)c(=O)c2cc1C(=O)NCCc1ccco1]

### 1. Synthesis of 6-benzyl-N-(2-(furan-2-yl)ethyl)-2-methyl-5-oxo-5,6-dihydro-1,6-naphthyridine-3-carboxamide

6-benzyl-N-(2-(furan-2-yl)ethyl)-2-methyl-5-oxo-5,6-dihydro-1,6-naphthyridine-3-carboxamide was synthesized in a similar way as Example 1 as a white solid (65.0 mg). LCMS (ESI+): m/z calcd. for C₂₃H₂₂N₃O₃ [M+H]⁺, 388.1, found, 388.1. ¹HNMR (400 MHz, DMSO-d₆, δ): 8.72-8.69 (m, 1H), 8.41 (s, 1H), 7.90 (d, J=7.6 Hz, 1H), 7.55 (s, 1H), 7.37-7.29 (m, 5H), 6.71 (d, J=7.6 Hz, 1H), 6.39-6.38 (m, 1H), 6.21 (d, J=3.2 Hz, 1H), 5.22 (s, 2H), 3.56-3.51 (m, 2H), 2.92-2.89 (m, 2H), 2.62 (s, 3H).

### Example 74: 2-amino-6-benzyl-5-oxo-N-(pyridin-2-ylmethyl)-5,6-dihydro-1,6-naphthyridine-3-carboxamide

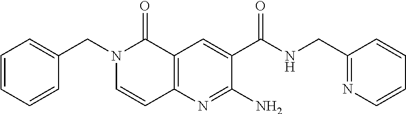 [SMILES: Nc1nc2ccn(Cc3ccccc3)c(=O)c2cc1C(=O)NCc1ccccn1]

### 1. Synthesis of diethyl 2-methylpyridine-3,5-dicarboxylate

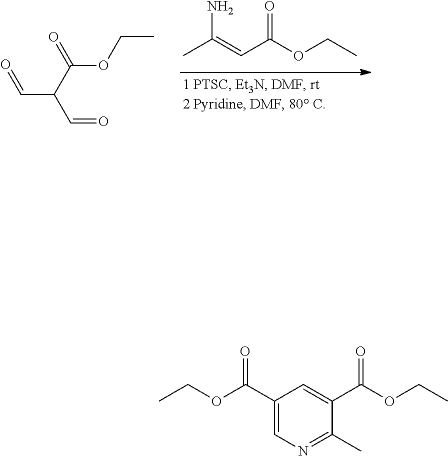

A solution of ethyl 2-formyl-3-oxopropanoate (10.0 g, 69.38 mmol) in ether (100 mL) was added Et₃N (7.7 g, 76.09 mmol) dropwise at 0° C., the resulting mixture was stirred for 2 h at room temperature under nitrogen atmosphere. The resulting mixture was concentrated under reduced pressure and solved in DMF (150 mL) followed by addition of 4-methylbenzene-1-sulfonyl chloride (14.6 g, 76.58 mmol) dropwise at 0° C., the resulting mixture was stirred for 4 h at room temperature. ethyl (2Z)-3-aminobut-2-enoate (9.4 g, 72.77 mmol) and Pyridine (21.8 g, 275.60 mmol) was added to the mixture. The reaction was stirred for overnight at 80° C. under nitrogen atmosphere. The reaction was quenched with H₂O at 0° C. and extracted with EtOAc. The combined organic layers were washed with NaCl (aq.), dried over anhydrous Na₂SO₄. After filtration, the filtrate was concentrated under reduced pressure and purified by silica gel column chromatography, eluted with EtOAc in petroleum ether (0-50%) to afford diethyl 2-methylpyridine-3,5-dicarboxylate (8.4 g, yield: 51.0%) as a yellow solid. LCMS (ESI+): m/z calcd. for C₁₂H₁₆NO₄ [M+H]⁺, 238, found, 238.

### 2. Synthesis of diethyl (E)-2-(2-(dimethylamino)vinyl)pyridine-3,5-dicarboxylate

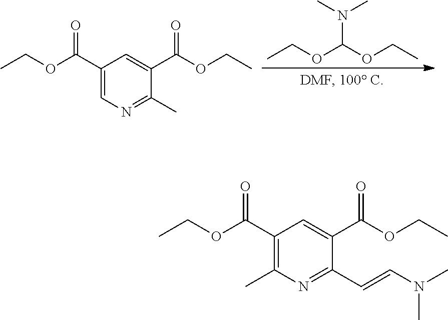

A solution of diethyl 2-methylpyridine-3,5-dicarboxylate (2.0 g, 8.43 mmol) and (diethoxymethyl)dimethylamine (1.5 g, 10.2 mmol) in DMF (20 mL) was stirred for overnight at 100° C. under nitrogen atmosphere. The reaction solution was used for next step without further treatment. LCMS (ESI+): m/z calcd. for C₁₅H₂₁N₂O₄ [M+H]⁺, 293, found, 293.

### 3. Synthesis of ethyl 6-benzyl-5-oxo-5,6-dihydro-1,6-naphthyridine-3-carboxylate

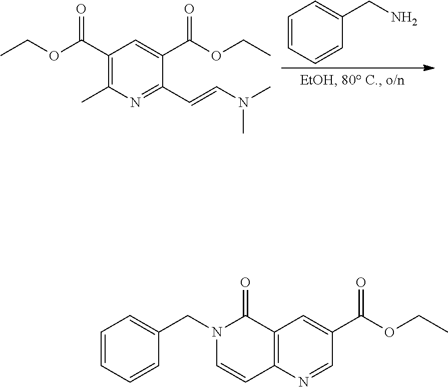

A solution of diethyl (E)-2-(2-(dimethylamino)vinyl)pyridine-3,5-dicarboxylate (from the previous step, 20 mL DMF solution) and benzylamine (1.1 g, 10.26 mmol) in EtOH (10 mL) was stirred for overnight at 80° C. under nitrogen atmosphere. The mixture was allowed to cool down to 0° C., the precipitated solids were collected by filtration and washed with H₂O to afford ethyl 6-benzyl-5-oxo-5,6-dihydro-1,6-naphthyridine-3-carboxylate (2.2 g, yield: 88.3%) as a white solid. The crude was used for next step without further purification. LCMS (ESI+): m/z calcd. for C₁₈H₁₇N₂O₃ [M+H]⁺, 309, found, 309.

### 4. Synthesis of 6-benzyl-3-(ethoxycarbonyl)-5-oxo-5,6-dihydro-1,6-naphthyridine 1-oxide

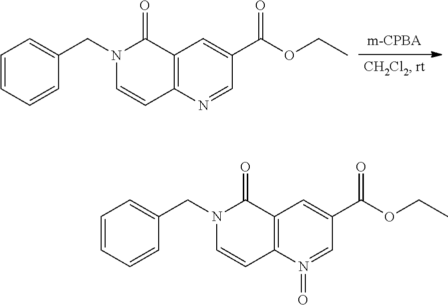 [SMILES: CCOC(=O)c1cnc2ccn(Cc3ccccc3)c(=O)c2c1 ; CCOC(=O)c1cc2c(=O)n(Cc3ccccc3)ccc2[n+]([O-])c1]

A solution of ethyl 6-benzyl-5-oxo-5,6-dihydro-1,6-naphthyridine-3-carboxylate (1 g, 3.24 mmol) in DCM (10 mL) was added to m-CPBA (2.7 g, 15.6 mmol) in portions at 0° C., the solution was stirred for 1 h at room temperature under nitrogen atmosphere. The resulting mixture was diluted with H₂O and quenched by the addition of Na₂SO₃ at 0° C. The resulting mixture was extracted with CH₂Cl₂, the organic layers were dried over anhydrous sodium sulfate. After filtration, the filtrate was concentrated under reduced pressure and purified by silica gel column chromatography, eluted with MeOH in CH₂Cl₂ (0-10%) to afford 880 mg (crude) of 6-benzyl-3-(ethoxycarbonyl)-5-oxo-5,6-dihydro-1,6-naphthyridine 1-oxide as a yellow solid. LCMS (ESI+): m/z calcd. for C₁₈H₁₇N₂O₄ [M+H]⁺, 325, found, 325.

### 5. Synthesis of ethyl 6-benzyl-2-chloro-5-oxo-5,6-dihydro-1,6-naphthyridine-3-carboxylate

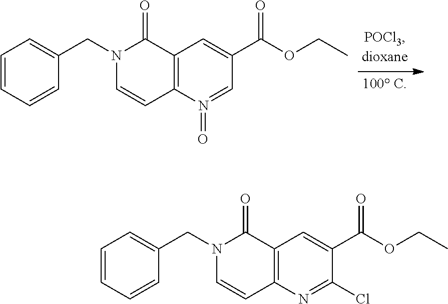 [SMILES: CCOC(=O)c1cc2c(=O)n(Cc3ccccc3)ccc2[n+]([O-])c1 ; CCOC(=O)c1cc2c(=O)n(Cc3ccccc3)ccc2nc1Cl]

A solution of 6-benzyl-3-(ethoxycarbonyl)-5-oxo-5,6-dihydro-1,6-naphthyridine 1-oxide (880 mg, 2.71 mmol) in POCl₃ (6 mL) and dioxane (4 mL) was stirred for 4 h at 100° C. under nitrogen atmosphere. The reaction was quenched by the addition of water at 0° C. and extracted with EtOAc, dried over anhydrous sodium sulfate. After filtration, the filtrate was concentrated under reduced pressure and purified by reverse flash chromatography, eluted with MeCN in water (0.1% FA), 5% to 100% to afford ethyl 6-benzyl-2-chloro-5-oxo-5,6-dihydro-1,6-naphthyridine-3-carboxylate (400 mg, yield: 47.3%) as a yellow solid. LCMS (ESI+): m/z calcd. for C₁₈H₁₆ClN₂O₃[M+H]⁺, 343, found, 343.

### 6. Synthesis of ethyl 6-benzyl-2-((tert-butoxycarbonyl)amino)-5-oxo-5,6-dihydro-1,6-naphthyridine-3-carboxylate

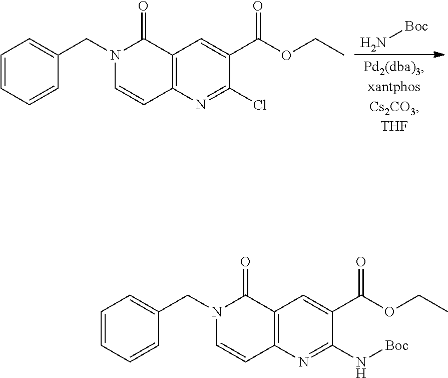

A solution of ethyl 6-benzyl-2-chloro-5-oxo-5,6-dihydro-1,6-naphthyridine-3-carboxylate (400 mg, 1.16 mmol), tert-butyl carbamate (290 mg, 2.47 mmol), Pd₂(dba)₃ (110 mg, 0.12 mmol), XantPhos (140 mg, 0.24 mmol) and Cs₂CO₃ (1.2 g, 3.68 mmol) in THF (10 mL) was stirred for 2 h at 60° C. under nitrogen atmosphere. The resulting mixture was filtered, extracted with EtOAc, dried over anhydrous sodium sulfate. After filtration, the filtrate was concentrated under reduced pressure and purified by reverse flash chromatography eluted with MeCN in water (0.1% FA), 5% to 100% to afford ethyl 6-benzyl-2-((tert-butoxycarbonyl)amino)-5-oxo-5,6-dihydro-1,6-naphthyridine-3-carboxylate (200 mg, yield: 40.4%) as a yellow solid. LCMS (ESI+): m/z calcd. for C₂₃H₂₆N₃O₅ [M+H]⁺, 424, found, 424.

### 7. Synthesis of 6-benzyl-2-((tert-butoxycarbonyl)amino)-5-oxo-5,6-dihydro-1,6-naphthyridine-3-carboxylic acid

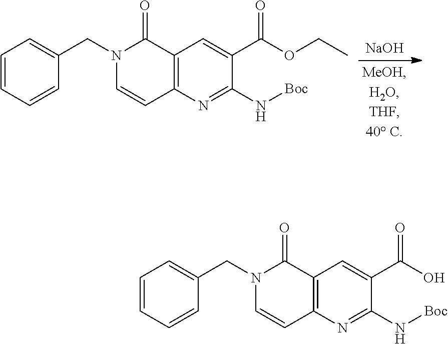

A solution of ethyl 6-benzyl-2-((tert-butoxycarbonyl)amino)-5-oxo-5,6-dihydro-1,6-naphthyridine-3-carboxylate (200 mg, 0.47 mmol) and NaOH (114 mg, 2.85 mmol) in MeOH (1.5 mL), THF (1.5 mL) and H₂O (0.5 mL) was stirred for 1 h at 40° C. The mixture was acidified to pH 7 with conc. HCl (1 mol/L) and purified by reverse flash chromatography eluted with MeCN in water (0.1% FA), 5% to 100% to afford 6-benzyl-2-((tert-butoxycarbonyl)amino)-5-oxo-5,6-dihydro-1,6-naphthyridine-3-carboxylic acid (55 mg, yield: 29.4%) of as a yellow solid. LCMS (ESI+): m/z calcd. for C₂₁H₂₂N₃O₅ [M+H]⁺, 396, found, 396.

### 8. Synthesis of tert-butyl (6-benzyl-5-oxo-3-((pyridin-2-ylmethyl)carbamoyl)-5,6-dihydro-1,6-naphthyridin-2-yl)carbamate

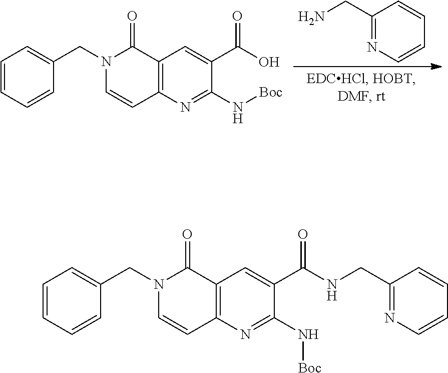 [SMILES: CC(C)(C)OC(=O)Nc1nc2ccn(Cc3ccccc3)c(=O)c2cc1C(=O)O ; NCc1ccccn1 ; CC(C)(C)OC(=O)Nc1nc2ccn(Cc3ccccc3)c(=O)c2cc1C(=O)NCc1ccccn1]

To a solution of 6-benzyl-2-((tert-butoxycarbonyl)amino)-5-oxo-5,6-dihydro-1,6-naphthyridine-3-carboxylic acid (55 mg, 0.13 mmol) in DMF (1 mL) at 0° C. was added EDC·HCl (40 mg, 0.20 mmol) and HOBT (28 mg, 0.20 mmol), followed by addition of 2-pyridinemethaneamine (23 mg, 0.21 mmol). The reaction mixture was stirred at room temperature for 0.5 h. Then crude was purified by reverse flash chromatography eluted with MeOH in water (0.5% NH₄HCO₃), 5% to 100% to afford tert-butyl (6-benzyl-5-oxo-3-((pyridin-2-ylmethyl)carbamoyl)-5,6-dihydro-1,6-naphthyridin-2-yl)carbamate (45 mg, yield: 66.6%) of as a yellow solid. LCMS (ESI+): m/z calcd. for C₂₇H₂₈N₅O₄ [M+H]⁺, 486, found, 486.

### 9. Synthesis of 2-amino-6-benzyl-5-oxo-N-(pyridin-2-ylmethyl)-5,6-dihydro-1,6-naphthyridine-3-carboxamide

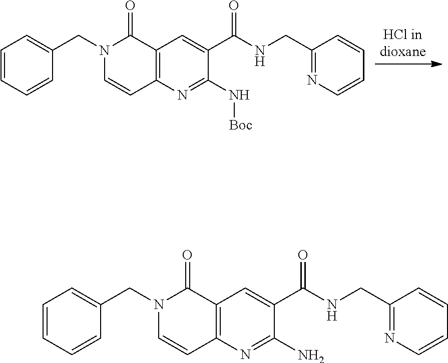 [SMILES: CC(C)(C)OC(=O)Nc1nc2ccn(Cc3ccccc3)c(=O)c2cc1C(=O)NCc1ccccn1 ; Nc1nc2ccn(Cc3ccccc3)c(=O)c2cc1C(=O)NCc1ccccn1]

A solution of tert-butyl (6-benzyl-5-oxo-3-((pyridin-2-ylmethyl)carbamoyl)-5,6-dihydro-1,6-naphthyridin-2-yl)carbamate (45 mg, 0.09 mmol) in HCl (gas) in 1,4-dioxane (1 mL) was stirred for 3 h at 30° C. The resulting mixture was concentrated under reduced pressure and purified by reverse flash chromatography eluted with MeOH in water (0.5% NH₄HCO₃), 5% to 100% to afford 2-amino-6-benzyl-5-oxo-N-(pyridin-2-ylmethyl)-5,6-dihydro-1,6-naphthyridine-3-carboxamide (5.9 mg, yield: 16.4%) as a white solid. LCMS (ESI+): m/z calcd. for C₂₂H₂₀N₅O₂ [M+H]⁺, 386.1, found, 386.1. ¹HNMR (400 MHz, DMSO-d₆, δ): 9.43 (t, J=5.8 Hz, 1H), 8.76 (s, 1H), 8.52 (d, J=4.8 Hz, 1H), 7.79-7.74 (m, 4H), 7.37-7.26 (m, 7H), 6.31 (d, J=7.6 Hz, 1H), 5.14 (s, 2H), 4.54 (d, J=5.6 Hz, 2H).

### Example 75: 6-(4-chloro-3-fluorobenzyl)-2-methyl-5-oxo-N-(pyridin-2-ylmethyl)-5,6-dihydro-1,6-naphthyridine-3-carboxamide

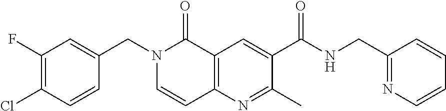 [SMILES: Cc1nc2ccn(Cc3ccc(Cl)c(F)c3)c(=O)c2cc1C(=O)NCc1ccccn1]

### 1. Synthesis of 6-(4-chloro-3-fluorobenzyl)-2-methyl-5-oxo-N-(pyridin-2-ylmethyl)-5,6-dihydro-1,6-naphthyridine-3-carboxamide

6-(4-chloro-3-fluorobenzyl)-2-methyl-5-oxo-N-(pyridin-2-ylmethyl)-5,6-dihydro-1,6-naphthyridine-3-carboxamide was synthesized in a similar way as Example 61 as a white solid (20.5 mg). LCMS (ESI+): m/z calcd. for C₂₃H₁₉ClFN₄O₂[M+H]⁺, 437.1, found, 437.1. ¹HNMR (400 MHz, DMSO-d₆, δ): 9.25-9.22 (m, 1H), 8.56-8.53 (m, 2H), 7.95 (d, J=7.6 Hz, 1H), 7.84-7.79 (m, 1H), 7.60-7.56 (m, 1H), 7.43-7.40 (m, 2H), 7.34-7.29 (m, 1H), 7.20-7.18 (m, 1H), 6.75 (d, J=7.6 Hz, 1H), 5.22 (s, 2H), 4.59 (d, J=6.0 Hz, 2H), 2.69 (s, 3H).

### Example 76: 6-(3,4-difluorobenzyl)-2-methyl-N-((2-methyloxazol-4-yl)methyl)-5-oxo-5,6-dihydro-1,6-naphthyridine-3-carboxamide

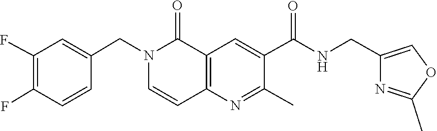 [SMILES: Cc1nc(CNC(=O)c2cc3c(=O)n(Cc4ccc(F)c(F)c4)ccc3nc2C)co1]

### 1. Synthesis of 6-(3,4-difluorobenzyl)-2-methyl-N-((2-methyloxazol-4-yl)methyl)-5-oxo-5,6-dihydro-1,6-naphthyridine-3-carboxamide

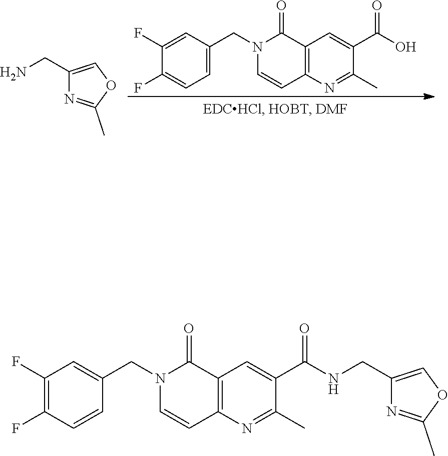

To a solution of 6-(3,4-difluorobenzyl)-2-methyl-5-oxo-5,6-dihydro-1,6-naphthyridine-3-carboxylic acid (66 mg, 0.200 mmol) in DMF (2 mL) at 0° C. was added EDC·HCl (57.4 mg, 0.300 mmol) and HOBT (40.5 mg, 0.300 mmol), followed by addition of (2-methyloxazol-4-yl)methanamine (33.6 mg, 0.300 mmol). The reaction mixture was stirred at room temperature for 1 h. Then crude was purified by reverse phase column (gradient: 0-50% ACN/H₂O (0.5% NH₄HCO₃)) to afford 6-(3,4-difluorobenzyl)-2-methyl-N-((2-methyloxazol-4-yl)methyl)-5-oxo-5,6-dihydro-1,6-naphthyridine-3-carboxamide (49.1 mg, yield: 57%) as a white solid. LCMS (ESI+): m/z calcd. for C₂₂H₁₉F₂N₄O₃[M+H]⁺, 425.1, found, 425.1. ¹HNMR (400 MHz, DMSO-d₆, δ): 9.04-9.01 (m, 1H), 8.44 (s, 1H), 7.94 (d, J=7.6 Hz, 1H), 7.85 (s, 1H), 7.46-7.37 (m, 2H), 7.19 (d, J=8.3 Hz, 1H), 6.73 (d, J=7.6 Hz, 1H), 5.18 (s, 2H), 4.32 (d, J=5.6 Hz, 2H), 2.65 (s, 3H), 2.40 (s, 3H).

### Example 77: 6-(4-chlorobenzyl)-N-((5-(2-(2-methoxyethoxy)ethoxy)pyridin-2-yl)methyl)-2-methyl-5-oxo-5,6-dihydro-1,6-naphthyridine-3-carboxamide

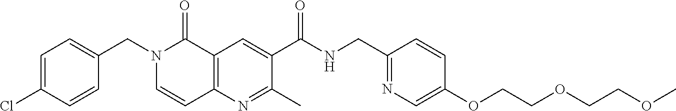 [SMILES: COCCOCCOc1ccc(CNC(=O)c2cc3c(=O)n(Cc4ccc(Cl)cc4)ccc3nc2C)nc1]

6-(4-chlorobenzyl)-N-((5-(2-(2-methoxyethoxy)ethoxy)pyridin-2-yl)methyl)-2-methyl-5-oxo-5,6-dihydro-1,6-naphthyridine-3-carboxamide was synthesized in a similar way as Example 51 as a white solid (42.0 mg). LCMS (ESI+): m/z calcd. for C₂₈H₃₀ClN₄O₅[M+H]⁺, 537.2, found, 537.2.

### Example 78: 6-(4-fluorobenzyl)-2-methyl-N-((2-methyloxazol-4-yl)methyl)-5-oxo-5,6-dihydro-1,6-naphthyridine-3-carboxamide

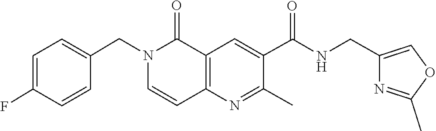 [SMILES: Cc1nc(CNC(=O)c2cc3c(=O)n(Cc4ccc(F)cc4)ccc3nc2C)co1]

### 1. Synthesis of 6-(4-fluorobenzyl)-2-methyl-N-((2-methyloxazol-4-yl)methyl)-5-oxo-5,6-dihydro-1,6-naphthyridine-3-carboxamide

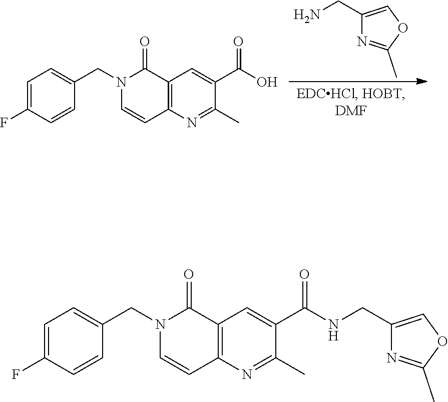

To a solution of 6-(4-fluorobenzyl)-2-methyl-5-oxo-5,6-dihydro-1,6-naphthyridine-3-carboxylic acid (50 mg, 0.160 mmol) in DMF (2 mL) at 0° C. was added EDC·HCl (50 mg, 0.261 mmol) and HOBT (34 mg, 0.252 mmol), followed by addition of (2-methyloxazol-4-yl)methanamine (60 mg, 0.535 mmol). The reaction mixture was stirred at room temperature for 2 h. The residue was purified by reverse flash chromatography, eluted with MeCN in water (0.5% NH₄HCO₃), 10% to 100% gradient in 40 min to afford 6-(4-fluorobenzyl)-2-methyl-N-((2-methyloxazol-4-yl)methyl)-5-oxo-5,6-dihydro-1,6-naphthyridine-3-carboxamide as a white solid (27.9 mg, yield: 47.6%). LCMS (ESI+): m/z calcd. for C₂₂H₂₀FN₄O₃[M+H]⁺, 407.1, found, 407.2. ¹HNMR (400 MHz, DMSO-d₆, δ): 9.03-9.00 (m, 1H), 8.45 (s, 1H), 7.93 (d, J=7.6 Hz, 1H), 7.86 (s, 1H), 7.42-7.38 (m, 2H), 7.20-7.15 (m, 2H), 6.72 (d, J=7.2 Hz, 1H), 5.19 (s, 2H), 4.33-4.31 (m, 2H), 2.64 (s, 3H), 2.40 (s, 3H).

### Example 79: 6-(3-fluorobenzyl)-2-methyl-N-((2-methyloxazol-4-yl)methyl)-5-oxo-5,6-dihydro-1,6-naphthyridine-3-carboxamide

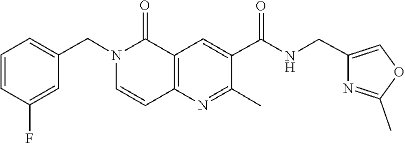 [SMILES: Cc1nc(CNC(=O)c2cc3c(=O)n(Cc4cccc(F)c4)ccc3nc2C)co1]

### 1. Synthesis of 6-(3-fluorobenzyl)-2-methyl-N-((2-methyloxazol-4-yl)methyl)-5-oxo-5,6-dihydro-1,6-naphthyridine-3-carboxamide

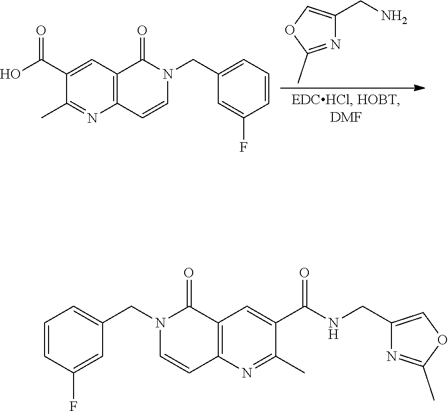

To a solution of 6-(3-fluorobenzyl)-2-methyl-5-oxo-5,6-dihydro-1,6-naphthyridine-3-carboxylic acid (50 mg, 0.160 mmol) and in DMF (2 mL) at 0° C. was added EDC·HCl (46.0 mg, 0.240 mmol) and HOBT (12.9 mg, 0.096 mmol), followed by addition of (2-methyloxazol-4-yl)methanamine (26.9 mg, 0.240 mmol). The reaction mixture was stirred at room temperature for 2 h. The residue was purified by reverse flash chromatography, eluted with MeCN in water (0.5% NH₄HCO₃), 10% to 100% gradient in 40 min to afford 6-(3-fluorobenzyl)-2-methyl-N-((2-methyloxazol-4-yl)methyl)-5-oxo-5,6-dihydro-1,6-naphthyridine-3-carboxamide as a white solid (22.3 mg, yield: 34.2%). LCMS (ESI+): m/z calcd. for C₂₂H₂₀FN₄O₃[M+H]⁺, 407.1, found, 407.1. ¹HNMR (400 MHz, DMSO-d₆, δ): 9.02-9.01 (m, 1H), 8.45 (s, 1H), 7.94 (d, J=7.6 Hz, 1H), 7.86 (s, 1H), 7.42-7.38 (m, 1H), 7.24-7.11 (m, 3H), 6.72 (d, J=7.2 Hz, 1H), 5.23 (s, 2H), 4.32 (d, J=5.6 Hz, 2H), 2.66 (s, 3H), 2.40 (s, 3H).

### Example 80: 6-(4-chlorobenzyl)-2-methyl-N-((2-methyloxazol-4-yl)methyl)-5-oxo-5,6-dihydro-1,6-naphthyridine-3-carboxamide

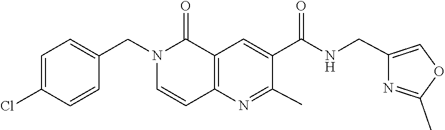 [SMILES: Cc1nc(CNC(=O)c2cc3c(=O)n(Cc4ccc(Cl)cc4)ccc3nc2C)co1]

### 1. Synthesis of 6-(4-chlorobenzyl)-2-methyl-N-((2-methyloxazol-4-yl)methyl)-5-oxo-5,6-dihydro-1,6-naphthyridine-3-carboxamide

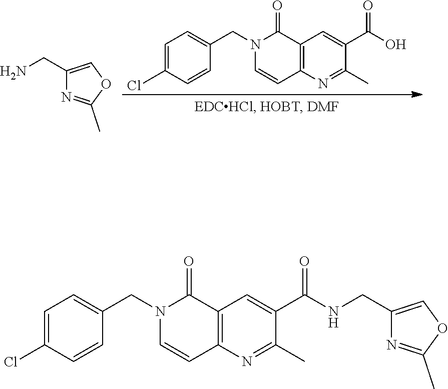

To a solution of 6-(4-chlorobenzyl)-2-methyl-5-oxo-5,6-dihydro-1,6-naphthyridine-3-carboxylic acid (100 mg, 0.304 mmol) in DMF (2 mL) at 0° C. was added EDC·HCl (87 mg, 0.456 mmol) and HOBT (61 mg, 0.456 mmol), followed by addition of (2-methyloxazol-4-yl)methanamine (51 mg, 0.456 mmol). The reaction mixture was stirred at room temperature for 1 h. Then crude was purified by reverse phase column (gradient: 0˜50% ACN/H₂O (0.5% NH₄HCO₃)) to afford 6-(4-chlorobenzyl)-2-methyl-N-((2-methyloxazol-4-yl)methyl)-5-oxo-5,6-dihydro-1,6-naphthyridine-3-carboxamide as a white solid (55.9 mg, yield: 45.9%). LCMS (ESI+): m/z calcd. for C₂₂H₂₀ClN₄O₃[M+H]⁺, 423.1, found, 423.1. ¹HNMR (400 MHz, DMSO-d₆, δ): 9.02-8.99 (m, 1H), 8.44 (s, 1H), 7.92 (d, J=7.6 Hz, 1H), 7.85 (s, 1H), 7.42-7.34 (m, 4H), 6.73-6.71 (m, 1H), 5.20 (s, 2H) 4.32 (d, J=4.8 Hz, 2H), 2.66 (s, 3H), 2.40 (s, 3H).

### Example 81: 6-(3,5-difluorobenzyl)-2-methyl-N-((2-methyloxazol-4-yl)methyl)-5-oxo-5,6-dihydro-1,6-naphthyridine-3-carboxamide

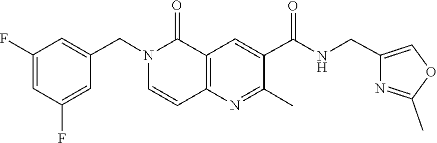 [SMILES: Cc1nc(CNC(=O)c2cc3c(=O)n(Cc4cc(F)cc(F)c4)ccc3nc2C)co1]

### 1. Synthesis of ethyl 6-(3,5-difluorobenzyl)-2-methyl-5-oxo-5,6-dihydro-1,6-naphthyridine-3-carboxylate

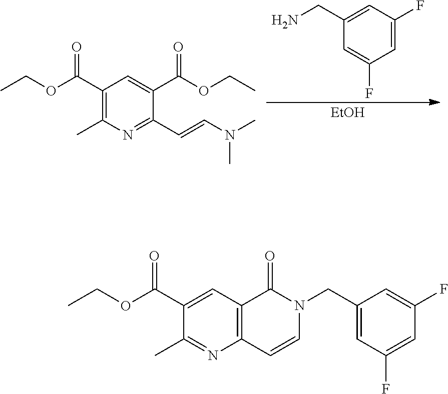 [SMILES: "EtOH" ; CCOC(=O)c1cc(C(=O)OCC)c(/C=C/N(C)C)nc1C ; NCc1cc(F)cc(F)c1 ; CCOC(=O)c1cc2c(=O)n(Cc3cc(F)cc(F)c3)ccc2nc1C]

A solution of diethyl (E)-2-(2-(dimethylamino)vinyl)-6-methylpyridine-3,5-dicarboxylate (250 mg, 0.816 mmol) and 1-(3,5-difluorophenyl)methanamine (233.6 mg, 1.63 mmol) in EtOH (2.5 mL) was stirred for overnight at 80° C. under nitrogen atmosphere. The mixture was allowed to cool down to 0° C., the precipitated solids were collected by filtration and washed with H₂O (3×2 mL) to afford ethyl 6-(3,5-difluorobenzyl)-2-methyl-5-oxo-5,6-dihydro-1,6-naphthyridine-3-carboxylate as a yellow solid (170 mg, 58.1%). LCMS (ESI+): m/z calcd. for C₁₉H₁₇F₂N₂O₃[M+H]⁺, 359, found, 359.

### 2. Synthesis of 6-(3,5-difluorobenzyl)-2-methyl-5-oxo-5,6-dihydro-1,6-naphthyridine-3-carboxylic acid

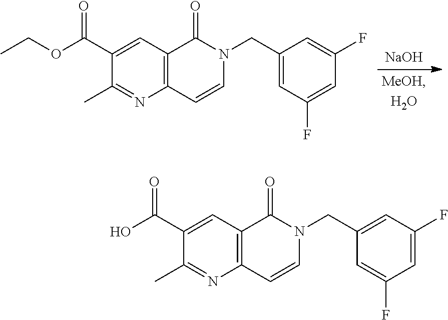

A solution of ethyl 6-(3,5-difluorobenzyl)-2-methyl-5-oxo-5,6-dihydro-1,6-naphthyridine-3-carboxylate (180 mg, 0.502 mmol) and NaOH (60 mg, 1.50 mmol) in MeOH (3 mL) and H₂O (1 mL) was stirred for 8 h at 40° C. The residue was acidified to pH 4 with conc. HCl, and the aqueous layer was extracted with EtOAc. The combined organic layers were dried over Na₂SO₄ and concentrated in vacuo to afford 6-(3,5-difluorobenzyl)-2-methyl-5-oxo-5,6-dihydro-1,6-naphthyridine-3-carboxylic acid as a white solid (120 mg, yield: 72.3%). LCMS (ESI+): m/z calcd. for C₁₇H₁₃F₂N₂O₃[M+H]⁺, 331, found, 331.

### 3. Synthesis of 6-(3,5-difluorobenzyl)-2-methyl-N-((2-methyloxazol-4-yl)methyl)-5-oxo-5,6-dihydro-1,6-naphthyridine-3-carboxamide

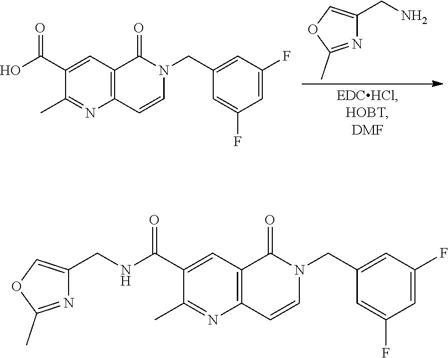

To a solution of 6-(3,5-difluorobenzyl)-2-methyl-5-oxo-5,6-dihydro-1,6-naphthyridine-3-carboxylic acid (50 mg, 0.151 mmol) in DMF (2 mL) at 0° C. was added EDC·HCl (46 mg, 0.240 mmol) and HOBT (32 mg, 0.237 mmol), followed by addition of (2-methyloxazol-4-yl)methanamine (64 mg, 0.571 mmol). The reaction mixture was stirred at room temperature for 1 h. Then crude was purified by reverse phase column (gradient: 0˜50% ACN/H₂O (0.5% NH₄HCO₃)) to afford 6-(3,5-difluorobenzyl)-2-methyl-N-((2-methyloxazol-4-yl)methyl)-5-oxo-5,6-dihydro-1,6-naphthyridine-3-carboxamide as a white solid (25.5 mg, yield: 38.5%). LCMS (ESI+): m/z calcd. for C₂₂H₁₉F₂N₄O₃[M+H]⁺, 425.1, found, 425.1. ¹HNMR (400 MHz, DMSO-d₆, δ): 9.02-9.01 (m, 1H), 8.45 (s, 1H), 7.92 (d, J=7.6 Hz, 1H), 7.86 (d, J=1.1 Hz, 1H), 7.20-7.14 (m, 1H), 7.10-7.00 (m, 2H), 6.79-6.72 (m, 1H), 5.22 (s, 2H), 4.41-4.08 (m, 2H), 2.67 (s, 3H), 2.40 (s, 3H).

### BIOLOGICAL EXAMPLES

### Tfeb-Translocation Assay HEK-293T-oEX-Tfeb-GFP Cell Line

### 1. Experiment Materials

| | Vendor | Cat No. |
|---|---|---|
| Reagents | | |
| HEK-293T-oEX-TFEB-GFP 17# | Mitobridge | / |
| DMEM | Gibco | 11995-065 |
| DPBS | Invitrogen | 14190-144 |
| TrypLE Express | GIBCO | 12604-013 |
| Trypan Blue | Invitrogen | T10282 |
| FBS | Gibco | 10099-141C |
| DMSO | Sigma | D8418-1L |
| Penicillin-Streptomycin | GIBCO | 15140-122 |
| Blasticidin | Invitrogen | A1113903 |
| HEPES Buffer Solution 1M, | Invitrogen | 15630080 |
| liquid | | |
| GLUTAMAX I- 100X | Invitrogen | 35050061 |
| MEM NEAA- 100X | Invitrogen | 11140-050 |
| Hoechst 33342 | Invitrogen | H1399 |
| Consumables | | |
| Flask (T225) | Corning | 431081 |
| Flask (T75) | Corning | 430641 |
| Poly-D-Lysine Black/Clear | Corning | 356663 |
| Microtest (TM) Tissue-Culture | | |
| Treated Polystyrene, 384-well | | |
| plate (BD Biocoat) | | |
| Source plate 384-well LDV | Labcyte | LP-0200 |
| Instruments | | |
| Biosafety cabinet | Thermo Scientific | 1300 Series A2 |
| Centrifuge | Eppendorf | 5804R |
| CO₂ incubator | Thermo Scientific | 371 |
| Cell countess II | Invitrogen | C10281 |
| Microscope | Olympus | CKX41 |
| Operetta | PerkinElmer | CLS |
| | | 1600L20356 |
| Echo | Labcyte | 665 |

### 2. Cell Culturing and Compounds Dilution

### 2.1. Culturing the HIEK-293T-oEX-TFEB-GFP Cell Line

1). Added 2×10⁶ cell into a T75 flask with 15 mL DiVEM medium (with 10% FBS, 1% PS, 1×GLUTAMAX, 1×MEM NEAA, 1×HEPES Buffer Solution and 5 ug/mL blasticidin).
2). Culturing for 48-72 hours. Cells in logarithmic growth phase were taken for experiment. The other cells were passaged as described in step 1.
3). Seeded 1×10⁴ cells/30 μL/well into 384-well plate (BD 356663). Cultured overnight at 37° C. & 5% CO₂ incubator.
4). Note: Used cells <10 passage

### 2.2. Compound Dilution and Treatment

1). Compounds were firstly diluted by DMSO to the stock needed (10 mM), and aliquoted and stored at −20° C.
2). Took one aliquot each time and made 3-fold or 2-fold dilutions as indicated, total 10 doses plus one DMSO control well in the source plate.
3). Added 150 or 90 nL/well compounds solution to the assay plate by ECHO and incubated for 6 or 18 hours respectively at 37° C. & 5% CO₂ incubator.

### 3. Translocation Assay

### 3.1. Cell Fixing

1). Added 30 uL 8% PFA into the wells and incubated 20 min at room temperature.
2). Discarded the medium, washed the wells twice with DPBS to eliminate foam and bubbles in the well.

### 3.2. Nucleus Staining

1). Diluted Hoechest-333824 in DPBS to get 2 μg/mL solution and added 30 uL/well into assay plate, incubated at room temperature for 20 mins.
2). Discarded the supernatant, washed the wells twice with DPBS to eliminate foam and bubbles in the well. Then added DPBS 20 uL/well.

### 3). Scanned Plate by Operetta.

### 3.3. Plate Map for TFEB Translocation Assay

1). Reference compound: Torin 1
2). Test compounds: C1, C2, C3, C4, C5, C6.
3). Diluted compounds in 384 LDV-dilution plate according to the dilution plate map
4). Compounds starting concentration: Torin 1: 300 nM and Test compounds: 50 uM.

### 4. Data Analysis: TFEB Translocation Positive Cells Ratio

1). Selected all nucleus by Operetta as ‘All Cells’
2). Calculated the EGFP average intensity inside the Nucleus region.
3). Selected population that the EGFP mean intensity inside the Nucleus region>background as the EGFP-positive cells
4). Selected the Surrounding region of the nucleus and calculated the EGFP mean intensity of the region
5). Inside the EGFP-positive cell population, selected the cells with EGFP mean intensity inside the Nucleus region/the EGFP mean intensity inside the surrounding region=1.2 (This ratio could be slightly adjusted from time to time) as the Translocation positive cell.
6). Outputted the ratio of Translocation positive cell/EGFP-positive cells as the Translocation Positive Ratio.
7). Each time set the ratio of DMSO group (Control Group) as ˜15% and then normalized the compound group to the DMSO group.

The EC₅₀ of reference compound Torin 1 was 28.34 nM.

### Data for Examples

TABLE 1
TFEB Transloctaion EC₅₀ values for Example Compounds

| | | TFEB |
|---|---|---|
| Example | | Translocation |
| Number | Structure | EC₅₀ (μM) |
| 1 | 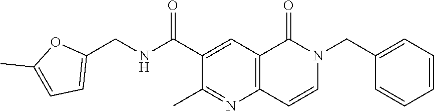 [SMILES: Cc1ccc(CNC(=O)c2cc3c(=O)n(Cc4ccccc4)ccc3nc2C)o1] | ++++ |
| 2 | 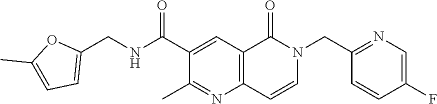 [SMILES: Cc1ccc(CNC(=O)c2cc3c(=O)n(Cc4ccc(F)cn4)ccc3nc2C)o1] | +++ |
| 3 | 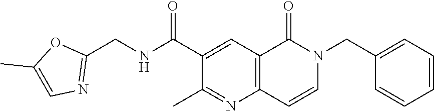 [SMILES: Cc1cnc(CNC(=O)c2cc3c(=O)n(Cc4ccccc4)ccc3nc2C)o1] | +++ |
| 4 | 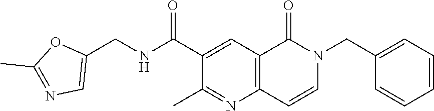 [SMILES: Cc1ncc(CNC(=O)c2cc3c(=O)n(Cc4ccccc4)ccc3nc2C)o1] | +++ |
| 5 | 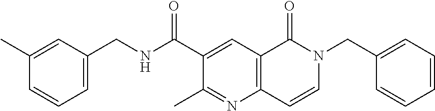 [SMILES: Cc1cccc(CNC(=O)c2cc3c(=O)n(Cc4ccccc4)ccc3nc2C)c1] | +++ |
| 6 | 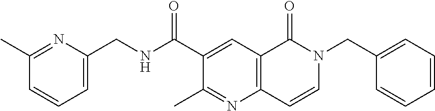 [SMILES: Cc1cccc(CNC(=O)c2cc3c(=O)n(Cc4ccccc4)ccc3nc2C)n1] | +++ |
| 7 | 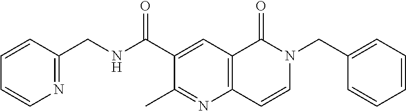 [SMILES: Cc1nc2ccn(Cc3ccccc3)c(=O)c2cc1C(=O)NCc1ccccn1] | +++ |
| 8 | 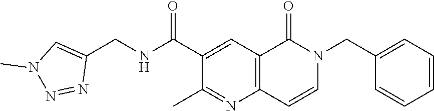 [SMILES: Cc1nc2ccn(Cc3ccccc3)c(=O)c2cc1C(=O)NCc1cn(C)nn1] | ++ |
| 9 | 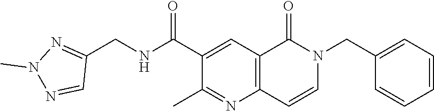 [SMILES: Cc1nc2ccn(Cc3ccccc3)c(=O)c2cc1C(=O)NCc1cnn(C)n1] | +++ |
| 10 | 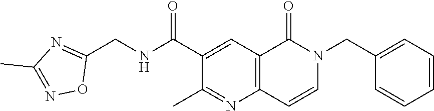 [SMILES: Cc1noc(CNC(=O)c2cc3c(=O)n(Cc4ccccc4)ccc3nc2C)n1] | +++ |
| 11 | 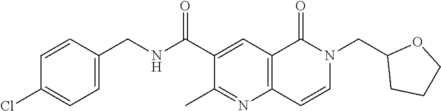 [SMILES: Cc1nc2ccn(CC3CCCO3)c(=O)c2cc1C(=O)NCc1ccc(Cl)cc1] | ++ |
| 12 | 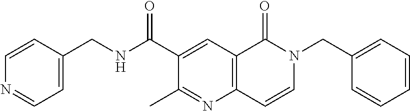 [SMILES: Cc1nc2ccn(Cc3ccccc3)c(=O)c2cc1C(=O)NCc1ccncc1] | ++ |
| 13 | 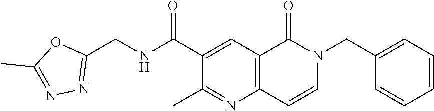 [SMILES: Cc1nnc(CNC(=O)c2cc3c(=O)n(Cc4ccccc4)ccc3nc2C)o1] | ++ |
| 14 | 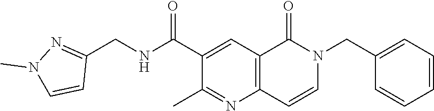 [SMILES: Cc1nc2ccn(Cc3ccccc3)c(=O)c2cc1C(=O)NCc1ccn(C)n1] | +++ |
| 15 | 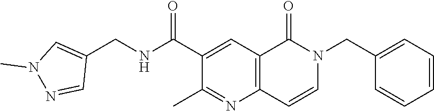 [SMILES: Cc1nc2ccn(Cc3ccccc3)c(=O)c2cc1C(=O)NCc1cnn(C)c1] | +++ |
| 16 | 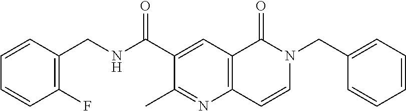 [SMILES: Cc1nc2ccn(Cc3ccccc3)c(=O)c2cc1C(=O)NCc1ccccc1F] | +++ |
| 17 | 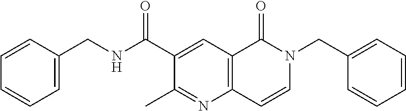 [SMILES: Cc1nc2ccn(Cc3ccccc3)c(=O)c2cc1C(=O)NCc1ccccc1] | +++ |
| 18 | 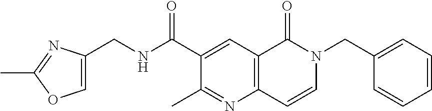 [SMILES: Cc1nc(CNC(=O)c2cc3c(=O)n(Cc4ccccc4)ccc3nc2C)co1] | +++ |
| 19 | 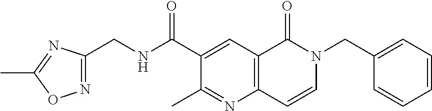 [SMILES: Cc1nc(CNC(=O)c2cc3c(=O)n(Cc4ccccc4)ccc3nc2C)no1] | ++ |
| 20 | 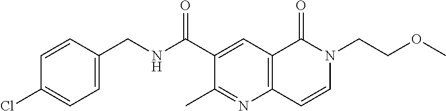 [SMILES: COCCn1ccc2nc(C)c(C(=O)NCc3ccc(Cl)cc3)cc2c1=O] | ++ |
| 21 | 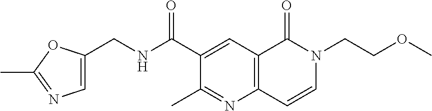 | + |
| 22 | 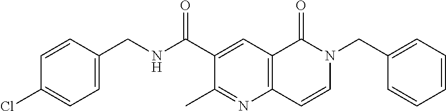 [SMILES: Cc1nc2ccn(Cc3ccccc3)c(=O)c2cc1C(=O)NCc1ccc(Cl)cc1] | ++++ |
| 23 | 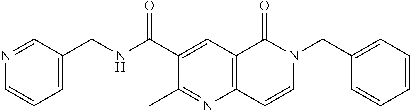 [SMILES: Cc1nc2ccn(Cc3ccccc3)c(=O)c2cc1C(=O)NCc1cccnc1] | +++ |
| 24 | 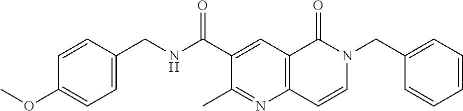 [SMILES: COc1ccc(CNC(=O)c2cc3c(=O)n(Cc4ccccc4)ccc3nc2C)cc1] | ++ |
| 25 | 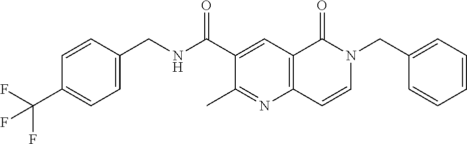 [SMILES: Cc1nc2ccn(Cc3ccccc3)c(=O)c2cc1C(=O)NCc1ccc(C(F)(F)F)cc1] | + |
| 26 | 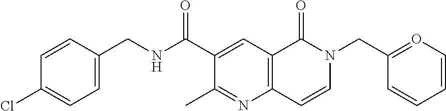 | +++ |
| 27 | 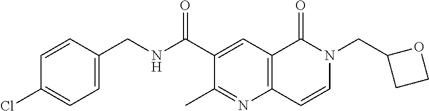 [SMILES: Cc1nc2ccn(CC3CCO3)c(=O)c2cc1C(=O)NCc1ccc(Cl)cc1] | ++ |
| 28 | 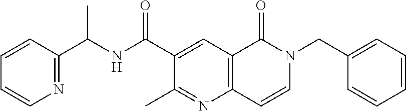 [SMILES: Cc1nc2ccn(Cc3ccccc3)c(=O)c2cc1C(=O)NC(C)c1ccccn1] | + |
| 29 | 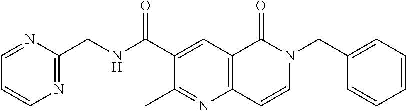 [SMILES: Cc1nc2ccn(Cc3ccccc3)c(=O)c2cc1C(=O)NCc1ncccn1] | + |
| 30 | 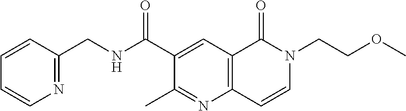 [SMILES: COCCn1ccc2nc(C)c(C(=O)NCc3ccccn3)cc2c1=O] | ++ |
| 31 | 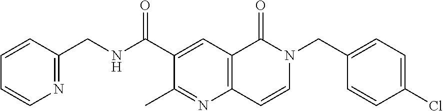 [SMILES: Cc1nc2ccn(Cc3ccc(Cl)cc3)c(=O)c2cc1C(=O)NCc1ccccn1] | ++++ |
| 32 | 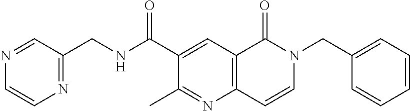 [SMILES: Cc1nc2ccn(Cc3ccccc3)c(=O)c2cc1C(=O)NCc1cnccn1] | +++ |
| 33 | 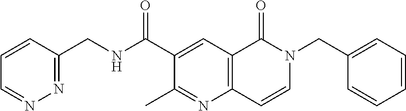 [SMILES: Cc1nc2ccn(Cc3ccccc3)c(=O)c2cc1C(=O)NCc1cccnn1] | ++ |
| 34 | 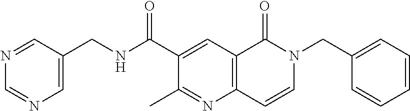 [SMILES: Cc1nc2ccn(Cc3ccccc3)c(=O)c2cc1C(=O)NCc1cncnc1] | +++ |
| 35 | 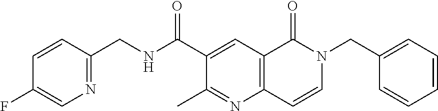 [SMILES: Cc1nc2ccn(Cc3ccccc3)c(=O)c2cc1C(=O)NCc1ccc(F)cn1] | ++++ |
| 36 | 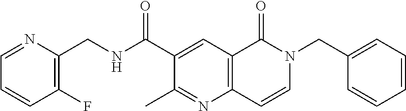 [SMILES: Cc1nc2ccn(Cc3ccccc3)c(=O)c2cc1C(=O)NCc1ncccc1F] | ++ |
| 37 | 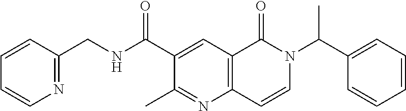 [SMILES: Cc1nc2ccn(C(C)c3ccccc3)c(=O)c2cc1C(=O)NCc1ccccn1] | +++ |
| 38 | 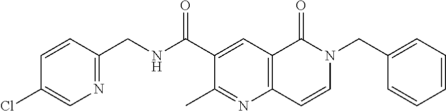 [SMILES: Cc1nc2ccn(Cc3ccccc3)c(=O)c2cc1C(=O)NCc1ccc(Cl)cn1] | ++++ |
| 39 | 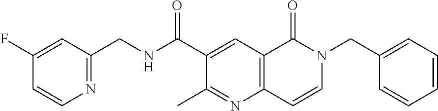 [SMILES: Cc1nc2ccn(Cc3ccccc3)c(=O)c2cc1C(=O)NCc1cc(F)ccn1] | +++ |
| 40 | 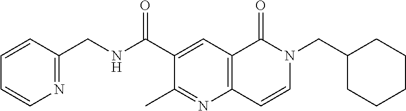 [SMILES: Cc1nc2ccn(CC3CCCCC3)c(=O)c2cc1C(=O)NCc1ccccn1] | + |
| 41 | 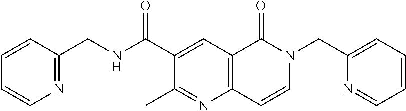 [SMILES: Cc1nc2ccn(Cc3ccccn3)c(=O)c2cc1C(=O)NCc1ccccn1] | ++ |
| 42 | 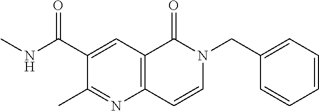 [SMILES: CNC(=O)c1cc2c(=O)n(Cc3ccccc3)ccc2nc1C] | + |
| 43 | 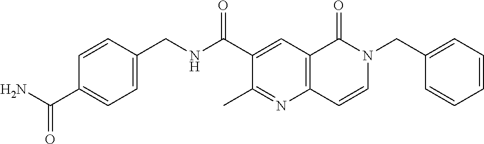 [SMILES: Cc1nc2ccn(Cc3ccccc3)c(=O)c2cc1C(=O)NCc1ccc(C(N)=O)cc1] | + |
| 44 | 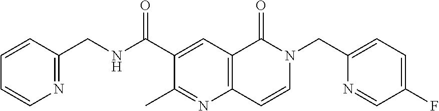 [SMILES: Cc1nc2ccn(Cc3ccc(F)cn3)c(=O)c2cc1C(=O)NCc1ccccn1] | ++ |
| 45 | 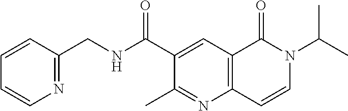 [SMILES: Cc1nc2ccn(C(C)C)c(=O)c2cc1C(=O)NCc1ccccn1] | +++ |
| 46 | 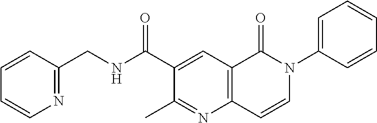 [SMILES: Cc1nc2ccn(-c3ccccc3)c(=O)c2cc1C(=O)NCc1ccccn1] | ++ |
| 47 | 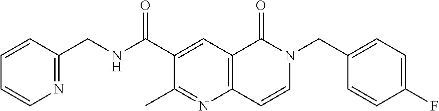 [SMILES: Cc1nc2ccn(Cc3ccc(F)cc3)c(=O)c2cc1C(=O)NCc1ccccn1] | ++++ |
| 48 | 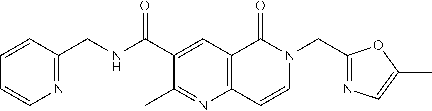 [SMILES: Cc1cnc(Cn2ccc3nc(C)c(C(=O)NCc4ccccn4)cc3c2=O)o1] | + |
| 49 | 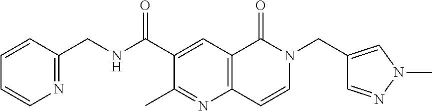 [SMILES: Cc1nc2ccn(Cc3cnn(C)c3)c(=O)c2cc1C(=O)NCc1ccccn1] | + |
| 50 | 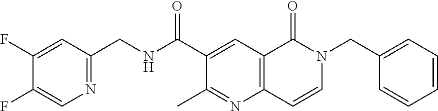 [SMILES: Cc1nc2ccn(Cc3ccccc3)c(=O)c2cc1C(=O)NCc1cc(F)c(F)cn1] | ++++ |
| 51 | 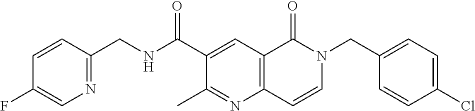 [SMILES: Cc1nc2ccn(Cc3ccc(Cl)cc3)c(=O)c2cc1C(=O)NCc1ccc(F)cn1] | ++++ |
| 52 | 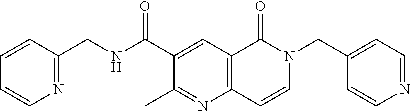 [SMILES: Cc1nc2ccn(Cc3ccncc3)c(=O)c2cc1C(=O)NCc1ccccn1] | + |
| 53 | 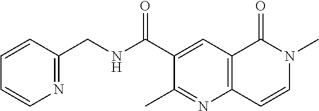 [SMILES: Cc1nc2ccn(C)c(=O)c2cc1C(=O)NCc1ccccn1] | + |
| 54 | 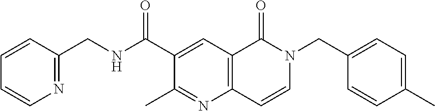 [SMILES: Cc1ccc(Cn2ccc3nc(C)c(C(=O)NCc4ccccn4)cc3c2=O)cc1] | ++++ |
| 55 | 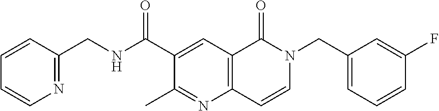 [SMILES: Cc1nc2ccn(Cc3cccc(F)c3)c(=O)c2cc1C(=O)NCc1ccccn1] | ++++ |
| 56 | 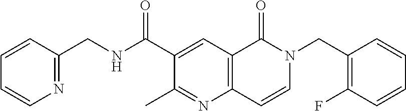 [SMILES: Cc1nc2ccn(Cc3ccccc3F)c(=O)c2cc1C(=O)NCc1ccccn1] | +++ |
| 57 | 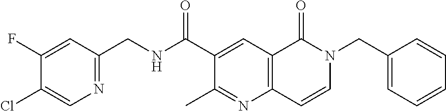 [SMILES: Cc1nc2ccn(Cc3ccccc3)c(=O)c2cc1C(=O)NCc1cc(F)c(Cl)cn1] | ++++ |
| 58 | 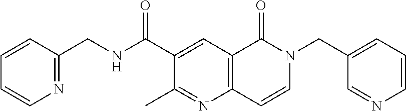 [SMILES: Cc1nc2ccn(Cc3cccnc3)c(=O)c2cc1C(=O)NCc1ccccn1] | + |
| 59 | 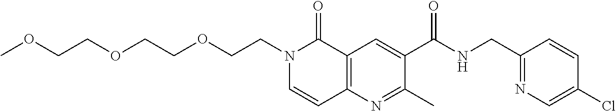 [SMILES: COCCOCCOCCn1ccc2nc(C)c(C(=O)NCc3ccc(Cl)cn3)cc2c1=O] | + |
| 60 | 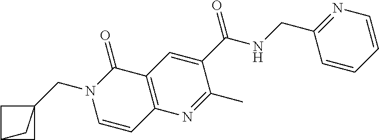 [SMILES: Cc1nc2ccn(CC34CC(C3)C4)c(=O)c2cc1C(=O)NCc1ccccn1] | ++ |
| 61 | 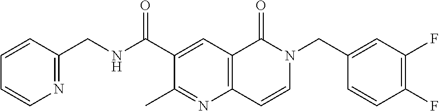 [SMILES: Cc1nc2ccn(Cc3ccc(F)c(F)c3)c(=O)c2cc1C(=O)NCc1ccccn1] | ++++ |
| 62 | 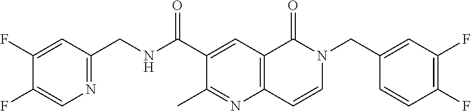 [SMILES: Cc1nc2ccn(Cc3ccc(F)c(F)c3)c(=O)c2cc1C(=O)NCc1cc(F)c(F)cn1] | ++++ |
| 63 | 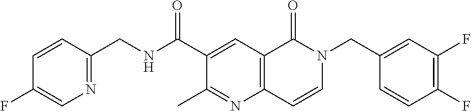 [SMILES: Cc1nc2ccn(Cc3ccc(F)c(F)c3)c(=O)c2cc1C(=O)NCc1ccc(F)cn1] | ++++ |
| 64 | 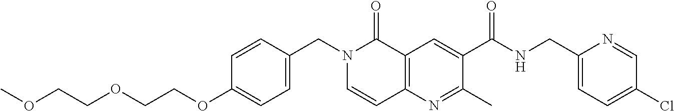 [SMILES: COCCOCCOc1ccc(Cn2ccc3nc(C)c(C(=O)NCc4ccc(Cl)cn4)cc3c2=O)cc1] | ++ |
| 65 | 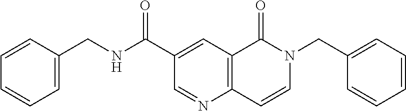 [SMILES: O=C(NCc1ccccc1)c1cnc2ccn(Cc3ccccc3)c(=O)c2c1] | + |
| 66 | 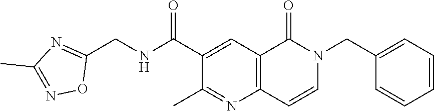 [SMILES: Cc1noc(CNC(=O)c2cc3c(=O)n(Cc4ccccc4)ccc3nc2C)n1] | +++ |
| 67 | 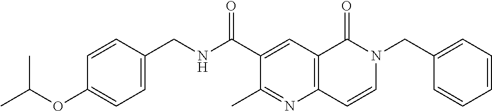 [SMILES: Cc1nc2ccn(Cc3ccccc3)c(=O)c2cc1C(=O)NCc1ccc(OC(C)C)cc1] | + |
| 68 | 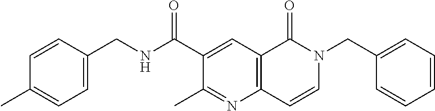 [SMILES: Cc1ccc(CNC(=O)c2cc3c(=O)n(Cc4ccccc4)ccc3nc2C)cc1] | +++ |
| 69 | 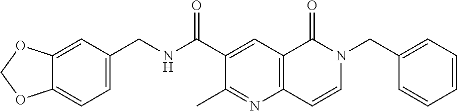 [SMILES: Cc1nc2ccn(Cc3ccccc3)c(=O)c2cc1C(=O)NCc1ccc2c(c1)OCO2] | ++++ |
| 70 | 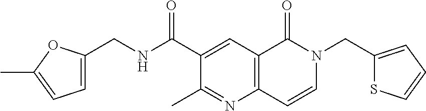 [SMILES: Cc1ccc(CNC(=O)c2cc3c(=O)n(Cc4cccs4)ccc3nc2C)o1] | ++++ |
| 71 | 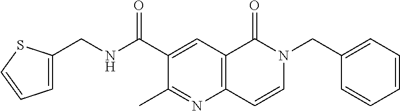 [SMILES: Cc1nc2ccn(Cc3ccccc3)c(=O)c2cc1C(=O)NCc1cccs1] | +++ |
| 72 | 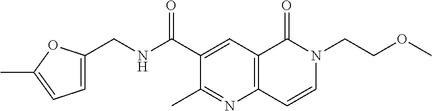 | +++ |
| 73 | 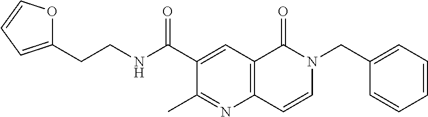 [SMILES: Cc1nc2ccn(Cc3ccccc3)c(=O)c2cc1C(=O)NCCc1ccco1] | ++ |
| 74 | 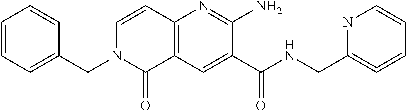 [SMILES: Nc1nc2ccn(Cc3ccccc3)c(=O)c2cc1C(=O)NCC1=C=CC=CN1] | ++ |
| 75 | 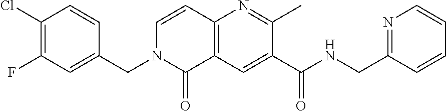 [SMILES: Cc1nc2ccn(Cc3ccc(Cl)c(F)c3)c(=O)c2cc1C(=O)NCc1ccccn1] | ++++ |
| 76 | 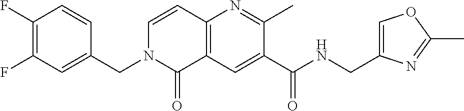 [SMILES: Cc1nc(CNC(=O)c2cc3c(=O)n(Cc4ccc(F)c(F)c4)ccc3nc2C)co1] | ++++ |
| 77 | 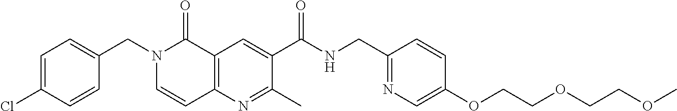 [SMILES: COCCOCCOc1ccc(CNC(=O)c2cc3c(=O)n(Cc4ccc(Cl)cc4)ccc3nc2C)nc1] | + |
| 78 | 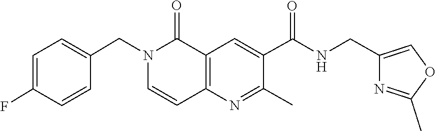 [SMILES: Cc1nc(CNC(=O)c2cc3c(=O)n(Cc4ccc(F)cc4)ccc3nc2C)co1] | ++++ |
| 79 | 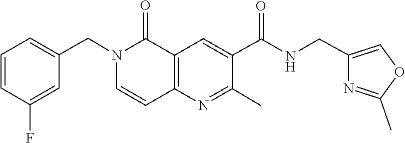 [SMILES: Cc1nc(CNC(=O)c2cc3c(=O)n(Cc4cccc(F)c4)ccc3nc2C)co1] | ++++ |
| 80 | 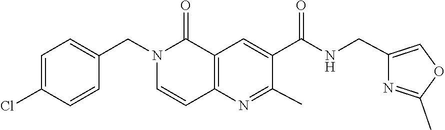 [SMILES: Cc1nc(CNC(=O)c2cc3c(=O)n(Cc4ccc(Cl)cc4)ccc3nc2C)co1] | ++++ |
| 81 | 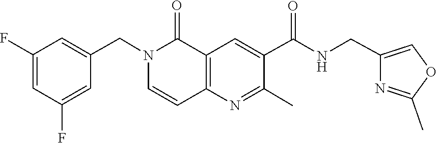 [SMILES: Cc1nc(CNC(=O)c2cc3c(=O)n(Cc4cc(F)cc(F)c4)ccc3nc2C)co1] | ++++ |
| Comparator 1 | 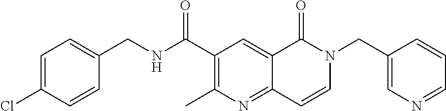 [SMILES: Cc1nc2ccn(Cc3cccnc3)c(=O)c2cc1C(=O)NCc1ccc(Cl)cc1] | ++ |

*NT = Not Tested; “++++” = EC₅₀ < 0.1 μM; “+++” = EC₅₀ ≥ 0.1 μM and < 1 μM; “++” = EC₅₀ ≥ 1 μM and < 10 μM; “+” = EC₅₀ ≥ 10 μM

### In Vitro Liver Microsomes Metabolic Stability Assay of Different Species

1. The liver microsomes were stored at −80° C. prior to use. The liver microsomes information is listed in Table 2.

TABLE 2
Liver Microsomes Information

| Species | Cat. No. | Lot. No. | Vendor |
|---|---|---|---|
| Human | 452117 | 38296 | Corning |
| Rat | 452501 | 1260001 | Corning |
| Mouse | 452701 | 0293002 | Corning |

2. The master solution was prepared according to Table 3.

TABLE 3
Preparation of Master Solution

| | Stock | | | | Final | |
|---|---|---|---|---|---|---|
| Reagent | Concentration | | Volume | | Concentration | |
| Phosphate buffer | 200 | mM | 200 | μL | 100 | mM |
| Ultra-pure H₂O | — | | 108 | μL | — | |
| MgCl₂ solution | 50 | mM | 40 | μL | 5 | mM |
| Microsomes | 20 | mg/mL | 10 | μL | 0.5 | mg/mL |

3. 40 μL of 10 mM NADPH solution was added to each well. The final concentrations of NADPH was 1 mM. The mixture was pre-warmed at 37° C. for 5 minutes. The negative control samples were prepared by replacing NADPH solutions with 40 μL of ultra-pure H₂O. The negative control was used to exclude the misleading factor that resulted from instability of chemical itself. Samples with NADPH were prepared in duplicate. Negative controls were prepared in singlet.

4. The reaction was started with the addition of 2 μL of 200 μM control compound or test compound solutions. Verapamil was used as positive control in this study. The final concentration of test compound or control compound was 1 μM.

5. Aliquots of 50 μL were taken from the reaction solution at 0 and 30 minutes. The reaction was stopped by the addition of 4 volumes of cold acetonitrile with Internal Standard (100 nM alprazolam, 200 nM imipramine, 200 nM labetalol and 2 μM ketoprofen). Samples were centrifuged at 3, 220 g for 40 minutes. Aliquot of 90 μL of the supernatant was mixed with 90 μL of ultra-pure H₂O and then used for LC-MS/MS analysis.

### 6. Data Analysis

All calculations were carried out using Microsoft Excel.

Peak areas were determined from extracted ion chromatograms. The slope value, k, was determined by linear regression of the natural logarithm of the remaining percentage of the parent drug vs. incubation time curve.

The in vitro half-life (in vitro t_{1/2}) was determined from the slope value:

$$in vitro {t}_{1 / 2} = - ( 0.693 / k )$$

Conversion of the in vitro t_{1/2} (min) into the in vitro intrinsic clearance (in vitro CLᵢₙₜ, in μL/min/mg protein) was done using the following equation (mean of duplicate determinations):

$$in vitro {CL}_{int} = ( \frac{0.693}{( {t}_{1 / 2} )} ) * ( \frac{volume of incubation ( μ L )}{amount of proteins ( mg )} )$$

Conversion of the in vitro t_{1/2} (min) into the scale-up unbound intrinsic clearance (Scale-up CLᵢₙₜ, in mL/min/kg) was done using the following equation (mean of duplicate determinations):

$$Scale - up {CL}_{int} = ( \frac{0.693}{{t}_{1 / 2}} ) * ( \frac{volume of incubation ( μ L )}{amount of proteins ( mg )} ) * Scaling Factor$$

TABLE 4
Scaling Factors for Intrinsic Clearance
Prediction in Liver Microsomes

| | Liver Weight (g | Microsomal | Liver blood | |
|---|---|---|---|---|
| | liver/kg body | Concentration | flow (Q, | Scaling |
| Species | weight)^{a} | (mg/g liver)^{b} | mL/min/kg)^{a} | Factor |
| Human | 25.7 | 48.8 | 20.7 | 1254.2 |
| Rat | 40.0 | 44.8 | 55.2 | 1792.0 |
| Mouse | 87.5 | 50.0 | 90.0 | 4375.0 |

^{a}Davies and Morris, 1993, Pharmaceutical Research, 10 (7), pp 1093-1095.
^{b}Barter et al, 2007, Current Drug Metabolism, 8 (1), pp 33-45; Iwatsubo et al, 1997, Journal of Pharmacology and Experimental Therapeutics, 283 (2), pp 462-469.

TABLE 5
Clearance Data for Select Compounds

| | | CLᵢₙₜ, | CLᵢₙₜ, | CLᵢₙₜ, |
|---|---|---|---|---|
| Exact | | Human LM | Rat LM | Mouse LM |
| Number | Structure | (mL/min/kg) | (mL/min/kg) | (mL/min/kg) |
| 1 | 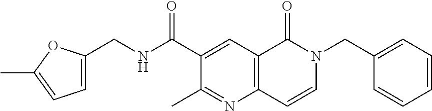 [SMILES: Cc1ccc(CNC(=O)c2cc3c(=O)n(Cc4ccccc4)ccc3nc2C)o1] | 57 | 319 | 760 |
| 5 | 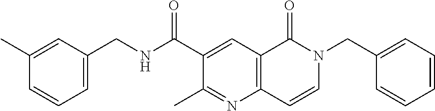 [SMILES: Cc1cccc(CNC(=O)c2cc3c(=O)n(Cc4ccccc4)ccc3nc2C)c1] | 171 | 1038 | 1796 |
| 6 | 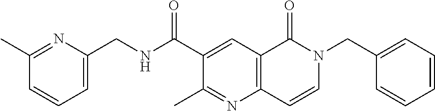 [SMILES: Cc1cccc(CNC(=O)c2cc3c(=O)n(Cc4ccccc4)ccc3nc2C)n1] | 16 | 217 | 414 |
| 7 | 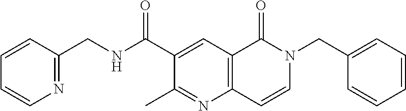 [SMILES: Cc1nc2ccn(Cc3ccccc3)c(=O)c2cc1C(=O)NCc1ccccn1] | 2 | 28 | 91 |
| 17 | 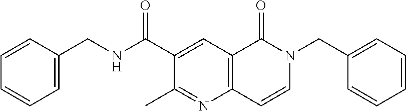 [SMILES: Cc1nc2ccn(Cc3ccccc3)c(=O)c2cc1C(=O)NCc1ccccc1] | 15 | 163 | 470 |
| 18 | 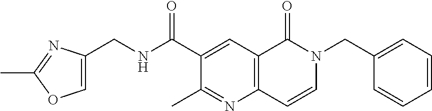 [SMILES: Cc1nc(CNC(=O)c2cc3c(=O)n(Cc4ccccc4)ccc3nc2C)co1] | 4 | 73 | 140 |
| 31 | 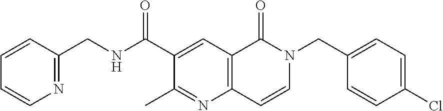 [SMILES: Cc1nc2ccn(Cc3ccc(Cl)cc3)c(=O)c2cc1C(=O)NCc1ccccn1] | 1 | 55 | 132 |
| 50 | 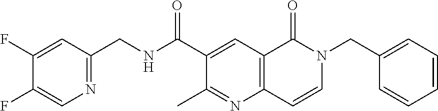 [SMILES: Cc1nc2ccn(Cc3ccccc3)c(=O)c2cc1C(=O)NCc1cc(F)c(F)cn1] | 3 | 70 | 124 |
| 51 | 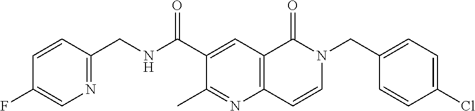 [SMILES: Cc1nc2ccn(Cc3ccc(Cl)cc3)c(=O)c2cc1C(=O)NCc1ccc(F)cn1] | 0 | 42 | 140 |
| Comparator 2 | 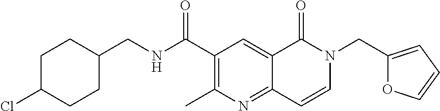 [SMILES: Cc1nc2ccn(Cc3ccco3)c(=O)c2cc1C(=O)NCC1CCC(Cl)CC1] | 77 | 318 | 939 |

1. A pharmaceutical composition comprising a pharmaceutically acceptable carrier or diluent and a compound represented by Formula (I): 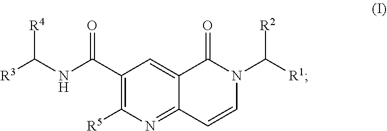
or a pharmaceutically acceptable salt thereof, wherein:
R¹ is H, halo, C₁₋₆alkyl, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the C₁₋₆alkyl is optionally substituted with one to three R^{1a}, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{1c};
R^{1a}, for each occurrence, is independently halo, —OR^{1b}, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{1c};
R^{1b} is H, C₁₋₆alkyl, or —[CH₂—CH₂—O]_{z}—CH₃;
z is 1 to 3;
R^{1c}, for each occurrence, is independently halo, C₁₋₆alkyl, C₁₋₆haloalkyl, —OR^{1b}, —NR^{1d}R^{1d}, —CN, —NO₂, —C(O)R^{1b}, —C(O)OR^{1b}, —C(O)NR^{1d}R^{1d}, or —NR^{1d}C(O)R^{1b};
R^{1d} is H or C₁₋₆alkyl;
R² is H or C₁₋₆alkyl;
or R¹ and R², together with the carbon to which they are attached, form a non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, each of which is optionally substituted with one to three R^{1c};
R³ is H, halo, C₁₋₆alkyl, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the C₁₋₆alkyl is optionally substituted with one to three R^{3a}, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{3c};
R^{3a}, for each occurrence, is independently halo, —OR^{3b}, non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, or 9 or 10-membered bicyclic heteroaryl, wherein the non-aromatic C₃₋₈carbocyclyl, 4 to 6-membered monocyclic heterocyclyl, 6 to 10-membered bicyclic heterocyclyl, phenyl, naphthyl, 5 or 6-membered monocyclic heteroaryl, and 9 or 10-membered bicyclic heteroaryl are each optionally substituted with one to three R^{3c};
R^{3b} is H, C₁₋₆alkyl, or —[CH₂—CH₂—O]_{y}—CH₃;
y is 1 to 3;
R^{3c}, for each occurrence, is independently halo, C₁₋₆alkyl, C₁₋₆haloalkyl, —OR^{3b}, —NR^{3d}R^{3d}, —CN, —NO₂, —C(O)R^{3b}, —C(O)OR^{3b}, —C(O)NR^{3d}R^{3d}, or —NR^{3d}C(O)R^{3b};
R^{3d} is H or C₁₋₆alkyl;
R⁴ is H or C₁₋₆alkyl;
R⁵ is H, halo, —NR^{5a}R^{5b}, C₁₋₆alkyl, or C₁₋₆haloalkyl;
R^{5a} and R^{5b} are each independently H or C₁₋₆alkyl;
provided that the pharmaceutical composition does not comprise a compound of the formulas below: 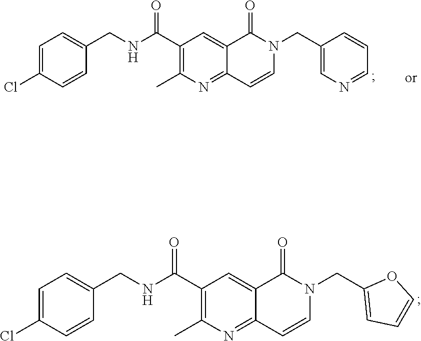 [SMILES: Cc1nc2ccn(Cc3cccnc3)c(=O)c2cc1C(=O)NCc1ccc(Cl)cc1 ; Cc1nc2ccn(Cc3ccco3)c(=O)c2cc1C(=O)NCc1ccc(Cl)cc1]
or a pharmaceutically acceptable salt thereof.

2. The pharmaceutical composition of claim 1, wherein R⁵ is —CH₃.

3. The pharmaceutical composition of claim 1, wherein R¹ is:
(i) H, C₁₋₆alkyl, non-aromatic C₃₋₈carbocyclyl, 4 to 6-membered monocyclic heterocyclyl, phenyl, or 5 or 6-membered monocyclic heteroaryl, wherein the C₁₋₆alkyl is optionally substituted with one to three R^{1a}, wherein the non-aromatic C₃₋₈carbocyclyl, 4 to 6-membered monocyclic heterocyclyl, phenyl, or 5 or 6-membered monocyclic heteroaryl are each optionally substituted with one or two R^{1c}, or wherein R¹ and R², together with the carbon to which they are attached, form phenyl optionally substituted with one or two R^{1c}; or
(ii) non-aromatic C₃₋₈carbocyclyl, 4 to 6-membered monocyclic heterocyclyl, phenyl, or 5 or 6-membered monocyclic heteroaryl, wherein the non-aromatic C₃₋₈carbocyclyl, 4 to 6-membered monocyclic heterocyclyl, phenyl, or 5 or 6-membered monocyclic heteroaryl are each optionally substituted with one or two R^{1c}; or
(iii) selected from the group consisting of bicyclo[1.1.1]pentanyl, phenyl, pyridyl, thiophenyl, tetrahydropyranyl, tetrahydrofuranyl, and oxetanyl, each of which is optionally substituted with one or two R^{1c}.

4.-6. (canceled)

7. The pharmaceutical composition of claim 2, wherein R^{1c}, for each occurrence, is C₁₋₆alkyl, —OR^{1b}, or halo, and wherein R^{1b} is —[CH₂—CH₂—O]_{z}—CH₃.

8. The pharmaceutical composition of claim 1, wherein R³ is:
(i) H, C₁₋₆alkyl, 6 to 10-membered bicyclic heterocyclyl, phenyl, or 5 or 6-membered monocyclic heteroaryl, wherein the C₁₋₆alkyl is optionally substituted with one to three R^{3a}, wherein the 6 to 10-membered bicyclic heterocyclyl, phenyl, or 5 or 6-membered monocyclic heteroaryl are each optionally substituted with one to three R^{3c}; or
(ii) phenyl, pyridyl, pyrimidinyl, pyridazinyl, pyrazinyl, thiophenyl, benzodioxoyl, furanyl, oxazoyl, oxadiazoyl, pyrazoyl, or triazoyl, wherein the phenyl, pyridyl, pyrimidinyl, pyridazinyl, pyrazinyl, thiophenyl, benzodioxoyl, furanyl, oxazoyl, oxadiazoyl, pyrazoyl, or triazoyl, are each optionally substituted with one to two R^{3c}.

9.-11. (canceled)

12. The pharmaceutical composition of claim 1, wherein the compound is represented by Formula (II): 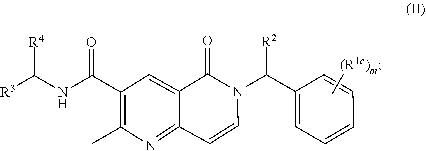
or a pharmaceutically acceptable salt thereof, wherein:
m is 0 to 2;
R^{1c} is independently halo, C₁₋₆alkyl, or —OR^{1b};
R^{1b} is —[CH₂—CH₂—O]_{z}—CH₃;
R³ is 5 or 6-membered monocyclic heteroaryl optionally substituted with one or two R^{3c};
R^{3c} is halo or C₁₋₆alkyl.

13. The pharmaceutical composition of claim 12, wherein R³ is pyridyl.

14. The pharmaceutical composition of claim 8, wherein R^{3c}, for each occurrence, is;
(i) halo, C₁₋₆alkyl, C₁₋₆haloalkyl, —OR^{3b}, or —C(O)N^{3d}R^{3d}; or
(ii) —CH₃, —CF₃, —Cl, —F, —OCH₃, —OCH(CH₃)₂, or —C(O)NH₂.

15. (canceled)

16. The pharmaceutical composition of claim 13, wherein R² and R⁴ are both H.

17. The pharmaceutical composition of claim 1, wherein R^{1a} is —OR^{1b}, and R^{3a}, for each occurrence, is independently halo, —OR^{3b}, or 5 or 6-membered monocyclic heteroaryl.

18. The pharmaceutical composition of claim 3, wherein R^{1c} is —F, —Cl, or —CH₃ and R^{3c} is —F, Cl, or —CH₃.

19. The pharmaceutical composition of claim 1, wherein the compound is selected from:
6-benzyl-2-methyl-N-((5-methylfuran-2-yl)methyl)-5-oxo-5,6-dihydro-1,6-naphthyridine-3-carboxamide;
6-((5-fluoropyridin-2-yl)methyl)-2-methyl-N-((5-methylfuran-2-yl)methyl)-5-oxo-5,6-dihydro-1,6-naphthyridine-3-carboxamide;
6-benzyl-2-methyl-N-((5-methyloxazol-2-yl)methyl)-5-oxo-5,6-dihydro-1,6-naphthyridine-3-carboxamide;
6-benzyl-2-methyl-N-((2-methyloxazol-5-yl)methyl)-5-oxo-5,6-dihydro-1,6-naphthyridine-3-carboxamide;
6-benzyl-2-methyl-N-(3-methylbenzyl)-5-oxo-5,6-dihydro-1,6-naphthyridine-3-carboxamide;
6-benzyl-2-methyl-N-((6-methylpyridin-2-yl)methyl)-5-oxo-5,6-dihydro-1,6-naphthyridine-3-carboxamide;
6-benzyl-2-methyl-5-oxo-N-(pyridin-2-ylmethyl)-5,6-dihydro-1,6-naphthyridine-3-carboxamide;
6-benzyl-2-methyl-N-((1-methyl-1H-1,2,3-triazol-4-yl)methyl)-5-oxo-5,6-dihydro-1,6-naphthyridine-3-carboxamide;
6-benzyl-2-methyl-N-((2-methyl-2H-1,2,3-triazol-4-yl)methyl)-5-oxo-5,6-dihydro-1,6-naphthyridine-3-carboxamide;
6-benzyl-2-methyl-N-((3-methyl-1,2,4-oxadiazol-5-yl)methyl)-5-oxo-5,6-dihydro-1,6-naphthyridine-3-carboxamide;
N-(4-chlorobenzyl)-2-methyl-5-oxo-6-((tetrahydrofuran-2-yl)methyl)-5,6-dihydro-1,6-naphthyridine-3-carboxamide;
6-benzyl-2-methyl-5-oxo-N-(pyridin-4-ylmethyl)-5,6-dihydro-1,6-naphthyridine-3-carboxamide;
6-benzyl-2-methyl-N-((5-methyl-1,3,4-oxadiazol-2-yl)methyl)-5-oxo-5,6-dihydro-1,6-naphthyridine-3-carboxamide;
6-benzyl-2-methyl-N-((1-methyl-1H-pyrazol-3-yl)methyl)-5-oxo-5,6-dihydro-1,6-naphthyridine-3-carboxamide;
6-benzyl-2-methyl-N-((1-methyl-1H-pyrazol-4-yl)methyl)-5-oxo-5,6-dihydro-1,6-naphthyridine-3-carboxamide;
6-benzyl-N-(2-fluorobenzyl)-2-methyl-5-oxo-5,6-dihydro-1,6-naphthyridine-3-carboxamide;
N,6-dibenzyl-2-methyl-5-oxo-5,6-dihydro-1,6-naphthyridine-3-carboxamide;
6-benzyl-2-methyl-N-((2-methyloxazol-4-yl)methyl)-5-oxo-5,6-dihydro-1,6-naphthyridine-3-carboxamide;
6-benzyl-2-methyl-N-((5-methyl-1,2,4-oxadiazol-3-yl)methyl)-5-oxo-5,6-dihydro-1,6-naphthyridine-3-carboxamide;
N-(4-chlorobenzyl)-6-(2-methoxyethyl)-2-methyl-5-oxo-5,6-dihydro-1,6-naphthyridine-3-carboxamide;
6-(2-methoxyethyl)-2-methyl-N-((2-methyloxazol-5-yl)methyl)-5-oxo-5,6-dihydro-1,6-naphthyridine-3-carboxamide;
6-benzyl-N-(4-chlorobenzyl)-2-methyl-5-oxo-5,6-dihydro-1,6-naphthyridine-3-carboxamide;
6-benzyl-2-methyl-5-oxo-N-(pyridin-3-ylmethyl)-5,6-dihydro-1,6-naphthyridine-3-carboxamide;
6-benzyl-N-(4-methoxybenzyl)-2-methyl-5-oxo-5,6-dihydro-1,6-naphthyridine-3-carboxamide;
6-benzyl-2-methyl-5-oxo-N-(4-(trifluoromethyl)benzyl)-5,6-dihydro-1,6-naphthyridine-3-carboxamide;
N-(4-chlorobenzyl)-2-methyl-5-oxo-6-((tetrahydro-2H-pyran-2-yl)methyl)-5,6-dihydro-1,6-naphthyridine-3-carboxamide;
N-(4-chlorobenzyl)-2-methyl-6-(oxetan-2-ylmethyl)-5-oxo-5,6-dihydro-1,6-naphthyridine-3-carboxamide;
6-benzyl-2-methyl-5-oxo-N-(1-(pyridin-2-yl)ethyl)-5,6-dihydro-1,6-naphthyridine-3-carboxamide;
6-benzyl-2-methyl-5-oxo-N-(pyrimidin-2-ylmethyl)-5,6-dihydro-1,6-naphthyridine-3-carboxamide;
6-(2-methoxyethyl)-2-methyl-5-oxo-N-(pyridin-2-ylmethyl)-5,6-dihydro-1,6-naphthyridine-3-carboxamide;
6-(4-chlorobenzyl)-2-methyl-5-oxo-N-(pyridin-2-ylmethyl)-5,6-dihydro-1,6-naphthyridine-3-carboxamide;
6-benzyl-2-methyl-5-oxo-N-(pyrazin-2-ylmethyl)-5,6-dihydro-1,6-naphthyridine-3-carboxamide;
6-benzyl-2-methyl-5-oxo-N-(pyridazin-3-ylmethyl)-5,6-dihydro-1,6-naphthyridine-3-carboxamide;
6-benzyl-2-methyl-5-oxo-N-(pyrimidin-5-ylmethyl)-5,6-dihydro-1,6-naphthyridine-3-carboxamide;
6-benzyl-N-((5-fluoropyridin-2-yl)methyl)-2-methyl-5-oxo-5,6-dihydro-1,6-naphthyridine-3-carboxamide;
6-benzyl-N-((3-fluoropyridin-2-yl)methyl)-2-methyl-5-oxo-5,6-dihydro-1,6-naphthyridine-3-carboxamide;
2-methyl-5-oxo-6-(1-phenylethyl)-N-(pyridin-2-ylmethyl)-5,6-dihydro-1,6-naphthyridine-3-carboxamide;
6-benzyl-N-((5-chloropyridin-2-yl)methyl)-2-methyl-5-oxo-5,6-dihydro-1,6-naphthyridine-3-carboxamide;
6-benzyl-N-((4-fluoropyridin-2-yl)methyl)-2-methyl-5-oxo-5,6-dihydro-1,6-naphthyridine-3-carboxamide;
6-(cyclohexylmethyl)-2-methyl-5-oxo-N-(pyridin-2-ylmethyl)-5,6-dihydro-1,6-naphthyridine-3-carboxamide;
2-methyl-5-oxo-N,6-bis(pyridin-2-ylmethyl)-5,6-dihydro-1,6-naphthyridine-3-carboxamide;
6-benzyl-N,2-dimethyl-5-oxo-5,6-dihydro-1,6-naphthyridine-3-carboxamide;
6-benzyl-N-(4-carbamoylbenzyl)-2-methyl-5-oxo-5,6-dihydro-1,6-naphthyridine-3-carboxamide;
6-((5-fluoropyridin-2-yl)methyl)-2-methyl-5-oxo-N-(pyridin-2-ylmethyl)-5,6-dihydro-1,6-naphthyridine-3-carboxamide;
6-isopropyl-2-methyl-5-oxo-N-(pyridin-2-ylmethyl)-5,6-dihydro-1,6-naphthyridine-3-carboxamide;
2-methyl-5-oxo-6-phenyl-N-(pyridin-2-ylmethyl)-5,6-dihydro-1,6-naphthyridine-3-carboxamide;
6-(4-fluorobenzyl)-2-methyl-5-oxo-N-(pyridin-2-ylmethyl)-5,6-dihydro-1,6-naphthyridine-3-carboxamide;
2-methyl-6-((5-methyloxazol-2-yl)methyl)-5-oxo-N-(pyridin-2-ylmethyl)-5,6-dihydro-1,6-naphthyridine-3-carboxamide;
2-methyl-6-((1-methyl-1H-pyrazol-4-yl)methyl)-5-oxo-N-(pyridin-2-ylmethyl)-5,6-dihydro-1,6-naphthyridine-3-carboxamide;
6-benzyl-N-((4,5-difluoropyridin-2-yl)methyl)-2-methyl-5-oxo-5,6-dihydro-1,6-naphthyridine-3-carboxamide;
6-(4-chlorobenzyl)-N-((5-fluoropyridin-2-yl)methyl)-2-methyl-5-oxo-5,6-dihydro-1,6-naphthyridine-3-carboxamide;
2-methyl-5-oxo-N-(pyridin-2-ylmethyl)-6-(pyridin-4-ylmethyl)-5,6-dihydro-1,6-naphthyridine-3-carboxamide;
2,6-dimethyl-5-oxo-N-(pyridin-2-ylmethyl)-5,6-dihydro-1,6-naphthyridine-3-carboxamide;
2-methyl-6-(4-methylbenzyl)-5-oxo-N-(pyridin-2-ylmethyl)-5,6-dihydro-1,6-naphthyridine-3-carboxamide;
6-(3-fluorobenzyl)-2-methyl-5-oxo-N-(pyridin-2-ylmethyl)-5,6-dihydro-1,6-naphthyridine-3-carboxamide;
6-(2-fluorobenzyl)-2-methyl-5-oxo-N-(pyridin-2-ylmethyl)-5,6-dihydro-1,6-naphthyridine-3-carboxamide;
6-benzyl-N-((5-chloro-4-fluoropyridin-2-yl)methyl)-2-methyl-5-oxo-5,6-dihydro-1,6-naphthyridine-3-carboxamide;
2-methyl-5-oxo-N-(pyridin-2-ylmethyl)-6-(pyridin-3-ylmethyl)-5,6-dihydro-1,6-naphthyridine-3-carboxamide;
N-((5-chloropyridin-2-yl)methyl)-6-(2-(2-(2-methoxyethoxy)ethoxy)ethyl)-2-methyl-5-oxo-5,6-dihydro-1,6-naphthyridine-3-carboxamide;
6-(bicyclo[1.1.1]pentan-1-ylmethyl)-2-methyl-5-oxo-N-(pyridin-2-ylmethyl)-5,6-dihydro-1,6-naphthyridine-3-carboxamide;
6-(3,4-difluorobenzyl)-2-methyl-5-oxo-N-(pyridin-2-ylmethyl)-5,6-dihydro-1,6-naphthyridine-3-carboxamide;
6-(3,4-difluorobenzyl)-N-((4,5-difluoropyridin-2-yl)methyl)-2-methyl-5-oxo-5,6-dihydro-1,6-naphthyridine-3-carboxamide;
6-(3,4-difluorobenzyl)-N-((5-fluoropyridin-2-yl)methyl)-2-methyl-5-oxo-5,6-dihydro-1,6-naphthyridine-3-carboxamide;
N-((5-chloropyridin-2-yl)methyl)-6-(4-(2-(2-methoxyethoxy)ethoxy)benzyl)-2-methyl-5-oxo-5,6-dihydro-1,6-naphthyridine-3-carboxamide;
6-benzyl-5-oxo-N-(pyridin-2-ylmethyl)-5,6-dihydro-1,6-naphthyridine-3-carboxamide;
6-benzyl-2-methyl-N-((4-methyloxazol-2-yl)methyl)-5-oxo-5,6-dihydro-1,6-naphthyridine-3-carboxamide;
6-benzyl-N-(4-isopropoxybenzyl)-2-methyl-5-oxo-5,6-dihydro-1,6-naphthyridine-3-carboxamide;
6-benzyl-2-methyl-N-(4-methylbenzyl)-5-oxo-5,6-dihydro-1,6-naphthyridine-3-carboxamide;
N-(benzo[d][1,3]dioxol-5-ylmethyl)-6-benzyl-2-methyl-5-oxo-5,6-dihydro-1,6-naphthyridine-3-carboxamide;
2-methyl-N-((5-methylfuran-2-yl)methyl)-5-oxo-6-(thiophen-2-ylmethyl)-5,6-dihydro-1,6-naphthyridine-3-carboxamide;
6-benzyl-2-methyl-5-oxo-N-(thiophen-2-ylmethyl)-5,6-dihydro-1,6-naphthyridine-3-carboxamide;
6-(2-methoxyethyl)-2-methyl-N-((5-methylfuran-2-yl)methyl)-5-oxo-5,6-dihydro-1,6-naphthyridine-3-carboxamide;
6-benzyl-N-(2-(furan-2-yl)ethyl)-2-methyl-5-oxo-5,6-dihydro-1,6-naphthyridine-3-carboxamide;
2-amino-6-benzyl-5-oxo-N-(pyridin-2-ylmethyl)-5,6-dihydro-1,6-naphthyridine-3-carboxamide;
6-(4-chloro-3-fluorobenzyl)-2-methyl-5-oxo-N-(pyridin-2-ylmethyl)-5,6-dihydro-1,6-naphthyridine-3-carboxamide;
6-(4-chlorobenzyl)-N-((5-(2-(2-methoxyethoxy)ethoxy)pyridin-2-yl)methyl)-2-methyl-5-oxo-5,6-dihydro-1,6-naphthyridine-3-carboxamide;
6-(3,4-difluorobenzyl)-2-methyl-N-((2-methyloxazol-4-yl)methyl)-5-oxo-5,6-dihydro-1,6-naphthyridine-3-carboxamide;
6-(4-fluorobenzyl)-2-methyl-N-((2-methyloxazol-4-yl)methyl)-5-oxo-5,6-dihydro-1,6-naphthyridine-3-carboxamide;
6-(3-fluorobenzyl)-2-methyl-N-((2-methyloxazol-4-yl)methyl)-5-oxo-5,6-dihydro-1,6-naphthyridine-3-carboxamide;
6-(4-chlorobenzyl)-2-methyl-N-((2-methyloxazol-4-yl)methyl)-5-oxo-5,6-dihydro-1,6-naphthyridine-3-carboxamide; and
6-(3,5-difluorobenzyl)-2-methyl-N-((2-methyloxazol-4-yl)methyl)-5-oxo-5,6-dihydro-1,6-naphthyridine-3-carboxamide;
or a pharmaceutically acceptable salt thereof.

20. A compound represented by Formula (II): 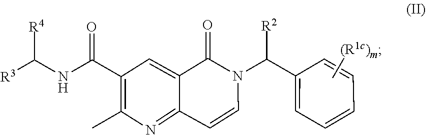
or a pharmaceutically acceptable salt thereof, wherein:
R² is H or C₁₋₆alkyl;
R⁴ is H or C₁₋₆alkyl;
R^{1c} is independently halo or C₁₋₆alkyl;
m is 0 to 2;
when m is 0, R³ is a 5-membered monocyclic heteroaryl selected from the group consisting of oxazoyl, oxadiazoyl, pyrazoyl, and triazoyl or a 6-membered monocyclic heteroaryl, each of which is optionally substituted with one or two R^{3c}; or
when m is 1 or 2, R³ is 5 or 6-membered monocyclic heteroaryl optionally substituted with one or two R^{3c};
R^{3c} is halo or C₁₋₆alkyl.

21. The compound of claim 20, or a pharmaceutically acceptable salt thereof, wherein R² and R⁴ are both H.

22. The compound of claim 20, or a pharmaceutically acceptable salt thereof, wherein R³ is pyridyl, pyrimidinyl, pyridazinyl, pyrazinyl, oxazoyl, oxadiazoyl, pyrazoyl, or triazoyl, each of which is optionally substituted with one to two R^{3c}.

23. The compound of claim 22, or a pharmaceutically acceptable salt thereof, wherein R³ is pyridyl.

24. The compound of claim 20, or a pharmaceutically acceptable salt thereof, wherein each R^{1c} is independently H, —Cl, —F, or —CH₃, and wherein each R^{3c} is independently —F, —Cl, or —CH₃.

25. (canceled)

26. A method of treating a disease mediated by Transcription factor EB, comprising administering to a subject an effective amount of a compound of claim 20, or a pharmaceutically acceptable salt thereof.

27. A method of treating a lysosomal storage disorder, infection, metabolic disease, muscle disease, neurodegenerative disease, renal disease, hematologic disease, or optical disease, comprising administering to a subject an effective amount of a compound of claim 20, or a pharmaceutically acceptable salt thereof.

28. (canceled)

29. A method of treating a disease mediated by Transcription factor EB, comprising administering to a subject an effective amount of the pharmaceutical composition of claim 1.

30. A method of treating a lysosomal storage disorder, infection, metabolic disease, muscle disease, neurodegenerative disease, renal disease, hematologic disease, or optical disease, comprising administering to a subject an effective amount of the pharmaceutical composition of claim 1.
